SEQUENCE LISTING (1400 sequences)

SEQ ID NO: 1 - protein, 8 aa, Artificial Sequence
  Native Peptide Sequence to quantitate RAS-RAF-MAPK Pathway protein
TSSISDLK

SEQ ID NO: 2 - protein, 12 aa, Artificial Sequence
  Native Peptide Sequence to quantitate RAS-RAF-MAPK Pathway protein
TSSISDLKEVPR

SEQ ID NO: 3 - protein, 8 aa, Artificial Sequence
  Native Peptide Sequence to quantitate RAS-RAF-MAPK Pathway protein
FNHQNIVR

SEQ ID NO: 4 - protein, 17 aa, Artificial Sequence
  Native Peptide Sequence to quantitate RAS-RAF-MAPK Pathway protein
  MOD_RES (4)..(6): carbamidomethylated cysteine
DIACGCQYLEENHFIHR

SEQ ID NO: 5 - protein, 11 aa, Artificial Sequence
  Native Peptide Sequence to quantitate RAS-RAF-MAPK Pathway protein
  MOD_RES (2)..(6): carbamidomethylated cysteine
NCLLTCPGPGR

SEQ ID NO: 6 - protein, 14 aa, Artificial Sequence
  Native Peptide Sequence to quantitate RAS-RAF-MAPK Pathway protein
SNQEVLEFVTSGGR

SEQ ID NO: 7 - protein, 8 aa, Artificial Sequence
  Native Peptide Sequence to quantitate RAS-RAF-MAPK Pathway protein
  MOD_RES (2)..(2): carbamidomethylated cysteine
NCPGPVYR

SEQ ID NO: 8 - protein, 15 aa, Artificial Sequence
  Native Peptide Sequence to quantitate RAS-RAF-MAPK Pathway protein
DPEGVPPLLVSQQAK

SEQ ID NO: 9 - protein, 19 aa, Artificial Sequence
  Native Peptide Sequence to quantitate RAS-RAF-MAPK Pathway protein
EEERSPAAPPPLPTTSSGK

SEQ ID NO: 10 - protein, 15 aa, Artificial Sequence
  Native Peptide Sequence to quantitate RAS-RAF-MAPK Pathway protein
SPAAPPPLPTTSSGK

SEQ ID NO: 11 - protein, 19 aa, Artificial Sequence
  Native Peptide Sequence to quantitate RAS-RAF-MAPK Pathway protein
  MOD_RES (9)..(9): carbamidomethylated cysteine
GNLGLEGSCTVPPNVATGR

SEQ ID NO: 12 - protein, 15 aa, Artificial Sequence
  Native Peptide Sequence to quantitate RAS-RAF-MAPK Pathway protein
  MOD_RES (9)..(9): carbamidomethylated cysteine
TQADELPACLLSAAR

SEQ ID NO: 13 - protein, 11 aa, Artificial Sequence
  Native Peptide Sequence to quantitate RAS-RAF-MAPK Pathway protein
IGTGSFGTVFR

SEQ ID NO: 14 - protein, 10 aa, Artificial Sequence
  Native Peptide Sequence to quantitate RAS-RAF-MAPK Pathway protein
IGDFGLATVK

SEQ ID NO: 15 - protein, 11 aa, Artificial Sequence
  Native Peptide Sequence to quantitate RAS-RAF-MAPK Pathway protein
  MOD_RES (6)..(7): carbamidomethylated cysteines
GLNQDCCVVYR

SEQ ID NO: 16 - protein, 13 aa, Artificial Sequence
  Native Peptide Sequence to quantitate RAS-RAF-MAPK Pathway protein
SNNIFLHEGLTVK

SEQ ID NO: 17 - protein, 12 aa, Artificial Sequence
  Native Peptide Sequence to quantitate RAS-RAF-MAPK Pathway protein
  MOD_RES (5)..(5): carbamidomethylated cysteine
VPTVCVDMSTNR

SEQ ID NO: 18 - protein, 13 aa, Artificial Sequence
  Native Peptide Sequence to quantitate RAS-RAF-MAPK Pathway protein
VSQPTAEQAQAFK

SEQ ID NO: 19 - protein, 21 aa, Artificial Sequence
  Native Peptide Sequence to quantitate RAS-RAF-MAPK Pathway protein
  MOD_RES (4)..(4): carbamidomethylated cysteine
TQHCDPEHFPFPAPANAPLQR

SEQ ID NO: 20 - protein, 15 aa, Artificial Sequence
  Native Peptide Sequence to quantitate RAS-RAF-MAPK Pathway protein
NDSVIVADQTPTPTR

SEQ ID NO: 21 - protein, 27 aa, Artificial Sequence
  Native Peptide Sequence to quantitate RAS-RAF-MAPK Pathway protein
IEEPSVVETTHQDSPLPHPESTTSDEK

SEQ ID NO: 22 - protein, 38 aa, Artificial Sequence
  Native Peptide Sequence to quantitate RAS-RAF-MAPK Pathway protein
TQSEESRPQSLQQPATSTTETPASPAHTTPQTQSTSGR

SEQ ID NO: 23 - protein, 20 aa, Artificial Sequence
  Native Peptide Sequence to quantitate RAS-RAF-MAPK Pathway protein
  MOD_RES (2)..(2): carbamidomethylated cysteine
NCEEVGLFNELASPFENEFK

SEQ ID NO: 24 - protein, 8 aa, Artificial Sequence
  Native Peptide Sequence to quantitate RAS-RAF-MAPK Pathway protein
VWVQSLEK

SEQ ID NO: 25 - protein, 16 aa, Artificial Sequence
  Native Peptide Sequence to quantitate RAS-RAF-MAPK Pathway protein
EVPLAQTAQPTSAIVR

SEQ ID NO: 26 - protein, 22 aa, Artificial Sequence
  Native Peptide Sequence to quantitate RAS-RAF-MAPK Pathway protein
SSSAPNVHINTIEPVNIDDLIR

SEQ ID NO: 27 - protein, 17 aa, Artificial Sequence
  Native Peptide Sequence to quantitate RAS-RAF-MAPK Pathway protein
  MOD_RES (13)..(13): carbamidomethylated cysteine
AGFQTEDFSLYACASPK

SEQ ID NO: 28 - protein, 7 aa, Artificial Sequence
  Native Peptide Sequence to quantitate RAS-RAF-MAPK Pathway protein
LLFQGFR

SEQ ID NO: 29 - protein, 14 aa, Artificial Sequence
  Native Peptide Sequence to quantitate RAS-RAF-MAPK Pathway protein
TPIQAGGYGAFPVH

SEQ ID NO: 30 - protein, 12 aa, Artificial Sequence
  Native Peptide Sequence to quantitate RAS-RAF-MAPK Pathway protein
LTQEHIEALLDK

SEQ ID NO: 31 - protein, 10 aa, Artificial Sequence
  Native Peptide Sequence to quantitate RAS-RAF-MAPK Pathway protein
IGDFGLATEK

SEQ ID NO: 32 - protein, 17 aa, Artificial Sequence
  Native Peptide Sequence to quantitate RAS-RAF-MAPK Pathway protein
EAFSLFDKDGDGTITTK

SEQ ID NO: 33 - protein, 7 aa, Artificial Sequence
  Native Peptide Sequence to quantitate RAS-RAF-MAPK Pathway protein
ELGTVMR

SEQ ID NO: 34 - protein, 13 aa, Artificial Sequence
  Native Peptide Sequence to quantitate RAS-RAF-MAPK Pathway protein
DTDSEEEIREAFR

SEQ ID NO: 35 - protein, 16 aa, Artificial Sequence
  Native Peptide Sequence to quantitate RAS-RAF-MAPK Pathway protein
VFDKDGNGYISAAELR

SEQ ID NO: 36 - protein, 12 aa, Artificial Sequence
  Native Peptide Sequence to quantitate RAS-RAF-MAPK Pathway protein
DGNGYISAAELR

SEQ ID NO: 37 - protein, 9 aa, Artificial Sequence
  Native Peptide Sequence to quantitate RAS-RAF-MAPK Pathway protein
HVMTNLGEK

SEQ ID NO: 38 - protein, 22 aa, Artificial Sequence
  Native Peptide Sequence to quantitate RAS-RAF-MAPK Pathway protein
EADIDGDGQVNYEEFVQMMTAK

SEQ ID NO: 39 - protein, 13 aa, Artificial Sequence
  Native Peptide Sequence to quantitate RAS-RAF-MAPK Pathway protein
FTEEYQLFEELGK

SEQ ID NO: 40 - protein, 10 aa, Artificial Sequence
  Native Peptide Sequence to quantitate RAS-RAF-MAPK Pathway protein
VLAGQEYAAK

SEQ ID NO: 41 - protein, 8 aa, Artificial Sequence
  Native Peptide Sequence to quantitate RAS-RAF-MAPK Pathway protein
ITAAEALK

SEQ ID NO: 42 - protein, 19 aa, Artificial Sequence
  Native Peptide Sequence to quantitate RAS-RAF-MAPK Pathway protein
VTEQLIEAISNGDFESYTK

SEQ ID NO: 43 - protein, 9 aa, Artificial Sequence
  Native Peptide Sequence to quantitate RAS-RAF-MAPK Pathway protein
FYFENLWSR

SEQ ID NO: 44 - protein, 12 aa, Artificial Sequence
  Native Peptide Sequence to quantitate RAS-RAF-MAPK Pathway protein
ITQYLDAGGIPR

SEQ ID NO: 45 - protein, 8 aa, Artificial Sequence
  Native Peptide Sequence to quantitate RAS-RAF-MAPK Pathway protein
WQIVHFHR

SEQ ID NO: 46 - protein, 9 aa, Artificial Sequence
  Native Peptide Sequence to quantitate RAS-RAF-MAPK Pathway protein
SGAPSVLPH

SEQ ID NO: 47 - protein, 11 aa, Artificial Sequence
  Native Peptide Sequence to quantitate RAS-RAF-MAPK Pathway protein
AYPDANLLNDR

SEQ ID NO: 48 - protein, 13 aa, Artificial Sequence
  Native Peptide Sequence to quantitate RAS-RAF-MAPK Pathway protein
  MOD_RES (5)..(5): carbamidomethylated cysteine
AEETCAPSVSYFK

SEQ ID NO: 49 - protein, 8 aa, Artificial Sequence
  Native Peptide Sequence to quantitate RAS-RAF-MAPK Pathway protein
FLSLEPVK

SEQ ID NO: 50 - protein, 9 aa, Artificial Sequence
  Native Peptide Sequence to quantitate RAS-RAF-MAPK Pathway protein
FLSLEPVKK

SEQ ID NO: 51 - protein, 9 aa, Artificial Sequence
  Native Peptide Sequence to quantitate RAS-RAF-MAPK Pathway protein
ETIPLTAEK

SEQ ID NO: 52 - protein, 15 aa, Artificial Sequence
  Native Peptide Sequence to quantitate RAS-RAF-MAPK Pathway protein
  MOD_RES (10)..(10): carbamidomethylated cysteine
KHAQTFVALCATDVK

SEQ ID NO: 53 - protein, 14 aa, Artificial Sequence
  Native Peptide Sequence to quantitate RAS-RAF-MAPK Pathway protein
  MOD_RES (9)..(9): carbamidomethylated cysteine
HAQTFVALCATDVK

SEQ ID NO: 54 - protein, 10 aa, Artificial Sequence
  Native Peptide Sequence to quantitate RAS-RAF-MAPK Pathway protein
SPNNFLSYYR

SEQ ID NO: 55 - protein, 15 aa, Artificial Sequence
  Native Peptide Sequence to quantitate RAS-RAF-MAPK Pathway protein
  MOD_RES (2)..(2): carbamidomethylated cysteine
ACQEQIEALLESSLR

SEQ ID NO: 56 - protein, 22 aa, Artificial Sequence
  Native Peptide Sequence to quantitate RAS-RAF-MAPK Pathway protein
  MOD_RES (16)..(16): carbamidomethylated cysteine
AAEEEEEEEEEVDLACTPTDVR

SEQ ID NO: 57 - protein, 9 aa, Artificial Sequence
  Native Peptide Sequence to quantitate RAS-RAF-MAPK Pathway protein
  MOD_RES (6)..(6): carbamidomethylated cysteine
VNFEDCTGR

SEQ ID NO: 58 - protein, 14 aa, Artificial Sequence
  Native Peptide Sequence to quantitate RAS-RAF-MAPK Pathway protein
  MOD_RES (9)..(9): carbamidomethylated cysteine
DWVIPPISCPENEK

SEQ ID NO: 59 - protein, 22 aa, Artificial Sequence
  Native Peptide Sequence to quantitate RAS-RAF-MAPK Pathway protein
VFYSITGQGADTPPVGVFIIER

SEQ ID NO: 60 - protein, 14 aa, Artificial Sequence
  Native Peptide Sequence to quantitate RAS-RAF-MAPK Pathway protein
NTGVISVVTTGLDR

SEQ ID NO: 61 - protein, 16 aa, Artificial Sequence
  Native Peptide Sequence to quantitate RAS-RAF-MAPK Pathway protein
GQVPENEANVVITTLK

SEQ ID NO: 62 - protein, 18 aa, Artificial Sequence
  Native Peptide Sequence to quantitate RAS-RAF-MAPK Pathway protein
DTANWLEINPDTGAISTR

SEQ ID NO: 63 - protein, 7 aa, Artificial Sequence
  Native Peptide Sequence to quantitate RAS-RAF-MAPK Pathway protein
  MOD_RES (5)..(5): carbamidomethylated cysteine
TIFFCER

SEQ ID NO: 64 - protein, 10 aa, Artificial Sequence
  Native Peptide Sequence to quantitate RAS-RAF-MAPK Pathway protein
GLDARPEVTR

SEQ ID NO: 65 - protein, 20 aa, Artificial Sequence
  Native Peptide Sequence to quantitate RAS-RAF-MAPK Pathway protein
YLPRPANPDEIGNFIDENLK

SEQ ID NO: 66 - protein, 14 aa, Artificial Sequence
  Native Peptide Sequence to quantitate RAS-RAF-MAPK Pathway protein
DWVIPPINLPENSR

SEQ ID NO: 67 - protein, 9 aa, Artificial Sequence
  Native Peptide Sequence to quantitate RAS-RAF-MAPK Pathway protein
GPFPQELVR

SEQ ID NO: 68 - protein, 26 aa, Artificial Sequence
  Native Peptide Sequence to quantitate RAS-RAF-MAPK Pathway protein
FAIQTDPNSNDGLVTVVKPIDFETNR

SEQ ID NO: 69 - protein, 9 aa, Artificial Sequence
  Native Peptide Sequence to quantitate RAS-RAF-MAPK Pathway protein
LSDPANWLK

SEQ ID NO: 70 - protein, 16 aa, Artificial Sequence
  Native Peptide Sequence to quantitate RAS-RAF-MAPK Pathway protein
IDPVNGQITTIAVLDR

SEQ ID NO: 71 - protein, 18 aa, Artificial Sequence
  Native Peptide Sequence to quantitate RAS-RAF-MAPK Pathway protein
SAAPHPGDIGDFINEGLK

SEQ ID NO: 72 - protein, 13 aa, Artificial Sequence
  Native Peptide Sequence to quantitate RAS-RAF-MAPK Pathway protein
RLFGPVDSEQLSR

SEQ ID NO: 73 - protein, 12 aa, Artificial Sequence
  Native Peptide Sequence to quantitate RAS-RAF-MAPK Pathway protein
LFGPVDSEQLSR

SEQ ID NO: 74 - protein, 21 aa, Artificial Sequence
  Native Peptide Sequence to quantitate RAS-RAF-MAPK Pathway protein
ERWNFDFVTETPLEGDFAWER

SEQ ID NO: 75 - protein, 19 aa, Artificial Sequence
  Native Peptide Sequence to quantitate RAS-RAF-MAPK Pathway protein
WNFDFVTETPLEGDFAWER

SEQ ID NO: 76 - protein, 8 aa, Artificial Sequence
  Native Peptide Sequence to quantitate RAS-RAF-MAPK Pathway protein
LYLPTGPR

SEQ ID NO: 77 - protein, 29 aa, Artificial Sequence
  Native Peptide Sequence to quantitate RAS-RAF-MAPK Pathway protein
  MOD_RES (24)..(24): carbamidomethylated cysteine
RPGTSPALLQGTAEEDHVDLSLSCTLVPR

SEQ ID NO: 78 - protein, 18 aa, Artificial Sequence
  Native Peptide Sequence to quantitate RAS-RAF-MAPK Pathway protein
SGEQAEGSPGGPGDSQGR

SEQ ID NO: 79 - protein, 10 aa, Artificial Sequence
  Native Peptide Sequence to quantitate RAS-RAF-MAPK Pathway protein
VSNGSPSLER

SEQ ID NO: 80 - protein, 13 aa, Artificial Sequence
  Native Peptide Sequence to quantitate RAS-RAF-MAPK Pathway protein
NLFGPVDHEELTR

SEQ ID NO: 81 - protein, 14 aa, Artificial Sequence
  Native Peptide Sequence to quantitate RAS-RAF-MAPK Pathway protein
WNFDFQNHKPLEGK

SEQ ID NO: 82 - protein, 8 aa, Artificial Sequence
  Native Peptide Sequence to quantitate RAS-RAF-MAPK Pathway protein
YEWQEVEK

SEQ ID NO: 83 - protein, 15 aa, Artificial Sequence
  Native Peptide Sequence to quantitate RAS-RAF-MAPK Pathway protein
GSLPEFYYRPPRPPK

SEQ ID NO: 84 - protein, 18 aa, Artificial Sequence
  Native Peptide Sequence to quantitate RAS-RAF-MAPK Pathway protein
  MOD_RES (14)..(14): carbamidomethylated cysteine
TDPSDSQTGLAEQCAGIR

SEQ ID NO: 85 - protein, 20 aa, Artificial Sequence
  Native Peptide Sequence to quantitate RAS-RAF-MAPK Pathway protein
TEENVSDGSPNAGSVEQTPK

SEQ ID NO: 86 - protein, 17 aa, Artificial Sequence
  Native Peptide Sequence to quantitate RAS-RAF-MAPK Pathway protein
ALLEAGALPNAPNSYGR

SEQ ID NO: 87 - protein, 12 aa, Artificial Sequence
  Native Peptide Sequence to quantitate RAS-RAF-MAPK Pathway protein
RPIQVMMMGSAR

SEQ ID NO: 88 - protein, 12 aa, Artificial Sequence
  Native Peptide Sequence to quantitate RAS-RAF-MAPK Pathway protein
LPVDLAEELGHR

SEQ ID NO: 89 - protein, 12 aa, Artificial Sequence
  Native Peptide Sequence to quantitate RAS-RAF-MAPK Pathway protein
IDAAEGPSDIPD

SEQ ID NO: 90 - protein, 14 aa, Artificial Sequence
  Native Peptide Sequence to quantitate RAS-RAF-MAPK Pathway protein
ILNDLSSDAPGVPR

SEQ ID NO: 91 - protein, 29 aa, Artificial Sequence
  Native Peptide Sequence to quantitate RAS-RAF-MAPK Pathway protein
TAPTSTIAPGVVMASSPALPTQPAEEAAR

SEQ ID NO: 92 - protein, 18 aa, Artificial Sequence
  Native Peptide Sequence to quantitate RAS-RAF-MAPK Pathway protein
HAVVNLINYQDDAELATR

SEQ ID NO: 93 - protein, 19 aa, Artificial Sequence
  Native Peptide Sequence to quantitate RAS-RAF-MAPK Pathway protein
AIPELTKLLNDEDQVVVNK

SEQ ID NO: 94 - protein, 12 aa, Artificial Sequence
  Native Peptide Sequence to quantitate RAS-RAF-MAPK Pathway protein
LLNDEDQVVVNK

SEQ ID NO: 95 - protein, 13 aa, Artificial Sequence
  Native Peptide Sequence to quantitate RAS-RAF-MAPK Pathway protein
  MOD_RES (1)..(1): carbamidomethylated cysteine
CTAGTLHNLSHHR

SEQ ID NO: 96 - protein, 9 aa, Artificial Sequence
  Native Peptide Sequence to quantitate RAS-RAF-MAPK Pathway protein
SGGIPALVK

SEQ ID NO: 97 - protein, 8 aa, Artificial Sequence
  Native Peptide Sequence to quantitate RAS-RAF-MAPK Pathway protein
NLSDAATK

SEQ ID NO: 98 - protein, 16 aa, Artificial Sequence
  Native Peptide Sequence to quantitate RAS-RAF-MAPK Pathway protein
  MOD_RES (13)..(13): carbamidomethylated cysteine
AGDREDITEPAICALR

SEQ ID NO: 99 - protein, 12 aa, Artificial Sequence
  Native Peptide Sequence to quantitate RAS-RAF-MAPK Pathway protein
  MOD_RES (9)..(9): carbamidomethylated cysteine
EDITEPAICALR

SEQ ID NO: 100 - protein, 10 aa, Artificial Sequence
  Native Peptide Sequence to quantitate RAS-RAF-MAPK Pathway protein
LHYGLPVVVK

SEQ ID NO: 101 - protein, 12 aa, Artificial Sequence
  Native Peptide Sequence to quantitate RAS-RAF-MAPK Pathway protein
LLHPPSHWPLIK

SEQ ID NO: 102 - protein, 13 aa, Artificial Sequence
  Native Peptide Sequence to quantitate RAS-RAF-MAPK Pathway protein
  MOD_RES (5)..(5): carbamidomethylated cysteine
NLALCPANHAPLR

SEQ ID NO: 103 - protein, 20 aa, Artificial Sequence
  Native Peptide Sequence to quantitate RAS-RAF-MAPK Pathway protein
  MOD_RES (5)..(5): carbamidomethylated cysteine
NLALCPANHAPLREQGAIPR

SEQ ID NO: 104 - protein, 7 aa, Artificial Sequence
  Native Peptide Sequence to quantitate RAS-RAF-MAPK Pathway protein
LVQLLVR

SEQ ID NO: 105 - protein, 22 aa, Artificial Sequence
  Native Peptide Sequence to quantitate RAS-RAF-MAPK Pathway protein
EAAEAIEAEGATAPLTELLHSR

SEQ ID NO: 106 - protein, 14 aa, Artificial Sequence
  Native Peptide Sequence to quantitate RAS-RAF-MAPK Pathway protein
NEGVATYAAAVLFR

SEQ ID NO: 107 - protein, 9 aa, Artificial Sequence
  Native Peptide Sequence to quantitate RAS-RAF-MAPK Pathway protein
DLEPPIVAR

SEQ ID NO: 108 - protein, 13 aa, Artificial Sequence
  Native Peptide Sequence to quantitate RAS-RAF-MAPK Pathway protein
IFPLRPDYQEPSR

SEQ ID NO: 109 - protein, 8 aa, Artificial Sequence
  Native Peptide Sequence to quantitate RAS-RAF-MAPK Pathway protein
TVSYTNLK

SEQ ID NO: 110 - protein, 9 aa, Artificial Sequence
  Native Peptide Sequence to quantitate RAS-RAF-MAPK Pathway protein
NLYSGDYYR

SEQ ID NO: 111 - protein, 10 aa, Artificial Sequence
  Native Peptide Sequence to quantitate RAS-RAF-MAPK Pathway protein
YLSSLNFVHR

SEQ ID NO: 112 - protein, 9 aa, Artificial Sequence
  Native Peptide Sequence to quantitate RAS-RAF-MAPK Pathway protein
DVAVEEFPR

SEQ ID NO: 113 - protein, 15 aa, Artificial Sequence
  Native Peptide Sequence to quantitate RAS-RAF-MAPK Pathway protein
SSSPSEQGSNSTYDR

SEQ ID NO: 114 - protein, 9 aa, Artificial Sequence
  Native Peptide Sequence to quantitate RAS-RAF-MAPK Pathway protein
FVTVPLHHR

SEQ ID NO: 115 - protein, 12 aa, Artificial Sequence
  Native Peptide Sequence to quantitate RAS-RAF-MAPK Pathway protein
HEPPNSSSSDVR

SEQ ID NO: 116 - protein, 16 aa, Artificial Sequence
  Native Peptide Sequence to quantitate RAS-RAF-MAPK Pathway protein
  MOD_RES (10)..(10): carbamidomethylated cysteine
QTYLPQPAICPDSVYK

SEQ ID NO: 117 - protein, 11 aa, Artificial Sequence
  Native Peptide Sequence to quantitate RAS-RAF-MAPK Pathway protein
ILPGLYIGNFK

SEQ ID NO: 118 - protein, 19 aa, Artificial Sequence
  Native Peptide Sequence to quantitate RAS-RAF-MAPK Pathway protein
VTHILSVHDSARPMLEGVK

SEQ ID NO: 119 - protein, 16 aa, Artificial Sequence
  Native Peptide Sequence to quantitate RAS-RAF-MAPK Pathway protein
  MOD_RES (3)..(3): carbamidomethylated cysteine
YLCIPAADSPSQNLTR

SEQ ID NO: 120 - protein, 6 aa, Artificial Sequence
  Native Peptide Sequence to quantitate RAS-RAF-MAPK Pathway protein
  MOD_RES (5)..(5): carbamidomethylated cysteine
FIHECR

SEQ ID NO: 121 - protein, 14 aa, Artificial Sequence
  Native Peptide Sequence to quantitate RAS-RAF-MAPK Pathway protein
  MOD_RES (4)..(8): carbamidomethylated cysteine
GESCLVHCLAGVSR

SEQ ID NO: 122 - protein, 11 aa, Artificial Sequence
  Native Peptide Sequence to quantitate RAS-RAF-MAPK Pathway protein
  MOD_RES (2)..(2): carbamidomethylated cysteine
SCANPNVGFQR

SEQ ID NO: 123 - protein, 15 aa, Artificial Sequence
  Native Peptide Sequence to quantitate RAS-RAF-MAPK Pathway protein
EEYGESPLQDAEEAK

SEQ ID NO: 124 - protein, 23 aa, Artificial Sequence
  Native Peptide Sequence to quantitate RAS-RAF-MAPK Pathway protein
DENGGGAGGSGSHGTLGLPSGGK

SEQ ID NO: 125 - protein, 24 aa, Artificial Sequence
  Native Peptide Sequence to quantitate RAS-RAF-MAPK Pathway protein
  MOD_RES (1)..(6): carbamidomethylated cysteine
CLLLDCRPFLAHSAGYILGSVNVR

SEQ ID NO: 126 - protein, 15 aa, Artificial Sequence
  Native Peptide Sequence to quantitate RAS-RAF-MAPK Pathway protein
LRSGLYSAVIVYDER

SEQ ID NO: 127 - protein, 13 aa, Artificial Sequence
  Native Peptide Sequence to quantitate RAS-RAF-MAPK Pathway protein
SGLYSAVIVYDER

SEQ ID NO: 128 - protein, 11 aa, Artificial Sequence
  Native Peptide Sequence to quantitate RAS-RAF-MAPK Pathway protein
  MOD_RES (9)..(9): carbamidomethylated cysteine
FSSEYPEFCSK

SEQ ID NO: 129 - protein, 11 aa, Artificial Sequence
  Native Peptide Sequence to quantitate RAS-RAF-MAPK Pathway protein
VRLEEAFEFVK

SEQ ID NO: 130 - protein, 9 aa, Artificial Sequence
  Native Peptide Sequence to quantitate RAS-RAF-MAPK Pathway protein
LEEAFEFVK

SEQ ID NO: 131 - protein, 15 aa, Artificial Sequence
  Native Peptide Sequence to quantitate RAS-RAF-MAPK Pathway protein
TVAWLNEQLELGNER

SEQ ID NO: 132 - protein, 10 aa, Artificial Sequence
  Native Peptide Sequence to quantitate RAS-RAF-MAPK Pathway protein
AFYLEGGFSK

SEQ ID NO: 133 - protein, 15 aa, Artificial Sequence
  Native Peptide Sequence to quantitate RAS-RAF-MAPK Pathway protein
ISSDSSSDIESDLDR

SEQ ID NO: 134 - protein, 14 aa, Artificial Sequence
  Native Peptide Sequence to quantitate RAS-RAF-MAPK Pathway protein
DSTNLDVLEEFGIK

SEQ ID NO: 135 - protein, 20 aa, Artificial Sequence
  Native Peptide Sequence to quantitate RAS-RAF-MAPK Pathway protein
YILNVTPNLPNLFENAGEFK

SEQ ID NO: 136 - protein, 14 aa, Artificial Sequence
  Native Peptide Sequence to quantitate RAS-RAF-MAPK Pathway protein
  MOD_RES (2)..(8): carbamidomethylated cysteine
NCGVLVHCLAGISR

SEQ ID NO: 137 - protein, 11 aa, Artificial Sequence
  Native Peptide Sequence to quantitate RAS-RAF-MAPK Pathway protein
  MOD_RES (8)..(8): carbamidomethylated cysteine
TLGLSSPCDNR

SEQ ID NO: 138 - protein, 11 aa, Artificial Sequence
  Native Peptide Sequence to quantitate RAS-RAF-MAPK Pathway protein
NLQEILHGAVR

SEQ ID NO: 139 - protein, 10 aa, Artificial Sequence
  Native Peptide Sequence to quantitate RAS-RAF-MAPK Pathway protein
IPLENLQIIR

SEQ ID NO: 140 - protein, 16 aa, Artificial Sequence
  Native Peptide Sequence to quantitate RAS-RAF-MAPK Pathway protein
LTQLGTFEDHFLSLQR

SEQ ID NO: 141 - protein, 9 aa, Artificial Sequence
  Native Peptide Sequence to quantitate RAS-RAF-MAPK Pathway protein
  MOD_RES (7)..(7): carbamidomethylated cysteine
YSFGATCVK

SEQ ID NO: 142 - protein, 9 aa, Artificial Sequence
  Native Peptide Sequence to quantitate RAS-RAF-MAPK Pathway protein
YLVIQGDER

SEQ ID NO: 143 - protein, 19 aa, Artificial Sequence
  Native Peptide Sequence to quantitate RAS-RAF-MAPK Pathway protein
ELVEPLTPSGEAPNQALLR

SEQ ID NO: 144 - protein, 19 aa, Artificial Sequence
  Native Peptide Sequence to quantitate RAS-RAF-MAPK Pathway protein
GSHQISLDNPDYQQDFFPK

SEQ ID NO: 145 - protein, 19 aa, Artificial Sequence
  Native Peptide Sequence to quantitate RAS-RAF-MAPK Pathway protein
  MOD_RES (7)..(7): phosphorylated threonine
ELVEPLTPSGEAPNQALLR

SEQ ID NO: 146 - protein, 19 aa, Artificial Sequence
  Native Peptide Sequence to quantitate RAS-RAF-MAPK Pathway protein
  MOD_RES (6)..(6): phosphorylated serine
GSHQISLDNPDYQQDFFPK

SEQ ID NO: 147 - protein, 12 aa, Artificial Sequence
  Native Peptide Sequence to quantitate RAS-RAF-MAPK Pathway protein
DGTLFAWGNGEK

SEQ ID NO: 148 - protein, 9 aa, Artificial Sequence
  Native Peptide Sequence to quantitate RAS-RAF-MAPK Pathway protein
VNIISVTNK

SEQ ID NO: 149 - protein, 9 aa, Artificial Sequence
  Native Peptide Sequence to quantitate RAS-RAF-MAPK Pathway protein
  MOD_RES (3)..(3): carbamidomethylated cysteine
AVCLEFSPK

SEQ ID NO: 150 - protein, 14 aa, Artificial Sequence
  Native Peptide Sequence to quantitate RAS-RAF-MAPK Pathway protein
NTVLATWQPYTTSK

SEQ ID NO: 151 - protein, 15 aa, Artificial Sequence
  Native Peptide Sequence to quantitate RAS-RAF-MAPK Pathway protein
DGTAGIPNLQLYDVK

SEQ ID NO: 152 - protein, 20 aa, Artificial Sequence
  Native Peptide Sequence to quantitate RAS-RAF-MAPK Pathway protein
NVNNEVHFFENNNFNTIANK

SEQ ID NO: 153 - protein, 14 aa, Artificial Sequence
  Native Peptide Sequence to quantitate RAS-RAF-MAPK Pathway protein
INDFVLSPGPQPYK

SEQ ID NO: 154 - protein, 17 aa, Artificial Sequence
  Native Peptide Sequence to quantitate RAS-RAF-MAPK Pathway protein
LYQYPNFAGPHAALANK

SEQ ID NO: 155 - protein, 7 aa, Artificial Sequence
  Native Peptide Sequence to quantitate RAS-RAF-MAPK Pathway protein
ATIFNLK

SEQ ID NO: 156 - protein, 12 aa, Artificial Sequence
  Native Peptide Sequence to quantitate RAS-RAF-MAPK Pathway protein
  MOD_RES (1)..(1): carbamidomethylated cysteine
CDPVFDFGTGPR

SEQ ID NO: 157 - protein, 21 aa, Artificial Sequence
  Native Peptide Sequence to quantitate RAS-RAF-MAPK Pathway protein
NAAYYSPHGHILVLAGFGNLR

SEQ ID NO: 158 - protein, 17 aa, Artificial Sequence
  Native Peptide Sequence to quantitate RAS-RAF-MAPK Pathway protein
TITYQAVPSEVPNEEPK

SEQ ID NO: 159 - protein, 11 aa, Artificial Sequence
  Native Peptide Sequence to quantitate RAS-RAF-MAPK Pathway protein
VATAYRPPALR

SEQ ID NO: 160 - protein, 7 aa, Artificial Sequence
  Native Peptide Sequence to quantitate RAS-RAF-MAPK Pathway protein
WLITLNK

SEQ ID NO: 161 - protein, 11 aa, Artificial Sequence
  Native Peptide Sequence to quantitate RAS-RAF-MAPK Pathway protein
  MOD_RES (8)..(8): carbamidomethylated cysteine
IAIWTTECENR

SEQ ID NO: 162 - protein, 8 aa, Artificial Sequence
  Native Peptide Sequence to quantitate RAS-RAF-MAPK Pathway protein
EAVTHIGR

SEQ ID NO: 163 - protein, 14 aa, Artificial Sequence
  Native Peptide Sequence to quantitate RAS-RAF-MAPK Pathway protein
IVIGYQSHADTATK

SEQ ID NO: 164 - protein, 15 aa, Artificial Sequence
  Native Peptide Sequence to quantitate RAS-RAF-MAPK Pathway protein
TESNQEVANPEHYIK

SEQ ID NO: 165 - protein, 7 aa, Artificial Sequence
  Native Peptide Sequence to quantitate RAS-RAF-MAPK Pathway protein
TWQANLR

SEQ ID NO: 166 - protein, 11 aa, Artificial Sequence
  Native Peptide Sequence to quantitate RAS-RAF-MAPK Pathway protein
DGIEPMWEDEK

SEQ ID NO: 167 - protein, 13 aa, Artificial Sequence
  Native Peptide Sequence to quantitate RAS-RAF-MAPK Pathway protein
  MOD_RES (5)..(5): carbamidomethylated cysteine
NPQLCYQDTILWK

SEQ ID NO: 168 - protein, 14 aa, Artificial Sequence
  Native Peptide Sequence to quantitate RAS-RAF-MAPK Pathway protein
  MOD_RES (1)..(9): carbamidomethylated cysteine
CWGESSEDCQSLTR

SEQ ID NO: 169 - protein, 14 aa, Artificial Sequence
  Native Peptide Sequence to quantitate RAS-RAF-MAPK Pathway protein
  MOD_RES (13)..(13): carbamidomethylated cysteine
AVTSANIQEFAGCK

SEQ ID NO: 170 - protein, 9 aa, Artificial Sequence
  Native Peptide Sequence to quantitate RAS-RAF-MAPK Pathway protein
  MOD_RES (4)..(8): carbamidomethylated cysteine
GQECVEECR

SEQ ID NO: 171 - protein, 7 aa, Artificial Sequence
  Native Peptide Sequence to quantitate RAS-RAF-MAPK Pathway protein
VLQGLPR

SEQ ID NO: 172 - protein, 11 aa, Artificial Sequence
  Native Peptide Sequence to quantitate RAS-RAF-MAPK Pathway protein
GIWIPDGENVK

SEQ ID NO: 173 - protein, 15 aa, Artificial Sequence
  Native Peptide Sequence to quantitate RAS-RAF-MAPK Pathway protein
LLDIDETEYHADGGK

SEQ ID NO: 174 - protein, 8 aa, Artificial Sequence
  Native Peptide Sequence to quantitate RAS-RAF-MAPK Pathway protein
ELVSEFSR

SEQ ID NO: 175 - protein, 21 aa, Artificial Sequence
  Native Peptide Sequence to quantitate RAS-RAF-MAPK Pathway protein
FVVIQNEDLGPASPLDSTFYR

SEQ ID NO: 176 - protein, 19 aa, Artificial Sequence
  Native Peptide Sequence to quantitate RAS-RAF-MAPK Pathway protein
SGGGDLTLGLEPSEEEAPR

SEQ ID NO: 177 - protein, 15 aa, Artificial Sequence
  Native Peptide Sequence to quantitate RAS-RAF-MAPK Pathway protein
GLQSLPTHDPSPLQR

SEQ ID NO: 178 - protein, 25 aa, Artificial Sequence
  Native Peptide Sequence to quantitate RAS-RAF-MAPK Pathway protein
  MOD_RES (8)..(19): carbamidomethylated cysteine
FPDEEGACQPCPINCTHSCVDLDDK

SEQ ID NO: 179 - protein, 13 aa, Artificial Sequence
  Native Peptide Sequence to quantitate RAS-RAF-MAPK Pathway protein
NNQLALTLIDTNR

SEQ ID NO: 180 - protein, 20 aa, Artificial Sequence
  Native Peptide Sequence to quantitate RAS-RAF-MAPK Pathway protein
  MOD_RES (7)..(16): carbamidomethylated cysteine
GPLPTDCCHEQCAAGCTGPK

SEQ ID NO: 181 - protein, 22 aa, Artificial Sequence
  Native Peptide Sequence to quantitate RAS-RAF-MAPK Pathway protein
  MOD_RES (2)..(2): carbamidomethylated cysteine
GCLASESSEGHVTGSEAELQEK

SEQ ID NO: 182 - protein, 16 aa, Artificial Sequence
  Native Peptide Sequence to quantitate RAS-RAF-MAPK Pathway protein
  MOD_RES (4)..(8): carbamidomethylated cysteine
GGVCVTHCNFLNGEPR

SEQ ID NO: 183 - protein, 12 aa, Artificial Sequence
  Native Peptide Sequence to quantitate RAS-RAF-MAPK Pathway protein
LSSLSDLEQQYR

SEQ ID NO: 184 - protein, 12 aa, Artificial Sequence
  Native Peptide Sequence to quantitate RAS-RAF-MAPK Pathway protein
DGNFGLQELGLK

SEQ ID NO: 185 - protein, 15 aa, Artificial Sequence
  Native Peptide Sequence to quantitate RAS-RAF-MAPK Pathway protein
  MOD_RES (3)..(3): carbamidomethylated cysteine
FLCYADTIHWQDIVR

SEQ ID NO: 186 - protein, 14 aa, Artificial Sequence
  Native Peptide Sequence to quantitate RAS-RAF-MAPK Pathway protein
  MOD_RES (1)..(12): carbamidomethylated cysteine
CPHNFVVDSSSCVR

SEQ ID NO: 187 - protein, 23 aa, Artificial Sequence
  Native Peptide Sequence to quantitate RAS-RAF-MAPK Pathway protein
FLETELVEPLTPSGTAPNQAQLR

SEQ ID NO: 188 - protein, 9 aa, Artificial Sequence
  Native Peptide Sequence to quantitate RAS-RAF-MAPK Pathway protein
ILNETTGPK

SEQ ID NO: 189 - protein, 8 aa, Artificial Sequence
  Native Peptide Sequence to quantitate RAS-RAF-MAPK Pathway protein
ELAAEFSR

SEQ ID NO: 190 - protein, 15 aa, Artificial Sequence
  Native Peptide Sequence to quantitate RAS-RAF-MAPK Pathway protein
DGGFAAEQGVSVPYR

SEQ ID NO: 191 - protein, 15 aa, Artificial Sequence
  Native Peptide Sequence to quantitate RAS-RAF-MAPK Pathway protein
KPVAPHVQEDSSTQR

SEQ ID NO: 192 - protein, 12 aa, Artificial Sequence
  Native Peptide Sequence to quantitate RAS-RAF-MAPK Pathway protein
YSADPTVFAPER

SEQ ID NO: 193 - protein, 15 aa, Artificial Sequence
  Native Peptide Sequence to quantitate RAS-RAF-MAPK Pathway protein
STLQHPDYLQEYSTK

SEQ ID NO: 194 - protein, 16 aa, Artificial Sequence
  Native Peptide Sequence to quantitate RAS-RAF-MAPK Pathway protein
NLTEILNGGVYVDQNK

SEQ ID NO: 195 - protein, 23 aa, Artificial Sequence
  Native Peptide Sequence to quantitate RAS-RAF-MAPK Pathway protein
  MOD_RES (2)..(22): carbamidomethylated cysteine
YCAVCNDYASGYHYGVWSCEGCK

SEQ ID NO: 196 - protein, 11 aa, Artificial Sequence
  Native Peptide Sequence to quantitate RAS-RAF-MAPK Pathway protein
LLFAPNLLLDR

SEQ ID NO: 197 - protein, 11 aa, Artificial Sequence
  Native Peptide Sequence to quantitate RAS-RAF-MAPK Pathway protein
AGLTLQQQHQR

SEQ ID NO: 198 - protein, 12 aa, Artificial Sequence
  Native Peptide Sequence to quantitate RAS-RAF-MAPK Pathway protein
LAQLLLILSHIR

SEQ ID NO: 199 - protein, 26 aa, Artificial Sequence
  Native Peptide Sequence to quantitate RAS-RAF-MAPK Pathway protein
GGASVEETDQSHLATAGSTSSHSLQK

SEQ ID NO: 200 - protein, 12 aa, Artificial Sequence
  Native Peptide Sequence to quantitate RAS-RAF-MAPK Pathway protein
AANLWPSPLMIK

SEQ ID NO: 201 - protein, 18 aa, Artificial Sequence
  Native Peptide Sequence to quantitate RAS-RAF-MAPK Pathway protein
RTWPLEVPETEPTLGHLR

SEQ ID NO: 202 - protein, 17 aa, Artificial Sequence
  Native Peptide Sequence to quantitate RAS-RAF-MAPK Pathway protein
TWPLEVPETEPTLGHLR

SEQ ID NO: 203 - protein, 11 aa, Artificial Sequence
  Native Peptide Sequence to quantitate RAS-RAF-MAPK Pathway protein
  MOD_RES (10)..(10): carbamidomethylated cysteine
LQLLPESFICK

SEQ ID NO: 204 - protein, 10 aa, Artificial Sequence
  Native Peptide Sequence to quantitate RAS-RAF-MAPK Pathway protein
LGENVANIYK

SEQ ID NO: 205 - protein, 14 aa, Artificial Sequence
  Native Peptide Sequence to quantitate RAS-RAF-MAPK Pathway protein
LGENVANIYKDLQK

SEQ ID NO: 206 - protein, 10 aa, Artificial Sequence
  Native Peptide Sequence to quantitate RAS-RAF-MAPK Pathway protein
  MOD_RES (9)..(9): carbamidomethylated cysteine
SVLSLSAVCR

SEQ ID NO: 207 - protein, 7 aa, Artificial Sequence
  Native Peptide Sequence to quantitate RAS-RAF-MAPK Pathway protein
VQDTDWK

SEQ ID NO: 208 - protein, 11 aa, Artificial Sequence
  Native Peptide Sequence to quantitate RAS-RAF-MAPK Pathway protein
VQDTDWKELYR

SEQ ID NO: 209 - protein, 17 aa, Artificial Sequence
  Native Peptide Sequence to quantitate RAS-RAF-MAPK Pathway protein
FDPVGPLPGPNPILPGR

SEQ ID NO: 210 - protein, 10 aa, Artificial Sequence
  Native Peptide Sequence to quantitate RAS-RAF-MAPK Pathway protein
DGVQLAESNR

SEQ ID NO: 211 - protein, 7 aa, Artificial Sequence
  Native Peptide Sequence to quantitate RAS-RAF-MAPK Pathway protein
EFKPDHR

SEQ ID NO: 212 - protein, 15 aa, Artificial Sequence
  Native Peptide Sequence to quantitate RAS-RAF-MAPK Pathway protein
SPHRPILQAGLPANK

SEQ ID NO: 213 - protein, 13 aa, Artificial Sequence
  Native Peptide Sequence to quantitate RAS-RAF-MAPK Pathway protein
VYSDPQPHIQWLK

SEQ ID NO: 214 - protein, 13 aa, Artificial Sequence
  Native Peptide Sequence to quantitate RAS-RAF-MAPK Pathway protein
IGPDNLPYVQILK

SEQ ID NO: 215 - protein, 12 aa, Artificial Sequence
  Native Peptide Sequence to quantitate RAS-RAF-MAPK Pathway protein
  MOD_RES (1)..(1): carbamidomethylated cysteine
CPSSGTPNPTLR

SEQ ID NO: 216 - protein, 11 aa, Artificial Sequence
  Native Peptide Sequence to quantitate RAS-RAF-MAPK Pathway protein
HPAQLANGGLK

SEQ ID NO: 217 - protein, 22 aa, Artificial Sequence
  Native Peptide Sequence to quantitate RAS-RAF-MAPK Pathway protein
  MOD_RES (8)..(8): carbamidomethylated cysteine
RPPGLEYCYNPSHNPEEQLSSK

SEQ ID NO: 218 - protein, 11 aa, Artificial Sequence
  Native Peptide Sequence to quantitate RAS-RAF-MAPK Pathway protein
TVLIGEYLQIK

SEQ ID NO: 219 - protein, 10 aa, Artificial Sequence
  Native Peptide Sequence to quantitate RAS-RAF-MAPK Pathway protein
YGPDGLPYLK

SEQ ID NO: 220 - protein, 17 aa, Artificial Sequence
  Native Peptide Sequence to quantitate RAS-RAF-MAPK Pathway protein
  MOD_RES (16)..(16): carbamidomethylated cysteine
LQVLNASHEDSGAYSCR

SEQ ID NO: 221 - protein, 11 aa, Artificial Sequence
  Native Peptide Sequence to quantitate RAS-RAF-MAPK Pathway protein
LLAVPAANTVR

SEQ ID NO: 222 - protein, 13 aa, Artificial Sequence
  Native Peptide Sequence to quantitate RAS-RAF-MAPK Pathway protein
LEIASFLPEDAGR

SEQ ID NO: 223 - protein, 21 aa, Artificial Sequence
  Native Peptide Sequence to quantitate RAS-RAF-MAPK Pathway protein
HIVINGSSFGADGFPYVQVLK

SEQ ID NO: 224 - protein, 17 aa, Artificial Sequence
  Native Peptide Sequence to quantitate RAS-RAF-MAPK Pathway protein
APHPFGVPAPSAGAYSR

SEQ ID NO: 225 - protein, 13 aa, Artificial Sequence
  Native Peptide Sequence to quantitate RAS-RAF-MAPK Pathway protein
GKVEQLSPEEEEK

SEQ ID NO: 226 - protein, 11 aa, Artificial Sequence
  Native Peptide Sequence to quantitate RAS-RAF-MAPK Pathway protein
VEQLSPEEEEK

SEQ ID NO: 227 - protein, 22 aa, Artificial Sequence
  Native Peptide Sequence to quantitate RAS-RAF-MAPK Pathway protein
TEPFDDFLFPASSRPSGSETAR

SEQ ID NO: 228 - protein, 12 aa, Artificial Sequence
  Native Peptide Sequence to quantitate RAS-RAF-MAPK Pathway protein
SALQTEIANLLK

SEQ ID NO: 229 - protein, 17 aa, Artificial Sequence
  Native Peptide Sequence to quantitate RAS-RAF-MAPK Pathway protein
ELTDTLQAETDQLEDEK

SEQ ID NO: 230 - protein, 12 aa, Artificial Sequence
  Native Peptide Sequence to quantitate RAS-RAF-MAPK Pathway protein
VEQLSPEEEEKR

SEQ ID NO: 231 - protein, 20 aa, Artificial Sequence
  Native Peptide Sequence to quantitate RAS-RAF-MAPK Pathway protein
ASLQSGQEGAGDSPGSQFSK

SEQ ID NO: 232 - protein, 10 aa, Artificial Sequence
  Native Peptide Sequence to quantitate RAS-RAF-MAPK Pathway protein
  MOD_RES (3)..(3): carbamidomethylated cysteine
LDCDMESIIR

SEQ ID NO: 233 - protein, 19 aa, Artificial Sequence
  Native Peptide Sequence to quantitate RAS-RAF-MAPK Pathway protein
AALQTAPESADDSPSQLSK

SEQ ID NO: 234 - protein, 12 aa, Artificial Sequence
  Native Peptide Sequence to quantitate RAS-RAF-MAPK Pathway protein
SSDELDAWTDFR

SEQ ID NO: 235 - protein, 9 aa, Artificial Sequence
  Native Peptide Sequence to quantitate RAS-RAF-MAPK Pathway protein
SFEDIHQYR

SEQ ID NO: 236 - protein, 12 aa, Artificial Sequence
  Native Peptide Sequence to quantitate RAS-RAF-MAPK Pathway protein
SYGIPYIETSAK

SEQ ID NO: 237 - protein, 15 aa, Artificial Sequence
  Native Peptide Sequence to quantitate RAS-RAF-MAPK Pathway protein
  MOD_RES (11)..(14): carbamidomethylated cysteine
LNPPDESGPGCMSCK

SEQ ID NO: 238 - protein, 10 aa, Artificial Sequence
  Native Peptide Sequence to quantitate RAS-RAF-MAPK Pathway protein
LFDQAFGLPR

SEQ ID NO: 239 - protein, 17 aa, Artificial Sequence
  Native Peptide Sequence to quantitate RAS-RAF-MAPK Pathway protein
LATQSNEITIPVTFESR

SEQ ID NO: 240 - protein, 16 aa, Artificial Sequence
  Native Peptide Sequence to quantitate RAS-RAF-MAPK Pathway protein
VSLDVNHFAPDELTVK

SEQ ID NO: 241 - protein, 8 aa, Artificial Sequence
  Native Peptide Sequence to quantitate RAS-RAF-MAPK Pathway protein
GPSWDPFR

SEQ ID NO: 242 - protein, 9 aa, Artificial Sequence
  Native Peptide Sequence to quantitate RAS-RAF-MAPK Pathway protein
DGVVEITGK

SEQ ID NO: 243 - protein, 7 aa, Artificial Sequence
  Native Peptide Sequence to quantitate RAS-RAF-MAPK Pathway protein
VPFSLLR

SEQ ID NO: 244 - protein, 10 aa, Artificial Sequence
  Native Peptide Sequence to quantitate RAS-RAF-MAPK Pathway protein
AQLGGPEAAK

SEQ ID NO: 245 - protein, 8 aa, Artificial Sequence
  Native Peptide Sequence to quantitate RAS-RAF-MAPK Pathway protein
LGNFFSPK

SEQ ID NO: 246 - protein, 7 aa, Artificial Sequence
  Native Peptide Sequence to quantitate RAS-RAF-MAPK Pathway protein
ATGLHFR

SEQ ID NO: 247 - protein, 13 aa, Artificial Sequence
  Native Peptide Sequence to quantitate RAS-RAF-MAPK Pathway protein
LGLAPQIQDLYGK

SEQ ID NO: 248 - protein, 10 aa, Artificial Sequence
  Native Peptide Sequence to quantitate RAS-RAF-MAPK Pathway protein
ALQSPALGLR

SEQ ID NO: 249 - protein, 7 aa, Artificial Sequence
  Native Peptide Sequence to quantitate RAS-RAF-MAPK Pathway protein
ELATLQR

SEQ ID NO: 250 - protein, 12 aa, Artificial Sequence
  Native Peptide Sequence to quantitate RAS-RAF-MAPK Pathway protein
ALESGDVNTVWK

SEQ ID NO: 251 - protein, 11 aa, Artificial Sequence
  Native Peptide Sequence to quantitate RAS-RAF-MAPK Pathway protein
TLQALQIPAAK

SEQ ID NO: 252 - protein, 7 aa, Artificial Sequence
  Native Peptide Sequence to quantitate RAS-RAF-MAPK Pathway protein
EEYLLLR

SEQ ID NO: 253 - protein, 17 aa, Artificial Sequence
  Native Peptide Sequence to quantitate RAS-RAF-MAPK Pathway protein
LPYDVTPEQALAHEEVK

SEQ ID NO: 254 - protein, 9 aa, Artificial Sequence
  Native Peptide Sequence to quantitate RAS-RAF-MAPK Pathway protein
IIGNLLYYR

SEQ ID NO: 255 - protein, 11 aa, Artificial Sequence
  Native Peptide Sequence to quantitate RAS-RAF-MAPK Pathway protein
LQQTYAALNSK

SEQ ID NO: 256 - protein, 12 aa, Artificial Sequence
  Native Peptide Sequence to quantitate RAS-RAF-MAPK Pathway protein
ATFYGEQVDYYK

SEQ ID NO: 257 - protein, 9 aa, Artificial Sequence
  Native Peptide Sequence to quantitate RAS-RAF-MAPK Pathway protein
  MOD_RES (2)..(2): carbamidomethylated cysteine
TCLDNLASK

SEQ ID NO: 258 - protein, 14 aa, Artificial Sequence
  Native Peptide Sequence to quantitate RAS-RAF-MAPK Pathway protein
  MOD_RES (12)..(12): carbamidomethylated cysteine
LGSGEYTAEELCIR

SEQ ID NO: 259 - protein, 17 aa, Artificial Sequence
  Native Peptide Sequence to quantitate RAS-RAF-MAPK Pathway protein
  MOD_RES (5)..(5): carbamidomethylated cysteine
ISPLCHNLFALYDENTK

SEQ ID NO: 260 - protein, 8 aa, Artificial Sequence
  Native Peptide Sequence to quantitate RAS-RAF-MAPK Pathway protein
YIPETLNK

SEQ ID NO: 261 - protein, 10 aa, Artificial Sequence
  Native Peptide Sequence to quantitate RAS-RAF-MAPK Pathway protein
YLATLETLTK

SEQ ID NO: 262 - protein, 8 aa, Artificial Sequence
  Native Peptide Sequence to quantitate RAS-RAF-MAPK Pathway protein
NFQIEVQK

SEQ ID NO: 263 - protein, 10 aa, Artificial Sequence
  Native Peptide Sequence to quantitate RAS-RAF-MAPK Pathway protein
EISNLLVATK

SEQ ID NO: 264 - protein, 11 aa, Artificial Sequence
  Native Peptide Sequence to quantitate RAS-RAF-MAPK Pathway protein
EISNLLVATKK

SEQ ID NO: 265 - protein, 11 aa, Artificial Sequence
  Native Peptide Sequence to quantitate RAS-RAF-MAPK Pathway protein
KDLVQGEHLGR

SEQ ID NO: 266 - protein, 10 aa, Artificial Sequence
  Native Peptide Sequence to quantitate RAS-RAF-MAPK Pathway protein
DLVQGEHLGR

SEQ ID NO: 267 - protein, 12 aa, Artificial Sequence
  Native Peptide Sequence to quantitate RAS-RAF-MAPK Pathway protein
  MOD_RES (7)..(7): carbamidomethylated cysteine
EGIDSECGPFIK

SEQ ID NO: 268 - protein, 13 aa, Artificial Sequence
  Native Peptide Sequence to quantitate RAS-RAF-MAPK Pathway protein
LSDPGIPITVLSR

SEQ ID NO: 269 - protein, 13 aa, Artificial Sequence
  Native Peptide Sequence to quantitate RAS-RAF-MAPK Pathway protein
  MOD_RES (8)..(8): carbamidomethylated cysteine
IPWIAPECVEDSK

SEQ ID NO: 270 - protein, 15 aa, Artificial Sequence
  Native Peptide Sequence to quantitate RAS-RAF-MAPK Pathway protein
YDPEGDNTGEQVAVK

SEQ ID NO: 271 - protein, 9 aa, Artificial Sequence
  Native Peptide Sequence to quantitate RAS-RAF-MAPK Pathway protein
NLYHENIVK

SEQ ID NO: 272 - protein, 11 aa, Artificial Sequence
  Native Peptide Sequence to quantitate RAS-RAF-MAPK Pathway protein
NVLVESEHQVK

SEQ ID NO: 273 - protein, 9 aa, Artificial Sequence
  Native Peptide Sequence to quantitate RAS-RAF-MAPK Pathway protein
HDFVHGWIK

SEQ ID NO: 274 - protein, 9 aa, Artificial Sequence
  Native Peptide Sequence to quantitate RAS-RAF-MAPK Pathway protein
IQDYHILTR

SEQ ID NO: 275 - protein, 10 aa, Artificial Sequence
  Native Peptide Sequence to quantitate RAS-RAF-MAPK Pathway protein
NLEIELSSLR

SEQ ID NO: 276 - protein, 11 aa, Artificial Sequence
  Native Peptide Sequence to quantitate RAS-RAF-MAPK Pathway protein
AGNQTGLYVLR

SEQ ID NO: 277 - protein, 9 aa, Artificial Sequence
  Native Peptide Sequence to quantitate RAS-RAF-MAPK Pathway protein
YFLTFAVER

SEQ ID NO: 278 - protein, 12 aa, Artificial Sequence
  Native Peptide Sequence to quantitate RAS-RAF-MAPK Pathway protein
NENEEYNLSGTK

SEQ ID NO: 279 - protein, 10 aa, Artificial Sequence
  Native Peptide Sequence to quantitate RAS-RAF-MAPK Pathway protein
SDNIIFQFTK

SEQ ID NO: 280 - protein, 16 aa, Artificial Sequence
  Native Peptide Sequence to quantitate RAS-RAF-MAPK Pathway protein
EVGDYGQLHETEVLLK

SEQ ID NO: 281 - protein, 13 aa, Artificial Sequence
  Native Peptide Sequence to quantitate RAS-RAF-MAPK Pathway protein
LSDPGISITVLPK

SEQ ID NO: 282 - protein, 13 aa, Artificial Sequence
  Native Peptide Sequence to quantitate RAS-RAF-MAPK Pathway protein
  MOD_RES (8)..(8): carbamidomethylated cysteine
IPWVPPECIENPK

SEQ ID NO: 283 - protein, 7 aa, Artificial Sequence
  Native Peptide Sequence to quantitate RAS-RAF-MAPK Pathway protein
FLQQLGK

SEQ ID NO: 284 - protein, 15 aa, Artificial Sequence
  Native Peptide Sequence to quantitate RAS-RAF-MAPK Pathway protein
YDPLQDNTGEVVAVK

SEQ ID NO: 285 - protein, 9 aa, Artificial Sequence
  Native Peptide Sequence to quantitate RAS-RAF-MAPK Pathway protein
SLQHDNIVK

SEQ ID NO: 286 - protein, 10 aa, Artificial Sequence
  Native Peptide Sequence to quantitate RAS-RAF-MAPK Pathway protein
  MOD_RES (9)..(9): carbamidomethylated cysteine
LLQYTSQICK

SEQ ID NO: 287 - protein, 9 aa, Artificial Sequence
  Native Peptide Sequence to quantitate RAS-RAF-MAPK Pathway protein
NILVENENR

SEQ ID NO: 288 - protein, 16 aa, Artificial Sequence
  Native Peptide Sequence to quantitate RAS-RAF-MAPK Pathway protein
ENDQTPLAIYNSISYK

SEQ ID NO: 289 - protein, 19 aa, Artificial Sequence
  Native Peptide Sequence to quantitate RAS-RAF-MAPK Pathway protein
EPGESPIFWYAPESLTESK

SEQ ID NO: 290 - protein, 15 aa, Artificial Sequence
  Native Peptide Sequence to quantitate RAS-RAF-MAPK Pathway protein
NVTDEQEGFAEGFVR

SEQ ID NO: 291 - protein, 14 aa, Artificial Sequence
  Native Peptide Sequence to quantitate RAS-RAF-MAPK Pathway protein
NSDLLTSPDVGLLK

SEQ ID NO: 292 - protein, 14 aa, Artificial Sequence
  Native Peptide Sequence to quantitate RAS-RAF-MAPK Pathway protein
AQNSELASTANMLR

SEQ ID NO: 293 - protein, 23 aa, Artificial Sequence
  Native Peptide Sequence to quantitate RAS-RAF-MAPK Pathway protein
  MOD_RES (21)..(21): carbamidomethylated cysteine
LIIQSSNGHITTTPTPTQFLCPK

SEQ ID NO: 294 - protein, 26 aa, Artificial Sequence
  Native Peptide Sequence to quantitate RAS-RAF-MAPK Pathway protein
EEPQTVPEMPGETPPLSPIDMESQER

SEQ ID NO: 295 - protein, 9 aa, Artificial Sequence
  Native Peptide Sequence to quantitate RAS-RAF-MAPK Pathway protein
SFEDIHHYR

SEQ ID NO: 296 - protein, 13 aa, Artificial Sequence
  Native Peptide Sequence to quantitate RAS-RAF-MAPK Pathway protein
DSEDVPMVLVGNK

SEQ ID NO: 297 - protein, 10 aa, Artificial Sequence
  Native Peptide Sequence to quantitate RAS-RAF-MAPK Pathway protein
VEDAFYTLVR

SEQ ID NO: 298 - protein, 12 aa, Artificial Sequence
  Native Peptide Sequence to quantitate RAS-RAF-MAPK Pathway protein
QGVDDAFYTLVR

SEQ ID NO: 299 - protein, 15 aa, Artificial Sequence
  Native Peptide Sequence to quantitate RAS-RAF-MAPK Pathway protein
  MOD_RES (7)..(7): carbamidomethylated cysteine
LSHDWLCYLAPEIVR

SEQ ID NO: 300 - protein, 18 aa, Artificial Sequence
  Native Peptide Sequence to quantitate RAS-RAF-MAPK Pathway protein
ADVLEAHEAEAEEPEAGK

SEQ ID NO: 301 - protein, 14 aa, Artificial Sequence
  Native Peptide Sequence to quantitate RAS-RAF-MAPK Pathway protein
ISELGAGNGGVVFK

SEQ ID NO: 302 - protein, 8 aa, Artificial Sequence
  Native Peptide Sequence to quantitate RAS-RAF-MAPK Pathway protein
IPEQILGK

SEQ ID NO: 303 - protein, 12 aa, Artificial Sequence
  Native Peptide Sequence to quantitate RAS-RAF-MAPK Pathway protein
VSHKPSGLVMAR

SEQ ID NO: 304 - protein, 11 aa, Artificial Sequence
  Native Peptide Sequence to quantitate RAS-RAF-MAPK Pathway protein
LEELELDEQQR

SEQ ID NO: 305 - protein, 7 aa, Artificial Sequence
  Native Peptide Sequence to quantitate RAS-RAF-MAPK Pathway protein
VSIAVIK

SEQ ID NO: 306 - protein, 14 aa, Artificial Sequence
  Native Peptide Sequence to quantitate RAS-RAF-MAPK Pathway protein
ISELGAGNGGVVTK

SEQ ID NO: 307 - protein, 12 aa, Artificial Sequence
  Native Peptide Sequence to quantitate RAS-RAF-MAPK Pathway protein
VQHRPSGLIMAR

SEQ ID NO: 308 - protein, 8 aa, Artificial Sequence
  Native Peptide Sequence to quantitate RAS-RAF-MAPK Pathway protein
IPEEILGK

SEQ ID NO: 309 - protein, 14 aa, Artificial Sequence
  Native Peptide Sequence to quantitate RAS-RAF-MAPK Pathway protein
  MOD_RES (13)..(13): carbamidomethylated cysteine
SEVEEVDFAGWLCK

SEQ ID NO: 310 - protein, 9 aa, Artificial Sequence
  Native Peptide Sequence to quantitate RAS-RAF-MAPK Pathway protein
LNQPGTPTR

SEQ ID NO: 311 - protein, 14 aa, Artificial Sequence
  Native Peptide Sequence to quantitate RAS-RAF-MAPK Pathway protein
  MOD_RES (12)..(12): carbamidomethylated cysteine
FSPEFVDFTAQCLR

SEQ ID NO: 312 - protein, 14 aa, Artificial Sequence
  Native Peptide Sequence to quantitate RAS-RAF-MAPK Pathway protein
FPYESWGTPFQQLK

SEQ ID NO: 313 - protein, 11 aa, Artificial Sequence
  Native Peptide Sequence to quantitate RAS-RAF-MAPK Pathway protein
DVKPSNVLINK

SEQ ID NO: 314 - protein, 8 aa, Artificial Sequence
  Native Peptide Sequence to quantitate RAS-RAF-MAPK Pathway protein
TFITIGDR

SEQ ID NO: 315 - protein, 8 aa, Artificial Sequence
  Native Peptide Sequence to quantitate RAS-RAF-MAPK Pathway protein
TDIAAFVK

SEQ ID NO: 316 - protein, 17 aa, Artificial Sequence
  Native Peptide Sequence to quantitate RAS-RAF-MAPK Pathway protein
NFEVEADDLVTISELGR

SEQ ID NO: 317 - protein, 16 aa, Artificial Sequence
  Native Peptide Sequence to quantitate RAS-RAF-MAPK Pathway protein
  MOD_RES (2)..(2): carbamidomethylated cysteine
LCDFGISGQLVDSIAK

SEQ ID NO: 318 - protein, 17 aa, Artificial Sequence
  Native Peptide Sequence to quantitate RAS-RAF-MAPK Pathway protein
FTLNPNPTGVQNPHIER

SEQ ID NO: 319 - protein, 13 aa, Artificial Sequence
  Native Peptide Sequence to quantitate RAS-RAF-MAPK Pathway protein
WNSVFDQLTQVVK

SEQ ID NO: 320 - protein, 17 aa, Artificial Sequence
  Native Peptide Sequence to quantitate RAS-RAF-MAPK Pathway protein
YVYSVLDDVIPEEILGK

SEQ ID NO: 321 - protein, 11 aa, Artificial Sequence
  Native Peptide Sequence to quantitate RAS-RAF-MAPK Pathway protein
DIKPSNILLDR

SEQ ID NO: 322 - protein, 12 aa, Artificial Sequence
  Native Peptide Sequence to quantitate RAS-RAF-MAPK Pathway protein
GDPPQLSNSEER

SEQ ID NO: 323 - protein, 10 aa, Artificial Sequence
  Native Peptide Sequence to quantitate RAS-RAF-MAPK Pathway protein
  MOD_RES (6)..(9): carbamidomethylated cysteine
AVEVACYVCK

SEQ ID NO: 324 - protein, 16 aa, Artificial Sequence
  Native Peptide Sequence to quantitate RAS-RAF-MAPK Pathway protein
DVKPSNVLINALGQVK

SEQ ID NO: 325 - protein, 14 aa, Artificial Sequence
  Native Peptide Sequence to quantitate RAS-RAF-MAPK Pathway protein
EAFEQPQTSSTPPR

SEQ ID NO: 326 - protein, 13 aa, Artificial Sequence
  Native Peptide Sequence to quantitate RAS-RAF-MAPK Pathway protein
  MOD_RES (2)..(2): carbamidomethylated cysteine
ACISIGNQNFEVK

SEQ ID NO: 327 - protein, 9 aa, Artificial Sequence
  Native Peptide Sequence to quantitate RAS-RAF-MAPK Pathway protein
GTDVASFVK

SEQ ID NO: 328 - protein, 7 aa, Artificial Sequence
  Native Peptide Sequence to quantitate RAS-RAF-MAPK Pathway protein
IAVSIVK

SEQ ID NO: 329 - protein, 14 aa, Artificial Sequence
  Native Peptide Sequence to quantitate RAS-RAF-MAPK Pathway protein
FPYDSWGTPFQQLK

SEQ ID NO: 330 - protein, 11 aa, Artificial Sequence
  Native Peptide Sequence to quantitate RAS-RAF-MAPK Pathway protein
GQTIPEDILGK

SEQ ID NO: 331 - protein, 14 aa, Artificial Sequence
  Native Peptide Sequence to quantitate RAS-RAF-MAPK Pathway protein
  MOD_RES (12)..(12): carbamidomethylated cysteine
FSAEFVDFTSQCLK

SEQ ID NO: 332 - protein, 18 aa, Artificial Sequence
  Native Peptide Sequence to quantitate RAS-RAF-MAPK Pathway protein
SPSSESSPQHPTPPARPR

SEQ ID NO: 333 - protein, 8 aa, Artificial Sequence
  Native Peptide Sequence to quantitate RAS-RAF-MAPK Pathway protein
TDFEVLTK

SEQ ID NO: 334 - protein, 12 aa, Artificial Sequence
  Native Peptide Sequence to quantitate RAS-RAF-MAPK Pathway protein
DVKPSNILLDER

SEQ ID NO: 335 - protein, 8 aa, Artificial Sequence
  Native Peptide Sequence to quantitate RAS-RAF-MAPK Pathway protein
LLEHSFIK

SEQ ID NO: 336 - protein, 14 aa, Artificial Sequence
  Native Peptide Sequence to quantitate RAS-RAF-MAPK Pathway protein
TSGVLSQPHLPFFR

SEQ ID NO: 337 - protein, 14 aa, Artificial Sequence
  Native Peptide Sequence to quantitate RAS-RAF-MAPK Pathway protein
IDPPDPTKPDYDIR

SEQ ID NO: 338 - protein, 8 aa, Artificial Sequence
  Native Peptide Sequence to quantitate RAS-RAF-MAPK Pathway protein
AASSLEQK

SEQ ID NO: 339 - protein, 9 aa, Artificial Sequence
  Native Peptide Sequence to quantitate RAS-RAF-MAPK Pathway protein
  MOD_RES (2)..(2): carbamidomethylated cysteine
LCDFGISGR

SEQ ID NO: 340 - protein, 9 aa, Artificial Sequence
  Native Peptide Sequence to quantitate RAS-RAF-MAPK Pathway protein
GQVFDVGPR

SEQ ID NO: 341 - protein, 14 aa, Artificial Sequence
  Native Peptide Sequence to quantitate RAS-RAF-MAPK Pathway protein
FRHENIIGINDIIR

SEQ ID NO: 342 - protein, 12 aa, Artificial Sequence
  Native Peptide Sequence to quantitate RAS-RAF-MAPK Pathway protein
HENIIGINDIIR

SEQ ID NO: 343 - protein, 18 aa, Artificial Sequence
  Native Peptide Sequence to quantitate RAS-RAF-MAPK Pathway protein
  MOD_RES (10)..(10): carbamidomethylated cysteine
TQHLSNDHICYFLYQILR

SEQ ID NO: 344 - protein, 16 aa, Artificial Sequence
  Native Peptide Sequence to quantitate RAS-RAF-MAPK Pathway protein
  MOD_RES (13)..(13): carbamidomethylated cysteine
DLKPSNLLLNTTCDLK

SEQ ID NO: 345 - protein, 19 aa, Artificial Sequence
  Native Peptide Sequence to quantitate RAS-RAF-MAPK Pathway protein
VADPDHDHTGFLTEYVATR

SEQ ID NO: 346 - protein, 9 aa, Artificial Sequence
  Native Peptide Sequence to quantitate RAS-RAF-MAPK Pathway protein
NYLLSLPHK

SEQ ID NO: 347 - protein, 7 aa, Artificial Sequence
  Native Peptide Sequence to quantitate RAS-RAF-MAPK Pathway protein
NKVPWNR

SEQ ID NO: 348 - protein, 8 aa, Artificial Sequence
  Native Peptide Sequence to quantitate RAS-RAF-MAPK Pathway protein
LFPNADSK

SEQ ID NO: 349 - protein, 7 aa, Artificial Sequence
  Native Peptide Sequence to quantitate RAS-RAF-MAPK Pathway protein
ALDLLDK

SEQ ID NO: 350 - protein, 8 aa, Artificial Sequence
  Native Peptide Sequence to quantitate RAS-RAF-MAPK Pathway protein
MLTFNPHK

SEQ ID NO: 351 - protein, 9 aa, Artificial Sequence
  Native Peptide Sequence to quantitate RAS-RAF-MAPK Pathway protein
MLTFNPHKR

SEQ ID NO: 352 - protein, 11 aa, Artificial Sequence
  Native Peptide Sequence to quantitate RAS-RAF-MAPK Pathway protein
LKELIFEETAR

SEQ ID NO: 353 - protein, 8 aa, Artificial Sequence
  Native Peptide Sequence to quantitate RAS-RAF-MAPK Pathway protein
TVWEVPQR

SEQ ID NO: 354 - protein, 22 aa, Artificial Sequence
  Native Peptide Sequence to quantitate RAS-RAF-MAPK Pathway protein
  MOD_RES (16)..(16): carbamidomethylated cysteine
LQGLRPVGSGAYGSVCSAYDAR

SEQ ID NO: 355 - protein, 18 aa, Artificial Sequence
  Native Peptide Sequence to quantitate RAS-RAF-MAPK Pathway protein
  MOD_RES (1)..(1): carbamidomethylated cysteine
CQALSDEHVQFLVYQLLR

SEQ ID NO: 356 - protein, 10 aa, Artificial Sequence
  Native Peptide Sequence to quantitate RAS-RAF-MAPK Pathway protein
YIHSAGIIHR

SEQ ID NO: 357 - protein, 16 aa, Artificial Sequence
  Native Peptide Sequence to quantitate RAS-RAF-MAPK Pathway protein
  MOD_RES (13)..(13): carbamidomethylated cysteine
DLKPSNVAVNEDCELR

SEQ ID NO: 358 - protein, 13 aa, Artificial Sequence
  Native Peptide Sequence to quantitate RAS-RAF-MAPK Pathway protein
ALFPGSDYIDQLK

SEQ ID NO: 359 - protein, 14 aa, Artificial Sequence
  Native Peptide Sequence to quantitate RAS-RAF-MAPK Pathway protein
ALFPGSDYIDQLKR

SEQ ID NO: 360 - protein, 15 aa, Artificial Sequence
  Native Peptide Sequence to quantitate RAS-RAF-MAPK Pathway protein
IMEVVGTPSPEVLAK

SEQ ID NO: 361 - protein, 12 aa, Artificial Sequence
  Native Peptide Sequence to quantitate RAS-RAF-MAPK Pathway protein
TYIQSLPPMPQK

SEQ ID NO: 362 - protein, 7 aa, Artificial Sequence
  Native Peptide Sequence to quantitate RAS-RAF-MAPK Pathway protein
DLSSIFR

SEQ ID NO: 363 - protein, 12 aa, Artificial Sequence
  Native Peptide Sequence to quantitate RAS-RAF-MAPK Pathway protein
GANPLAIDLLGR

SEQ ID NO: 364 - protein, 9 aa, Artificial Sequence
  Native Peptide Sequence to quantitate RAS-RAF-MAPK Pathway protein
MLVLDSDQR

SEQ ID NO: 365 - protein, 12 aa, Artificial Sequence
  Native Peptide Sequence to quantitate RAS-RAF-MAPK Pathway protein
LSRPFQSLIHAR

SEQ ID NO: 366 - protein, 10 aa, Artificial Sequence
  Native Peptide Sequence to quantitate RAS-RAF-MAPK Pathway protein
YDDSFDDVDR

SEQ ID NO: 367 - protein, 19 aa, Artificial Sequence
  Native Peptide Sequence to quantitate RAS-RAF-MAPK Pathway protein
DFASILTNASPLAVNLLEK

SEQ ID NO: 368 - protein, 26 aa, Artificial Sequence
  Native Peptide Sequence to quantitate RAS-RAF-MAPK Pathway protein
VTAGEALAHPYFESLHDTEDEPQVQK

SEQ ID NO: 369 - protein, 10 aa, Artificial Sequence
  Native Peptide Sequence to quantitate RAS-RAF-MAPK Pathway protein
YIHAAGIIHR

SEQ ID NO: 370 - protein, 12 aa, Artificial Sequence
  Native Peptide Sequence to quantitate RAS-RAF-MAPK Pathway protein
VTGTPPAEFVQR

SEQ ID NO: 371 - protein, 12 aa, Artificial Sequence
  Native Peptide Sequence to quantitate RAS-RAF-MAPK Pathway protein
LYRPFQSELFAK

SEQ ID NO: 372 - protein, 20 aa, Artificial Sequence
  Native Peptide Sequence to quantitate RAS-RAF-MAPK Pathway protein
  MOD_RES (14)..(14): carbamidomethylated cysteine
DLQPVGSGAYGAVCSAVDGR

SEQ ID NO: 373 - protein, 9 aa, Artificial Sequence
  Native Peptide Sequence to quantitate RAS-RAF-MAPK Pathway protein
EVLSFKPPR

SEQ ID NO: 374 - protein, 11 aa, Artificial Sequence
  Native Peptide Sequence to quantitate RAS-RAF-MAPK Pathway protein
ASPQAADLLEK

SEQ ID NO: 375 - protein, 11 aa, Artificial Sequence
  Native Peptide Sequence to quantitate RAS-RAF-MAPK Pathway protein
SYIQSLPQTPR

SEQ ID NO: 376 - protein, 12 aa, Artificial Sequence
  Native Peptide Sequence to quantitate RAS-RAF-MAPK Pathway protein
VTGVPGTEFVQK

SEQ ID NO: 377 - protein, 10 aa, Artificial Sequence
  Native Peptide Sequence to quantitate RAS-RAF-MAPK Pathway protein
YIHSAGVVHR

SEQ ID NO: 378 - protein, 22 aa, Artificial Sequence
  Native Peptide Sequence to quantitate RAS-RAF-MAPK Pathway protein
  MOD_RES (17)..(17): carbamidomethylated cysteine
TYVSPTHVGSGAYGSVCSAIDK

SEQ ID NO: 379 - protein, 11 aa, Artificial Sequence
  Native Peptide Sequence to quantitate RAS-RAF-MAPK Pathway protein
DYLDQLTQILK

SEQ ID NO: 380 - protein, 8 aa, Artificial Sequence
  Native Peptide Sequence to quantitate RAS-RAF-MAPK Pathway protein
DFTQLFPR

SEQ ID NO: 381 - protein, 7 aa, Artificial Sequence
  Native Peptide Sequence to quantitate RAS-RAF-MAPK Pathway protein
LTVDEWK

SEQ ID NO: 382 - protein, 16 aa, Artificial Sequence
  Native Peptide Sequence to quantitate RAS-RAF-MAPK Pathway protein
  MOD_RES (13)..(13): carbamidomethylated cysteine
DLKPSNLAVNEDCELK

SEQ ID NO: 383 - protein, 8 aa, Artificial Sequence
  Native Peptide Sequence to quantitate RAS-RAF-MAPK Pathway protein
DLLIDEWK

SEQ ID NO: 384 - protein, 15 aa, Artificial Sequence
  Native Peptide Sequence to quantitate RAS-RAF-MAPK Pathway protein
HENVIGLLDVFTPAR

SEQ ID NO: 385 - protein, 13 aa, Artificial Sequence
  Native Peptide Sequence to quantitate RAS-RAF-MAPK Pathway protein
TLFPGTDHIDQLK

SEQ ID NO: 386 - protein, 10 aa, Artificial Sequence
  Native Peptide Sequence to quantitate RAS-RAF-MAPK Pathway protein
YIHSADIIHR

SEQ ID NO: 387 - protein, 11 aa, Artificial Sequence
  Native Peptide Sequence to quantitate RAS-RAF-MAPK Pathway protein
LVGTPGAELLK

SEQ ID NO: 388 - protein, 15 aa, Artificial Sequence
  Native Peptide Sequence to quantitate RAS-RAF-MAPK Pathway protein
LTDDHVQFLIYQILR

SEQ ID NO: 389 - protein, 22 aa, Artificial Sequence
  Native Peptide Sequence to quantitate RAS-RAF-MAPK Pathway protein
  MOD_RES (16)..(16): carbamidomethylated cysteine
YQNLSPVGSGAYGSVCAAFDTK

SEQ ID NO: 390 - protein, 8 aa, Artificial Sequence
  Native Peptide Sequence to quantitate RAS-RAF-MAPK Pathway protein
TIWEVPER

SEQ ID NO: 391 - protein, 7 aa, Artificial Sequence
  Native Peptide Sequence to quantitate RAS-RAF-MAPK Pathway protein
ALDLLDR

SEQ ID NO: 392 - protein, 9 aa, Artificial Sequence
  Native Peptide Sequence to quantitate RAS-RAF-MAPK Pathway protein
NYLQSLPSK

SEQ ID NO: 393 - protein, 9 aa, Artificial Sequence
  Native Peptide Sequence to quantitate RAS-RAF-MAPK Pathway protein
GQPFDVGPR

SEQ ID NO: 394 - protein, 23 aa, Artificial Sequence
  Native Peptide Sequence to quantitate RAS-RAF-MAPK Pathway protein
YTQLQYIGEGAYGMVSSAYDHVR

SEQ ID NO: 395 - protein, 7 aa, Artificial Sequence
  Native Peptide Sequence to quantitate RAS-RAF-MAPK Pathway protein
EIQILLR

SEQ ID NO: 396 - protein, 16 aa, Artificial Sequence
  Native Peptide Sequence to quantitate RAS-RAF-MAPK Pathway protein
  MOD_RES (13)..(13): carbamidomethylated cysteine
DLKPSNLLINTTCDLK

SEQ ID NO: 397 - protein, 19 aa, Artificial Sequence
  Native Peptide Sequence to quantitate RAS-RAF-MAPK Pathway protein
IADPEHDHTGFLTEYVATR

SEQ ID NO: 398 - protein, 11 aa, Artificial Sequence
  Native Peptide Sequence to quantitate RAS-RAF-MAPK Pathway protein
LKELIFQETAR

SEQ ID NO: 399 - protein, 9 aa, Artificial Sequence
  Native Peptide Sequence to quantitate RAS-RAF-MAPK Pathway protein
ELIFQETAR

SEQ ID NO: 400 - protein, 9 aa, Artificial Sequence
  Native Peptide Sequence to quantitate RAS-RAF-MAPK Pathway protein
FQPGVLEAP

SEQ ID NO: 401 - protein, 19 aa, Artificial Sequence
  Native Peptide Sequence to quantitate RAS-RAF-MAPK Pathway protein
LGQYGFPNPEWSEVSEDAK

SEQ ID NO: 402 - protein, 17 aa, Artificial Sequence
  Native Peptide Sequence to quantitate RAS-RAF-MAPK Pathway protein
DIGTAIQFLHSHNIAHR

SEQ ID NO: 403 - protein, 25 aa, Artificial Sequence
  Native Peptide Sequence to quantitate RAS-RAF-MAPK Pathway protein
  MOD_RES (16)..(16): carbamidomethylated cysteine
YQNLKPIGSGAQGIVCAAYDAILER

SEQ ID NO: 404 - protein, 13 aa, Artificial Sequence
  Native Peptide Sequence to quantitate RAS-RAF-MAPK Pathway protein
NIIGLLNVFTPQK

SEQ ID NO: 405 - protein, 8 aa, Artificial Sequence
  Native Peptide Sequence to quantitate RAS-RAF-MAPK Pathway protein
YAGYSFEK

SEQ ID NO: 406 - protein, 15 aa, Artificial Sequence
  Native Peptide Sequence to quantitate RAS-RAF-MAPK Pathway protein
LFPDVLFPADSEHNK

SEQ ID NO: 407 - protein, 8 aa, Artificial Sequence
  Native Peptide Sequence to quantitate RAS-RAF-MAPK Pathway protein
EVMDLEER

SEQ ID NO: 408 - protein, 18 aa, Artificial Sequence
  Native Peptide Sequence to quantitate RAS-RAF-MAPK Pathway protein
DNNFYSVEIGDSTFTVLK

SEQ ID NO: 409 - protein, 8 aa, Artificial Sequence
  Native Peptide Sequence to quantitate RAS-RAF-MAPK Pathway protein
TYVENRPK

SEQ ID NO: 410 - protein, 27 aa, Artificial Sequence
  Native Peptide Sequence to quantitate RAS-RAF-MAPK Pathway protein
ISVDEALQHPYINVWYDPSEAEAPPPK

SEQ ID NO: 411 - protein, 19 aa, Artificial Sequence
  Native Peptide Sequence to quantitate RAS-RAF-MAPK Pathway protein
  MOD_RES (1)..(1): carbamidomethylated cysteine
CDSQFYSVQVADSTFTVLK

SEQ ID NO: 412 - protein, 20 aa, Artificial Sequence
  Native Peptide Sequence to quantitate RAS-RAF-MAPK Pathway protein
  MOD_RES (1)..(1): carbamidomethylated cysteine
CDSQFYSVQVADSTFTVLKR

SEQ ID NO: 413 - protein, 15 aa, Artificial Sequence
  Native Peptide Sequence to quantitate RAS-RAF-MAPK Pathway protein
  MOD_RES (3)..(3): carbamidomethylated cysteine
TACTNFMMTPYVVTR

SEQ ID NO: 414 - protein, 16 aa, Artificial Sequence
  Native Peptide Sequence to quantitate RAS-RAF-MAPK Pathway protein
  MOD_RES (2)..(2): carbamidomethylated cysteine
GCVIFQGTDHIDQWNK

SEQ ID NO: 415 - protein, 16 aa, Artificial Sequence
  Native Peptide Sequence to quantitate RAS-RAF-MAPK Pathway protein
FEELFPDWIFPSESER

SEQ ID NO: 416 - protein, 8 aa, Artificial Sequence
  Native Peptide Sequence to quantitate RAS-RAF-MAPK Pathway protein
ISVDEALR

SEQ ID NO: 417 - protein, 28 aa, Artificial Sequence
  Native Peptide Sequence to quantitate RAS-RAF-MAPK Pathway protein
HPYITVWYDPAEAEAPPPQIYDAQLEER

SEQ ID NO: 418 - protein, 14 aa, Artificial Sequence
  Native Peptide Sequence to quantitate RAS-RAF-MAPK Pathway protein
VIEQLGTPSAEFMK

SEQ ID NO: 419 - protein, 8 aa, Artificial Sequence
  Native Peptide Sequence to quantitate RAS-RAF-MAPK Pathway protein
EVMDWEER

SEQ ID NO: 420 - protein, 8 aa, Artificial Sequence
  Native Peptide Sequence to quantitate RAS-RAF-MAPK Pathway protein
EHAIEEWK

SEQ ID NO: 421 - protein, 17 aa, Artificial Sequence
  Native Peptide Sequence to quantitate RAS-RAF-MAPK Pathway protein
SIGEAIQYLHSINIAHR

SEQ ID NO: 422 - protein, 12 aa, Artificial Sequence
  Native Peptide Sequence to quantitate RAS-RAF-MAPK Pathway protein
IVDVYENLYAGR

SEQ ID NO: 423 - protein, 10 aa, Artificial Sequence
  Native Peptide Sequence to quantitate RAS-RAF-MAPK Pathway protein
VPQTPLHTSR

SEQ ID NO: 424 - protein, 27 aa, Artificial Sequence
  Native Peptide Sequence to quantitate RAS-RAF-MAPK Pathway protein
  MOD_RES (12)..(12): carbamidomethylated cysteine
ETTSHNSLTTPCYTPYYVAPEVLGPEK

SEQ ID NO: 425 - protein, 7 aa, Artificial Sequence
  Native Peptide Sequence to quantitate RAS-RAF-MAPK Pathway protein
VLQIFNK

SEQ ID NO: 426 - protein, 8 aa, Artificial Sequence
  Native Peptide Sequence to quantitate RAS-RAF-MAPK Pathway protein
NAIIDDYK

SEQ ID NO: 427 - protein, 9 aa, Artificial Sequence
  Native Peptide Sequence to quantitate RAS-RAF-MAPK Pathway protein
  MOD_RES (4)..(4): carbamidomethylated cysteine
ASQCPHIVR

SEQ ID NO: 428 - protein, 13 aa, Artificial Sequence
  Native Peptide Sequence to quantitate RAS-RAF-MAPK Pathway protein
VTSQVLGLGINGK

SEQ ID NO: 429 - protein, 19 aa, Artificial Sequence
  Native Peptide Sequence to quantitate RAS-RAF-MAPK Pathway protein
  MOD_RES (10)..(16): carbamidomethylated cysteine
TGPVLEHPDCFPCQDCSSK

SEQ ID NO: 430 - protein, 11 aa, Artificial Sequence
  Native Peptide Sequence to quantitate RAS-RAF-MAPK Pathway protein
ETLDAQTFHTR

SEQ ID NO: 431 - protein, 8 aa, Artificial Sequence
  Native Peptide Sequence to quantitate RAS-RAF-MAPK Pathway protein
YVNDFFNK

SEQ ID NO: 432 - protein, 9 aa, Artificial Sequence
  Native Peptide Sequence to quantitate RAS-RAF-MAPK Pathway protein
TEFTTALQR

SEQ ID NO: 433 - protein, 14 aa, Artificial Sequence
  Native Peptide Sequence to quantitate RAS-RAF-MAPK Pathway protein
GDLTIANLGTSEGR

SEQ ID NO: 434 - protein, 13 aa, Artificial Sequence
  Native Peptide Sequence to quantitate RAS-RAF-MAPK Pathway protein
VFPNSAPLEGGTR

SEQ ID NO: 435 - protein, 14 aa, Artificial Sequence
  Native Peptide Sequence to quantitate RAS-RAF-MAPK Pathway protein
SFISGGSTITGVGK

SEQ ID NO: 436 - protein, 9 aa, Artificial Sequence
  Native Peptide Sequence to quantitate RAS-RAF-MAPK Pathway protein
NLNSVSVPR

SEQ ID NO: 437 - protein, 8 aa, Artificial Sequence
  Native Peptide Sequence to quantitate RAS-RAF-MAPK Pathway protein
DLGSELVR

SEQ ID NO: 438 - protein, 11 aa, Artificial Sequence
  Native Peptide Sequence to quantitate RAS-RAF-MAPK Pathway protein
DLIGFGLQVAK

SEQ ID NO: 439 - protein, 8 aa, Artificial Sequence
  Native Peptide Sequence to quantitate RAS-RAF-MAPK Pathway protein
VADFGLAR

SEQ ID NO: 440 - protein, 20 aa, Artificial Sequence
  Native Peptide Sequence to quantitate RAS-RAF-MAPK Pathway protein
EVFNILQAAYVSKPGAQLAR

SEQ ID NO: 441 - protein, 12 aa, Artificial Sequence
  Native Peptide Sequence to quantitate RAS-RAF-MAPK Pathway protein
VAQLAVINSLEK

SEQ ID NO: 442 - protein, 21 aa, Artificial Sequence
  Native Peptide Sequence to quantitate RAS-RAF-MAPK Pathway protein
  MOD_RES (2)..(2): carbamidomethylated cysteine
ICLAQNSPSTFHYVLVNSLHR

SEQ ID NO: 443 - protein, 12 aa, Artificial Sequence
  Native Peptide Sequence to quantitate RAS-RAF-MAPK Pathway protein
  MOD_RES (6)..(6): carbamidomethylated cysteine
IDAVYCHSVELR

SEQ ID NO: 444 - protein, 7 aa, Artificial Sequence
  Native Peptide Sequence to quantitate RAS-RAF-MAPK Pathway protein
  MOD_RES (4)..(4): carbamidomethylated cysteine
LLLCNPR

SEQ ID NO: 445 - protein, 9 aa, Artificial Sequence
  Native Peptide Sequence to quantitate RAS-RAF-MAPK Pathway protein
  MOD_RES (3)..(3): carbamidomethylated cysteine
HLCEEADIR

SEQ ID NO: 446 - protein, 7 aa, Artificial Sequence
  Native Peptide Sequence to quantitate RAS-RAF-MAPK Pathway protein
LILNYPK

SEQ ID NO: 447 - protein, 7 aa, Artificial Sequence
  Native Peptide Sequence to quantitate RAS-RAF-MAPK Pathway protein
LLDPLLR

SEQ ID NO: 448 - protein, 17 aa, Artificial Sequence
  Native Peptide Sequence to quantitate RAS-RAF-MAPK Pathway protein
  MOD_RES (3)..(11): carbamidomethylated cysteine
SVCHCLYQATCHSLLNK

SEQ ID NO: 449 - protein, 12 aa, Artificial Sequence
  Native Peptide Sequence to quantitate RAS-RAF-MAPK Pathway protein
TLSIFYQAGTSK

SEQ ID NO: 450 - protein, 11 aa, Artificial Sequence
  Native Peptide Sequence to quantitate RAS-RAF-MAPK Pathway protein
AGNPIFYYVAR

SEQ ID NO: 451 - protein, 9 aa, Artificial Sequence
  Native Peptide Sequence to quantitate RAS-RAF-MAPK Pathway protein
  MOD_RES (6)..(6): carbamidomethylated cysteine
LVFIDCPGK

SEQ ID NO: 452 - protein, 13 aa, Artificial Sequence
  Native Peptide Sequence to quantitate RAS-RAF-MAPK Pathway protein
VGSTAVQVTSAER

SEQ ID NO: 453 - protein, 8 aa, Artificial Sequence
  Native Peptide Sequence to quantitate RAS-RAF-MAPK Pathway protein
SAAVIAFR

SEQ ID NO: 454 - protein, 12 aa, Artificial Sequence
  Native Peptide Sequence to quantitate RAS-RAF-MAPK Pathway protein
FAFQYNPSLQPR

SEQ ID NO: 455 - protein, 19 aa, Artificial Sequence
  Native Peptide Sequence to quantitate RAS-RAF-MAPK Pathway protein
GSEGYLAATYPTVGQTSPR

SEQ ID NO: 456 - protein, 11 aa, Artificial Sequence
  Native Peptide Sequence to quantitate RAS-RAF-MAPK Pathway protein
ISSSQQHPHLR

SEQ ID NO: 457 - protein, 12 aa, Artificial Sequence
  Native Peptide Sequence to quantitate RAS-RAF-MAPK Pathway protein
VFPVVHNLLDSK

SEQ ID NO: 458 - protein, 13 aa, Artificial Sequence
  Native Peptide Sequence to quantitate RAS-RAF-MAPK Pathway protein
  MOD_RES (5)..(5): carbamidomethylated cysteine
HGGTCHNEVGSYR

SEQ ID NO: 459 - protein, 12 aa, Artificial Sequence
  Native Peptide Sequence to quantitate RAS-RAF-MAPK Pathway protein
  MOD_RES (6)..(8): carbamidomethylated cysteine
VASFYCECPHGR

SEQ ID NO: 460 - protein, 23 aa, Artificial Sequence
  Native Peptide Sequence to quantitate RAS-RAF-MAPK Pathway protein
  MOD_RES (7)..(14): carbamidomethylated cysteine
GTQGVHCEINVDDCNPPVDPVSR

SEQ ID NO: 461 - protein, 14 aa, Artificial Sequence
  Native Peptide Sequence to quantitate RAS-RAF-MAPK Pathway protein
  MOD_RES (5)..(13): carbamidomethylated cysteine
AEGQCNPLYDQYCK

SEQ ID NO: 462 - protein, 16 aa, Artificial Sequence
  Native Peptide Sequence to quantitate RAS-RAF-MAPK Pathway protein
AAEGWAAPDALLGQVK

SEQ ID NO: 463 - protein, 17 aa, Artificial Sequence
  Native Peptide Sequence to quantitate RAS-RAF-MAPK Pathway protein
FEEPVVLPDLDDQTDHR

SEQ ID NO: 464 - protein, 11 aa, Artificial Sequence
  Native Peptide Sequence to quantitate RAS-RAF-MAPK Pathway protein
TGETALHLAAR

SEQ ID NO: 465 - protein, 9 aa, Artificial Sequence
  Native Peptide Sequence to quantitate RAS-RAF-MAPK Pathway protein
LLDEYNLVR

SEQ ID NO: 466 - protein, 15 aa, Artificial Sequence
  Native Peptide Sequence to quantitate RAS-RAF-MAPK Pathway protein
  MOD_RES (7)..(7): carbamidomethylated cysteine
TGEGFLCVFAINNSK

SEQ ID NO: 467 - protein, 9 aa, Artificial Sequence
  Native Peptide Sequence to quantitate RAS-RAF-MAPK Pathway protein
SFADINLYR

SEQ ID NO: 468 - protein, 7 aa, Artificial Sequence
  Native Peptide Sequence to quantitate RAS-RAF-MAPK Pathway protein
QAHELAK

SEQ ID NO: 469 - protein, 12 aa, Artificial Sequence
  Native Peptide Sequence to quantitate RAS-RAF-MAPK Pathway protein
QGVEDAFYTLVR

SEQ ID NO: 470 - protein, 19 aa, Artificial Sequence
  Native Peptide Sequence to quantitate RAS-RAF-MAPK Pathway protein
  MOD_RES (11)..(16): carbamidomethylated cysteine
LNSSDDGTQGCMGLPCVVM

SEQ ID NO: 471 - protein, 12 aa, Artificial Sequence
  Native Peptide Sequence to quantitate RAS-RAF-MAPK Pathway protein
SYGIPFIETSAK

SEQ ID NO: 472 - protein, 11 aa, Artificial Sequence
  Native Peptide Sequence to quantitate RAS-RAF-MAPK Pathway protein
NTSTMIGAGSK

SEQ ID NO: 473 - protein, 19 aa, Artificial Sequence
  Native Peptide Sequence to quantitate RAS-RAF-MAPK Pathway protein
DAGTLNHGSKPLPPNPEEK

SEQ ID NO: 474 - protein, 7 aa, Artificial Sequence
  Native Peptide Sequence to quantitate RAS-RAF-MAPK Pathway protein
SILPGDK

SEQ ID NO: 475 - protein, 14 aa, Artificial Sequence
  Native Peptide Sequence to quantitate RAS-RAF-MAPK Pathway protein
SAEDYNSSNALNVK

SEQ ID NO: 476 - protein, 12 aa, Artificial Sequence
  Native Peptide Sequence to quantitate RAS-RAF-MAPK Pathway protein
SVIEPLPVTPTR

SEQ ID NO: 477 - protein, 22 aa, Artificial Sequence
  Native Peptide Sequence to quantitate RAS-RAF-MAPK Pathway protein
DVATSPISPTENNTTPPDALTR

SEQ ID NO: 478 - protein, 24 aa, Artificial Sequence
  Native Peptide Sequence to quantitate RAS-RAF-MAPK Pathway protein
IGQGASGTVYTAMDVATGQEVAIK

SEQ ID NO: 479 - protein, 17 aa, Artificial Sequence
  Native Peptide Sequence to quantitate RAS-RAF-MAPK Pathway protein
  MOD_RES (2)..(2): carbamidomethylated cysteine
ECLQALEFLHSNQVIHR

SEQ ID NO: 480 - protein, 9 aa, Artificial Sequence
  Native Peptide Sequence to quantitate RAS-RAF-MAPK Pathway protein
ELLQHQFLK

SEQ ID NO: 481 - protein, 16 aa, Artificial Sequence
  Native Peptide Sequence to quantitate RAS-RAF-MAPK Pathway protein
IAKPLSSLTPLIAAAK

SEQ ID NO: 482 - protein, 13 aa, Artificial Sequence
  Native Peptide Sequence to quantitate RAS-RAF-MAPK Pathway protein
HPDVEVDGFSELR

SEQ ID NO: 483 - protein, 11 aa, Artificial Sequence
  Native Peptide Sequence to quantitate RAS-RAF-MAPK Pathway protein
TLGDFAAEYAK

SEQ ID NO: 484 - protein, 15 aa, Artificial Sequence
  Native Peptide Sequence to quantitate RAS-RAF-MAPK Pathway protein
EELGFRPEYSASQLK

SEQ ID NO: 485 - protein, 7 aa, Artificial Sequence
  Native Peptide Sequence to quantitate RAS-RAF-MAPK Pathway protein
ELLIFNK

SEQ ID NO: 486 - protein, 15 aa, Artificial Sequence
  Native Peptide Sequence to quantitate RAS-RAF-MAPK Pathway protein
  MOD_RES (6)..(6): carbamidomethylated cysteine
SDAYYCTGDVTAWTK

SEQ ID NO: 487 - protein, 15 aa, Artificial Sequence
  Native Peptide Sequence to quantitate RAS-RAF-MAPK Pathway protein
VVSEDFLQDVSASTK

SEQ ID NO: 488 - protein, 10 aa, Artificial Sequence
  Native Peptide Sequence to quantitate RAS-RAF-MAPK Pathway protein
AEPVEVVAPR

SEQ ID NO: 489 - protein, 13 aa, Artificial Sequence
  Native Peptide Sequence to quantitate RAS-RAF-MAPK Pathway protein
VFSATLGLVDIVK

SEQ ID NO: 490 - protein, 14 aa, Artificial Sequence
  Native Peptide Sequence to quantitate RAS-RAF-MAPK Pathway protein
KPPLLNNADSVQAK

SEQ ID NO: 491 - protein, 13 aa, Artificial Sequence
  Native Peptide Sequence to quantitate RAS-RAF-MAPK Pathway protein
TTNFAGILSQGLR

SEQ ID NO: 492 - protein, 16 aa, Artificial Sequence
  Native Peptide Sequence to quantitate RAS-RAF-MAPK Pathway protein
TQDQQSLSDVEGAYSR

SEQ ID NO: 493 - protein, 17 aa, Artificial Sequence
  Native Peptide Sequence to quantitate RAS-RAF-MAPK Pathway protein
  MOD_RES (16)..(16): carbamidomethylated cysteine
AEGAPPQQGPFAPPPCK

SEQ ID NO: 494 - protein, 11 aa, Artificial Sequence
  Native Peptide Sequence to quantitate RAS-RAF-MAPK Pathway protein
  MOD_RES (7)..(7): carbamidomethylated cysteine
APGASGCLLPR

SEQ ID NO: 495 - protein, 7 aa, Artificial Sequence
  Native Peptide Sequence to quantitate RAS-RAF-MAPK Pathway protein
  MOD_RES (3)..(3): carbamidomethylated cysteine
NDCIVDK

SEQ ID NO: 496 - protein, 10 aa, Artificial Sequence
  Native Peptide Sequence to quantitate RAS-RAF-MAPK Pathway protein
LLDNLHDLVK

SEQ ID NO: 497 - protein, 16 aa, Artificial Sequence
  Native Peptide Sequence to quantitate RAS-RAF-MAPK Pathway protein
  MOD_RES (5)..(5): carbamidomethylated cysteine
INHDCVPEQVIAEAIR

SEQ ID NO: 498 - protein, 9 aa, Artificial Sequence
  Native Peptide Sequence to quantitate RAS-RAF-MAPK Pathway protein
  MOD_RES (2)..(6): carbamidomethylated cysteine
LCLSICSVK

SEQ ID NO: 499 - protein, 9 aa, Artificial Sequence
  Native Peptide Sequence to quantitate RAS-RAF-MAPK Pathway protein
LINLTDILK

SEQ ID NO: 500 - protein, 18 aa, Artificial Sequence
  Native Peptide Sequence to quantitate RAS-RAF-MAPK Pathway protein
  MOD_RES (11)..(11): carbamidomethylated cysteine
TGIYHGGEPLCDNVNTQR

SEQ ID NO: 501 - protein, 9 aa, Artificial Sequence
  Native Peptide Sequence to quantitate RAS-RAF-MAPK Pathway protein
  MOD_RES (2)..(2): carbamidomethylated cysteine
LCVLEYQGK

SEQ ID NO: 502 - protein, 10 aa, Artificial Sequence
  Native Peptide Sequence to quantitate RAS-RAF-MAPK Pathway protein
SLWVINSALR

SEQ ID NO: 503 - protein, 14 aa, Artificial Sequence
  Native Peptide Sequence to quantitate RAS-RAF-MAPK Pathway protein
FSAASSDNTENLIK

SEQ ID NO: 504 - protein, 11 aa, Artificial Sequence
  Native Peptide Sequence to quantitate RAS-RAF-MAPK Pathway protein
  MOD_RES (6)..(6): carbamidomethylated cysteine
IFEEQCQTQER

SEQ ID NO: 505 - protein, 20 aa, Artificial Sequence
  Native Peptide Sequence to quantitate RAS-RAF-MAPK Pathway protein
YGFSDPLTFSSVVELINHYR

SEQ ID NO: 506 - protein, 11 aa, Artificial Sequence
  Native Peptide Sequence to quantitate RAS-RAF-MAPK Pathway protein
TQSSSNLAELR

SEQ ID NO: 507 - protein, 9 aa, Artificial Sequence
  Native Peptide Sequence to quantitate RAS-RAF-MAPK Pathway protein
TWNVGSSNR

SEQ ID NO: 508 - protein, 12 aa, Artificial Sequence
  Native Peptide Sequence to quantitate RAS-RAF-MAPK Pathway protein
ALGATFGPLLLR

SEQ ID NO: 509 - protein, 20 aa, Artificial Sequence
  Native Peptide Sequence to quantitate RAS-RAF-MAPK Pathway protein
SFLLALPAPLVTPEASAEAR

SEQ ID NO: 510 - protein, 14 aa, Artificial Sequence
  Native Peptide Sequence to quantitate RAS-RAF-MAPK Pathway protein
AALQALGVAEGGER

SEQ ID NO: 511 - protein, 21 aa, Artificial Sequence
  Native Peptide Sequence to quantitate RAS-RAF-MAPK Pathway protein
TDWSLSDVDQWDTAALADGIK

SEQ ID NO: 512 - protein, 11 aa, Artificial Sequence
  Native Peptide Sequence to quantitate RAS-RAF-MAPK Pathway protein
EYDQLYEEYTR

SEQ ID NO: 513 - protein, 10 aa, Artificial Sequence
  Native Peptide Sequence to quantitate RAS-RAF-MAPK Pathway protein
TSLEVSLLEK

SEQ ID NO: 514 - protein, 7 aa, Artificial Sequence
  Native Peptide Sequence to quantitate RAS-RAF-MAPK Pathway protein
  MOD_RES (6)..(6): carbamidomethylated cysteine
LWNEWCR

SEQ ID NO: 515 - protein, 20 aa, Artificial Sequence
  Native Peptide Sequence to quantitate RAS-RAF-MAPK Pathway protein
  MOD_RES (14)..(14): carbamidomethylated cysteine
TTDTTHLSSTEAFCVFYHLK

SEQ ID NO: 516 - protein, 7 aa, Artificial Sequence
  Native Peptide Sequence to quantitate RAS-RAF-MAPK Pathway protein
  MOD_RES (5)..(5): carbamidomethylated cysteine
VFGSCPR

SEQ ID NO: 517 - protein, 6 aa, Artificial Sequence
  Native Peptide Sequence to quantitate RAS-RAF-MAPK Pathway protein
HVTDYK

SEQ ID NO: 518 - protein, 14 aa, Artificial Sequence
  Native Peptide Sequence to quantitate RAS-RAF-MAPK Pathway protein
TSESTGSLPSPFLR

SEQ ID NO: 519 - protein, 16 aa, Artificial Sequence
  Native Peptide Sequence to quantitate RAS-RAF-MAPK Pathway protein
LDLITVEAFKPILSTR

SEQ ID NO: 520 - protein, 10 aa, Artificial Sequence
  Native Peptide Sequence to quantitate RAS-RAF-MAPK Pathway protein
NIPQSTEILK

SEQ ID NO: 521 - protein, 23 aa, Artificial Sequence
  Native Peptide Sequence to quantitate RAS-RAF-MAPK Pathway protein
YSTPHAFTFNTSSPSSEGSLSQR

SEQ ID NO: 522 - protein, 17 aa, Artificial Sequence
  Native Peptide Sequence to quantitate RAS-RAF-MAPK Pathway protein
  MOD_RES (10)..(10): carbamidomethylated cysteine
AAHTEDINACTLTTSPR

SEQ ID NO: 523 - protein, 14 aa, Artificial Sequence
  Native Peptide Sequence to quantitate RAS-RAF-MAPK Pathway protein
LLYPVAPPEPVEDR

SEQ ID NO: 524 - protein, 11 aa, Artificial Sequence
  Native Peptide Sequence to quantitate RAS-RAF-MAPK Pathway protein
VPDTDEISFLK

SEQ ID NO: 525 - protein, 13 aa, Artificial Sequence
  Native Peptide Sequence to quantitate RAS-RAF-MAPK Pathway protein
YYNSIGDIIDHYR

SEQ ID NO: 526 - protein, 17 aa, Artificial Sequence
  Native Peptide Sequence to quantitate RAS-RAF-MAPK Pathway protein
GHATDEWFLLSSHIPLK

SEQ ID NO: 527 - protein, 9 aa, Artificial Sequence
  Native Peptide Sequence to quantitate RAS-RAF-MAPK Pathway protein
VVSGFVFLR

SEQ ID NO: 528 - protein, 10 aa, Artificial Sequence
  Native Peptide Sequence to quantitate RAS-RAF-MAPK Pathway protein
  MOD_RES (3)..(3): carbamidomethylated cysteine
LICPAILNPR

SEQ ID NO: 529 - protein, 17 aa, Artificial Sequence
  Native Peptide Sequence to quantitate RAS-RAF-MAPK Pathway protein
  MOD_RES (9)..(9): carbamidomethylated cysteine
DLAALHEICVAHSDELR

SEQ ID NO: 530 - protein, 11 aa, Artificial Sequence
  Native Peptide Sequence to quantitate RAS-RAF-MAPK Pathway protein
LLAITELLQQK

SEQ ID NO: 531 - protein, 12 aa, Artificial Sequence
  Native Peptide Sequence to quantitate RAS-RAF-MAPK Pathway protein
YLHLLQIVETEK

SEQ ID NO: 532 - protein, 8 aa, Artificial Sequence
  Native Peptide Sequence to quantitate RAS-RAF-MAPK Pathway protein
AEITSLLK

SEQ ID NO: 533 - protein, 8 aa, Artificial Sequence
  Native Peptide Sequence to quantitate RAS-RAF-MAPK Pathway protein
TIIQDYIR

SEQ ID NO: 534 - protein, 7 aa, Artificial Sequence
  Native Peptide Sequence to quantitate RAS-RAF-MAPK Pathway protein
  MOD_RES (5)..(5): carbamidomethylated cysteine
HLIICTR

SEQ ID NO: 535 - protein, 15 aa, Artificial Sequence
  Native Peptide Sequence to quantitate RAS-RAF-MAPK Pathway protein
  MOD_RES (10)..(10): carbamidomethylated cysteine
AAWTSDISQCVDNIR

SEQ ID NO: 536 - protein, 13 aa, Artificial Sequence
  Native Peptide Sequence to quantitate RAS-RAF-MAPK Pathway protein
  MOD_RES (7)..(7): carbamidomethylated cysteine
SDASLYCDDVDIR

SEQ ID NO: 537 - protein, 9 aa, Artificial Sequence
  Native Peptide Sequence to quantitate RAS-RAF-MAPK Pathway protein
KPISAIPAR

SEQ ID NO: 538 - protein, 20 aa, Artificial Sequence
  Native Peptide Sequence to quantitate RAS-RAF-MAPK Pathway protein
IPDEGDTTPEKPEDPSALSK

SEQ ID NO: 539 - protein, 23 aa, Artificial Sequence
  Native Peptide Sequence to quantitate RAS-RAF-MAPK Pathway protein
SALSAASAFAIATAGANEGTPNK

SEQ ID NO: 540 - protein, 25 aa, Artificial Sequence
  Native Peptide Sequence to quantitate RAS-RAF-MAPK Pathway protein
SALSAASAFAIATAGANEGTPNKEK

SEQ ID NO: 541 - protein, 12 aa, Artificial Sequence
  Native Peptide Sequence to quantitate RAS-RAF-MAPK Pathway protein
HSQDFETNDELK

SEQ ID NO: 542 - protein, 14 aa, Artificial Sequence
  Native Peptide Sequence to quantitate RAS-RAF-MAPK Pathway protein
  MOD_RES (13)..(13): carbamidomethylated cysteine
HSQDFETNDELKCK

SEQ ID NO: 543 - protein, 12 aa, Artificial Sequence
  Native Peptide Sequence to quantitate RAS-RAF-MAPK Pathway protein
YIHLVQIVETEK

SEQ ID NO: 544 - protein, 12 aa, Artificial Sequence
  Native Peptide Sequence to quantitate RAS-RAF-MAPK Pathway protein
HQLEDQDTEIER

SEQ ID NO: 545 - protein, 7 aa, Artificial Sequence
  Native Peptide Sequence to quantitate RAS-RAF-MAPK Pathway protein
  MOD_RES (5)..(5): carbamidomethylated cysteine
HFLICTR

SEQ ID NO: 546 - protein, 15 aa, Artificial Sequence
  Native Peptide Sequence to quantitate RAS-RAF-MAPK Pathway protein
  MOD_RES (10)..(10): carbamidomethylated cysteine
AAWMSDISQCVDNIR

SEQ ID NO: 547 - protein, 15 aa, Artificial Sequence
  Native Peptide Sequence to quantitate RAS-RAF-MAPK Pathway protein
FLSIDFLNTFLHTYR

SEQ ID NO: 548 - protein, 11 aa, Artificial Sequence
  Native Peptide Sequence to quantitate RAS-RAF-MAPK Pathway protein
IFTTAAVVLGK

SEQ ID NO: 549 - protein, 11 aa, Artificial Sequence
  Native Peptide Sequence to quantitate RAS-RAF-MAPK Pathway protein
FSSPPPLAVSR

SEQ ID NO: 550 - protein, 20 aa, Artificial Sequence
  Native Peptide Sequence to quantitate RAS-RAF-MAPK Pathway protein
GLSSPEQSPGTVEENVDNPR

SEQ ID NO: 551 - protein, 10 aa, Artificial Sequence
  Native Peptide Sequence to quantitate RAS-RAF-MAPK Pathway protein
AVLESAPADR

SEQ ID NO: 552 - protein, 18 aa, Artificial Sequence
  Native Peptide Sequence to quantitate RAS-RAF-MAPK Pathway protein
  MOD_RES (17)..(17): carbamidomethylated cysteine
AGVESSPAADTTELSPCR

SEQ ID NO: 553 - protein, 12 aa, Artificial Sequence
  Native Peptide Sequence to quantitate RAS-RAF-MAPK Pathway protein
HAQDFELNNELK

SEQ ID NO: 554 - protein, 13 aa, Artificial Sequence
  Native Peptide Sequence to quantitate RAS-RAF-MAPK Pathway protein
ALSQDDQDDIHLK

SEQ ID NO: 555 - protein, 14 aa, Artificial Sequence
  Native Peptide Sequence to quantitate RAS-RAF-MAPK Pathway protein
SIPYEEFLGQGWMK

SEQ ID NO: 556 - protein, 8 aa, Artificial Sequence
  Native Peptide Sequence to quantitate RAS-RAF-MAPK Pathway protein
EILQNVAR

SEQ ID NO: 557 - protein, 11 aa, Artificial Sequence
  Native Peptide Sequence to quantitate RAS-RAF-MAPK Pathway protein
  MOD_RES (3)..(3): carbamidomethylated cysteine
LLCDIGHSEEK

SEQ ID NO: 558 - protein, 8 aa, Artificial Sequence
  Native Peptide Sequence to quantitate RAS-RAF-MAPK Pathway protein
LALLNEAK

SEQ ID NO: 559 - protein, 11 aa, Artificial Sequence
  Native Peptide Sequence to quantitate RAS-RAF-MAPK Pathway protein
YLIQDSSILQK

SEQ ID NO: 560 - protein, 11 aa, Artificial Sequence
  Native Peptide Sequence to quantitate RAS-RAF-MAPK Pathway protein
ILAPYTDFHYR

SEQ ID NO: 561 - protein, 11 aa, Artificial Sequence
  Native Peptide Sequence to quantitate RAS-RAF-MAPK Pathway protein
LSDGFVAAEAK

SEQ ID NO: 562 - protein, 11 aa, Artificial Sequence
  Native Peptide Sequence to quantitate RAS-RAF-MAPK Pathway protein
LYANLDLDFAK

SEQ ID NO: 563 - protein, 10 aa, Artificial Sequence
  Native Peptide Sequence to quantitate RAS-RAF-MAPK Pathway protein
LTVSSLDYSR

SEQ ID NO: 564 - protein, 10 aa, Artificial Sequence
  Native Peptide Sequence to quantitate RAS-RAF-MAPK Pathway protein
  MOD_RES (9)..(9): carbamidomethylated cysteine
ILTAATDACR

SEQ ID NO: 565 - protein, 17 aa, Artificial Sequence
  Native Peptide Sequence to quantitate RAS-RAF-MAPK Pathway protein
  MOD_RES (3)..(16): carbamidomethylated cysteine
TGCHLLEVQNIITELCR

SEQ ID NO: 566 - protein, 17 aa, Artificial Sequence
  Native Peptide Sequence to quantitate RAS-RAF-MAPK Pathway protein
HIEDTGSTPSIGENDLK

SEQ ID NO: 567 - protein, 16 aa, Artificial Sequence
  Native Peptide Sequence to quantitate RAS-RAF-MAPK Pathway protein
TSHYLTPQSNHLSLSK

SEQ ID NO: 568 - protein, 17 aa, Artificial Sequence
  Native Peptide Sequence to quantitate RAS-RAF-MAPK Pathway protein
SNSVSLVPPGSSHTLPR

SEQ ID NO: 569 - protein, 8 aa, Artificial Sequence
  Native Peptide Sequence to quantitate RAS-RAF-MAPK Pathway protein
DIPYFQTK

SEQ ID NO: 570 - protein, 18 aa, Artificial Sequence
  Native Peptide Sequence to quantitate RAS-RAF-MAPK Pathway protein
AFAHDAGGLPSGTGGLVK

SEQ ID NO: 571 - protein, 9 aa, Artificial Sequence
  Native Peptide Sequence to quantitate RAS-RAF-MAPK Pathway protein
STELLLGVK

SEQ ID NO: 572 - protein, 9 aa, Artificial Sequence
  Native Peptide Sequence to quantitate RAS-RAF-MAPK Pathway protein
EISITHHVK

SEQ ID NO: 573 - protein, 13 aa, Artificial Sequence
  Native Peptide Sequence to quantitate RAS-RAF-MAPK Pathway protein
DILADVNHPFVVK

SEQ ID NO: 574 - protein, 11 aa, Artificial Sequence
  Native Peptide Sequence to quantitate RAS-RAF-MAPK Pathway protein
HVFYSTIDWNK

SEQ ID NO: 575 - protein, 17 aa, Artificial Sequence
  Native Peptide Sequence to quantitate RAS-RAF-MAPK Pathway protein
GFSFVATGLMEDDGKPR

SEQ ID NO: 576 - protein, 16 aa, Artificial Sequence
  Native Peptide Sequence to quantitate RAS-RAF-MAPK Pathway protein
APQAPLHSVVQQLHGK

SEQ ID NO: 577 - protein, 11 aa, Artificial Sequence
  Native Peptide Sequence to quantitate RAS-RAF-MAPK Pathway protein
NLVFSDGYVVK

SEQ ID NO: 578 - protein, 9 aa, Artificial Sequence
  Native Peptide Sequence to quantitate RAS-RAF-MAPK Pathway protein
ATNMEYAVK

SEQ ID NO: 579 - protein, 11 aa, Artificial Sequence
  Native Peptide Sequence to quantitate RAS-RAF-MAPK Pathway protein
EASFVLHTIGK

SEQ ID NO: 580 - protein, 13 aa, Artificial Sequence
  Native Peptide Sequence to quantitate RAS-RAF-MAPK Pathway protein
TVEYLHSQGVVHR

SEQ ID NO: 581 - protein, 20 aa, Artificial Sequence
  Native Peptide Sequence to quantitate RAS-RAF-MAPK Pathway protein
  MOD_RES (18)..(18): carbamidomethylated cysteine
DLKPSNILYVDESGNPECLR

SEQ ID NO: 582 - protein, 9 aa, Artificial Sequence
  Native Peptide Sequence to quantitate RAS-RAF-MAPK Pathway protein
VLGQGSYGK

SEQ ID NO: 583 - protein, 13 aa, Artificial Sequence
  Native Peptide Sequence to quantitate RAS-RAF-MAPK Pathway protein
DILAEVNHPFIVK

SEQ ID NO: 584 - protein, 12 aa, Artificial Sequence
  Native Peptide Sequence to quantitate RAS-RAF-MAPK Pathway protein
LGAGIDGVEEIK

SEQ ID NO: 585 - protein, 13 aa, Artificial Sequence
  Native Peptide Sequence to quantitate RAS-RAF-MAPK Pathway protein
LGAGIDGVEEIKR

SEQ ID NO: 586 - protein, 14 aa, Artificial Sequence
  Native Peptide Sequence to quantitate RAS-RAF-MAPK Pathway protein
HPFFVTIDWNTLYR

SEQ ID NO: 587 - protein, 19 aa, Artificial Sequence
  Native Peptide Sequence to quantitate RAS-RAF-MAPK Pathway protein
TPTDSPGVPPSANAHHLFR

SEQ ID NO: 588 - protein, 9 aa, Artificial Sequence
  Native Peptide Sequence to quantitate RAS-RAF-MAPK Pathway protein
ATDTEYAVK

SEQ ID NO: 589 - protein, 11 aa, Artificial Sequence
  Native Peptide Sequence to quantitate RAS-RAF-MAPK Pathway protein
  MOD_RES (7)..(7): carbamidomethylated cysteine
EASDVLCTITK

SEQ ID NO: 590 - protein, 16 aa, Artificial Sequence
  Native Peptide Sequence to quantitate RAS-RAF-MAPK Pathway protein
YALSGGNWDSISDAAK

SEQ ID NO: 591 - protein, 12 aa, Artificial Sequence
  Native Peptide Sequence to quantitate RAS-RAF-MAPK Pathway protein
LEPVLSSNLAQR

SEQ ID NO: 592 - protein, 11 aa, Artificial Sequence
  Native Peptide Sequence to quantitate RAS-RAF-MAPK Pathway protein
HSFFSTIDWNK

SEQ ID NO: 593 - protein, 26 aa, Artificial Sequence
  Native Peptide Sequence to quantitate RAS-RAF-MAPK Pathway protein
EIHPPFKPATGRPEDTFYFDPEFTAK

SEQ ID NO: 594 - protein, 16 aa, Artificial Sequence
  Native Peptide Sequence to quantitate RAS-RAF-MAPK Pathway protein
DSPGIPPSANAHQLFR

SEQ ID NO: 595 - protein, 12 aa, Artificial Sequence
  Native Peptide Sequence to quantitate RAS-RAF-MAPK Pathway protein
NSIQFTDGYEVK

SEQ ID NO: 596 - protein, 9 aa, Artificial Sequence
  Native Peptide Sequence to quantitate RAS-RAF-MAPK Pathway protein
ATNMEFAVK

SEQ ID NO: 597 - protein, 11 aa, Artificial Sequence
  Native Peptide Sequence to quantitate RAS-RAF-MAPK Pathway protein
EASAVLFTITK

SEQ ID NO: 598 - protein, 13 aa, Artificial Sequence
  Native Peptide Sequence to quantitate RAS-RAF-MAPK Pathway protein
TVEYLHAQGVVHR

SEQ ID NO: 599 - protein, 20 aa, Artificial Sequence
  Native Peptide Sequence to quantitate RAS-RAF-MAPK Pathway protein
DLKPSNILYVDESGNPESIR

SEQ ID NO: 600 - protein, 8 aa, Artificial Sequence
  Native Peptide Sequence to quantitate RAS-RAF-MAPK Pathway protein
LTAALVLR

SEQ ID NO: 601 - protein, 17 aa, Artificial Sequence
  Native Peptide Sequence to quantitate RAS-RAF-MAPK Pathway protein
HPWIVHWDQLPQYQLNR

SEQ ID NO: 602 - protein, 12 aa, Artificial Sequence
  Native Peptide Sequence to quantitate RAS-RAF-MAPK Pathway protein
GAMAATYSALNR

SEQ ID NO: 603 - protein, 11 aa, Artificial Sequence
  Native Peptide Sequence to quantitate RAS-RAF-MAPK Pathway protein
NQSPVLEPVGR

SEQ ID NO: 604 - protein, 10 aa, Artificial Sequence
  Native Peptide Sequence to quantitate RAS-RAF-MAPK Pathway protein
VGIENFELLK

SEQ ID NO: 605 - protein, 19 aa, Artificial Sequence
  Native Peptide Sequence to quantitate RAS-RAF-MAPK Pathway protein
DLKPENLLFTDENDNLEIK

SEQ ID NO: 606 - protein, 9 aa, Artificial Sequence
  Native Peptide Sequence to quantitate RAS-RAF-MAPK Pathway protein
EFVADETER

SEQ ID NO: 607 - protein, 13 aa, Artificial Sequence
  Native Peptide Sequence to quantitate RAS-RAF-MAPK Pathway protein
DSPFYQHYDLDLK

SEQ ID NO: 608 - protein, 10 aa, Artificial Sequence
  Native Peptide Sequence to quantitate RAS-RAF-MAPK Pathway protein
  MOD_RES (2)..(2): carbamidomethylated cysteine
LCEGHPNIVK

SEQ ID NO: 609 - protein, 13 aa, Artificial Sequence
  Native Peptide Sequence to quantitate RAS-RAF-MAPK Pathway protein
  MOD_RES (12)..(12): carbamidomethylated cysteine
DKPLGEGSFSICR

SEQ ID NO: 610 - protein, 13 aa, Artificial Sequence
  Native Peptide Sequence to quantitate RAS-RAF-MAPK Pathway protein
DLIQGLLTVDPNK

SEQ ID NO: 611 - protein, 26 aa, Artificial Sequence
  Native Peptide Sequence to quantitate RAS-RAF-MAPK Pathway protein
AAGLEPVGHYEEVELTETSVNVGPER

SEQ ID NO: 612 - protein, 10 aa, Artificial Sequence
  Native Peptide Sequence to quantitate RAS-RAF-MAPK Pathway protein
  MOD_RES (5)..(5): carbamidomethylated cysteine
IGPHCFELLR

SEQ ID NO: 613 - protein, 7 aa, Artificial Sequence
  Native Peptide Sequence to quantitate RAS-RAF-MAPK Pathway protein
NILESVK

SEQ ID NO: 614 - protein, 16 aa, Artificial Sequence
  Native Peptide Sequence to quantitate RAS-RAF-MAPK Pathway protein
HPFIVELAYAFQTGGK

SEQ ID NO: 615 - protein, 13 aa, Artificial Sequence
  Native Peptide Sequence to quantitate RAS-RAF-MAPK Pathway protein
IGGGPGDAADVQR

SEQ ID NO: 616 - protein, 21 aa, Artificial Sequence
  Native Peptide Sequence to quantitate RAS-RAF-MAPK Pathway protein
  MOD_RES (8)..(8): carbamidomethylated cysteine
VDPPFRPCLQSEEDVSQFDTR

SEQ ID NO: 617 - protein, 8 aa, Artificial Sequence
  Native Peptide Sequence to quantitate RAS-RAF-MAPK Pathway protein
EGFSFQPK

SEQ ID NO: 618 - protein, 11 aa, Artificial Sequence
  Native Peptide Sequence to quantitate RAS-RAF-MAPK Pathway protein
ALETIGANLQK

SEQ ID NO: 619 - protein, 9 aa, Artificial Sequence
  Native Peptide Sequence to quantitate RAS-RAF-MAPK Pathway protein
IWQNFDSAR

SEQ ID NO: 620 - protein, 13 aa, Artificial Sequence
  Native Peptide Sequence to quantitate RAS-RAF-MAPK Pathway protein
LYSLLSDPIPEVR

SEQ ID NO: 621 - protein, 9 aa, Artificial Sequence
  Native Peptide Sequence to quantitate RAS-RAF-MAPK Pathway protein
SVSSYGNIR

SEQ ID NO: 622 - protein, 18 aa, Artificial Sequence
  Native Peptide Sequence to quantitate RAS-RAF-MAPK Pathway protein
VLDTSSLTQSAPASPTNK

SEQ ID NO: 623 - protein, 9 aa, Artificial Sequence
  Native Peptide Sequence to quantitate RAS-RAF-MAPK Pathway protein
LDDQIFLNR

SEQ ID NO: 624 - protein, 21 aa, Artificial Sequence
  Native Peptide Sequence to quantitate RAS-RAF-MAPK Pathway protein
  MOD_RES (11)..(17): carbamidomethylated cysteine
VQDIPTGADSCVTSLSCDSHR

SEQ ID NO: 625 - protein, 13 aa, Artificial Sequence
  Native Peptide Sequence to quantitate RAS-RAF-MAPK Pathway protein
SLIVAGLGDGSIR

SEQ ID NO: 626 - protein, 9 aa, Artificial Sequence
  Native Peptide Sequence to quantitate RAS-RAF-MAPK Pathway protein
  MOD_RES (2)..(2): carbamidomethylated cysteine
YCQYAFDLK

SEQ ID NO: 627 - protein, 13 aa, Artificial Sequence
  Native Peptide Sequence to quantitate RAS-RAF-MAPK Pathway protein
  MOD_RES (1)..(5): carbamidomethylated cysteine
CDSVCVNPYHYER

SEQ ID NO: 628 - protein, 11 aa, Artificial Sequence
  Native Peptide Sequence to quantitate RAS-RAF-MAPK Pathway protein
  MOD_RES (2)..(2): carbamidomethylated cysteine
FCLGQLSNVHR

SEQ ID NO: 629 - protein, 8 aa, Artificial Sequence
  Native Peptide Sequence to quantitate RAS-RAF-MAPK Pathway protein
GEGDVWVR

SEQ ID NO: 630 - protein, 16 aa, Artificial Sequence
  Native Peptide Sequence to quantitate RAS-RAF-MAPK Pathway protein
  MOD_RES (1)..(1): carbamidomethylated cysteine
CLSDHAVFVQSYYLDR

SEQ ID NO: 631 - protein, 8 aa, Artificial Sequence
  Native Peptide Sequence to quantitate RAS-RAF-MAPK Pathway protein
IYPSAYIK

SEQ ID NO: 632 - protein, 8 aa, Artificial Sequence
  Native Peptide Sequence to quantitate RAS-RAF-MAPK Pathway protein
GWGPDYPR

SEQ ID NO: 633 - protein, 15 aa, Artificial Sequence
  Native Peptide Sequence to quantitate RAS-RAF-MAPK Pathway protein
IAIENGDQPLSAELK

SEQ ID NO: 634 - protein, 13 aa, Artificial Sequence
  Native Peptide Sequence to quantitate RAS-RAF-MAPK Pathway protein
LFSANDVENIFSR

SEQ ID NO: 635 - protein, 10 aa, Artificial Sequence
  Native Peptide Sequence to quantitate RAS-RAF-MAPK Pathway protein
IVDIHELSVK

SEQ ID NO: 636 - protein, 11 aa, Artificial Sequence
  Native Peptide Sequence to quantitate RAS-RAF-MAPK Pathway protein
LDHTFEQIPSR

SEQ ID NO: 637 - protein, 9 aa, Artificial Sequence
  Native Peptide Sequence to quantitate RAS-RAF-MAPK Pathway protein
VTPTFHHWK

SEQ ID NO: 638 - protein, 13 aa, Artificial Sequence
  Native Peptide Sequence to quantitate RAS-RAF-MAPK Pathway protein
  MOD_RES (9)..(9): carbamidomethylated cysteine
AIEDISQGCPESK

SEQ ID NO: 639 - protein, 22 aa, Artificial Sequence
  Native Peptide Sequence to quantitate RAS-RAF-MAPK Pathway protein
NEAEGADDLQANEEDSSSSLVK

SEQ ID NO: 640 - protein, 16 aa, Artificial Sequence
  Native Peptide Sequence to quantitate RAS-RAF-MAPK Pathway protein
DHLFQQETVVTSEPYR

SEQ ID NO: 641 - protein, 15 aa, Artificial Sequence
  Native Peptide Sequence to quantitate RAS-RAF-MAPK Pathway protein
SSNIRPSPFEDLNAR

SEQ ID NO: 642 - protein, 12 aa, Artificial Sequence
  Native Peptide Sequence to quantitate RAS-RAF-MAPK Pathway protein
HVSFQDEDEIVR

SEQ ID NO: 643 - protein, 7 aa, Artificial Sequence
  Native Peptide Sequence to quantitate RAS-RAF-MAPK Pathway protein
TQPSSLK

SEQ ID NO: 644 - protein, 8 aa, Artificial Sequence
  Native Peptide Sequence to quantitate RAS-RAF-MAPK Pathway protein
FNHEENVR

SEQ ID NO: 645 - protein, 17 aa, Artificial Sequence
  Native Peptide Sequence to quantitate RAS-RAF-MAPK Pathway protein
DDSSGGWFPQEGGGISR

SEQ ID NO: 646 - protein, 9 aa, Artificial Sequence
  Native Peptide Sequence to quantitate RAS-RAF-MAPK Pathway protein
  MOD_RES (6)..(6): carbamidomethylated cysteine
LVVLECYVR

SEQ ID NO: 647 - protein, 11 aa, Artificial Sequence
  Native Peptide Sequence to quantitate RAS-RAF-MAPK Pathway protein
  MOD_RES (8)..(8): carbamidomethylated cysteine
TISSPTSCEHR

SEQ ID NO: 648 - protein, 12 aa, Artificial Sequence
  Native Peptide Sequence to quantitate RAS-RAF-MAPK Pathway protein
EKIWMTGYEDYR

SEQ ID NO: 649 - protein, 10 aa, Artificial Sequence
  Native Peptide Sequence to quantitate RAS-RAF-MAPK Pathway protein
IWMTGYEDYR

SEQ ID NO: 650 - protein, 16 aa, Artificial Sequence
  Native Peptide Sequence to quantitate RAS-RAF-MAPK Pathway protein
GKYPDPSEDADSSYVR

SEQ ID NO: 651 - protein, 14 aa, Artificial Sequence
  Native Peptide Sequence to quantitate RAS-RAF-MAPK Pathway protein
YPDPSEDADSSYVR

SEQ ID NO: 652 - protein, 20 aa, Artificial Sequence
  Native Peptide Sequence to quantitate RAS-RAF-MAPK Pathway protein
HDYNYPYVDSSDFGLGEDPK

SEQ ID NO: 653 - protein, 11 aa, Artificial Sequence
  Native Peptide Sequence to quantitate RAS-RAF-MAPK Pathway protein
  MOD_RES (3)..(3): carbamidomethylated cysteine
GHCQDAPDSVR

SEQ ID NO: 654 - protein, 14 aa, Artificial Sequence
  Native Peptide Sequence to quantitate RAS-RAF-MAPK Pathway protein
EIQPTAILSLDQIK

SEQ ID NO: 655 - protein, 13 aa, Artificial Sequence
  Native Peptide Sequence to quantitate RAS-RAF-MAPK Pathway protein
GSNEYTEGPSVVK

SEQ ID NO: 656 - protein, 15 aa, Artificial Sequence
  Native Peptide Sequence to quantitate RAS-RAF-MAPK Pathway protein
HTSHLGHAVLPSNAR

SEQ ID NO: 657 - protein, 8 aa, Artificial Sequence
  Native Peptide Sequence to quantitate RAS-RAF-MAPK Pathway protein
  MOD_RES (3)..(6): carbamidomethylated cysteine
FICEQCGK

SEQ ID NO: 658 - protein, 11 aa, Artificial Sequence
  Native Peptide Sequence to quantitate RAS-RAF-MAPK Pathway protein
SPPTRPVPGHR

SEQ ID NO: 659 - protein, 16 aa, Artificial Sequence
  Native Peptide Sequence to quantitate RAS-RAF-MAPK Pathway protein
DALTQQVHVLSLDQIR

SEQ ID NO: 660 - protein, 8 aa, Artificial Sequence
  Native Peptide Sequence to quantitate RAS-RAF-MAPK Pathway protein
LHGLPEHR

SEQ ID NO: 661 - protein, 17 aa, Artificial Sequence
  Native Peptide Sequence to quantitate RAS-RAF-MAPK Pathway protein
LLGSSFSSGPVADGIIR

SEQ ID NO: 662 - protein, 9 aa, Artificial Sequence
  Native Peptide Sequence to quantitate RAS-RAF-MAPK Pathway protein
EDLGLHAYR

SEQ ID NO: 663 - protein, 13 aa, Artificial Sequence
  Native Peptide Sequence to quantitate RAS-RAF-MAPK Pathway protein
SELKPGELKPLSK

SEQ ID NO: 664 - protein, 11 aa, Artificial Sequence
  Native Peptide Sequence to quantitate RAS-RAF-MAPK Pathway protein
LQHSQVHSSAR

SEQ ID NO: 665 - protein, 17 aa, Artificial Sequence
  Native Peptide Sequence to quantitate RAS-RAF-MAPK Pathway protein
  MOD_RES (16)..(16): carbamidomethylated cysteine
STHASNDYVERPPAPCK

SEQ ID NO: 666 - protein, 16 aa, Artificial Sequence
  Native Peptide Sequence to quantitate RAS-RAF-MAPK Pathway protein
LLASITPSPSGQSIIR

SEQ ID NO: 667 - protein, 14 aa, Artificial Sequence
  Native Peptide Sequence to quantitate RAS-RAF-MAPK Pathway protein
QALSSPSLIVQTHK

SEQ ID NO: 668 - protein, 19 aa, Artificial Sequence
  Native Peptide Sequence to quantitate RAS-RAF-MAPK Pathway protein
  MOD_RES (1)..(17): carbamidomethylated cysteine
CLCSAESLLDYGTCLCCVK

SEQ ID NO: 669 - protein, 14 aa, Artificial Sequence
  Native Peptide Sequence to quantitate RAS-RAF-MAPK Pathway protein
LQHPLTILPIDQVK

SEQ ID NO: 670 - protein, 21 aa, Artificial Sequence
  Native Peptide Sequence to quantitate RAS-RAF-MAPK Pathway protein
TSHVENDYIDNPSLALTTGPK

SEQ ID NO: 671 - protein, 14 aa, Artificial Sequence
  Native Peptide Sequence to quantitate RAS-RAF-MAPK Pathway protein
TRGGAPELAPTPAR

SEQ ID NO: 672 - protein, 12 aa, Artificial Sequence
  Native Peptide Sequence to quantitate RAS-RAF-MAPK Pathway protein
GGAPELAPTPAR

SEQ ID NO: 673 - protein, 30 aa, Artificial Sequence
  Native Peptide Sequence to quantitate RAS-RAF-MAPK Pathway protein
  MOD_RES (1)..(1): carbamidomethylated cysteine
CDQDVTHHWISFSGRPSSVSSSSSTSSDQR

SEQ ID NO: 674 - protein, 17 aa, Artificial Sequence
  Native Peptide Sequence to quantitate RAS-RAF-MAPK Pathway protein
LLDHMAPPPVADQASPR

SEQ ID NO: 675 - protein, 10 aa, Artificial Sequence
  Native Peptide Sequence to quantitate RAS-RAF-MAPK Pathway protein
  MOD_RES (4)..(4): carbamidomethylated cysteine
VVHCQPLDLK

SEQ ID NO: 676 - protein, 10 aa, Artificial Sequence
  Native Peptide Sequence to quantitate RAS-RAF-MAPK Pathway protein
  MOD_RES (5)..(8): carbamidomethylated cysteine
HFLLCEACGK

SEQ ID NO: 677 - protein, 8 aa, Artificial Sequence
  Native Peptide Sequence to quantitate RAS-RAF-MAPK Pathway protein
  MOD_RES (7)..(7): carbamidomethylated cysteine
HTNSVICK

SEQ ID NO: 678 - protein, 14 aa, Artificial Sequence
  Native Peptide Sequence to quantitate RAS-RAF-MAPK Pathway protein
FLQESNVLYQHNLR

SEQ ID NO: 679 - protein, 7 aa, Artificial Sequence
  Native Peptide Sequence to quantitate RAS-RAF-MAPK Pathway protein
  MOD_RES (1)..(1): carbamidomethylated cysteine
CLWEESR

SEQ ID NO: 680 - protein, 26 aa, Artificial Sequence
  Native Peptide Sequence to quantitate RAS-RAF-MAPK Pathway protein
LLQTAATAAQQGGQANHPTAAVVTEK

SEQ ID NO: 681 - protein, 7 aa, Artificial Sequence
  Native Peptide Sequence to quantitate RAS-RAF-MAPK Pathway protein
SAFVVER

SEQ ID NO: 682 - protein, 9 aa, Artificial Sequence
  Native Peptide Sequence to quantitate RAS-RAF-MAPK Pathway protein
FPELNYQLK

SEQ ID NO: 683 - protein, 9 aa, Artificial Sequence
  Native Peptide Sequence to quantitate RAS-RAF-MAPK Pathway protein
FNILGTNTK

SEQ ID NO: 684 - protein, 13 aa, Artificial Sequence
  Native Peptide Sequence to quantitate RAS-RAF-MAPK Pathway protein
GLSIEQLTTLAEK

SEQ ID NO: 685 - protein, 14 aa, Artificial Sequence
  Native Peptide Sequence to quantitate RAS-RAF-MAPK Pathway protein
AILSTKPPGTFLLR

SEQ ID NO: 686 - protein, 11 aa, Artificial Sequence
  Native Peptide Sequence to quantitate RAS-RAF-MAPK Pathway protein
TQIQSVEPYTK

SEQ ID NO: 687 - protein, 11 aa, Artificial Sequence
  Native Peptide Sequence to quantitate RAS-RAF-MAPK Pathway protein
IDSTEVIYQPR

SEQ ID NO: 688 - protein, 20 aa, Artificial Sequence
  Native Peptide Sequence to quantitate RAS-RAF-MAPK Pathway protein
  MOD_RES (19)..(19): carbamidomethylated cysteine
ISDLGVAEALHPFAADDTCR

SEQ ID NO: 689 - protein, 18 aa, Artificial Sequence
  Native Peptide Sequence to quantitate RAS-RAF-MAPK Pathway protein
  MOD_RES (9)..(9): carbamidomethylated cysteine
GSYAIPGDCGPPLSDLLK

SEQ ID NO: 690 - protein, 14 aa, Artificial Sequence
  Native Peptide Sequence to quantitate RAS-RAF-MAPK Pathway protein
NVIQLVDVLYNEEK

SEQ ID NO: 691 - protein, 9 aa, Artificial Sequence
  Native Peptide Sequence to quantitate RAS-RAF-MAPK Pathway protein
TYQGSYGFR

SEQ ID NO: 692 - protein, 10 aa, Artificial Sequence
  Native Peptide Sequence to quantitate RAS-RAF-MAPK Pathway protein
LGFLHSGTAK

SEQ ID NO: 693 - protein, 12 aa, Artificial Sequence
  Native Peptide Sequence to quantitate RAS-RAF-MAPK Pathway protein
  MOD_RES (4)..(4): carbamidomethylated cysteine
SVTCTYSPALNK

SEQ ID NO: 694 - protein, 17 aa, Artificial Sequence
  Native Peptide Sequence to quantitate RAS-RAF-MAPK Pathway protein
  MOD_RES (2)..(2): carbamidomethylated cysteine
TCPVQLWVDSTPPPGTR

SEQ ID NO: 695 - protein, 15 aa, Artificial Sequence
  Native Peptide Sequence to quantitate RAS-RAF-MAPK Pathway protein
  MOD_RES (1)..(1): carbamidomethylated cysteine
CSDSDGLAPPQHLIR

SEQ ID NO: 696 - protein, 7 aa, Artificial Sequence
  Native Peptide Sequence to quantitate RAS-RAF-MAPK Pathway protein
VEYLDDR

SEQ ID NO: 697 - protein, 19 aa, Artificial Sequence
  Native Peptide Sequence to quantitate RAS-RAF-MAPK Pathway protein
RPILTIITLEDSSGNLLGR

SEQ ID NO: 698 - protein, 13 aa, Artificial Sequence
  Native Peptide Sequence to quantitate RAS-RAF-MAPK Pathway protein
ALPNNTSSSPQPK

SEQ ID NO: 699 - protein, 9 aa, Artificial Sequence
  Native Peptide Sequence to quantitate RAS-RAF-MAPK Pathway protein
ELNEALELK

SEQ ID NO: 700 - protein, 13 aa, Artificial Sequence
  Native Peptide Sequence to quantitate RAS-RAF-MAPK Pathway protein
  MOD_RES (9)..(9): phosphorylated serine
ALPNNTSSSPQPK

SEQ ID NO: 701 - protein, 8 aa, Artificial Sequence
  Internal Standard Peptide Sequence to quantitate RAS-RAF-MAPK Pathway protein
  MOD_RES (8)..(8): heavy isotope labeled amino acid
TSSISDLK

SEQ ID NO: 702 - protein, 12 aa, Artificial Sequence
  Internal Standard Peptide Sequence to quantitate RAS-RAF-MAPK Pathway protein
  MOD_RES (12)..(12): heavy isotope labeled amino acid
TSSISDLKEVPR

SEQ ID NO: 703 - protein, 8 aa, Artificial Sequence
  Internal Standard Peptide Sequence to quantitate RAS-RAF-MAPK Pathway protein
  MOD_RES (8)..(8): heavy isotope labeled amino acid
FNHQNIVR

SEQ ID NO: 704 - protein, 17 aa, Artificial Sequence
  Internal Standard Peptide Sequence to quantitate RAS-RAF-MAPK Pathway protein
  MOD_RES (4)..(6): carbamidomethylated cysteine
  MOD_RES (17)..(17): heavy isotope labeled amino acid
DIACGCQYLEENHFIHR

SEQ ID NO: 705 - protein, 11 aa, Artificial Sequence
  Internal Standard Peptide Sequence to quantitate RAS-RAF-MAPK Pathway protein
  MOD_RES (2)..(6): carbamidomethylated cysteine
  MOD_RES (11)..(11): heavy isotope labeled amino acid
NCLLTCPGPGR

SEQ ID NO: 706 - protein, 14 aa, Artificial Sequence
  Internal Standard Peptide Sequence to quantitate RAS-RAF-MAPK Pathway protein
  MOD_RES (14)..(14): heavy isotope labeled amino acid
SNQEVLEFVTSGGR

SEQ ID NO: 707 - protein, 8 aa, Artificial Sequence
  Internal Standard Peptide Sequence to quantitate RAS-RAF-MAPK Pathway protein
  MOD_RES (2)..(2): carbamidomethylated cysteine
  MOD_RES (8)..(8): heavy isotope labeled amino acid
NCPGPVYR

SEQ ID NO: 708 - protein, 15 aa, Artificial Sequence
  Internal Standard Peptide Sequence to quantitate RAS-RAF-MAPK Pathway protein
  MOD_RES (15)..(15): heavy isotope labeled amino acid
DPEGVPPLLVSQQAK

SEQ ID NO: 709 - protein, 19 aa, Artificial Sequence
  Internal Standard Peptide Sequence to quantitate RAS-RAF-MAPK Pathway protein
  MOD_RES (19)..(19): heavy isotope labeled amino acid
EEERSPAAPPPLPTTSSGK

SEQ ID NO: 710 - protein, 15 aa, Artificial Sequence
  Internal Standard Peptide Sequence to quantitate RAS-RAF-MAPK Pathway protein
  MOD_RES (15)..(15): heavy isotope labeled amino acid
SPAAPPPLPTTSSGK

SEQ ID NO: 711 - protein, 19 aa, Artificial Sequence
  Internal Standard Peptide Sequence to quantitate RAS-RAF-MAPK Pathway protein
  MOD_RES (9)..(9): carbamidomethylated cysteine
  MOD_RES (19)..(19): heavy isotope labeled amino acid
GNLGLEGSCTVPPNVATGR

SEQ ID NO: 712 - protein, 15 aa, Artificial Sequence
  Internal Standard Peptide Sequence to quantitate RAS-RAF-MAPK Pathway protein
  MOD_RES (9)..(9): carbamidomethylated cysteine
  MOD_RES (15)..(15): heavy isotope labeled amino acid
TQADELPACLLSAAR

SEQ ID NO: 713 - protein, 11 aa, Artificial Sequence
  Internal Standard Peptide Sequence to quantitate RAS-RAF-MAPK Pathway protein
  MOD_RES (11)..(11): heavy isotope labeled amino acid
IGTGSFGTVFR

SEQ ID NO: 714 - protein, 10 aa, Artificial Sequence
  Internal Standard Peptide Sequence to quantitate RAS-RAF-MAPK Pathway protein
  MOD_RES (10)..(10): heavy isotope labeled amino acid
IGDFGLATVK

SEQ ID NO: 715 - protein, 11 aa, Artificial Sequence
  Internal Standard Peptide Sequence to quantitate RAS-RAF-MAPK Pathway protein
  MOD_RES (6)..(7): carbamidomethylated cysteines
  MOD_RES (11)..(11): heavy isotope labeled amino acid
GLNQDCCVVYR

SEQ ID NO: 716 - protein, 13 aa, Artificial Sequence
  Internal Standard Peptide Sequence to quantitate RAS-RAF-MAPK Pathway protein
  MOD_RES (13)..(13): heavy isotope labeled amino acid
SNNIFLHEGLTVK

SEQ ID NO: 717 - protein, 12 aa, Artificial Sequence
  Internal Standard Peptide Sequence to quantitate RAS-RAF-MAPK Pathway protein
  MOD_RES (5)..(5): carbamidomethylated cysteine
  MOD_RES (12)..(12): heavy isotope labeled amino acid
VPTVCVDMSTNR

SEQ ID NO: 718 - protein, 13 aa, Artificial Sequence
  Internal Standard Peptide Sequence to quantitate RAS-RAF-MAPK Pathway protein
  MOD_RES (13)..(13): heavy isotope labeled amino acid
VSQPTAEQAQAFK

SEQ ID NO: 719 - protein, 21 aa, Artificial Sequence
  Internal Standard Peptide Sequence to quantitate RAS-RAF-MAPK Pathway protein
  MOD_RES (4)..(4): carbamidomethylated cysteine
  MOD_RES (21)..(21): heavy isotope labeled amino acid
TQHCDPEHFPFPAPANAPLQR

SEQ ID NO: 720 - protein, 15 aa, Artificial Sequence
  Internal Standard Peptide Sequence to quantitate RAS-RAF-MAPK Pathway protein
  MOD_RES (15)..(15): heavy isotope labeled amino acid
NDSVIVADQTPTPTR

SEQ ID NO: 721 - protein, 27 aa, Artificial Sequence
  Internal Standard Peptide Sequence to quantitate RAS-RAF-MAPK Pathway protein
  MOD_RES (27)..(27): heavy isotope labeled amino acid
IEEPSVVETTHQDSPLPHPESTTSDEK

SEQ ID NO: 722 - protein, 38 aa, Artificial Sequence; identical to SEQ ID NO: 22
  Internal Standard Peptide Sequence to quantitate RAS-RAF-MAPK Pathway protein
  MOD_RES (38)..(38): heavy isotope labeled amino acid

SEQ ID NO: 723 - protein, 20 aa, Artificial Sequence
  Internal Standard Peptide Sequence to quantitate RAS-RAF-MAPK Pathway protein
  MOD_RES (2)..(2): carbamidomethylated cysteine
  MOD_RES (20)..(20): heavy isotope labeled amino acid
NCEEVGLFNELASPFENEFK

SEQ ID NO: 724 - protein, 8 aa, Artificial Sequence
  Internal Standard Peptide Sequence to quantitate RAS-RAF-MAPK Pathway protein
  MOD_RES (8)..(8): heavy isotope labeled amino acid
VWVQSLEK

SEQ ID NO: 725 - protein, 16 aa, Artificial Sequence
  Internal Standard Peptide Sequence to quantitate RAS-RAF-MAPK Pathway protein
  MOD_RES (16)..(16): heavy isotope labeled amino acid
EVPLAQTAQPTSAIVR

SEQ ID NO: 726 - protein, 22 aa, Artificial Sequence
  Internal Standard Peptide Sequence to quantitate RAS-RAF-MAPK Pathway protein
  MOD_RES (22)..(22): heavy isotope labeled amino acid
SSSAPNVHINTIEPVNIDDLIR

SEQ ID NO: 727 - protein, 17 aa, Artificial Sequence
  Internal Standard Peptide Sequence to quantitate RAS-RAF-MAPK Pathway protein
  MOD_RES (13)..(13): carbamidomethylated cysteine
  MOD_RES (17)..(17): heavy isotope labeled amino acid
AGFQTEDFSLYACASPK

SEQ ID NO: 728 - protein, 7 aa, Artificial Sequence
  Internal Standard Peptide Sequence to quantitate RAS-RAF-MAPK Pathway protein
  MOD_RES (7)..(7): heavy isotope labeled amino acid
LLFQGFR

SEQ ID NO: 729 - protein, 14 aa, Artificial Sequence
  Internal Standard Peptide Sequence to quantitate RAS-RAF-MAPK Pathway protein
  MOD_RES (13)..(13): heavy isotope labeled amino acid
TPIQAGGYGAFPVH

SEQ ID NO: 730 - protein, 12 aa, Artificial Sequence
  Internal Standard Peptide Sequence to quantitate RAS-RAF-MAPK Pathway protein
  MOD_RES (12)..(12): heavy isotope labeled amino acid
LTQEHIEALLDK

SEQ ID NO: 731 - protein, 10 aa, Artificial Sequence
  Internal Standard Peptide Sequence to quantitate RAS-RAF-MAPK Pathway protein
  MOD_RES (10)..(10): heavy isotope labeled amino acid
IGDFGLATEK

SEQ ID NO: 732 - protein, 17 aa, Artificial Sequence
  Internal Standard Peptide Sequence to quantitate RAS-RAF-MAPK Pathway protein
  MOD_RES (17)..(17): heavy isotope labeled amino acid
EAFSLFDKDGDGTITTK

SEQ ID NO: 733 - protein, 7 aa, Artificial Sequence
  Internal Standard Peptide Sequence to quantitate RAS-RAF-MAPK Pathway protein
  MOD_RES (7)..(7): heavy isotope labeled amino acid
ELGTVMR

SEQ ID NO: 734 - protein, 13 aa, Artificial Sequence
  Internal Standard Peptide Sequence to quantitate RAS-RAF-MAPK Pathway protein
  MOD_RES (13)..(13): heavy isotope labeled amino acid
DTDSEEEIREAFR

SEQ ID NO: 735 - protein, 16 aa, Artificial Sequence
  Internal Standard Peptide Sequence to quantitate RAS-RAF-MAPK Pathway protein
  MOD_RES (16)..(16): heavy isotope labeled amino acid
VFDKDGNGYISAAELR

SEQ ID NO: 736 - protein, 12 aa, Artificial Sequence
  Internal Standard Peptide Sequence to quantitate RAS-RAF-MAPK Pathway protein
  MOD_RES (12)..(12): heavy isotope labeled amino acid
DGNGYISAAELR

SEQ ID NO: 737 - protein, 9 aa, Artificial Sequence
  Internal Standard Peptide Sequence to quantitate RAS-RAF-MAPK Pathway protein
  MOD_RES (9)..(9): heavy isotope labeled amino acid
HVMTNLGEK

SEQ ID NO: 738 - protein, 22 aa, Artificial Sequence
  Internal Standard Peptide Sequence to quantitate RAS-RAF-MAPK Pathway protein
  MOD_RES (22)..(22): heavy isotope labeled amino acid
EADIDGDGQVNYEEFVQMMTAK

SEQ ID NO: 739 - protein, 13 aa, Artificial Sequence
  Internal Standard Peptide Sequence to quantitate RAS-RAF-MAPK Pathway protein
  MOD_RES (13)..(13): heavy isotope labeled amino acid
FTEEYQLFEELGK

SEQ ID NO: 740 - protein, 10 aa, Artificial Sequence
  Internal Standard Peptide Sequence to quantitate RAS-RAF-MAPK Pathway protein
  MOD_RES (10)..(10): heavy isotope labeled amino acid
VLAGQEYAAK

SEQ ID NO: 741 - protein, 8 aa, Artificial Sequence
  Internal Standard Peptide Sequence to quantitate RAS-RAF-MAPK Pathway protein
  MOD_RES (8)..(8): heavy isotope labeled amino acid
ITAAEALK

SEQ ID NO: 742 - protein, 19 aa, Artificial Sequence
  Internal Standard Peptide Sequence to quantitate RAS-RAF-MAPK Pathway protein
  MOD_RES (19)..(19): heavy isotope labeled amino acid
VTEQLIEAISNGDFESYTK

SEQ ID NO: 743 - protein, 9 aa, Artificial Sequence
  Internal Standard Peptide Sequence to quantitate RAS-RAF-MAPK Pathway protein
  MOD_RES (9)..(9): heavy isotope labeled amino acid
FYFENLWSR

SEQ ID NO: 744 - protein, 12 aa, Artificial Sequence
  Internal Standard Peptide Sequence to quantitate RAS-RAF-MAPK Pathway protein
  MOD_RES (12)..(12): heavy isotope labeled amino acid
ITQYLDAGGIPR

SEQ ID NO: 745 - protein, 8 aa, Artificial Sequence
  Internal Standard Peptide Sequence to quantitate RAS-RAF-MAPK Pathway protein
  MOD_RES (8)..(8): heavy isotope labeled amino acid
WQIVHFHR

SEQ ID NO: 746 - protein, 9 aa, Artificial Sequence
  Internal Standard Peptide Sequence to quantitate RAS-RAF-MAPK Pathway protein
  MOD_RES (8)..(8): heavy isotope labeled amino acid
SGAPSVLPH

SEQ ID NO: 747 - protein, 11 aa, Artificial Sequence
  Internal Standard Peptide Sequence to quantitate RAS-RAF-MAPK Pathway protein
  MOD_RES (11)..(11): heavy isotope labeled amino acid
AYPDANLLNDR

SEQ ID NO: 748 - protein, 13 aa, Artificial Sequence
  Internal Standard Peptide Sequence to quantitate RAS-RAF-MAPK Pathway protein
  MOD_RES (5)..(5): carbamidomethylated cysteine
  MOD_RES (13)..(13): heavy isotope labeled amino acid
AEETCAPSVSYFK

SEQ ID NO: 749 - protein, 8 aa, Artificial Sequence
  Internal Standard Peptide Sequence to quantitate RAS-RAF-MAPK Pathway protein
  MOD_RES (8)..(8): heavy isotope labeled amino acid
FLSLEPVK

SEQ ID NO: 750 - protein, 9 aa, Artificial Sequence
  Internal Standard Peptide Sequence to quantitate RAS-RAF-MAPK Pathway protein
  MOD_RES (9)..(9): heavy isotope labeled amino acid
FLSLEPVKK

SEQ ID NO: 751 - protein, 9 aa, Artificial Sequence
  Internal Standard Peptide Sequence to quantitate RAS-RAF-MAPK Pathway protein
  MOD_RES (9)..(9): heavy isotope labeled amino acid
ETIPLTAEK

SEQ ID NO: 752 - protein, 15 aa, Artificial Sequence
  Internal Standard Peptide Sequence to quantitate RAS-RAF-MAPK Pathway protein
  MOD_RES (10)..(10): carbamidomethylated cysteine
  MOD_RES (15)..(15): heavy isotope labeled amino acid
KHAQTFVALCATDVK

SEQ ID NO: 753 - protein, 14 aa, Artificial Sequence
  Internal Standard Peptide Sequence to quantitate RAS-RAF-MAPK Pathway protein
  MOD_RES (9)..(9): carbamidomethylated cysteine
  MOD_RES (14)..(14): heavy isotope labeled amino acid
HAQTFVALCATDVK

SEQ ID NO: 754 - protein, 10 aa, Artificial Sequence
  Internal Standard Peptide Sequence to quantitate RAS-RAF-MAPK Pathway protein
  MOD_RES (10)..(10): heavy isotope labeled amino acid
SPNNFLSYYR

SEQ ID NO: 755 - protein, 15 aa, Artificial Sequence
  Internal Standard Peptide Sequence to quantitate RAS-RAF-MAPK Pathway protein
  MOD_RES (2)..(2): carbamidomethylated cysteine
  MOD_RES (15)..(15): heavy isotope labeled amino acid
ACQEQIEALLESSLR

SEQ ID NO: 756 - protein, 22 aa, Artificial Sequence
  Internal Standard Peptide Sequence to quantitate RAS-RAF-MAPK Pathway protein
  MOD_RES (16)..(16): carbamidomethylated cysteine
  MOD_RES (22)..(22): heavy isotope labeled amino acid
AAEEEEEEEEEVDLACTPTDVR

SEQ ID NO: 757 - protein, 9 aa, Artificial Sequence
  Internal Standard Peptide Sequence to quantitate RAS-RAF-MAPK Pathway protein
  MOD_RES (6)..(6): carbamidomethylated cysteine
  MOD_RES (9)..(9): heavy isotope labeled amino acid
VNFEDCTGR

SEQ ID NO: 758 - protein, 14 aa, Artificial Sequence
  Internal Standard Peptide Sequence to quantitate RAS-RAF-MAPK Pathway protein
  MOD_RES (9)..(9): carbamidomethylated cysteine
  MOD_RES (14)..(14): heavy isotope labeled amino acid
DWVIPPISCPENEK

SEQ ID NO: 759 - protein, 22 aa, Artificial Sequence
  Internal Standard Peptide Sequence to quantitate RAS-RAF-MAPK Pathway protein
  MOD_RES (22)..(22): heavy isotope labeled amino acid
VFYSITGQGADTPPVGVFIIER

SEQ ID NO: 760 - protein, 14 aa, Artificial Sequence
  Internal Standard Peptide Sequence to quantitate RAS-RAF-MAPK Pathway protein
  MOD_RES (14)..(14): heavy isotope labeled amino acid
NTGVISVVTTGLDR

SEQ ID NO: 761 - protein, 16 aa, Artificial Sequence
  Internal Standard Peptide Sequence to quantitate RAS-RAF-MAPK Pathway protein
  MOD_RES (16)..(16): heavy isotope labeled amino acid
GQVPENEANVVITTLK

SEQ ID NO: 762 - protein, 18 aa, Artificial Sequence
  Internal Standard Peptide Sequence to quantitate RAS-RAF-MAPK Pathway protein
  MOD_RES (18)..(18): heavy isotope labeled amino acid
DTANWLEINPDTGAISTR

SEQ ID NO: 763 - protein, 7 aa, Artificial Sequence
  Internal Standard Peptide Sequence to quantitate RAS-RAF-MAPK Pathway protein
  MOD_RES (5)..(5): carbamidomethylated cysteine
  MOD_RES (7)..(7): heavy isotope labeled amino acid
TIFFCER

SEQ ID NO: 764 - protein, 10 aa, Artificial Sequence
  Internal Standard Peptide Sequence to quantitate RAS-RAF-MAPK Pathway protein
  MOD_RES (10)..(10): heavy isotope labeled amino acid
GLDARPEVTR

SEQ ID NO: 765 - protein, 20 aa, Artificial Sequence
  Internal Standard Peptide Sequence to quantitate RAS-RAF-MAPK Pathway protein
  MOD_RES (20)..(20): heavy isotope labeled amino acid
YLPRPANPDEIGNFIDENLK

SEQ ID NO: 766 - protein, 14 aa, Artificial Sequence
  Internal Standard Peptide Sequence to quantitate RAS-RAF-MAPK Pathway protein
  MOD_RES (14)..(14): heavy isotope labeled amino acid
DWVIPPINLPENSR

SEQ ID NO: 767 - protein, 9 aa, Artificial Sequence
  Internal Standard Peptide Sequence to quantitate RAS-RAF-MAPK Pathway protein
  MOD_RES (9)..(9): heavy isotope labeled amino acid
GPFPQELVR

SEQ ID NO: 768 - protein, 26 aa, Artificial Sequence
  Internal Standard Peptide Sequence to quantitate RAS-RAF-MAPK Pathway protein
  MOD_RES (26)..(26): heavy isotope labeled amino acid
FAIQTDPNSNDGLVTVVKPIDFETNR

SEQ ID NO: 769 - protein, 9 aa, Artificial Sequence
  Internal Standard Peptide Sequence to quantitate RAS-RAF-MAPK Pathway protein
  MOD_RES (9)..(9): heavy isotope labeled amino acid
LSDPANWLK

SEQ ID NO: 770 - protein, 16 aa, Artificial Sequence
  Internal Standard Peptide Sequence to quantitate RAS-RAF-MAPK Pathway protein
  MOD_RES (16)..(16): heavy isotope labeled amino acid
IDPVNGQITTIAVLDR

SEQ ID NO: 771 - protein, 18 aa, Artificial Sequence
  Internal Standard Peptide Sequence to quantitate RAS-RAF-MAPK Pathway protein
  MOD_RES (18)..(18): heavy isotope labeled amino acid
SAAPHPGDIGDFINEGLK

SEQ ID NO: 772 - protein, 13 aa, Artificial Sequence
  Internal Standard Peptide Sequence to quantitate RAS-RAF-MAPK Pathway protein
  MOD_RES (13)..(13): heavy isotope labeled amino acid
RLFGPVDSEQLSR

SEQ ID NO: 773 - protein, 12 aa, Artificial Sequence
  Internal Standard Peptide Sequence to quantitate RAS-RAF-MAPK Pathway protein
  MOD_RES (12)..(12): heavy isotope labeled amino acid
LFGPVDSEQLSR

SEQ ID NO: 774 - protein, 21 aa, Artificial Sequence
  Internal Standard Peptide Sequence to quantitate RAS-RAF-MAPK Pathway protein
  MOD_RES (21)..(21): heavy isotope labeled amino acid
ERWNFDFVTETPLEGDFAWER

SEQ ID NO: 775 - protein, 19 aa, Artificial Sequence
  Internal Standard Peptide Sequence to quantitate RAS-RAF-MAPK Pathway protein
  MOD_RES (19)..(19): heavy isotope labeled amino acid
WNFDFVTETPLEGDFAWER

SEQ ID NO: 776 - protein, 8 aa, Artificial Sequence
  Internal Standard Peptide Sequence to quantitate RAS-RAF-MAPK Pathway protein
  MOD_RES (8)..(8): heavy isotope labeled amino acid
LYLPTGPR

SEQ ID NO: 777 - protein, 29 aa, Artificial Sequence
  Internal Standard Peptide Sequence to quantitate RAS-RAF-MAPK Pathway protein
  MOD_RES (24)..(24): carbamidomethylated cysteine
  MOD_RES (29)..(29): heavy isotope labeled amino acid
RPGTSPALLQGTAEEDHVDLSLSCTLVPR

SEQ ID NO: 778 - protein, 18 aa, Artificial Sequence
  Internal Standard Peptide Sequence to quantitate RAS-RAF-MAPK Pathway protein
  MOD_RES (18)..(18): heavy isotope labeled amino acid
SGEQAEGSPGGPGDSQGR

SEQ ID NO: 779 - protein, 10 aa, Artificial Sequence
  Internal Standard Peptide Sequence to quantitate RAS-RAF-MAPK Pathway protein
  MOD_RES (10)..(10): heavy isotope labeled amino acid
VSNGSPSLER

SEQ ID NO: 780 - protein, 13 aa, Artificial Sequence
  Internal Standard Peptide Sequence to quantitate RAS-RAF-MAPK Pathway protein
  MOD_RES (13)..(13): heavy isotope labeled amino acid
NLFGPVDHEELTR

SEQ ID NO: 781 - protein, 14 aa, Artificial Sequence
  Internal Standard Peptide Sequence to quantitate RAS-RAF-MAPK Pathway protein
  MOD_RES (14)..(14): heavy isotope labeled amino acid
WNFDFQNHKPLEGK

SEQ ID NO: 782 - protein, 8 aa, Artificial Sequence
  Internal Standard Peptide Sequence to quantitate RAS-RAF-MAPK Pathway protein
  MOD_RES (8)..(8): heavy isotope labeled amino acid
YEWQEVEK

SEQ ID NO: 783 - protein, 15 aa, Artificial Sequence
  Internal Standard Peptide Sequence to quantitate RAS-RAF-MAPK Pathway protein
  MOD_RES (15)..(15): heavy isotope labeled amino acid
GSLPEFYYRPPRPPK

SEQ ID NO: 784 - protein, 18 aa, Artificial Sequence
  Internal Standard Peptide Sequence to quantitate RAS-RAF-MAPK Pathway protein
  MOD_RES (14)..(14): carbamidomethylated cysteine
  MOD_RES (18)..(18): heavy isotope labeled amino acid
TDPSDSQTGLAEQCAGIR

SEQ ID NO: 785 - protein, 20 aa, Artificial Sequence
  Internal Standard Peptide Sequence to quantitate RAS-RAF-MAPK Pathway protein
  MOD_RES (20)..(20): heavy isotope labeled amino acid
TEENVSDGSPNAGSVEQTPK

SEQ ID NO: 786 - protein, 17 aa, Artificial Sequence
  Internal Standard Peptide Sequence to quantitate RAS-RAF-MAPK Pathway protein
  MOD_RES (17)..(17): heavy isotope labeled amino acid
ALLEAGALPNAPNSYGR

SEQ ID NO: 787 - protein, 12 aa, Artificial Sequence
  Internal Standard Peptide Sequence to quantitate RAS-RAF-MAPK Pathway protein
  MOD_RES (12)..(12): heavy isotope labeled amino acid
RPIQVMMMGSAR

SEQ ID NO: 788 - protein, 12 aa, Artificial Sequence
  Internal Standard Peptide Sequence to quantitate RAS-RAF-MAPK Pathway protein
  MOD_RES (12)..(12): heavy isotope labeled amino acid
LPVDLAEELGHR

SEQ ID NO: 789 - protein, 12 aa, Artificial Sequence
  Internal Standard Peptide Sequence to quantitate RAS-RAF-MAPK Pathway protein
  MOD_RES (11)..(11): heavy isotope labeled amino acid
IDAAEGPSDIPD

SEQ ID NO: 790 - protein, 14 aa, Artificial Sequence
  Internal Standard Peptide Sequence to quantitate RAS-RAF-MAPK Pathway protein
  MOD_RES (14)..(14): heavy isotope labeled amino acid
ILNDLSSDAPGVPR

SEQ ID NO: 791 - protein, 29 aa, Artificial Sequence
  Internal Standard Peptide Sequence to quantitate RAS-RAF-MAPK Pathway protein
  MOD_RES (29)..(29): heavy isotope labeled amino acid
TAPTSTIAPGVVMASSPALPTQPAEEAAR

SEQ ID NO: 792 - protein, 18 aa, Artificial Sequence
  Internal Standard Peptide Sequence to quantitate RAS-RAF-MAPK Pathway protein
  MOD_RES (18)..(18): heavy isotope labeled amino acid
HAVVNLINYQDDAELATR

SEQ ID NO: 793 - protein, 19 aa, Artificial Sequence
  Internal Standard Peptide Sequence to quantitate RAS-RAF-MAPK Pathway protein
  MOD_RES (19)..(19): heavy isotope labeled amino acid
AIPELTKLLNDEDQVVVNK

SEQ ID NO: 794 - protein, 12 aa, Artificial Sequence
  Internal Standard Peptide Sequence to quantitate RAS-RAF-MAPK Pathway protein
  MOD_RES (12)..(12): heavy isotope labeled amino acid
LLNDEDQVVVNK

SEQ ID NO: 795 - protein, 13 aa, Artificial Sequence
  Internal Standard Peptide Sequence to quantitate RAS-RAF-MAPK Pathway protein
  MOD_RES (1)..(1): carbamidomethylated cysteine
  MOD_RES (13)..(13): heavy isotope labeled amino acid
CTAGTLHNLSHHR

SEQ ID NO: 796 - protein, 9 aa, Artificial Sequence
  Internal Standard Peptide Sequence to quantitate RAS-RAF-MAPK Pathway protein
  MOD_RES (9)..(9): heavy isotope labeled amino acid
SGGIPALVK

SEQ ID NO: 797 - protein, 8 aa, Artificial Sequence
  Internal Standard Peptide Sequence to quantitate RAS-RAF-MAPK Pathway protein
  MOD_RES (8)..(8): heavy isotope labeled amino acid
NLSDAATK

SEQ ID NO: 798 - protein, 16 aa, Artificial Sequence
  Internal Standard Peptide Sequence to quantitate RAS-RAF-MAPK Pathway protein
  MOD_RES (13)..(13): carbamidomethylated cysteine
  MOD_RES (16)..(16): heavy isotope labeled amino acid
AGDREDITEPAICALR

SEQ ID NO: 799 - protein, 12 aa, Artificial Sequence
  Internal Standard Peptide Sequence to quantitate RAS-RAF-MAPK Pathway protein
  MOD_RES (9)..(9): carbamidomethylated cysteine
  MOD_RES (12)..(12): heavy isotope labeled amino acid
EDITEPAICALR

SEQ ID NO: 800 - protein, 10 aa, Artificial Sequence
  Internal Standard Peptide Sequence to quantitate RAS-RAF-MAPK Pathway protein
  MOD_RES (10)..(10): heavy isotope labeled amino acid
LHYGLPVVVK

SEQ ID NO: 801 - protein, 12 aa, Artificial Sequence
  Internal Standard Peptide Sequence to quantitate RAS-RAF-MAPK Pathway protein
  MOD_RES (12)..(12): heavy isotope labeled amino acid
LLHPPSHWPLIK

SEQ ID NO: 802 - protein, 13 aa, Artificial Sequence
  Internal Standard Peptide Sequence to quantitate RAS-RAF-MAPK Pathway protein
  MOD_RES (5)..(5): carbamidomethylated cysteine
  MOD_RES (13)..(13): heavy isotope labeled amino acid
NLALCPANHAPLR

SEQ ID NO: 803 - protein, 20 aa, Artificial Sequence
  Internal Standard Peptide Sequence to quantitate RAS-RAF-MAPK Pathway protein
  MOD_RES (5)..(5): carbamidomethylated cysteine
  MOD_RES (20)..(20): heavy isotope labeled amino acid
NLALCPANHAPLREQGAIPR

SEQ ID NO: 804 - protein, 7 aa, Artificial Sequence
  Internal Standard Peptide Sequence to quantitate RAS-RAF-MAPK Pathway protein
  MOD_RES (7)..(7): heavy isotope labeled amino acid
LVQLLVR

SEQ ID NO: 805 - protein, 22 aa, Artificial Sequence
  Internal Standard Peptide Sequence to quantitate RAS-RAF-MAPK Pathway protein
  MOD_RES (22)..(22): heavy isotope labeled amino acid
EAAEAIEAEGATAPLTELLHSR

SEQ ID NO: 806 - protein, 14 aa, Artificial Sequence
  Internal Standard Peptide Sequence to quantitate RAS-RAF-MAPK Pathway protein
  MOD_RES (14)..(14): heavy isotope labeled amino acid
NEGVATYAAAVLFR

SEQ ID NO: 807 - protein, 9 aa, Artificial Sequence
  Internal Standard Peptide Sequence to quantitate RAS-RAF-MAPK Pathway protein
  MOD_RES (9)..(9): heavy isotope labeled amino acid
DLEPPIVAR

SEQ ID NO: 808 - protein, 13 aa, Artificial Sequence
  Internal Standard Peptide Sequence to quantitate RAS-RAF-MAPK Pathway protein
  MOD_RES (13)..(13): heavy isotope labeled amino acid
IFPLRPDYQEPSR

SEQ ID NO: 809 - protein, 8 aa, Artificial Sequence
  Internal Standard Peptide Sequence to quantitate RAS-RAF-MAPK Pathway protein
  MOD_RES (8)..(8): heavy isotope labeled amino acid
TVSYTNLK

SEQ ID NO: 810 - protein, 9 aa, Artificial Sequence
  Internal Standard Peptide Sequence to quantitate RAS-RAF-MAPK Pathway protein
  MOD_RES (9)..(9): heavy isotope labeled amino acid
NLYSGDYYR

SEQ ID NO: 811 - protein, 10 aa, Artificial Sequence
  Internal Standard Peptide Sequence to quantitate RAS-RAF-MAPK Pathway protein
  MOD_RES (10)..(10): heavy isotope labeled amino acid
YLSSLNFVHR

SEQ ID NO: 812 - protein, 9 aa, Artificial Sequence
  Internal Standard Peptide Sequence to quantitate RAS-RAF-MAPK Pathway protein
  MOD_RES (9)..(9): heavy isotope labeled amino acid
DVAVEEFPR

SEQ ID NO: 813 - protein, 15 aa, Artificial Sequence
  Internal Standard Peptide Sequence to quantitate RAS-RAF-MAPK Pathway protein
  MOD_RES (15)..(15): heavy isotope labeled amino acid
SSSPSEQGSNSTYDR

SEQ ID NO: 814 - protein, 9 aa, Artificial Sequence
  Internal Standard Peptide Sequence to quantitate RAS-RAF-MAPK Pathway protein
  MOD_RES (9)..(9): heavy isotope labeled amino acid
FVTVPLHHR

SEQ ID NO: 815 - protein, 12 aa, Artificial Sequence
  Internal Standard Peptide Sequence to quantitate RAS-RAF-MAPK Pathway protein
  MOD_RES (12)..(12): heavy isotope labeled amino acid
HEPPNSSSSDVR

SEQ ID NO: 816 - protein, 16 aa, Artificial Sequence
  Internal Standard Peptide Sequence to quantitate RAS-RAF-MAPK Pathway protein
  MOD_RES (10)..(10): carbamidomethylated cysteine
  MOD_RES (16)..(16): heavy isotope labeled amino acid
QTYLPQPAICPDSVYK

SEQ ID NO: 817 - protein, 11 aa, Artificial Sequence
  Internal Standard Peptide Sequence to quantitate RAS-RAF-MAPK Pathway protein
  MOD_RES (11)..(11): heavy isotope labeled amino acid
ILPGLYIGNFK

SEQ ID NO: 818 - protein, 19 aa, Artificial Sequence
  Internal Standard Peptide Sequence to quantitate RAS-RAF-MAPK Pathway protein
  MOD_RES (19)..(19): heavy isotope labeled amino acid
VTHILSVHDSARPMLEGVK

SEQ ID NO: 819 - protein, 16 aa, Artificial Sequence
  Internal Standard Peptide Sequence to quantitate RAS-RAF-MAPK Pathway protein
  MOD_RES (3)..(3): carbamidomethylated cysteine
  MOD_RES (16)..(16): heavy isotope labeled amino acid
YLCIPAADSPSQNLTR

SEQ ID NO: 820 - protein, 6 aa, Artificial Sequence
  Internal Standard Peptide Sequence to quantitate RAS-RAF-MAPK Pathway protein
  MOD_RES (5)..(5): carbamidomethylated cysteine
  MOD_RES (6)..(6): heavy isotope labeled amino acid
FIHECR

SEQ ID NO: 821 - protein, 14 aa, Artificial Sequence
  Internal Standard Peptide Sequence to quantitate RAS-RAF-MAPK Pathway protein
  MOD_RES (4)..(8): carbamidomethylated cysteine
  MOD_RES (14)..(14): heavy isotope labeled amino acid
GESCLVHCLAGVSR

SEQ ID NO: 822 - protein, 11 aa, Artificial Sequence
  Internal Standard Peptide Sequence to quantitate RAS-RAF-MAPK Pathway protein
  MOD_RES (2)..(2): carbamidomethylated cysteine
  MOD_RES (11)..(11): heavy isotope labeled amino acid
SCANPNVGFQR

SEQ ID NO: 823 - protein, 15 aa, Artificial Sequence
  Internal Standard Peptide Sequence to quantitate RAS-RAF-MAPK Pathway protein
  MOD_RES (15)..(15): heavy isotope labeled amino acid
EEYGESPLQDAEEAK

SEQ ID NO: 824 - protein, 23 aa, Artificial Sequence
  Internal Standard Peptide Sequence to quantitate RAS-RAF-MAPK Pathway protein
  MOD_RES (23)..(23): heavy isotope labeled amino acid
DENGGGAGGSGSHGTLGLPSGGK

SEQ ID NO: 825 - protein, 24 aa, Artificial Sequence
  Internal Standard Peptide Sequence to quantitate RAS-RAF-MAPK Pathway protein
  MOD_RES (1)..(6): carbamidomethylated cysteine
  MOD_RES (24)..(24): heavy isotope labeled amino acid
CLLLDCRPFLAHSAGYILGSVNVR

SEQ ID NO: 826 - protein, 15 aa, Artificial Sequence
  Internal Standard Peptide Sequence to quantitate RAS-RAF-MAPK Pathway protein
  MOD_RES (15)..(15): heavy isotope labeled amino acid
LRSGLYSAVIVYDER

SEQ ID NO: 827 - protein, 13 aa, Artificial Sequence
  Internal Standard Peptide Sequence to quantitate RAS-RAF-MAPK Pathway protein
  MOD_RES (13)..(13): heavy isotope labeled amino acid
SGLYSAVIVYDER

SEQ ID NO: 828 - protein, 11 aa, Artificial Sequence
  Internal Standard Peptide Sequence to quantitate RAS-RAF-MAPK Pathway protein
  MOD_RES (9)..(9): carbamidomethylated cysteine
  MOD_RES (11)..(11): heavy isotope labeled amino acid
FSSEYPEFCSK

SEQ ID NO: 829 - protein, 11 aa, Artificial Sequence
  Internal Standard Peptide Sequence to quantitate RAS-RAF-MAPK Pathway protein
  MOD_RES (11)..(11): heavy isotope labeled amino acid
VRLEEAFEFVK

SEQ ID NO: 830 - protein, 9 aa, Artificial Sequence
  Internal Standard Peptide Sequence to quantitate RAS-RAF-MAPK Pathway protein
  MOD_RES (9)..(9): heavy isotope labeled amino acid
LEEAFEFVK

SEQ ID NO: 831 - protein, 15 aa, Artificial Sequence
  Internal Standard Peptide Sequence to quantitate RAS-RAF-MAPK Pathway protein
  MOD_RES (15)..(15): heavy isotope labeled amino acid
TVAWLNEQLELGNER

SEQ ID NO: 832 - protein, 10 aa, Artificial Sequence
  Internal Standard Peptide Sequence to quantitate RAS-RAF-MAPK Pathway protein
  MOD_RES (10)..(10): heavy isotope labeled amino acid
AFYLEGGFSK

SEQ ID NO: 833 - protein, 15 aa, Artificial Sequence
  Internal Standard Peptide Sequence to quantitate RAS-RAF-MAPK Pathway protein
  MOD_RES (15)..(15): heavy isotope labeled amino acid
ISSDSSSDIESDLDR

SEQ ID NO: 834 - protein, 14 aa, Artificial Sequence
  Internal Standard Peptide Sequence to quantitate RAS-RAF-MAPK Pathway protein
  MOD_RES (14)..(14): heavy isotope labeled amino acid
DSTNLDVLEEFGIK

SEQ ID NO: 835 - protein, 20 aa, Artificial Sequence
  Internal Standard Peptide Sequence to quantitate RAS-RAF-MAPK Pathway protein
  MOD_RES (20)..(20): heavy isotope labeled amino acid
YILNVTPNLPNLFENAGEFK

SEQ ID NO: 836 - protein, 14 aa, Artificial Sequence
  Internal Standard Peptide Sequence to quantitate RAS-RAF-MAPK Pathway protein
  MOD_RES (2)..(8): carbamidomethylated cysteine
  MOD_RES (14)..(14): heavy isotope labeled amino acid
NCGVLVHCLAGISR

SEQ ID NO: 837 - protein, 11 aa, Artificial Sequence
  Internal Standard Peptide Sequence to quantitate RAS-RAF-MAPK Pathway protein
  MOD_RES (8)..(8): carbamidomethylated cysteine
  MOD_RES (11)..(11): heavy isotope labeled amino acid
TLGLSSPCDNR

SEQ ID NO: 838 - protein, 11 aa, Artificial Sequence
  Internal Standard Peptide Sequence to quantitate RAS-RAF-MAPK Pathway protein
  MOD_RES (11)..(11): heavy isotope labeled amino acid
NLQEILHGAVR

SEQ ID NO: 839 - protein, 10 aa, Artificial Sequence
  Internal Standard Peptide Sequence to quantitate RAS-RAF-MAPK Pathway protein
  MOD_RES (10)..(10): heavy isotope labeled amino acid
IPLENLQIIR

SEQ ID NO: 840 - protein, 16 aa, Artificial Sequence
  Internal Standard Peptide Sequence to quantitate RAS-RAF-MAPK Pathway protein
  MOD_RES (16)..(16): heavy isotope labeled amino acid
LTQLGTFEDHFLSLQR

SEQ ID NO: 841 - protein, 9 aa, Artificial Sequence
  Internal Standard Peptide Sequence to quantitate RAS-RAF-MAPK Pathway protein
  MOD_RES (7)..(7): carbamidomethylated cysteine
  MOD_RES (9)..(9): heavy isotope labeled amino acid
YSFGATCVK

SEQ ID NO: 842 - protein, 9 aa, Artificial Sequence
  Internal Standard Peptide Sequence to quantitate RAS-RAF-MAPK Pathway protein
  MOD_RES (9)..(9): heavy isotope labeled amino acid
YLVIQGDER

SEQ ID NO: 843 - protein, 19 aa, Artificial Sequence
  Internal Standard Peptide Sequence to quantitate RAS-RAF-MAPK Pathway protein
  MOD_RES (19)..(19): heavy isotope labeled amino acid
ELVEPLTPSGEAPNQALLR

SEQ ID NO: 844 - protein, 19 aa, Artificial Sequence
  Internal Standard Peptide Sequence to quantitate RAS-RAF-MAPK Pathway protein
  MOD_RES (19)..(19): heavy isotope labeled amino acid
GSHQISLDNPDYQQDFFPK

SEQ ID NO: 845 - protein, 19 aa, Artificial Sequence
  Internal Standard Peptide Sequence to quantitate RAS-RAF-MAPK Pathway protein
  MOD_RES (7)..(7): phosphorylated threonine
  MOD_RES (19)..(19): heavy isotope labeled amino acid
ELVEPLTPSGEAPNQALLR

SEQ ID NO: 846 - protein, 19 aa, Artificial Sequence
  Internal Standard Peptide Sequence to quantitate RAS-RAF-MAPK Pathway protein
  MOD_RES (6)..(6): phosphorylated serine
  MOD_RES (19)..(19): heavy isotope labeled amino acid
GSHQISLDNPDYQQDFFPK

SEQ ID NO: 847 - protein, 12 aa, Artificial Sequence
  Internal Standard Peptide Sequence to quantitate RAS-RAF-MAPK Pathway protein
  MOD_RES (12)..(12): heavy isotope labeled amino acid
DGTLFAWGNGEK

SEQ ID NO: 848 - protein, 9 aa, Artificial Sequence
  Internal Standard Peptide Sequence to quantitate RAS-RAF-MAPK Pathway protein
  MOD_RES (9)..(9): heavy isotope labeled amino acid
VNIISVTNK

SEQ ID NO: 849 - protein, 9 aa, Artificial Sequence
  Internal Standard Peptide Sequence to quantitate RAS-RAF-MAPK Pathway protein
  MOD_RES (3)..(3): carbamidomethylated cysteine
  MOD_RES (9)..(9): heavy isotope labeled amino acid
AVCLEFSPK

SEQ ID NO: 850 - protein, 14 aa, Artificial Sequence
  Internal Standard Peptide Sequence to quantitate RAS-RAF-MAPK Pathway protein
  MOD_RES (14)..(14): heavy isotope labeled amino acid
NTVLATWQPYTTSK

SEQ ID NO: 851 - protein, 15 aa, Artificial Sequence
  Internal Standard Peptide Sequence to quantitate RAS-RAF-MAPK Pathway protein
  MOD_RES (15)..(15): heavy isotope labeled amino acid
DGTAGIPNLQLYDVK

SEQ ID NO: 852 - protein, 20 aa, Artificial Sequence
  Internal Standard Peptide Sequence to quantitate RAS-RAF-MAPK Pathway protein
  MOD_RES (20)..(20): heavy isotope labeled amino acid
NVNNEVHFFENNNFNTIANK

SEQ ID NO: 853 - protein, 14 aa, Artificial Sequence
  Internal Standard Peptide Sequence to quantitate RAS-RAF-MAPK Pathway protein
  MOD_RES (14)..(14): heavy isotope labeled amino acid
INDFVLSPGPQPYK

SEQ ID NO: 854 - protein, 17 aa, Artificial Sequence
  Internal Standard Peptide Sequence to quantitate RAS-RAF-MAPK Pathway protein
  MOD_RES (17)..(17): heavy isotope labeled amino acid
LYQYPNFAGPHAALANK

SEQ ID NO: 855 - protein, 7 aa, Artificial Sequence
  Internal Standard Peptide Sequence to quantitate RAS-RAF-MAPK Pathway protein
  MOD_RES (7)..(7): heavy isotope labeled amino acid
ATIFNLK

SEQ ID NO: 856 - protein, 12 aa, Artificial Sequence
  Internal Standard Peptide Sequence to quantitate RAS-RAF-MAPK Pathway protein
  MOD_RES (1)..(1): carbamidomethylated cysteine
  MOD_RES (12)..(12): heavy isotope labeled amino acid
CDPVFDFGTGPR

SEQ ID NO: 857 - protein, 21 aa, Artificial Sequence
  Internal Standard Peptide Sequence to quantitate RAS-RAF-MAPK Pathway protein
  MOD_RES (21)..(21): heavy isotope labeled amino acid
NAAYYSPHGHILVLAGFGNLR

SEQ ID NO: 858 - protein, 17 aa, Artificial Sequence
  Internal Standard Peptide Sequence to quantitate RAS-RAF-MAPK Pathway protein
  MOD_RES (17)..(17): heavy isotope labeled amino acid
TITYQAVPSEVPNEEPK

SEQ ID NO: 859 - protein, 11 aa, Artificial Sequence
  Internal Standard Peptide Sequence to quantitate RAS-RAF-MAPK Pathway protein
  MOD_RES (11)..(11): heavy isotope labeled amino acid
VATAYRPPALR

SEQ ID NO: 860 - protein, 7 aa, Artificial Sequence
  Internal Standard Peptide Sequence to quantitate RAS-RAF-MAPK Pathway protein
  MOD_RES (7)..(7): heavy isotope labeled amino acid
WLITLNK

SEQ ID NO: 861 - protein, 11 aa, Artificial Sequence
  Internal Standard Peptide Sequence to quantitate RAS-RAF-MAPK Pathway protein
  MOD_RES (8)..(8): carbamidomethylated cysteine
  MOD_RES (11)..(11): heavy isotope labeled amino acid
IAIWTTECENR

SEQ ID NO: 862 - protein, 8 aa, Artificial Sequence
  Internal Standard Peptide Sequence to quantitate RAS-RAF-MAPK Pathway protein
  MOD_RES (8)..(8): heavy isotope labeled amino acid
EAVTHIGR

SEQ ID NO: 863 - protein, 14 aa, Artificial Sequence
  Internal Standard Peptide Sequence to quantitate RAS-RAF-MAPK Pathway protein
  MOD_RES (14)..(14): heavy isotope labeled amino acid
IVIGYQSHADTATK

SEQ ID NO: 864 - protein, 15 aa, Artificial Sequence
  Internal Standard Peptide Sequence to quantitate RAS-RAF-MAPK Pathway protein
  MOD_RES (15)..(15): heavy isotope labeled amino acid
TESNQEVANPEHYIK

SEQ ID NO: 865 - protein, 7 aa, Artificial Sequence
  Internal Standard Peptide Sequence to quantitate RAS-RAF-MAPK Pathway protein
  MOD_RES (7)..(7): heavy isotope labeled amino acid
TWQANLR

SEQ ID NO: 866 - protein, 11 aa, Artificial Sequence
  Internal Standard Peptide Sequence to quantitate RAS-RAF-MAPK Pathway protein
  MOD_RES (11)..(11): heavy isotope labeled amino acid
DGIEPMWEDEK

SEQ ID NO: 867 - protein, 13 aa, Artificial Sequence
  Internal Standard Peptide Sequence to quantitate RAS-RAF-MAPK Pathway protein
  MOD_RES (5)..(5): carbamidomethylated cysteine
  MOD_RES (13)..(13): heavy isotope labeled amino acid
NPQLCYQDTILWK

SEQ ID NO: 868 - protein, 14 aa, Artificial Sequence
  Internal Standard Peptide Sequence to quantitate RAS-RAF-MAPK Pathway protein
  MOD_RES (1)..(9): carbamidomethylated cysteine
  MOD_RES (14)..(14): heavy isotope labeled amino acid
CWGESSEDCQSLTR

SEQ ID NO: 869 - protein, 14 aa, Artificial Sequence
  Internal Standard Peptide Sequence to quantitate RAS-RAF-MAPK Pathway protein
  MOD_RES (13)..(13): carbamidomethylated cysteine
  MOD_RES (14)..(14): heavy isotope labeled amino acid
AVTSANIQEFAGCK

SEQ ID NO: 870 - protein, 9 aa, Artificial Sequence
  Internal Standard Peptide Sequence to quantitate RAS-RAF-MAPK Pathway protein
  MOD_RES (4)..(8): carbamidomethylated cysteine
  MOD_RES (9)..(9): heavy isotope labeled amino acid
GQECVEECR

SEQ ID NO: 871 - protein, 7 aa, Artificial Sequence
  Internal Standard Peptide Sequence to quantitate RAS-RAF-MAPK Pathway protein
  MOD_RES (7)..(7): heavy isotope labeled amino acid
VLQGLPR

SEQ ID NO: 872 - protein, 11 aa, Artificial Sequence
  Internal Standard Peptide Sequence to quantitate RAS-RAF-MAPK Pathway protein
  MOD_RES (11)..(11): heavy isotope labeled amino acid
GIWIPDGENVK

SEQ ID NO: 873 - protein, 15 aa, Artificial Sequence
  Internal Standard Peptide Sequence to quantitate RAS-RAF-MAPK Pathway protein
  MOD_RES (15)..(15): heavy isotope labeled amino acid
LLDIDETEYHADGGK

SEQ ID NO: 874 - protein, 8 aa, Artificial Sequence
  Internal Standard Peptide Sequence to quantitate RAS-RAF-MAPK Pathway protein
  MOD_RES (8)..(8): heavy isotope labeled amino acid
ELVSEFSR

SEQ ID NO: 875 - protein, 21 aa, Artificial Sequence
  Internal Standard Peptide Sequence to quantitate RAS-RAF-MAPK Pathway protein
  MOD_RES (21)..(21): heavy isotope labeled amino acid
FVVIQNEDLGPASPLDSTFYR

SEQ ID NO: 876 - protein, 19 aa, Artificial Sequence
  Internal Standard Peptide Sequence to quantitate RAS-RAF-MAPK Pathway protein
  MOD_RES (19)..(19): heavy isotope labeled amino acid
SGGGDLTLGLEPSEEEAPR

SEQ ID NO: 877 - protein, 15 aa, Artificial Sequence
  Internal Standard Peptide Sequence to quantitate RAS-RAF-MAPK Pathway protein
  MOD_RES (15)..(15): heavy isotope labeled amino acid
GLQSLPTHDPSPLQR

SEQ ID NO: 878 - protein, 25 aa, Artificial Sequence
  Internal Standard Peptide Sequence to quantitate RAS-RAF-MAPK Pathway protein
  MOD_RES (8)..(19): carbamidomethylated cysteine
  MOD_RES (25)..(25): heavy isotope labeled amino acid
FPDEEGACQPCPINCTHSCVDLDDK

SEQ ID NO: 879 - protein, 13 aa, Artificial Sequence
  Internal Standard Peptide Sequence to quantitate RAS-RAF-MAPK Pathway protein
  MOD_RES (13)..(13): heavy isotope labeled amino acid
NNQLALTLIDTNR

SEQ ID NO: 880 - protein, 20 aa, Artificial Sequence
  Internal Standard Peptide Sequence to quantitate RAS-RAF-MAPK Pathway protein
  MOD_RES (7)..(16): carbamidomethylated cysteine
  MOD_RES (20)..(20): heavy isotope labeled amino acid
GPLPTDCCHEQCAAGCTGPK

SEQ ID NO: 881 - protein, 22 aa, Artificial Sequence
  Internal Standard Peptide Sequence to quantitate RAS-RAF-MAPK Pathway protein
  MOD_RES (2)..(2): carbamidomethylated cysteine
  MOD_RES (22)..(22): heavy isotope labeled amino acid
GCLASESSEGHVTGSEAELQEK

SEQ ID NO: 882 - protein, 16 aa, Artificial Sequence
  Internal Standard Peptide Sequence to quantitate RAS-RAF-MAPK Pathway protein
  MOD_RES (4)..(8): carbamidomethylated cysteine
  MOD_RES (16)..(16): heavy isotope labeled amino acid
GGVCVTHCNFLNGEPR

SEQ ID NO: 883 - protein, 12 aa, Artificial Sequence
  Internal Standard Peptide Sequence to quantitate RAS-RAF-MAPK Pathway protein
  MOD_RES (12)..(12): heavy isotope labeled amino acid
LSSLSDLEQQYR

SEQ ID NO: 884 - protein, 12 aa, Artificial Sequence
  Internal Standard Peptide Sequence to quantitate RAS-RAF-MAPK Pathway protein
  MOD_RES (12)..(12): heavy isotope labeled amino acid
DGNFGLQELGLK

SEQ ID NO: 885 - protein, 15 aa, Artificial Sequence
  Internal Standard Peptide Sequence to quantitate RAS-RAF-MAPK Pathway protein
  MOD_RES (3)..(3): carbamidomethylated cysteine
  MOD_RES (15)..(15): heavy isotope labeled amino acid
FLCYADTIHWQDIVR

SEQ ID NO: 886 - protein, 14 aa, Artificial Sequence
  Internal Standard Peptide Sequence to quantitate RAS-RAF-MAPK Pathway protein
  MOD_RES (1)..(12): heavy isotope labeled amino acid
  MOD_RES (3)..(3): carbamidomethylated cysteine
CPHNFVVDSSSCVR

SEQ ID NO: 887 - protein, 23 aa, Artificial Sequence
  Internal Standard Peptide Sequence to quantitate RAS-RAF-MAPK Pathway protein
  MOD_RES (23)..(23): heavy isotope labeled amino acid
FLETELVEPLTPSGTAPNQAQLR

SEQ ID NO: 888 - protein, 9 aa, Artificial Sequence
  Internal Standard Peptide Sequence to quantitate RAS-RAF-MAPK Pathway protein
  MOD_RES (9)..(9): heavy isotope labeled amino acid
ILNETTGPK

SEQ ID NO: 889 - protein, 8 aa, Artificial Sequence
  Internal Standard Peptide Sequence to quantitate RAS-RAF-MAPK Pathway protein
  MOD_RES (8)..(8): heavy isotope labeled amino acid
ELAAEFSR

SEQ ID NO: 890 - protein, 15 aa, Artificial Sequence
  Internal Standard Peptide Sequence to quantitate RAS-RAF-MAPK Pathway protein
  MOD_RES (15)..(15): heavy isotope labeled amino acid
DGGFAAEQGVSVPYR

SEQ ID NO: 891 - protein, 15 aa, Artificial Sequence
  Internal Standard Peptide Sequence to quantitate RAS-RAF-MAPK Pathway protein
  MOD_RES (15)..(15): heavy isotope labeled amino acid
KPVAPHVQEDSSTQR

SEQ ID NO: 892 - protein, 12 aa, Artificial Sequence
  Internal Standard Peptide Sequence to quantitate RAS-RAF-MAPK Pathway protein
  MOD_RES (12)..(12): heavy isotope labeled amino acid
YSADPTVFAPER

SEQ ID NO: 893 - protein, 15 aa, Artificial Sequence
  Internal Standard Peptide Sequence to quantitate RAS-RAF-MAPK Pathway protein
  MOD_RES (15)..(15): heavy isotope labeled amino acid
STLQHPDYLQEYSTK

SEQ ID NO: 894 - protein, 16 aa, Artificial Sequence
  Internal Standard Peptide Sequence to quantitate RAS-RAF-MAPK Pathway protein
  MOD_RES (16)..(16): heavy isotope labeled amino acid
NLTEILNGGVYVDQNK

SEQ ID NO: 895 - protein, 23 aa, Artificial Sequence
  Internal Standard Peptide Sequence to quantitate RAS-RAF-MAPK Pathway protein
  MOD_RES (2)..(22): carbamidomethylated cysteine
  MOD_RES (23)..(23): heavy isotope labeled amino acid
YCAVCNDYASGYHYGVWSCEGCK

SEQ ID NO: 896 - protein, 11 aa, Artificial Sequence
  Internal Standard Peptide Sequence to quantitate RAS-RAF-MAPK Pathway protein
  MOD_RES (11)..(11): heavy isotope labeled amino acid
LLFAPNLLLDR

SEQ ID NO: 897 - protein, 11 aa, Artificial Sequence
  Internal Standard Peptide Sequence to quantitate RAS-RAF-MAPK Pathway protein
  MOD_RES (11)..(11): heavy isotope labeled amino acid
AGLTLQQQHQR

SEQ ID NO: 898 - protein, 12 aa, Artificial Sequence
  Internal Standard Peptide Sequence to quantitate RAS-RAF-MAPK Pathway protein
  MOD_RES (12)..(12): heavy isotope labeled amino acid
LAQLLLILSHIR

SEQ ID NO: 899 - protein, 26 aa, Artificial Sequence
  Internal Standard Peptide Sequence to quantitate RAS-RAF-MAPK Pathway protein
  MOD_RES (26)..(26): heavy isotope labeled amino acid
GGASVEETDQSHLATAGSTSSHSLQK

SEQ ID NO: 900 - protein, 12 aa, Artificial Sequence
  Internal Standard Peptide Sequence to quantitate RAS-RAF-MAPK Pathway protein
  MOD_RES (12)..(12): heavy isotope labeled amino acid
AANLWPSPLMIK

SEQ ID NO: 901 - protein, 18 aa, Artificial Sequence
  Internal Standard Peptide Sequence to quantitate RAS-RAF-MAPK Pathway protein
  MOD_RES (18)..(18): heavy isotope labeled amino acid
RTWPLEVPETEPTLGHLR

SEQ ID NO: 902 - protein, 17 aa, Artificial Sequence
  Internal Standard Peptide Sequence to quantitate RAS-RAF-MAPK Pathway protein
  MOD_RES (17)..(17): heavy isotope labeled amino acid
TWPLEVPETEPTLGHLR

SEQ ID NO: 903 - protein, 11 aa, Artificial Sequence
  Internal Standard Peptide Sequence to quantitate RAS-RAF-MAPK Pathway protein
  MOD_RES (10)..(10): carbamidomethylated cysteine
  MOD_RES (11)..(11): heavy isotope labeled amino acid
LQLLPESFICK

SEQ ID NO: 904 - protein, 10 aa, Artificial Sequence
  Internal Standard Peptide Sequence to quantitate RAS-RAF-MAPK Pathway protein
  MOD_RES (10)..(10): heavy isotope labeled amino acid
LGENVANIYK

SEQ ID NO: 905 - protein, 14 aa, Artificial Sequence
  Internal Standard Peptide Sequence to quantitate RAS-RAF-MAPK Pathway protein
  MOD_RES (14)..(14): heavy isotope labeled amino acid
LGENVANIYKDLQK

SEQ ID NO: 906 - protein, 10 aa, Artificial Sequence
  Internal Standard Peptide Sequence to quantitate RAS-RAF-MAPK Pathway protein
  MOD_RES (9)..(9): carbamidomethylated cysteine
  MOD_RES (10)..(10): heavy isotope labeled amino acid
SVLSLSAVCR

SEQ ID NO: 907 - protein, 7 aa, Artificial Sequence
  Internal Standard Peptide Sequence to quantitate RAS-RAF-MAPK Pathway protein
  MOD_RES (7)..(7): heavy isotope labeled amino acid
VQDTDWK

SEQ ID NO: 908 - protein, 11 aa, Artificial Sequence
  Internal Standard Peptide Sequence to quantitate RAS-RAF-MAPK Pathway protein
  MOD_RES (11)..(11): heavy isotope labeled amino acid
VQDTDWKELYR

SEQ ID NO: 909 - protein, 17 aa, Artificial Sequence
  Internal Standard Peptide Sequence to quantitate RAS-RAF-MAPK Pathway protein
  MOD_RES (17)..(17): heavy isotope labeled amino acid
FDPVGPLPGPNPILPGR

SEQ ID NO: 910 - protein, 10 aa, Artificial Sequence
  Internal Standard Peptide Sequence to quantitate RAS-RAF-MAPK Pathway protein
  MOD_RES (10)..(10): heavy isotope labeled amino acid
DGVQLAESNR

SEQ ID NO: 911 - protein, 7 aa, Artificial Sequence
  Internal Standard Peptide Sequence to quantitate RAS-RAF-MAPK Pathway protein
  MOD_RES (7)..(7): heavy isotope labeled amino acid
EFKPDHR

SEQ ID NO: 912 - protein, 15 aa, Artificial Sequence
  Internal Standard Peptide Sequence to quantitate RAS-RAF-MAPK Pathway protein
  MOD_RES (15)..(15): heavy isotope labeled amino acid
SPHRPILQAGLPANK

SEQ ID NO: 913 - protein, 13 aa, Artificial Sequence
  Internal Standard Peptide Sequence to quantitate RAS-RAF-MAPK Pathway protein
  MOD_RES (13)..(13): heavy isotope labeled amino acid
VYSDPQPHIQWLK

SEQ ID NO: 914 - protein, 13 aa, Artificial Sequence
  Internal Standard Peptide Sequence to quantitate RAS-RAF-MAPK Pathway protein
  MOD_RES (13)..(13): heavy isotope labeled amino acid
IGPDNLPYVQILK

SEQ ID NO: 915 - protein, 12 aa, Artificial Sequence
  Internal Standard Peptide Sequence to quantitate RAS-RAF-MAPK Pathway protein
  MOD_RES (1)..(1): carbamidomethylated cysteine
  MOD_RES (12)..(12): heavy isotope labeled amino acid
CPSSGTPNPTLR

SEQ ID NO: 916 - protein, 11 aa, Artificial Sequence
  Internal Standard Peptide Sequence to quantitate RAS-RAF-MAPK Pathway protein
  MOD_RES (11)..(11): heavy isotope labeled amino acid
HPAQLANGGLK

SEQ ID NO: 917 - protein, 22 aa, Artificial Sequence
  Internal Standard Peptide Sequence to quantitate RAS-RAF-MAPK Pathway protein
  MOD_RES (8)..(8): carbamidomethylated cysteine
  MOD_RES (22)..(22): heavy isotope labeled amino acid
RPPGLEYCYNPSHNPEEQLSSK

SEQ ID NO: 918 - protein, 11 aa, Artificial Sequence
  Internal Standard Peptide Sequence to quantitate RAS-RAF-MAPK Pathway protein
  MOD_RES (11)..(11): heavy isotope labeled amino acid
TVLIGEYLQIK

SEQ ID NO: 919 - protein, 10 aa, Artificial Sequence
  Internal Standard Peptide Sequence to quantitate RAS-RAF-MAPK Pathway protein
  MOD_RES (10)..(10): heavy isotope labeled amino acid
YGPDGLPYLK

SEQ ID NO: 920 - protein, 17 aa, Artificial Sequence
  Internal Standard Peptide Sequence to quantitate RAS-RAF-MAPK Pathway protein
  MOD_RES (16)..(16): carbamidomethylated cysteine
  MOD_RES (17)..(17): heavy isotope labeled amino acid
LQVLNASHEDSGAYSCR

SEQ ID NO: 921 - protein, 11 aa, Artificial Sequence
  Internal Standard Peptide Sequence to quantitate RAS-RAF-MAPK Pathway protein
  MOD_RES (11)..(11): heavy isotope labeled amino acid
LLAVPAANTVR

SEQ ID NO: 922 - protein, 13 aa, Artificial Sequence
  Internal Standard Peptide Sequence to quantitate RAS-RAF-MAPK Pathway protein
  MOD_RES (13)..(13): heavy isotope labeled amino acid
LEIASFLPEDAGR

SEQ ID NO: 923 - protein, 21 aa, Artificial Sequence
  Internal Standard Peptide Sequence to quantitate RAS-RAF-MAPK Pathway protein
  MOD_RES (21)..(21): heavy isotope labeled amino acid
HIVINGSSFGADGFPYVQVLK

SEQ ID NO: 924 - protein, 17 aa, Artificial Sequence
  Internal Standard Peptide Sequence to quantitate RAS-RAF-MAPK Pathway protein
  MOD_RES (17)..(17): heavy isotope labeled amino acid
APHPFGVPAPSAGAYSR

SEQ ID NO: 925 - protein, 13 aa, Artificial Sequence
  Internal Standard Peptide Sequence to quantitate RAS-RAF-MAPK Pathway protein
  MOD_RES (13)..(13): heavy isotope labeled amino acid
GKVEQLSPEEEEK

SEQ ID NO: 926 - protein, 11 aa, Artificial Sequence
  Internal Standard Peptide Sequence to quantitate RAS-RAF-MAPK Pathway protein
  MOD_RES (11)..(11): heavy isotope labeled amino acid
VEQLSPEEEEK

SEQ ID NO: 927 - protein, 22 aa, Artificial Sequence
  Internal Standard Peptide Sequence to quantitate RAS-RAF-MAPK Pathway protein
  MOD_RES (22)..(22): heavy isotope labeled amino acid
TEPFDDFLFPASSRPSGSETAR

SEQ ID NO: 928 - protein, 12 aa, Artificial Sequence
  Internal Standard Peptide Sequence to quantitate RAS-RAF-MAPK Pathway protein
  MOD_RES (12)..(12): heavy isotope labeled amino acid
SALQTEIANLLK

SEQ ID NO: 929 - protein, 17 aa, Artificial Sequence
  Internal Standard Peptide Sequence to quantitate RAS-RAF-MAPK Pathway protein
  MOD_RES (17)..(17): heavy isotope labeled amino acid
ELTDTLQAETDQLEDEK

SEQ ID NO: 930 - protein, 12 aa, Artificial Sequence
  Internal Standard Peptide Sequence to quantitate RAS-RAF-MAPK Pathway protein
  MOD_RES (12)..(12): heavy isotope labeled amino acid
VEQLSPEEEEKR

SEQ ID NO: 931 - protein, 20 aa, Artificial Sequence
  Internal Standard Peptide Sequence to quantitate RAS-RAF-MAPK Pathway protein
  MOD_RES (20)..(20): heavy isotope labeled amino acid
ASLQSGQEGAGDSPGSQFSK

SEQ ID NO: 932 - protein, 10 aa, Artificial Sequence
  Internal Standard Peptide Sequence to quantitate RAS-RAF-MAPK Pathway protein
  MOD_RES (3)..(3): carbamidomethylated cysteine
  MOD_RES (10)..(10): heavy isotope labeled amino acid
LDCDMESIIR

SEQ ID NO: 933 - protein, 19 aa, Artificial Sequence
  Internal Standard Peptide Sequence to quantitate RAS-RAF-MAPK Pathway protein
  MOD_RES (19)..(19): heavy isotope labeled amino acid
AALQTAPESADDSPSQLSK

SEQ ID NO: 934 - protein, 12 aa, Artificial Sequence
  Internal Standard Peptide Sequence to quantitate RAS-RAF-MAPK Pathway protein
  MOD_RES (12)..(12): heavy isotope labeled amino acid
SSDELDAWTDFR

SEQ ID NO: 935 - protein, 9 aa, Artificial Sequence
  Internal Standard Peptide Sequence to quantitate RAS-RAF-MAPK Pathway protein
  MOD_RES (9)..(9): heavy isotope labeled amino acid
SFEDIHQYR

SEQ ID NO: 936 - protein, 12 aa, Artificial Sequence
  Internal Standard Peptide Sequence to quantitate RAS-RAF-MAPK Pathway protein
  MOD_RES (12)..(12): heavy isotope labeled amino acid
SYGIPYIETSAK

SEQ ID NO: 937 - protein, 15 aa, Artificial Sequence
  Internal Standard Peptide Sequence to quantitate RAS-RAF-MAPK Pathway protein
  MOD_RES (11)..(14): carbamidomethylated cysteine
  MOD_RES (15)..(15): heavy isotope labeled amino acid
LNPPDESGPGCMSCK

SEQ ID NO: 938 - protein, 10 aa, Artificial Sequence
  Internal Standard Peptide Sequence to quantitate RAS-RAF-MAPK Pathway protein
  MOD_RES (10)..(10): heavy isotope labeled amino acid
LFDQAFGLPR

SEQ ID NO: 939 - protein, 17 aa, Artificial Sequence
  Internal Standard Peptide Sequence to quantitate RAS-RAF-MAPK Pathway protein
  MOD_RES (17)..(17): heavy isotope labeled amino acid
LATQSNEITIPVTFESR

SEQ ID NO: 940 - protein, 16 aa, Artificial Sequence
  Internal Standard Peptide Sequence to quantitate RAS-RAF-MAPK Pathway protein
  MOD_RES (16)..(16): heavy isotope labeled amino acid
VSLDVNHFAPDELTVK

SEQ ID NO: 941 - protein, 8 aa, Artificial Sequence
  Internal Standard Peptide Sequence to quantitate RAS-RAF-MAPK Pathway protein
  MOD_RES (8)..(8): heavy isotope labeled amino acid
GPSWDPFR

SEQ ID NO: 942 - protein, 9 aa, Artificial Sequence
  Internal Standard Peptide Sequence to quantitate RAS-RAF-MAPK Pathway protein
  MOD_RES (9)..(9): heavy isotope labeled amino acid
DGVVEITGK

SEQ ID NO: 943 - protein, 7 aa, Artificial Sequence
  Internal Standard Peptide Sequence to quantitate RAS-RAF-MAPK Pathway protein
  MOD_RES (7)..(7): heavy isotope labeled amino acid
VPFSLLR

SEQ ID NO: 944 - protein, 10 aa, Artificial Sequence
  Internal Standard Peptide Sequence to quantitate RAS-RAF-MAPK Pathway protein
  MOD_RES (10)..(10): heavy isotope labeled amino acid
AQLGGPEAAK

SEQ ID NO: 945 - protein, 8 aa, Artificial Sequence
  Internal Standard Peptide Sequence to quantitate RAS-RAF-MAPK Pathway protein
  MOD_RES (8)..(8): heavy isotope labeled amino acid
LGNFFSPK

SEQ ID NO: 946 - protein, 7 aa, Artificial Sequence
  Internal Standard Peptide Sequence to quantitate RAS-RAF-MAPK Pathway protein
  MOD_RES (7)..(7): heavy isotope labeled amino acid
ATGLHFR

SEQ ID NO: 947 - protein, 13 aa, Artificial Sequence
  Internal Standard Peptide Sequence to quantitate RAS-RAF-MAPK Pathway protein
  MOD_RES (13)..(13): heavy isotope labeled amino acid
LGLAPQIQDLYGK

SEQ ID NO: 948 - protein, 10 aa, Artificial Sequence
  Internal Standard Peptide Sequence to quantitate RAS-RAF-MAPK Pathway protein
  MOD_RES (10)..(10): heavy isotope labeled amino acid
ALQSPALGLR

SEQ ID NO: 949 - protein, 7 aa, Artificial Sequence
  Internal Standard Peptide Sequence to quantitate RAS-RAF-MAPK Pathway protein
  MOD_RES (7)..(7): heavy isotope labeled amino acid
ELATLQR

SEQ ID NO: 950 - protein, 12 aa, Artificial Sequence
  Internal Standard Peptide Sequence to quantitate RAS-RAF-MAPK Pathway protein
  MOD_RES (12)..(12): heavy isotope labeled amino acid
ALESGDVNTVWK

SEQ ID NO: 951 - protein, 11 aa, Artificial Sequence
  Internal Standard Peptide Sequence to quantitate RAS-RAF-MAPK Pathway protein
  MOD_RES (11)..(11): heavy isotope labeled amino acid
TLQALQIPAAK

SEQ ID NO: 952 - protein, 7 aa, Artificial Sequence
  Internal Standard Peptide Sequence to quantitate RAS-RAF-MAPK Pathway protein
  MOD_RES (7)..(7): heavy isotope labeled amino acid
EEYLLLR

SEQ ID NO: 953 - protein, 17 aa, Artificial Sequence
  Internal Standard Peptide Sequence to quantitate RAS-RAF-MAPK Pathway protein
  MOD_RES (17)..(17): heavy isotope labeled amino acid
LPYDVTPEQALAHEEVK

SEQ ID NO: 954 - protein, 9 aa, Artificial Sequence
  Internal Standard Peptide Sequence to quantitate RAS-RAF-MAPK Pathway protein
  MOD_RES (9)..(9): heavy isotope labeled amino acid
IIGNLLYYR

SEQ ID NO: 955 - protein, 11 aa, Artificial Sequence
  Internal Standard Peptide Sequence to quantitate RAS-RAF-MAPK Pathway protein
  MOD_RES (11)..(11): heavy isotope labeled amino acid
LQQTYAALNSK

SEQ ID NO: 956 - protein, 12 aa, Artificial Sequence
  Internal Standard Peptide Sequence to quantitate RAS-RAF-MAPK Pathway protein
  MOD_RES (12)..(12): heavy isotope labeled amino acid
ATFYGEQVDYYK

SEQ ID NO: 957 - protein, 9 aa, Artificial Sequence
  Internal Standard Peptide Sequence to quantitate RAS-RAF-MAPK Pathway protein
  MOD_RES (2)..(2): carbamidomethylated cysteine
  MOD_RES (9)..(9): heavy isotope labeled amino acid
TCLDNLASK

SEQ ID NO: 958 - protein, 14 aa, Artificial Sequence
  Internal Standard Peptide Sequence to quantitate RAS-RAF-MAPK Pathway protein
  MOD_RES (12)..(12): carbamidomethylated cysteine
  MOD_RES (14)..(14): heavy isotope labeled amino acid
LGSGEYTAEELCIR

SEQ ID NO: 959 - protein, 17 aa, Artificial Sequence
  Internal Standard Peptide Sequence to quantitate RAS-RAF-MAPK Pathway protein
  MOD_RES (5)..(5): carbamidomethylated cysteine
  MOD_RES (17)..(17): heavy isotope labeled amino acid
ISPLCHNLFALYDENTK

SEQ ID NO: 960 - protein, 8 aa, Artificial Sequence
  Internal Standard Peptide Sequence to quantitate RAS-RAF-MAPK Pathway protein
  MOD_RES (8)..(8): heavy isotope labeled amino acid
YIPETLNK

SEQ ID NO: 961 - protein, 10 aa, Artificial Sequence
  Internal Standard Peptide Sequence to quantitate RAS-RAF-MAPK Pathway protein
  MOD_RES (10)..(10): heavy isotope labeled amino acid
YLATLETLTK

SEQ ID NO: 962 - protein, 8 aa, Artificial Sequence
  Internal Standard Peptide Sequence to quantitate RAS-RAF-MAPK Pathway protein
  MOD_RES (8)..(8): heavy isotope labeled amino acid
NFQIEVQK

SEQ ID NO: 963 - protein, 10 aa, Artificial Sequence
  Internal Standard Peptide Sequence to quantitate RAS-RAF-MAPK Pathway protein
  MOD_RES (10)..(10): heavy isotope labeled amino acid
EISNLLVATK

SEQ ID NO: 964 - protein, 11 aa, Artificial Sequence
  Internal Standard Peptide Sequence to quantitate RAS-RAF-MAPK Pathway protein
  MOD_RES (11)..(11): heavy isotope labeled amino acid
EISNLLVATKK

SEQ ID NO: 965 - protein, 11 aa, Artificial Sequence
  Internal Standard Peptide Sequence to quantitate RAS-RAF-MAPK Pathway protein
  MOD_RES (11)..(11): heavy isotope labeled amino acid
KDLVQGEHLGR

SEQ ID NO: 966 - protein, 10 aa, Artificial Sequence
  Internal Standard Peptide Sequence to quantitate RAS-RAF-MAPK Pathway protein
  MOD_RES (10)..(10): heavy isotope labeled amino acid
DLVQGEHLGR

SEQ ID NO: 967 - protein, 12 aa, Artificial Sequence
  Internal Standard Peptide Sequence to quantitate RAS-RAF-MAPK Pathway protein
  MOD_RES (7)..(7): carbamidomethylated cysteine
  MOD_RES (12)..(12): heavy isotope labeled amino acid
EGIDSECGPFIK

SEQ ID NO: 968 - protein, 13 aa, Artificial Sequence
  Internal Standard Peptide Sequence to quantitate RAS-RAF-MAPK Pathway protein
  MOD_RES (13)..(13): heavy isotope labeled amino acid
LSDPGIPITVLSR

SEQ ID NO: 969 - protein, 13 aa, Artificial Sequence
  Internal Standard Peptide Sequence to quantitate RAS-RAF-MAPK Pathway protein
  MOD_RES (8)..(8): carbamidomethylated cysteine
  MOD_RES (13)..(13): heavy isotope labeled amino acid
IPWIAPECVEDSK

SEQ ID NO: 970 - protein, 15 aa, Artificial Sequence
  Internal Standard Peptide Sequence to quantitate RAS-RAF-MAPK Pathway protein
  MOD_RES (15)..(15): heavy isotope labeled amino acid
YDPEGDNTGEQVAVK

SEQ ID NO: 971 - protein, 9 aa, Artificial Sequence
  Internal Standard Peptide Sequence to quantitate RAS-RAF-MAPK Pathway protein
  MOD_RES (9)..(9): heavy isotope labeled amino acid
NLYHENIVK

SEQ ID NO: 972 - protein, 11 aa, Artificial Sequence
  Internal Standard Peptide Sequence to quantitate RAS-RAF-MAPK Pathway protein
  MOD_RES (11)..(11): heavy isotope labeled amino acid
NVLVESEHQVK

SEQ ID NO: 973 - protein, 9 aa, Artificial Sequence
  Internal Standard Peptide Sequence to quantitate RAS-RAF-MAPK Pathway protein
  MOD_RES (9)..(9): heavy isotope labeled amino acid
HDFVHGWIK

SEQ ID NO: 974 - protein, 9 aa, Artificial Sequence
  Internal Standard Peptide Sequence to quantitate RAS-RAF-MAPK Pathway protein
  MOD_RES (9)..(9): heavy isotope labeled amino acid
IQDYHILTR

SEQ ID NO: 975 - protein, 10 aa, Artificial Sequence
  Internal Standard Peptide Sequence to quantitate RAS-RAF-MAPK Pathway protein
  MOD_RES (10)..(10): heavy isotope labeled amino acid
NLEIELSSLR

SEQ ID NO: 976 - protein, 11 aa, Artificial Sequence
  Internal Standard Peptide Sequence to quantitate RAS-RAF-MAPK Pathway protein
  MOD_RES (11)..(11): heavy isotope labeled amino acid
AGNQTGLYVLR

SEQ ID NO: 977 - protein, 9 aa, Artificial Sequence
  Internal Standard Peptide Sequence to quantitate RAS-RAF-MAPK Pathway protein
  MOD_RES (9)..(9): heavy isotope labeled amino acid
YFLTFAVER

SEQ ID NO: 978 - protein, 12 aa, Artificial Sequence
  Internal Standard Peptide Sequence to quantitate RAS-RAF-MAPK Pathway protein
  MOD_RES (12)..(12): heavy isotope labeled amino acid
NENEEYNLSGTK

SEQ ID NO: 979 - protein, 10 aa, Artificial Sequence
  Internal Standard Peptide Sequence to quantitate RAS-RAF-MAPK Pathway protein
  MOD_RES (10)..(10): heavy isotope labeled amino acid
SDNIIFQFTK

SEQ ID NO: 980 - protein, 16 aa, Artificial Sequence
  Internal Standard Peptide Sequence to quantitate RAS-RAF-MAPK Pathway protein
  MOD_RES (16)..(16): heavy isotope labeled amino acid
EVGDYGQLHETEVLLK

SEQ ID NO: 981 - protein, 13 aa, Artificial Sequence
  Internal Standard Peptide Sequence to quantitate RAS-RAF-MAPK Pathway protein
  MOD_RES (13)..(13): heavy isotope labeled amino acid
LSDPGISITVLPK

SEQ ID NO: 982 - protein, 13 aa, Artificial Sequence
  Internal Standard Peptide Sequence to quantitate RAS-RAF-MAPK Pathway protein
  MOD_RES (8)..(8): carbamidomethylated cysteine
  MOD_RES (13)..(13): heavy isotope labeled amino acid
IPWVPPECIENPK

SEQ ID NO: 983 - protein, 7 aa, Artificial Sequence
  Internal Standard Peptide Sequence to quantitate RAS-RAF-MAPK Pathway protein
  MOD_RES (7)..(7): heavy isotope labeled amino acid
FLQQLGK

SEQ ID NO: 984 - protein, 15 aa, Artificial Sequence
  Internal Standard Peptide Sequence to quantitate RAS-RAF-MAPK Pathway protein
  MOD_RES (15)..(15): heavy isotope labeled amino acid
YDPLQDNTGEVVAVK

SEQ ID NO: 985 - protein, 9 aa, Artificial Sequence
  Internal Standard Peptide Sequence to quantitate RAS-RAF-MAPK Pathway protein
  MOD_RES (9)..(9): heavy isotope labeled amino acid
SLQHDNIVK

SEQ ID NO: 986 - protein, 10 aa, Artificial Sequence
  Internal Standard Peptide Sequence to quantitate RAS-RAF-MAPK Pathway protein
  MOD_RES (9)..(9): carbamidomethylated cysteine
  MOD_RES (10)..(10): heavy isotope labeled amino acid
LLQYTSQICK

SEQ ID NO: 987 - protein, 9 aa, Artificial Sequence
  Internal Standard Peptide Sequence to quantitate RAS-RAF-MAPK Pathway protein
  MOD_RES (9)..(9): heavy isotope labeled amino acid
NILVENENR

SEQ ID NO: 988 - protein, 16 aa, Artificial Sequence
  Internal Standard Peptide Sequence to quantitate RAS-RAF-MAPK Pathway protein
  MOD_RES (16)..(16): heavy isotope labeled amino acid
ENDQTPLAIYNSISYK

SEQ ID NO: 989 - protein, 19 aa, Artificial Sequence
  Internal Standard Peptide Sequence to quantitate RAS-RAF-MAPK Pathway protein
  MOD_RES (19)..(19): heavy isotope labeled amino acid
EPGESPIFWYAPESLTESK

SEQ ID NO: 990 - protein, 15 aa, Artificial Sequence
  Internal Standard Peptide Sequence to quantitate RAS-RAF-MAPK Pathway protein
  MOD_RES (15)..(15): heavy isotope labeled amino acid
NVTDEQEGFAEGFVR

SEQ ID NO: 991 - protein, 14 aa, Artificial Sequence
  Internal Standard Peptide Sequence to quantitate RAS-RAF-MAPK Pathway protein
  MOD_RES (14)..(14): heavy isotope labeled amino acid
NSDLLTSPDVGLLK

SEQ ID NO: 992 - protein, 14 aa, Artificial Sequence
  Internal Standard Peptide Sequence to quantitate RAS-RAF-MAPK Pathway protein
  MOD_RES (14)..(14): heavy isotope labeled amino acid
AQNSELASTANMLR

SEQ ID NO: 993 - protein, 23 aa, Artificial Sequence
  Internal Standard Peptide Sequence to quantitate RAS-RAF-MAPK Pathway protein
  MOD_RES (21)..(21): carbamidomethylated cysteine
  MOD_RES (23)..(23): heavy isotope labeled amino acid
LIIQSSNGHITTTPTPTQFLCPK

SEQ ID NO: 994 - protein, 26 aa, Artificial Sequence
  Internal Standard Peptide Sequence to quantitate RAS-RAF-MAPK Pathway protein
  MOD_RES (26)..(26): heavy isotope labeled amino acid
EEPQTVPEMPGETPPLSPIDMESQER

SEQ ID NO: 995 - protein, 9 aa, Artificial Sequence
  Internal Standard Peptide Sequence to quantitate RAS-RAF-MAPK Pathway protein
  MOD_RES (9)..(9): heavy isotope labeled amino acid
SFEDIHHYR

SEQ ID NO: 996 - protein, 13 aa, Artificial Sequence
  Internal Standard Peptide Sequence to quantitate RAS-RAF-MAPK Pathway protein
  MOD_RES (13)..(13): heavy isotope labeled amino acid
DSEDVPMVLVGNK

SEQ ID NO: 997 - protein, 10 aa, Artificial Sequence
  Internal Standard Peptide Sequence to quantitate RAS-RAF-MAPK Pathway protein
  MOD_RES (10)..(10): heavy isotope labeled amino acid
VEDAFYTLVR

SEQ ID NO: 998 - protein, 12 aa, Artificial Sequence
  Internal Standard Peptide Sequence to quantitate RAS-RAF-MAPK Pathway protein
  MOD_RES (12)..(12): heavy isotope labeled amino acid
QGVDDAFYTLVR

SEQ ID NO: 999 - protein, 15 aa, Artificial Sequence
  Internal Standard Peptide Sequence to quantitate RAS-RAF-MAPK Pathway protein
  MOD_RES (7)..(7): carbamidomethylated cysteine
  MOD_RES (15)..(15): heavy isotope labeled amino acid
LSHDWLCYLAPEIVR

SEQ ID NO: 1000 - protein, 18 aa, Artificial Sequence
  Internal Standard Peptide Sequence to quantitate RAS-RAF-MAPK Pathway protein
  MOD_RES (18)..(18): heavy isotope labeled amino acid
ADVLEAHEAEAEEPEAGK

SEQ ID NO: 1001 - protein, 14 aa, Artificial Sequence
  Internal Standard Peptide Sequence to quantitate RAS-RAF-MAPK Pathway protein
  MOD_RES (14)..(14): heavy isotope labeled amino acid
ISELGAGNGGVVFK

SEQ ID NO: 1002 - protein, 8 aa, Artificial Sequence
  Internal Standard Peptide Sequence to quantitate RAS-RAF-MAPK Pathway protein
  MOD_RES (8)..(8): heavy isotope labeled amino acid
IPEQILGK

SEQ ID NO: 1003 - protein, 12 aa, Artificial Sequence
  Internal Standard Peptide Sequence to quantitate RAS-RAF-MAPK Pathway protein
  MOD_RES (12)..(12): heavy isotope labeled amino acid
VSHKPSGLVMAR

SEQ ID NO: 1004 - protein, 11 aa, Artificial Sequence
  Internal Standard Peptide Sequence to quantitate RAS-RAF-MAPK Pathway protein
  MOD_RES (11)..(11): heavy isotope labeled amino acid
LEELELDEQQR

SEQ ID NO: 1005 - protein, 7 aa, Artificial Sequence
  Internal Standard Peptide Sequence to quantitate RAS-RAF-MAPK Pathway protein
  MOD_RES (7)..(7): heavy isotope labeled amino acid
VSIAVIK

SEQ ID NO: 1006 - protein, 14 aa, Artificial Sequence
  Internal Standard Peptide Sequence to quantitate RAS-RAF-MAPK Pathway protein
  MOD_RES (14)..(14): heavy isotope labeled amino acid
ISELGAGNGGVVTK

SEQ ID NO: 1007 - protein, 12 aa, Artificial Sequence
  Internal Standard Peptide Sequence to quantitate RAS-RAF-MAPK Pathway protein
  MOD_RES (12)..(12): heavy isotope labeled amino acid
VQHRPSGLIMAR

SEQ ID NO: 1008 - protein, 8 aa, Artificial Sequence
  Internal Standard Peptide Sequence to quantitate RAS-RAF-MAPK Pathway protein
  MOD_RES (8)..(8): heavy isotope labeled amino acid
IPEEILGK

SEQ ID NO: 1009 - protein, 14 aa, Artificial Sequence
  Internal Standard Peptide Sequence to quantitate RAS-RAF-MAPK Pathway protein
  MOD_RES (13)..(13): carbamidomethylated cysteine
  MOD_RES (14)..(14): heavy isotope labeled amino acid
SEVEEVDFAGWLCK

SEQ ID NO: 1010 - protein, 9 aa, Artificial Sequence
  Internal Standard Peptide Sequence to quantitate RAS-RAF-MAPK Pathway protein
  MOD_RES (9)..(9): heavy isotope labeled amino acid
LNQPGTPTR

SEQ ID NO: 1011 - protein, 14 aa, Artificial Sequence
  Internal Standard Peptide Sequence to quantitate RAS-RAF-MAPK Pathway protein
  MOD_RES (12)..(12): carbamidomethylated cysteine
  MOD_RES (14)..(14): heavy isotope labeled amino acid
FSPEFVDFTAQCLR

SEQ ID NO: 1012 - protein, 14 aa, Artificial Sequence
  Internal Standard Peptide Sequence to quantitate RAS-RAF-MAPK Pathway protein
  MOD_RES (14)..(14): heavy isotope labeled amino acid
FPYESWGTPFQQLK

SEQ ID NO: 1013 - protein, 11 aa, Artificial Sequence
  Internal Standard Peptide Sequence to quantitate RAS-RAF-MAPK Pathway protein
  MOD_RES (11)..(11): heavy isotope labeled amino acid
DVKPSNVLINK

SEQ ID NO: 1014 - protein, 8 aa, Artificial Sequence
  Internal Standard Peptide Sequence to quantitate RAS-RAF-MAPK Pathway protein
  MOD_RES (8)..(8): heavy isotope labeled amino acid
TFITIGDR

SEQ ID NO: 1015 - protein, 8 aa, Artificial Sequence
  Internal Standard Peptide Sequence to quantitate RAS-RAF-MAPK Pathway protein
  MOD_RES (8)..(8): heavy isotope labeled amino acid
TDIAAFVK

SEQ ID NO: 1016 - protein, 17 aa, Artificial Sequence
  Internal Standard Peptide Sequence to quantitate RAS-RAF-MAPK Pathway protein
  MOD_RES (17)..(17): heavy isotope labeled amino acid
NFEVEADDLVTISELGR

SEQ ID NO: 1017 - protein, 16 aa, Artificial Sequence
  Internal Standard Peptide Sequence to quantitate RAS-RAF-MAPK Pathway protein
  MOD_RES (2)..(2): carbamidomethylated cysteine
  MOD_RES (16)..(16): heavy isotope labeled amino acid
LCDFGISGQLVDSIAK

SEQ ID NO: 1018 - protein, 17 aa, Artificial Sequence
  Internal Standard Peptide Sequence to quantitate RAS-RAF-MAPK Pathway protein
  MOD_RES (17)..(17): heavy isotope labeled amino acid
FTLNPNPTGVQNPHIER

SEQ ID NO: 1019 - protein, 13 aa, Artificial Sequence
  Internal Standard Peptide Sequence to quantitate RAS-RAF-MAPK Pathway protein
  MOD_RES (13)..(13): heavy isotope labeled amino acid
WNSVFDQLTQVVK

SEQ ID NO: 1020 - protein, 17 aa, Artificial Sequence
  Internal Standard Peptide Sequence to quantitate RAS-RAF-MAPK Pathway protein
  MOD_RES (17)..(17): heavy isotope labeled amino acid
YVYSVLDDVIPEEILGK

SEQ ID NO: 1021 - protein, 11 aa, Artificial Sequence
  Internal Standard Peptide Sequence to quantitate RAS-RAF-MAPK Pathway protein
  MOD_RES (11)..(11): heavy isotope labeled amino acid
DIKPSNILLDR

SEQ ID NO: 1022 - protein, 12 aa, Artificial Sequence
  Internal Standard Peptide Sequence to quantitate RAS-RAF-MAPK Pathway protein
  MOD_RES (12)..(12): heavy isotope labeled amino acid
GDPPQLSNSEER

SEQ ID NO: 1023 - protein, 10 aa, Artificial Sequence
  Internal Standard Peptide Sequence to quantitate RAS-RAF-MAPK Pathway protein
  MOD_RES (6)..(9): carbamidomethylated cysteine
  MOD_RES (10)..(10): heavy isotope labeled amino acid
AVEVACYVCK

SEQ ID NO: 1024 - protein, 16 aa, Artificial Sequence
  Internal Standard Peptide Sequence to quantitate RAS-RAF-MAPK Pathway protein
  MOD_RES (16)..(16): heavy isotope labeled amino acid
DVKPSNVLINALGQVK

SEQ ID NO: 1025 - protein, 14 aa, Artificial Sequence
  Internal Standard Peptide Sequence to quantitate RAS-RAF-MAPK Pathway protein
  MOD_RES (14)..(14): heavy isotope labeled amino acid
EAFEQPQTSSTPPR

SEQ ID NO: 1026 - protein, 13 aa, Artificial Sequence
  Internal Standard Peptide Sequence to quantitate RAS-RAF-MAPK Pathway protein
  MOD_RES (2)..(2): carbamidomethylated cysteine
  MOD_RES (13)..(13): heavy isotope labeled amino acid
ACISIGNQNFEVK

SEQ ID NO: 1027 - protein, 9 aa, Artificial Sequence
  Internal Standard Peptide Sequence to quantitate RAS-RAF-MAPK Pathway protein
  MOD_RES (9)..(9): heavy isotope labeled amino acid
GTDVASFVK

SEQ ID NO: 1028 - protein, 7 aa, Artificial Sequence
  Internal Standard Peptide Sequence to quantitate RAS-RAF-MAPK Pathway protein
  MOD_RES (7)..(7): heavy isotope labeled amino acid
IAVSIVK

SEQ ID NO: 1029 - protein, 14 aa, Artificial Sequence
  Internal Standard Peptide Sequence to quantitate RAS-RAF-MAPK Pathway protein
  MOD_RES (14)..(14): heavy isotope labeled amino acid
FPYDSWGTPFQQLK

SEQ ID NO: 1030 - protein, 11 aa, Artificial Sequence
  Internal Standard Peptide Sequence to quantitate RAS-RAF-MAPK Pathway protein
  MOD_RES (11)..(11): heavy isotope labeled amino acid
GQTIPEDILGK

SEQ ID NO: 1031 - protein, 14 aa, Artificial Sequence
  Internal Standard Peptide Sequence to quantitate RAS-RAF-MAPK Pathway protein
  MOD_RES (12)..(12): carbamidomethylated cysteine
  MOD_RES (14)..(14): heavy isotope labeled amino acid
FSAEFVDFTSQCLK

SEQ ID NO: 1032 - protein, 18 aa, Artificial Sequence
  Internal Standard Peptide Sequence to quantitate RAS-RAF-MAPK Pathway protein
  MOD_RES (18)..(18): heavy isotope labeled amino acid
SPSSESSPQHPTPPARPR

SEQ ID NO: 1033 - protein, 8 aa, Artificial Sequence
  Internal Standard Peptide Sequence to quantitate RAS-RAF-MAPK Pathway protein
  MOD_RES (8)..(8): heavy isotope labeled amino acid
TDFEVLTK

SEQ ID NO: 1034 - protein, 12 aa, Artificial Sequence
  Internal Standard Peptide Sequence to quantitate RAS-RAF-MAPK Pathway protein
  MOD_RES (12)..(12): heavy isotope labeled amino acid
DVKPSNILLDER

SEQ ID NO: 1035 - protein, 8 aa, Artificial Sequence
  Internal Standard Peptide Sequence to quantitate RAS-RAF-MAPK Pathway protein
  MOD_RES (8)..(8): heavy isotope labeled amino acid
LLEHSFIK

SEQ ID NO: 1036 - protein, 14 aa, Artificial Sequence
  Internal Standard Peptide Sequence to quantitate RAS-RAF-MAPK Pathway protein
  MOD_RES (14)..(14): heavy isotope labeled amino acid
TSGVLSQPHLPFFR

SEQ ID NO: 1037 - protein, 14 aa, Artificial Sequence
  Internal Standard Peptide Sequence to quantitate RAS-RAF-MAPK Pathway protein
  MOD_RES (14)..(14): heavy isotope labeled amino acid
IDPPDPTKPDYDIR

SEQ ID NO: 1038 - protein, 8 aa, Artificial Sequence
  Internal Standard Peptide Sequence to quantitate RAS-RAF-MAPK Pathway protein
  MOD_RES (8)..(8): heavy isotope labeled amino acid
AASSLEQK

SEQ ID NO: 1039 - protein, 9 aa, Artificial Sequence
  Internal Standard Peptide Sequence to quantitate RAS-RAF-MAPK Pathway protein
  MOD_RES (2)..(2): carbamidomethylated cysteine
  MOD_RES (9)..(9): heavy isotope labeled amino acid
LCDFGISGR

SEQ ID NO: 1040 - protein, 9 aa, Artificial Sequence
  Internal Standard Peptide Sequence to quantitate RAS-RAF-MAPK Pathway protein
  MOD_RES (9)..(9): heavy isotope labeled amino acid
GQVFDVGPR

SEQ ID NO: 1041 - protein, 14 aa, Artificial Sequence
  Internal Standard Peptide Sequence to quantitate RAS-RAF-MAPK Pathway protein
  MOD_RES (14)..(14): heavy isotope labeled amino acid
FRHENIIGINDIIR

SEQ ID NO: 1042 - protein, 12 aa, Artificial Sequence
  Internal Standard Peptide Sequence to quantitate RAS-RAF-MAPK Pathway protein
  MOD_RES (12)..(12): heavy isotope labeled amino acid
HENIIGINDIIR

SEQ ID NO: 1043 - protein, 18 aa, Artificial Sequence
  Internal Standard Peptide Sequence to quantitate RAS-RAF-MAPK Pathway protein
  MOD_RES (10)..(10): carbamidomethylated cysteine
  MOD_RES (18)..(18): heavy isotope labeled amino acid
TQHLSNDHICYFLYQILR

SEQ ID NO: 1044 - protein, 16 aa, Artificial Sequence
  Internal Standard Peptide Sequence to quantitate RAS-RAF-MAPK Pathway protein
  MOD_RES (13)..(13): carbamidomethylated cysteine
  MOD_RES (16)..(16): heavy isotope labeled amino acid
DLKPSNLLLNTTCDLK

SEQ ID NO: 1045 - protein, 19 aa, Artificial Sequence
  Internal Standard Peptide Sequence to quantitate RAS-RAF-MAPK Pathway protein
  MOD_RES (19)..(19): heavy isotope labeled amino acid
VADPDHDHTGFLTEYVATR

SEQ ID NO: 1046 - protein, 9 aa, Artificial Sequence
  Internal Standard Peptide Sequence to quantitate RAS-RAF-MAPK Pathway protein
  MOD_RES (9)..(9): heavy isotope labeled amino acid
NYLLSLPHK

SEQ ID NO: 1047 - protein, 7 aa, Artificial Sequence
  Internal Standard Peptide Sequence to quantitate RAS-RAF-MAPK Pathway protein
  MOD_RES (7)..(7): heavy isotope labeled amino acid
NKVPWNR

SEQ ID NO: 1048 - protein, 8 aa, Artificial Sequence
  Internal Standard Peptide Sequence to quantitate RAS-RAF-MAPK Pathway protein
  MOD_RES (8)..(8): heavy isotope labeled amino acid
LFPNADSK

SEQ ID NO: 1049 - protein, 7 aa, Artificial Sequence
  Internal Standard Peptide Sequence to quantitate RAS-RAF-MAPK Pathway protein
  MOD_RES (7)..(7): heavy isotope labeled amino acid
ALDLLDK

SEQ ID NO: 1050 - protein, 8 aa, Artificial Sequence
  Internal Standard Peptide Sequence to quantitate RAS-RAF-MAPK Pathway protein
  MOD_RES (8)..(8): heavy isotope labeled amino acid
MLTFNPHK

SEQ ID NO: 1051 - protein, 9 aa, Artificial Sequence
  Internal Standard Peptide Sequence to quantitate RAS-RAF-MAPK Pathway protein
  MOD_RES (9)..(9): heavy isotope labeled amino acid
MLTFNPHKR

SEQ ID NO: 1052 - protein, 11 aa, Artificial Sequence
  Internal Standard Peptide Sequence to quantitate RAS-RAF-MAPK Pathway protein
  MOD_RES (11)..(11): heavy isotope labeled amino acid
LKELIFEETAR

SEQ ID NO: 1053 - protein, 8 aa, Artificial Sequence
  Internal Standard Peptide Sequence to quantitate RAS-RAF-MAPK Pathway protein
  MOD_RES (8)..(8): heavy isotope labeled amino acid
TVWEVPQR

SEQ ID NO: 1054 - protein, 22 aa, Artificial Sequence
  Internal Standard Peptide Sequence to quantitate RAS-RAF-MAPK Pathway protein
  MOD_RES (16)..(16): carbamidomethylated cysteine
  MOD_RES (22)..(22): heavy isotope labeled amino acid
LQGLRPVGSGAYGSVCSAYDAR

SEQ ID NO: 1055 - protein, 18 aa, Artificial Sequence
  Internal Standard Peptide Sequence to quantitate RAS-RAF-MAPK Pathway protein
  MOD_RES (1)..(1): carbamidomethylated cysteine
  MOD_RES (18)..(18): heavy isotope labeled amino acid
CQALSDEHVQFLVYQLLR

SEQ ID NO: 1056 - protein, 10 aa, Artificial Sequence
  Internal Standard Peptide Sequence to quantitate RAS-RAF-MAPK Pathway protein
  MOD_RES (10)..(10): heavy isotope labeled amino acid
YIHSAGIIHR

SEQ ID NO: 1057 - protein, 16 aa, Artificial Sequence
  Internal Standard Peptide Sequence to quantitate RAS-RAF-MAPK Pathway protein
  MOD_RES (13)..(13): carbamidomethylated cysteine
  MOD_RES (16)..(16): heavy isotope labeled amino acid
DLKPSNVAVNEDCELR

SEQ ID NO: 1058 - protein, 13 aa, Artificial Sequence
  Internal Standard Peptide Sequence to quantitate RAS-RAF-MAPK Pathway protein
  MOD_RES (13)..(13): heavy isotope labeled amino acid
ALFPGSDYIDQLK

SEQ ID NO: 1059 - protein, 14 aa, Artificial Sequence
  Internal Standard Peptide Sequence to quantitate RAS-RAF-MAPK Pathway protein
  MOD_RES (14)..(14): heavy isotope labeled amino acid
ALFPGSDYIDQLKR

SEQ ID NO: 1060 - protein, 15 aa, Artificial Sequence
  Internal Standard Peptide Sequence to quantitate RAS-RAF-MAPK Pathway protein
  MOD_RES (15)..(15): heavy isotope labeled amino acid
IMEVVGTPSPEVLAK

SEQ ID NO: 1061 - protein, 12 aa, Artificial Sequence
  Internal Standard Peptide Sequence to quantitate RAS-RAF-MAPK Pathway protein
  MOD_RES (12)..(12): heavy isotope labeled amino acid
TYIQSLPPMPQK

SEQ ID NO: 1062 - protein, 7 aa, Artificial Sequence
  Internal Standard Peptide Sequence to quantitate RAS-RAF-MAPK Pathway protein
  MOD_RES (7)..(7): heavy isotope labeled amino acid
DLSSIFR

SEQ ID NO: 1063 - protein, 12 aa, Artificial Sequence
  Internal Standard Peptide Sequence to quantitate RAS-RAF-MAPK Pathway protein
  MOD_RES (12)..(12): heavy isotope labeled amino acid
GANPLAIDLLGR

SEQ ID NO: 1064 - protein, 9 aa, Artificial Sequence
  Internal Standard Peptide Sequence to quantitate RAS-RAF-MAPK Pathway protein
  MOD_RES (9)..(9): heavy isotope labeled amino acid
MLVLDSDQR

SEQ ID NO: 1065 - protein, 12 aa, Artificial Sequence
  Internal Standard Peptide Sequence to quantitate RAS-RAF-MAPK Pathway protein
  MOD_RES (12)..(12): heavy isotope labeled amino acid
LSRPFQSLIHAR

SEQ ID NO: 1066 - protein, 10 aa, Artificial Sequence
  Internal Standard Peptide Sequence to quantitate RAS-RAF-MAPK Pathway protein
  MOD_RES (10)..(10): heavy isotope labeled amino acid
YDDSFDDVDR

SEQ ID NO: 1067 - protein, 19 aa, Artificial Sequence
  Internal Standard Peptide Sequence to quantitate RAS-RAF-MAPK Pathway protein
  MOD_RES (19)..(19): heavy isotope labeled amino acid
DFASILTNASPLAVNLLEK

SEQ ID NO: 1068 - protein, 26 aa, Artificial Sequence
  Internal Standard Peptide Sequence to quantitate RAS-RAF-MAPK Pathway protein
  MOD_RES (26)..(26): heavy isotope labeled amino acid
VTAGEALAHPYFESLHDTEDEPQVQK

SEQ ID NO: 1069 - protein, 10 aa, Artificial Sequence
  Internal Standard Peptide Sequence to quantitate RAS-RAF-MAPK Pathway protein
  MOD_RES (10)..(10): heavy isotope labeled amino acid
YIHAAGIIHR

SEQ ID NO: 1070 - protein, 12 aa, Artificial Sequence
  Internal Standard Peptide Sequence to quantitate RAS-RAF-MAPK Pathway protein
  MOD_RES (12)..(12): heavy isotope labeled amino acid
VTGTPPAEFVQR

SEQ ID NO: 1071 - protein, 12 aa, Artificial Sequence
  Internal Standard Peptide Sequence to quantitate RAS-RAF-MAPK Pathway protein
  MOD_RES (12)..(12): heavy isotope labeled amino acid
LYRPFQSELFAK

SEQ ID NO: 1072 - protein, 20 aa, Artificial Sequence
  Internal Standard Peptide Sequence to quantitate RAS-RAF-MAPK Pathway protein
  MOD_RES (14)..(14): carbamidomethylated cysteine
  MOD_RES (20)..(20): heavy isotope labeled amino acid
DLQPVGSGAYGAVCSAVDGR

SEQ ID NO: 1073 - protein, 9 aa, Artificial Sequence
  Internal Standard Peptide Sequence to quantitate RAS-RAF-MAPK Pathway protein
  MOD_RES (9)..(9): heavy isotope labeled amino acid
EVLSFKPPR

SEQ ID NO: 1074 - protein, 11 aa, Artificial Sequence
  Internal Standard Peptide Sequence to quantitate RAS-RAF-MAPK Pathway protein
  MOD_RES (11)..(11): heavy isotope labeled amino acid
ASPQAADLLEK

SEQ ID NO: 1075 - protein, 11 aa, Artificial Sequence
  Internal Standard Peptide Sequence to quantitate RAS-RAF-MAPK Pathway protein
  MOD_RES (11)..(11): heavy isotope labeled amino acid
SYIQSLPQTPR

SEQ ID NO: 1076 - protein, 12 aa, Artificial Sequence
  Internal Standard Peptide Sequence to quantitate RAS-RAF-MAPK Pathway protein
  MOD_RES (12)..(12): heavy isotope labeled amino acid
VTGVPGTEFVQK

SEQ ID NO: 1077 - protein, 10 aa, Artificial Sequence
  Internal Standard Peptide Sequence to quantitate RAS-RAF-MAPK Pathway protein
  MOD_RES (10)..(10): heavy isotope labeled amino acid
YIHSAGVVHR

SEQ ID NO: 1078 - protein, 22 aa, Artificial Sequence
  Internal Standard Peptide Sequence to quantitate RAS-RAF-MAPK Pathway protein
  MOD_RES (17)..(17): carbamidomethylated cysteine
  MOD_RES (22)..(22): heavy isotope labeled amino acid
TYVSPTHVGSGAYGSVCSAIDK

SEQ ID NO: 1079 - protein, 11 aa, Artificial Sequence
  Internal Standard Peptide Sequence to quantitate RAS-RAF-MAPK Pathway protein
  MOD_RES (11)..(11): heavy isotope labeled amino acid
DYLDQLTQILK

SEQ ID NO: 1080 - protein, 8 aa, Artificial Sequence
  Internal Standard Peptide Sequence to quantitate RAS-RAF-MAPK Pathway protein
  MOD_RES (8)..(8): heavy isotope labeled amino acid
DFTQLFPR

SEQ ID NO: 1081 - protein, 7 aa, Artificial Sequence
  Internal Standard Peptide Sequence to quantitate RAS-RAF-MAPK Pathway protein
  MOD_RES (7)..(7): heavy isotope labeled amino acid
LTVDEWK

SEQ ID NO: 1082 - protein, 16 aa, Artificial Sequence
  Internal Standard Peptide Sequence to quantitate RAS-RAF-MAPK Pathway protein
  MOD_RES (13)..(13): carbamidomethylated cysteine
  MOD_RES (16)..(16): heavy isotope labeled amino acid
DLKPSNLAVNEDCELK

SEQ ID NO: 1083 - protein, 8 aa, Artificial Sequence
  Internal Standard Peptide Sequence to quantitate RAS-RAF-MAPK Pathway protein
  MOD_RES (8)..(8): heavy isotope labeled amino acid
DLLIDEWK

SEQ ID NO: 1084 - protein, 15 aa, Artificial Sequence
  Internal Standard Peptide Sequence to quantitate RAS-RAF-MAPK Pathway protein
  MOD_RES (15)..(15): heavy isotope labeled amino acid
HENVIGLLDVFTPAR

SEQ ID NO: 1085 - protein, 13 aa, Artificial Sequence
  Internal Standard Peptide Sequence to quantitate RAS-RAF-MAPK Pathway protein
  MOD_RES (13)..(13): heavy isotope labeled amino acid
TLFPGTDHIDQLK

SEQ ID NO: 1086 - protein, 10 aa, Artificial Sequence
  Internal Standard Peptide Sequence to quantitate RAS-RAF-MAPK Pathway protein
  MOD_RES (10)..(10): heavy isotope labeled amino acid
YIHSADIIHR

SEQ ID NO: 1087 - protein, 11 aa, Artificial Sequence
  Internal Standard Peptide Sequence to quantitate RAS-RAF-MAPK Pathway protein
  MOD_RES (11)..(11): heavy isotope labeled amino acid
LVGTPGAELLK

SEQ ID NO: 1088 - protein, 15 aa, Artificial Sequence
  Internal Standard Peptide Sequence to quantitate RAS-RAF-MAPK Pathway protein
  MOD_RES (15)..(15): heavy isotope labeled amino acid
LTDDHVQFLIYQILR

SEQ ID NO: 1089 - protein, 22 aa, Artificial Sequence
  Internal Standard Peptide Sequence to quantitate RAS-RAF-MAPK Pathway protein
  MOD_RES (16)..(16): carbamidomethylated cysteine
  MOD_RES (22)..(22): heavy isotope labeled amino acid
YQNLSPVGSGAYGSVCAAFDTK

SEQ ID NO: 1090 - protein, 8 aa, Artificial Sequence
  Internal Standard Peptide Sequence to quantitate RAS-RAF-MAPK Pathway protein
  MOD_RES (8)..(8): heavy isotope labeled amino acid
TIWEVPER

SEQ ID NO: 1091 - protein, 7 aa, Artificial Sequence
  Internal Standard Peptide Sequence to quantitate RAS-RAF-MAPK Pathway protein
  MOD_RES (7)..(7): heavy isotope labeled amino acid
ALDLLDR

SEQ ID NO: 1092 - protein, 9 aa, Artificial Sequence
  Internal Standard Peptide Sequence to quantitate RAS-RAF-MAPK Pathway protein
  MOD_RES (9)..(9): heavy isotope labeled amino acid
NYLQSLPSK

SEQ ID NO: 1093 - protein, 9 aa, Artificial Sequence
  Internal Standard Peptide Sequence to quantitate RAS-RAF-MAPK Pathway protein
  MOD_RES (9)..(9): heavy isotope labeled amino acid
GQPFDVGPR

SEQ ID NO: 1094 - protein, 23 aa, Artificial Sequence
  Internal Standard Peptide Sequence to quantitate RAS-RAF-MAPK Pathway protein
  MOD_RES (23)..(23): heavy isotope labeled amino acid
YTQLQYIGEGAYGMVSSAYDHVR

SEQ ID NO: 1095 - protein, 7 aa, Artificial Sequence
  Internal Standard Peptide Sequence to quantitate RAS-RAF-MAPK Pathway protein
  MOD_RES (7)..(7): heavy isotope labeled amino acid
EIQILLR

SEQ ID NO: 1096 - protein, 16 aa, Artificial Sequence
  Internal Standard Peptide Sequence to quantitate RAS-RAF-MAPK Pathway protein
  MOD_RES (13)..(13): carbamidomethylated cysteine
  MOD_RES (16)..(16): heavy isotope labeled amino acid
DLKPSNLLINTTCDLK

SEQ ID NO: 1097 - protein, 19 aa, Artificial Sequence
  Internal Standard Peptide Sequence to quantitate RAS-RAF-MAPK Pathway protein
  MOD_RES (19)..(19): heavy isotope labeled amino acid
IADPEHDHTGFLTEYVATR

SEQ ID NO: 1098 - protein, 11 aa, Artificial Sequence
  Internal Standard Peptide Sequence to quantitate RAS-RAF-MAPK Pathway protein
  MOD_RES (11)..(11): heavy isotope labeled amino acid
LKELIFQETAR

SEQ ID NO: 1099 - protein, 9 aa, Artificial Sequence
  Internal Standard Peptide Sequence to quantitate RAS-RAF-MAPK Pathway protein
  MOD_RES (9)..(9): heavy isotope labeled amino acid
ELIFQETAR

SEQ ID NO: 1100 - protein, 9 aa, Artificial Sequence
  Internal Standard Peptide Sequence to quantitate RAS-RAF-MAPK Pathway protein
  MOD_RES (9)..(9): heavy isotope labeled amino acid
FQPGVLEAP

SEQ ID NO: 1101 - protein, 19 aa, Artificial Sequence
  Internal Standard Peptide Sequence to quantitate RAS-RAF-MAPK Pathway protein
  MOD_RES (19)..(19): heavy isotope labeled amino acid
LGQYGFPNPEWSEVSEDAK

SEQ ID NO: 1102 - protein, 17 aa, Artificial Sequence
  Internal Standard Peptide Sequence to quantitate RAS-RAF-MAPK Pathway protein
  MOD_RES (17)..(17): heavy isotope labeled amino acid
DIGTAIQFLHSHNIAHR

SEQ ID NO: 1103 - protein, 25 aa, Artificial Sequence
  Internal Standard Peptide Sequence to quantitate RAS-RAF-MAPK Pathway protein
  MOD_RES (16)..(16): carbamidomethylated cysteine
  MOD_RES (25)..(25): heavy isotope labeled amino acid
YQNLKPIGSGAQGIVCAAYDAILER

SEQ ID NO: 1104 - protein, 13 aa, Artificial Sequence
  Internal Standard Peptide Sequence to quantitate RAS-RAF-MAPK Pathway protein
  MOD_RES (13)..(13): heavy isotope labeled amino acid
NIIGLLNVFTPQK

SEQ ID NO: 1105 - protein, 8 aa, Artificial Sequence
  Internal Standard Peptide Sequence to quantitate RAS-RAF-MAPK Pathway protein
  MOD_RES (8)..(8): heavy isotope labeled amino acid
YAGYSFEK

SEQ ID NO: 1106 - protein, 15 aa, Artificial Sequence
  Internal Standard Peptide Sequence to quantitate RAS-RAF-MAPK Pathway protein
  MOD_RES (15)..(15): heavy isotope labeled amino acid
LFPDVLFPADSEHNK

SEQ ID NO: 1107 - protein, 8 aa, Artificial Sequence
  Internal Standard Peptide Sequence to quantitate RAS-RAF-MAPK Pathway protein
  MOD_RES (8)..(8): heavy isotope labeled amino acid
EVMDLEER

SEQ ID NO: 1108 - protein, 18 aa, Artificial Sequence
  Internal Standard Peptide Sequence to quantitate RAS-RAF-MAPK Pathway protein
  MOD_RES (18)..(18): heavy isotope labeled amino acid
DNNFYSVEIGDSTFTVLK

SEQ ID NO: 1109 - protein, 8 aa, Artificial Sequence
  Internal Standard Peptide Sequence to quantitate RAS-RAF-MAPK Pathway protein
  MOD_RES (8)..(8): heavy isotope labeled amino acid
TYVENRPK

SEQ ID NO: 1110 - protein, 27 aa, Artificial Sequence
  Internal Standard Peptide Sequence to quantitate RAS-RAF-MAPK Pathway protein
  MOD_RES (27)..(27): heavy isotope labeled amino acid
ISVDEALQHPYINVWYDPSEAEAPPPK

SEQ ID NO: 1111 - protein, 19 aa, Artificial Sequence
  Internal Standard Peptide Sequence to quantitate RAS-RAF-MAPK Pathway protein
  MOD_RES (1)..(1): carbamidomethylated cysteine
  MOD_RES (19)..(19): heavy isotope labeled amino acid
CDSQFYSVQVADSTFTVLK

SEQ ID NO: 1112 - protein, 20 aa, Artificial Sequence
  Internal Standard Peptide Sequence to quantitate RAS-RAF-MAPK Pathway protein
  MOD_RES (1)..(1): carbamidomethylated cysteine
  MOD_RES (20)..(20): heavy isotope labeled amino acid
CDSQFYSVQVADSTFTVLKR

SEQ ID NO: 1113 - protein, 15 aa, Artificial Sequence
  Internal Standard Peptide Sequence to quantitate RAS-RAF-MAPK Pathway protein
  MOD_RES (3)..(3): carbamidomethylated cysteine
  MOD_RES (15)..(15): heavy isotope labeled amino acid
TACTNFMMTPYVVTR

SEQ ID NO: 1114 - protein, 16 aa, Artificial Sequence
  Internal Standard Peptide Sequence to quantitate RAS-RAF-MAPK Pathway protein
  MOD_RES (2)..(2): carbamidomethylated cysteine
  MOD_RES (16)..(16): heavy isotope labeled amino acid
GCVIFQGTDHIDQWNK

SEQ ID NO: 1115 - protein, 16 aa, Artificial Sequence
  Internal Standard Peptide Sequence to quantitate RAS-RAF-MAPK Pathway protein
  MOD_RES (16)..(16): heavy isotope labeled amino acid
FEELFPDWIFPSESER

SEQ ID NO: 1116 - protein, 8 aa, Artificial Sequence
  Internal Standard Peptide Sequence to quantitate RAS-RAF-MAPK Pathway protein
  MOD_RES (8)..(8): heavy isotope labeled amino acid
ISVDEALR

SEQ ID NO: 1117 - protein, 28 aa, Artificial Sequence
  Internal Standard Peptide Sequence to quantitate RAS-RAF-MAPK Pathway protein
  MOD_RES (28)..(28): heavy isotope labeled amino acid
HPYITVWYDPAEAEAPPPQIYDAQLEER

SEQ ID NO: 1118 - protein, 14 aa, Artificial Sequence
  Internal Standard Peptide Sequence to quantitate RAS-RAF-MAPK Pathway protein
  MOD_RES (14)..(14): heavy isotope labeled amino acid
VIEQLGTPSAEFMK

SEQ ID NO: 1119 - protein, 8 aa, Artificial Sequence
  Internal Standard Peptide Sequence to quantitate RAS-RAF-MAPK Pathway protein
  MOD_RES (8)..(8): heavy isotope labeled amino acid
EVMDWEER

SEQ ID NO: 1120 - protein, 8 aa, Artificial Sequence
  Internal Standard Peptide Sequence to quantitate RAS-RAF-MAPK Pathway protein
  MOD_RES (8)..(8): heavy isotope labeled amino acid
EHAIEEWK

SEQ ID NO: 1121 - protein, 17 aa, Artificial Sequence
  Internal Standard Peptide Sequence to quantitate RAS-RAF-MAPK Pathway protein
  MOD_RES (17)..(17): heavy isotope labeled amino acid
SIGEAIQYLHSINIAHR

SEQ ID NO: 1122 - protein, 12 aa, Artificial Sequence
  Internal Standard Peptide Sequence to quantitate RAS-RAF-MAPK Pathway protein
  MOD_RES (12)..(12): heavy isotope labeled amino acid
IVDVYENLYAGR

SEQ ID NO: 1123 - protein, 10 aa, Artificial Sequence
  Internal Standard Peptide Sequence to quantitate RAS-RAF-MAPK Pathway protein
  MOD_RES (10)..(10): heavy isotope labeled amino acid
VPQTPLHTSR

SEQ ID NO: 1124 - protein, 27 aa, Artificial Sequence
  Internal Standard Peptide Sequence to quantitate RAS-RAF-MAPK Pathway protein
  MOD_RES (12)..(12): carbamidomethylated cysteine
  MOD_RES (27)..(27): heavy isotope labeled amino acid
ETTSHNSLTTPCYTPYYVAPEVLGPEK

SEQ ID NO: 1125 - protein, 7 aa, Artificial Sequence
  Internal Standard Peptide Sequence to quantitate RAS-RAF-MAPK Pathway protein
  MOD_RES (7)..(7): heavy isotope labeled amino acid
VLQIFNK

SEQ ID NO: 1126 - protein, 8 aa, Artificial Sequence
  Internal Standard Peptide Sequence to quantitate RAS-RAF-MAPK Pathway protein
  MOD_RES (8)..(8): heavy isotope labeled amino acid
NAIIDDYK

SEQ ID NO: 1127 - protein, 9 aa, Artificial Sequence
  Internal Standard Peptide Sequence to quantitate RAS-RAF-MAPK Pathway protein
  MOD_RES (4)..(4): carbamidomethylated cysteine
  MOD_RES (9)..(9): heavy isotope labeled amino acid
ASQCPHIVR

SEQ ID NO: 1128 - protein, 13 aa, Artificial Sequence
  Internal Standard Peptide Sequence to quantitate RAS-RAF-MAPK Pathway protein
  MOD_RES (13)..(13): heavy isotope labeled amino acid
VTSQVLGLGINGK

SEQ ID NO: 1129 - protein, 19 aa, Artificial Sequence
  Internal Standard Peptide Sequence to quantitate RAS-RAF-MAPK Pathway protein
  MOD_RES (10)..(16): carbamidomethylated cysteine
  MOD_RES (19)..(19): heavy isotope labeled amino acid
TGPVLEHPDCFPCQDCSSK

SEQ ID NO: 1130 - protein, 11 aa, Artificial Sequence
  Internal Standard Peptide Sequence to quantitate RAS-RAF-MAPK Pathway protein
  MOD_RES (11)..(11): heavy isotope labeled amino acid
ETLDAQTFHTR

SEQ ID NO: 1131 - protein, 8 aa, Artificial Sequence
  Internal Standard Peptide Sequence to quantitate RAS-RAF-MAPK Pathway protein
  MOD_RES (8)..(8): heavy isotope labeled amino acid
YVNDFFNK

SEQ ID NO: 1132 - protein, 9 aa, Artificial Sequence
  Internal Standard Peptide Sequence to quantitate RAS-RAF-MAPK Pathway protein
  MOD_RES (9)..(9): heavy isotope labeled amino acid
TEFTTALQR

SEQ ID NO: 1133 - protein, 14 aa, Artificial Sequence
  Internal Standard Peptide Sequence to quantitate RAS-RAF-MAPK Pathway protein
  MOD_RES (14)..(14): heavy isotope labeled amino acid
GDLTIANLGTSEGR

SEQ ID NO: 1134 - protein, 13 aa, Artificial Sequence
  Internal Standard Peptide Sequence to quantitate RAS-RAF-MAPK Pathway protein
  MOD_RES (13)..(13): heavy isotope labeled amino acid
VFPNSAPLEGGTR

SEQ ID NO: 1135 - protein, 14 aa, Artificial Sequence
  Internal Standard Peptide Sequence to quantitate RAS-RAF-MAPK Pathway protein
  MOD_RES (14)..(14): heavy isotope labeled amino acid
SFISGGSTITGVGK

SEQ ID NO: 1136 - protein, 9 aa, Artificial Sequence
  Internal Standard Peptide Sequence to quantitate RAS-RAF-MAPK Pathway protein
  MOD_RES (9)..(9): heavy isotope labeled amino acid
NLNSVSVPR

SEQ ID NO: 1137 - protein, 8 aa, Artificial Sequence
  Internal Standard Peptide Sequence to quantitate RAS-RAF-MAPK Pathway protein
  MOD_RES (8)..(8): heavy isotope labeled amino acid
DLGSELVR

SEQ ID NO: 1138 - protein, 11 aa, Artificial Sequence
  Internal Standard Peptide Sequence to quantitate RAS-RAF-MAPK Pathway protein
  MOD_RES (11)..(11): heavy isotope labeled amino acid
DLIGFGLQVAK

SEQ ID NO: 1139 - protein, 8 aa, Artificial Sequence
  Internal Standard Peptide Sequence to quantitate RAS-RAF-MAPK Pathway protein
  MOD_RES (8)..(8): heavy isotope labeled amino acid
VADFGLAR

SEQ ID NO: 1140 - protein, 20 aa, Artificial Sequence
  Internal Standard Peptide Sequence to quantitate RAS-RAF-MAPK Pathway protein
  MOD_RES (20)..(20): heavy isotope labeled amino acid
EVFNILQAAYVSKPGAQLAR

SEQ ID NO: 1141 - protein, 12 aa, Artificial Sequence
  Internal Standard Peptide Sequence to quantitate RAS-RAF-MAPK Pathway protein
  MOD_RES (12)..(12): heavy isotope labeled amino acid
VAQLAVINSLEK

SEQ ID NO: 1142 - protein, 21 aa, Artificial Sequence
  Internal Standard Peptide Sequence to quantitate RAS-RAF-MAPK Pathway protein
  MOD_RES (2)..(2): carbamidomethylated cysteine
  MOD_RES (21)..(21): heavy isotope labeled amino acid
ICLAQNSPSTFHYVLVNSLHR

SEQ ID NO: 1143 - protein, 12 aa, Artificial Sequence
  Internal Standard Peptide Sequence to quantitate RAS-RAF-MAPK Pathway protein
  MOD_RES (6)..(6): carbamidomethylated cysteine
  MOD_RES (12)..(12): heavy isotope labeled amino acid
IDAVYCHSVELR

SEQ ID NO: 1144 - protein, 7 aa, Artificial Sequence
  Internal Standard Peptide Sequence to quantitate RAS-RAF-MAPK Pathway protein
  MOD_RES (4)..(4): carbamidomethylated cysteine
  MOD_RES (7)..(7): heavy isotope labeled amino acid
LLLCNPR

SEQ ID NO: 1145 - protein, 9 aa, Artificial Sequence
  Internal Standard Peptide Sequence to quantitate RAS-RAF-MAPK Pathway protein
  MOD_RES (3)..(3): carbamidomethylated cysteine
  MOD_RES (9)..(9): heavy isotope labeled amino acid
HLCEEADIR

SEQ ID NO: 1146 - protein, 7 aa, Artificial Sequence
  Internal Standard Peptide Sequence to quantitate RAS-RAF-MAPK Pathway protein
  MOD_RES (7)..(7): heavy isotope labeled amino acid
LILNYPK

SEQ ID NO: 1147 - protein, 7 aa, Artificial Sequence
  Internal Standard Peptide Sequence to quantitate RAS-RAF-MAPK Pathway protein
  MOD_RES (7)..(7): heavy isotope labeled amino acid
LLDPLLR

SEQ ID NO: 1148 - protein, 17 aa, Artificial Sequence
  Internal Standard Peptide Sequence to quantitate RAS-RAF-MAPK Pathway protein
  MOD_RES (3)..(11): carbamidomethylated cysteine
  MOD_RES (17)..(17): heavy isotope labeled amino acid
SVCHCLYQATCHSLLNK

SEQ ID NO: 1149 - protein, 12 aa, Artificial Sequence
  Internal Standard Peptide Sequence to quantitate RAS-RAF-MAPK Pathway protein
  MOD_RES (12)..(12): heavy isotope labeled amino acid
TLSIFYQAGTSK

SEQ ID NO: 1150 - protein, 11 aa, Artificial Sequence
  Internal Standard Peptide Sequence to quantitate RAS-RAF-MAPK Pathway protein
  MOD_RES (11)..(11): heavy isotope labeled amino acid
AGNPIFYYVAR

SEQ ID NO: 1151 - protein, 9 aa, Artificial Sequence
  Internal Standard Peptide Sequence to quantitate RAS-RAF-MAPK Pathway protein
  MOD_RES (6)..(6): carbamidomethylated cysteine
  MOD_RES (9)..(9): heavy isotope labeled amino acid
LVFIDCPGK

SEQ ID NO: 1152 - protein, 13 aa, Artificial Sequence
  Internal Standard Peptide Sequence to quantitate RAS-RAF-MAPK Pathway protein
  MOD_RES (13)..(13): heavy isotope labeled amino acid
VGSTAVQVTSAER

SEQ ID NO: 1153 - protein, 8 aa, Artificial Sequence
  Internal Standard Peptide Sequence to quantitate RAS-RAF-MAPK Pathway protein
  MOD_RES (8)..(8): heavy isotope labeled amino acid
SAAVIAFR

SEQ ID NO: 1154 - protein, 12 aa, Artificial Sequence
  Internal Standard Peptide Sequence to quantitate RAS-RAF-MAPK Pathway protein
  MOD_RES (12)..(12): heavy isotope labeled amino acid
FAFQYNPSLQPR

SEQ ID NO: 1155 - protein, 19 aa, Artificial Sequence
  Internal Standard Peptide Sequence to quantitate RAS-RAF-MAPK Pathway protein
  MOD_RES (19)..(19): heavy isotope labeled amino acid
GSEGYLAATYPTVGQTSPR

SEQ ID NO: 1156 - protein, 11 aa, Artificial Sequence
  Internal Standard Peptide Sequence to quantitate RAS-RAF-MAPK Pathway protein
  MOD_RES (11)..(11): heavy isotope labeled amino acid
ISSSQQHPHLR

SEQ ID NO: 1157 - protein, 12 aa, Artificial Sequence
  Internal Standard Peptide Sequence to quantitate RAS-RAF-MAPK Pathway protein
  MOD_RES (12)..(12): heavy isotope labeled amino acid
VFPVVHNLLDSK

SEQ ID NO: 1158 - protein, 13 aa, Artificial Sequence
  Internal Standard Peptide Sequence to quantitate RAS-RAF-MAPK Pathway protein
  MOD_RES (5)..(5): carbamidomethylated cysteine
  MOD_RES (13)..(13): heavy isotope labeled amino acid
HGGTCHNEVGSYR

SEQ ID NO: 1159 - protein, 12 aa, Artificial Sequence
  Internal Standard Peptide Sequence to quantitate RAS-RAF-MAPK Pathway protein
  MOD_RES (6)..(8): carbamidomethylated cysteine
  MOD_RES (12)..(12): heavy isotope labeled amino acid
VASFYCECPHGR

SEQ ID NO: 1160 - protein, 23 aa, Artificial Sequence
  Internal Standard Peptide Sequence to quantitate RAS-RAF-MAPK Pathway protein
  MOD_RES (7)..(14): carbamidomethylated cysteine
  MOD_RES (23)..(23): heavy isotope labeled amino acid
GTQGVHCEINVDDCNPPVDPVSR

SEQ ID NO: 1161 - protein, 14 aa, Artificial Sequence
  Internal Standard Peptide Sequence to quantitate RAS-RAF-MAPK Pathway protein
  MOD_RES (5)..(13): carbamidomethylated cysteine
  MOD_RES (14)..(14): heavy isotope labeled amino acid
AEGQCNPLYDQYCK

SEQ ID NO: 1162 - protein, 16 aa, Artificial Sequence
  Internal Standard Peptide Sequence to quantitate RAS-RAF-MAPK Pathway protein
  MOD_RES (16)..(16): heavy isotope labeled amino acid
AAEGWAAPDALLGQVK

SEQ ID NO: 1163 - protein, 17 aa, Artificial Sequence
  Internal Standard Peptide Sequence to quantitate RAS-RAF-MAPK Pathway protein
  MOD_RES (17)..(17): heavy isotope labeled amino acid
FEEPVVLPDLDDQTDHR

SEQ ID NO: 1164 - protein, 11 aa, Artificial Sequence
  Internal Standard Peptide Sequence to quantitate RAS-RAF-MAPK Pathway protein
  MOD_RES (11)..(11): heavy isotope labeled amino acid
TGETALHLAAR

SEQ ID NO: 1165 - protein, 9 aa, Artificial Sequence
  Internal Standard Peptide Sequence to quantitate RAS-RAF-MAPK Pathway protein
  MOD_RES (9)..(9): heavy isotope labeled amino acid
LLDEYNLVR

SEQ ID NO: 1166 - protein, 15 aa, Artificial Sequence
  Internal Standard Peptide Sequence to quantitate RAS-RAF-MAPK Pathway protein
  MOD_RES (7)..(7): carbamidomethylated cysteine
  MOD_RES (15)..(15): heavy isotope labeled amino acid
TGEGFLCVFAINNSK

SEQ ID NO: 1167 - protein, 9 aa, Artificial Sequence
  Internal Standard Peptide Sequence to quantitate RAS-RAF-MAPK Pathway protein
  MOD_RES (9)..(9): heavy isotope labeled amino acid
SFADINLYR

SEQ ID NO: 1168 - protein, 7 aa, Artificial Sequence
  Internal Standard Peptide Sequence to quantitate RAS-RAF-MAPK Pathway protein
  MOD_RES (7)..(7): heavy isotope labeled amino acid
QAHELAK

SEQ ID NO: 1169 - protein, 12 aa, Artificial Sequence
  Internal Standard Peptide Sequence to quantitate RAS-RAF-MAPK Pathway protein
  MOD_RES (12)..(12): heavy isotope labeled amino acid
QGVEDAFYTLVR

SEQ ID NO: 1170 - protein, 19 aa, Artificial Sequence
  Internal Standard Peptide Sequence to quantitate RAS-RAF-MAPK Pathway protein
  MOD_RES (11)..(16): carbamidomethylated cysteine
  MOD_RES (18)..(18): heavy isotope labeled amino acid
LNSSDDGTQGCMGLPCVVM

SEQ ID NO: 1171 - protein, 12 aa, Artificial Sequence
  Internal Standard Peptide Sequence to quantitate RAS-RAF-MAPK Pathway protein
  MOD_RES (12)..(12): heavy isotope labeled amino acid
SYGIPFIETSAK

SEQ ID NO: 1172 - protein, 11 aa, Artificial Sequence
  Internal Standard Peptide Sequence to quantitate RAS-RAF-MAPK Pathway protein
  MOD_RES (11)..(11): heavy isotope labeled amino acid
NTSTMIGAGSK

SEQ ID NO: 1173 - protein, 19 aa, Artificial Sequence
  Internal Standard Peptide Sequence to quantitate RAS-RAF-MAPK Pathway protein
  MOD_RES (19)..(19): heavy isotope labeled amino acid
DAGTLNHGSKPLPPNPEEK

SEQ ID NO: 1174 - protein, 7 aa, Artificial Sequence
  Internal Standard Peptide Sequence to quantitate RAS-RAF-MAPK Pathway protein
  MOD_RES (7)..(7): heavy isotope labeled amino acid
SILPGDK

SEQ ID NO: 1175 - protein, 14 aa, Artificial Sequence
  Internal Standard Peptide Sequence to quantitate RAS-RAF-MAPK Pathway protein
  MOD_RES (14)..(14): heavy isotope labeled amino acid
SAEDYNSSNALNVK

SEQ ID NO: 1176 - protein, 12 aa, Artificial Sequence
  Internal Standard Peptide Sequence to quantitate RAS-RAF-MAPK Pathway protein
  MOD_RES (12)..(12): heavy isotope labeled amino acid
SVIEPLPVTPTR

SEQ ID NO: 1177 - protein, 22 aa, Artificial Sequence
  Internal Standard Peptide Sequence to quantitate RAS-RAF-MAPK Pathway protein
  MOD_RES (22)..(22): heavy isotope labeled amino acid
DVATSPISPTENNTTPPDALTR

SEQ ID NO: 1178 - protein, 24 aa, Artificial Sequence
  Internal Standard Peptide Sequence to quantitate RAS-RAF-MAPK Pathway protein
  MOD_RES (24)..(24): heavy isotope labeled amino acid
IGQGASGTVYTAMDVATGQEVAIK

SEQ ID NO: 1179 - protein, 17 aa, Artificial Sequence
  Internal Standard Peptide Sequence to quantitate RAS-RAF-MAPK Pathway protein
  MOD_RES (2)..(2): carbamidomethylated cysteine
  MOD_RES (17)..(17): heavy isotope labeled amino acid
ECLQALEFLHSNQVIHR

SEQ ID NO: 1180 - protein, 9 aa, Artificial Sequence
  Internal Standard Peptide Sequence to quantitate RAS-RAF-MAPK Pathway protein
  MOD_RES (9)..(9): heavy isotope labeled amino acid
ELLQHQFLK

SEQ ID NO: 1181 - protein, 16 aa, Artificial Sequence
  Internal Standard Peptide Sequence to quantitate RAS-RAF-MAPK Pathway protein
  MOD_RES (16)..(16): heavy isotope labeled amino acid
IAKPLSSLTPLIAAAK

SEQ ID NO: 1182 - protein, 13 aa, Artificial Sequence
  Internal Standard Peptide Sequence to quantitate RAS-RAF-MAPK Pathway protein
  MOD_RES (13)..(13): heavy isotope labeled amino acid
HPDVEVDGFSELR

SEQ ID NO: 1183 - protein, 11 aa, Artificial Sequence
  Internal Standard Peptide Sequence to quantitate RAS-RAF-MAPK Pathway protein
  MOD_RES (11)..(11): heavy isotope labeled amino acid
TLGDFAAEYAK

SEQ ID NO: 1184 - protein, 15 aa, Artificial Sequence
  Internal Standard Peptide Sequence to quantitate RAS-RAF-MAPK Pathway protein
  MOD_RES (15)..(15): heavy isotope labeled amino acid
EELGFRPEYSASQLK

SEQ ID NO: 1185 - protein, 7 aa, Artificial Sequence
  Internal Standard Peptide Sequence to quantitate RAS-RAF-MAPK Pathway protein
  MOD_RES (7)..(7): heavy isotope labeled amino acid
ELLIFNK

SEQ ID NO: 1186 - protein, 15 aa, Artificial Sequence
  Internal Standard Peptide Sequence to quantitate RAS-RAF-MAPK Pathway protein
  MOD_RES (6)..(6): carbamidomethylated cysteine
  MOD_RES (15)..(15): heavy isotope labeled amino acid
SDAYYCTGDVTAWTK

SEQ ID NO: 1187 - protein, 15 aa, Artificial Sequence
  Internal Standard Peptide Sequence to quantitate RAS-RAF-MAPK Pathway protein
  MOD_RES (15)..(15): heavy isotope labeled amino acid
VVSEDFLQDVSASTK

SEQ ID NO: 1188 - protein, 10 aa, Artificial Sequence
  Internal Standard Peptide Sequence to quantitate RAS-RAF-MAPK Pathway protein
  MOD_RES (10)..(10): heavy isotope labeled amino acid
AEPVEVVAPR

SEQ ID NO: 1189 - protein, 13 aa, Artificial Sequence
  Internal Standard Peptide Sequence to quantitate RAS-RAF-MAPK Pathway protein
  MOD_RES (13)..(13): heavy isotope labeled amino acid
VFSATLGLVDIVK

SEQ ID NO: 1190 - protein, 14 aa, Artificial Sequence
  Internal Standard Peptide Sequence to quantitate RAS-RAF-MAPK Pathway protein
  MOD_RES (14)..(14): heavy isotope labeled amino acid
KPPLLNNADSVQAK

SEQ ID NO: 1191 - protein, 13 aa, Artificial Sequence
  Internal Standard Peptide Sequence to quantitate RAS-RAF-MAPK Pathway protein
  MOD_RES (13)..(13): heavy isotope labeled amino acid
TTNFAGILSQGLR

SEQ ID NO: 1192 - protein, 16 aa, Artificial Sequence
  Internal Standard Peptide Sequence to quantitate RAS-RAF-MAPK Pathway protein
  MOD_RES (16)..(16): heavy isotope labeled amino acid
TQDQQSLSDVEGAYSR

SEQ ID NO: 1193 - protein, 17 aa, Artificial Sequence
  Internal Standard Peptide Sequence to quantitate RAS-RAF-MAPK Pathway protein
  MOD_RES (16)..(16): carbamidomethylated cysteine
  MOD_RES (17)..(17): heavy isotope labeled amino acid
AEGAPPQQGPFAPPPCK

SEQ ID NO: 1194 - protein, 11 aa, Artificial Sequence
  Internal Standard Peptide Sequence to quantitate RAS-RAF-MAPK Pathway protein
  MOD_RES (7)..(7): carbamidomethylated cysteine
  MOD_RES (11)..(11): heavy isotope labeled amino acid
APGASGCLLPR

SEQ ID NO: 1195 - protein, 7 aa, Artificial Sequence
  Internal Standard Peptide Sequence to quantitate RAS-RAF-MAPK Pathway protein
  MOD_RES (3)..(3): carbamidomethylated cysteine
  MOD_RES (7)..(7): heavy isotope labeled amino acid
NDCIVDK

SEQ ID NO: 1196 - protein, 10 aa, Artificial Sequence
  Internal Standard Peptide Sequence to quantitate RAS-RAF-MAPK Pathway protein
  MOD_RES (10)..(10): heavy isotope labeled amino acid
LLDNLHDLVK

SEQ ID NO: 1197 - protein, 16 aa, Artificial Sequence
  Internal Standard Peptide Sequence to quantitate RAS-RAF-MAPK Pathway protein
  MOD_RES (5)..(5): carbamidomethylated cysteine
  MOD_RES (16)..(16): heavy isotope labeled amino acid
INHDCVPEQVIAEAIR

SEQ ID NO: 1198 - protein, 9 aa, Artificial Sequence
  Internal Standard Peptide Sequence to quantitate RAS-RAF-MAPK Pathway protein
  MOD_RES (2)..(6): carbamidomethylated cysteine
  MOD_RES (9)..(9): heavy isotope labeled amino acid
LCLSICSVK

SEQ ID NO: 1199 - protein, 9 aa, Artificial Sequence
  Internal Standard Peptide Sequence to quantitate RAS-RAF-MAPK Pathway protein
  MOD_RES (9)..(9): heavy isotope labeled amino acid
LINLTDILK

SEQ ID NO: 1200 - protein, 18 aa, Artificial Sequence
  Internal Standard Peptide Sequence to quantitate RAS-RAF-MAPK Pathway protein
  MOD_RES (11)..(11): carbamidomethylated cysteine
  MOD_RES (18)..(18): heavy isotope labeled amino acid
TGIYHGGEPLCDNVNTQR

SEQ ID NO: 1201 - protein, 9 aa, Artificial Sequence
  Internal Standard Peptide Sequence to quantitate RAS-RAF-MAPK Pathway protein
  MOD_RES (2)..(2): carbamidomethylated cysteine
  MOD_RES (9)..(9): heavy isotope labeled amino acid
LCVLEYQGK

SEQ ID NO: 1202 - protein, 10 aa, Artificial Sequence
  Internal Standard Peptide Sequence to quantitate RAS-RAF-MAPK Pathway protein
  MOD_RES (10)..(10): heavy isotope labeled amino acid
SLWVINSALR

SEQ ID NO: 1203 - protein, 14 aa, Artificial Sequence
  Internal Standard Peptide Sequence to quantitate RAS-RAF-MAPK Pathway protein
  MOD_RES (14)..(14): heavy isotope labeled amino acid
FSAASSDNTENLIK

SEQ ID NO: 1204 - protein, 11 aa, Artificial Sequence
  Internal Standard Peptide Sequence to quantitate RAS-RAF-MAPK Pathway protein
  MOD_RES (6)..(6): carbamidomethylated cysteine
  MOD_RES (11)..(11): heavy isotope labeled amino acid
IFEEQCQTQER

SEQ ID NO: 1205 - protein, 20 aa, Artificial Sequence
  Internal Standard Peptide Sequence to quantitate RAS-RAF-MAPK Pathway protein
  MOD_RES (20)..(20): heavy isotope labeled amino acid
YGFSDPLTFSSVVELINHYR

SEQ ID NO: 1206 - protein, 11 aa, Artificial Sequence
  Internal Standard Peptide Sequence to quantitate RAS-RAF-MAPK Pathway protein
  MOD_RES (11)..(11): heavy isotope labeled amino acid
TQSSSNLAELR

SEQ ID NO: 1207 - protein, 9 aa, Artificial Sequence
  Internal Standard Peptide Sequence to quantitate RAS-RAF-MAPK Pathway protein
  MOD_RES (9)..(9): heavy isotope labeled amino acid
TWNVGSSNR

SEQ ID NO: 1208 - protein, 12 aa, Artificial Sequence
  Internal Standard Peptide Sequence to quantitate RAS-RAF-MAPK Pathway protein
  MOD_RES (12)..(12): heavy isotope labeled amino acid
ALGATFGPLLLR

SEQ ID NO: 1209 - protein, 20 aa, Artificial Sequence
  Internal Standard Peptide Sequence to quantitate RAS-RAF-MAPK Pathway protein
  MOD_RES (20)..(20): heavy isotope labeled amino acid
SFLLALPAPLVTPEASAEAR

SEQ ID NO: 1210 - protein, 14 aa, Artificial Sequence
  Internal Standard Peptide Sequence to quantitate RAS-RAF-MAPK Pathway protein
  MOD_RES (14)..(14): heavy isotope labeled amino acid
AALQALGVAEGGER

SEQ ID NO: 1211 - protein, 21 aa, Artificial Sequence
  Internal Standard Peptide Sequence to quantitate RAS-RAF-MAPK Pathway protein
  MOD_RES (21)..(21): heavy isotope labeled amino acid
TDWSLSDVDQWDTAALADGIK

SEQ ID NO: 1212 - protein, 11 aa, Artificial Sequence
  Internal Standard Peptide Sequence to quantitate RAS-RAF-MAPK Pathway protein
  MOD_RES (11)..(11): heavy isotope labeled amino acid
EYDQLYEEYTR

SEQ ID NO: 1213 - protein, 10 aa, Artificial Sequence
  Internal Standard Peptide Sequence to quantitate RAS-RAF-MAPK Pathway protein
  MOD_RES (10)..(10): heavy isotope labeled amino acid
TSLEVSLLEK

SEQ ID NO: 1214 - protein, 7 aa, Artificial Sequence
  Internal Standard Peptide Sequence to quantitate RAS-RAF-MAPK Pathway protein
  MOD_RES (6)..(6): carbamidomethylated cysteine
  MOD_RES (7)..(7): heavy isotope labeled amino acid
LWNEWCR

SEQ ID NO: 1215 - protein, 20 aa, Artificial Sequence
  Internal Standard Peptide Sequence to quantitate RAS-RAF-MAPK Pathway protein
  MOD_RES (14)..(14): carbamidomethylated cysteine
  MOD_RES (20)..(20): heavy isotope labeled amino acid
TTDTTHLSSTEAFCVFYHLK

SEQ ID NO: 1216 - protein, 7 aa, Artificial Sequence
  Internal Standard Peptide Sequence to quantitate RAS-RAF-MAPK Pathway protein
  MOD_RES (5)..(5): carbamidomethylated cysteine
  MOD_RES (7)..(7): heavy isotope labeled amino acid
VFGSCPR

SEQ ID NO: 1217 - protein, 6 aa, Artificial Sequence
  Internal Standard Peptide Sequence to quantitate RAS-RAF-MAPK Pathway protein
  MOD_RES (6)..(6): heavy isotope labeled amino acid
HVTDYK

SEQ ID NO: 1218 - protein, 14 aa, Artificial Sequence
  Internal Standard Peptide Sequence to quantitate RAS-RAF-MAPK Pathway protein
  MOD_RES (14)..(14): heavy isotope labeled amino acid
TSESTGSLPSPFLR

SEQ ID NO: 1219 - protein, 16 aa, Artificial Sequence
  Internal Standard Peptide Sequence to quantitate RAS-RAF-MAPK Pathway protein
  MOD_RES (16)..(16): heavy isotope labeled amino acid
LDLITVEAFKPILSTR

SEQ ID NO: 1220 - protein, 10 aa, Artificial Sequence
  Internal Standard Peptide Sequence to quantitate RAS-RAF-MAPK Pathway protein
  MOD_RES (10)..(10): heavy isotope labeled amino acid
NIPQSTEILK

SEQ ID NO: 1221 - protein, 23 aa, Artificial Sequence
  Internal Standard Peptide Sequence to quantitate RAS-RAF-MAPK Pathway protein
  MOD_RES (23)..(23): heavy isotope labeled amino acid
YSTPHAFTFNTSSPSSEGSLSQR

SEQ ID NO: 1222 - protein, 17 aa, Artificial Sequence
  Internal Standard Peptide Sequence to quantitate RAS-RAF-MAPK Pathway protein
  MOD_RES (10)..(10): carbamidomethylated cysteine
  MOD_RES (17)..(17): heavy isotope labeled amino acid
AAHTEDINACTLTTSPR

SEQ ID NO: 1223 - protein, 14 aa, Artificial Sequence
  Internal Standard Peptide Sequence to quantitate RAS-RAF-MAPK Pathway protein
  MOD_RES (14)..(14): heavy isotope labeled amino acid
LLYPVAPPEPVEDR

SEQ ID NO: 1224 - protein, 11 aa, Artificial Sequence
  Internal Standard Peptide Sequence to quantitate RAS-RAF-MAPK Pathway protein
  MOD_RES (11)..(11): heavy isotope labeled amino acid
VPDTDEISFLK

SEQ ID NO: 1225 - protein, 13 aa, Artificial Sequence
  Internal Standard Peptide Sequence to quantitate RAS-RAF-MAPK Pathway protein
  MOD_RES (13)..(13): heavy isotope labeled amino acid
YYNSIGDIIDHYR

SEQ ID NO: 1226 - protein, 17 aa, Artificial Sequence
  Internal Standard Peptide Sequence to quantitate RAS-RAF-MAPK Pathway protein
  MOD_RES (17)..(17): heavy isotope labeled amino acid
GHATDEWFLLSSHIPLK

SEQ ID NO: 1227 - protein, 9 aa, Artificial Sequence
  Internal Standard Peptide Sequence to quantitate RAS-RAF-MAPK Pathway protein
  MOD_RES (9)..(9): heavy isotope labeled amino acid
VVSGFVFLR

SEQ ID NO: 1228 - protein, 10 aa, Artificial Sequence
  Internal Standard Peptide Sequence to quantitate RAS-RAF-MAPK Pathway protein
  MOD_RES (3)..(3): carbamidomethylated cysteine
  MOD_RES (10)..(10): heavy isotope labeled amino acid
LICPAILNPR

SEQ ID NO: 1229 - protein, 17 aa, Artificial Sequence
  Internal Standard Peptide Sequence to quantitate RAS-RAF-MAPK Pathway protein
  MOD_RES (9)..(9): carbamidomethylated cysteine
  MOD_RES (17)..(17): heavy isotope labeled amino acid
DLAALHEICVAHSDELR

SEQ ID NO: 1230 - protein, 11 aa, Artificial Sequence
  Internal Standard Peptide Sequence to quantitate RAS-RAF-MAPK Pathway protein
  MOD_RES (11)..(11): heavy isotope labeled amino acid
LLAITELLQQK

SEQ ID NO: 1231 - protein, 12 aa, Artificial Sequence
  Internal Standard Peptide Sequence to quantitate RAS-RAF-MAPK Pathway protein
  MOD_RES (12)..(12): heavy isotope labeled amino acid
YLHLLQIVETEK

SEQ ID NO: 1232 - protein, 8 aa, Artificial Sequence
  Internal Standard Peptide Sequence to quantitate RAS-RAF-MAPK Pathway protein
  MOD_RES (8)..(8): heavy isotope labeled amino acid
AEITSLLK

SEQ ID NO: 1233 - protein, 8 aa, Artificial Sequence
  Internal Standard Peptide Sequence to quantitate RAS-RAF-MAPK Pathway protein
  MOD_RES (8)..(8): heavy isotope labeled amino acid
TIIQDYIR

SEQ ID NO: 1234 - protein, 7 aa, Artificial Sequence
  Internal Standard Peptide Sequence to quantitate RAS-RAF-MAPK Pathway protein
  MOD_RES (5)..(5): carbamidomethylated cysteine
  MOD_RES (7)..(7): heavy isotope labeled amino acid
HLIICTR

SEQ ID NO: 1235 - protein, 15 aa, Artificial Sequence
  Internal Standard Peptide Sequence to quantitate RAS-RAF-MAPK Pathway protein
  MOD_RES (10)..(10): carbamidomethylated cysteine
  MOD_RES (15)..(15): heavy isotope labeled amino acid
AAWTSDISQCVDNIR

SEQ ID NO: 1236 - protein, 13 aa, Artificial Sequence
  Internal Standard Peptide Sequence to quantitate RAS-RAF-MAPK Pathway protein
  MOD_RES (7)..(7): carbamidomethylated cysteine
  MOD_RES (13)..(13): heavy isotope labeled amino acid
SDASLYCDDVDIR

SEQ ID NO: 1237 - protein, 9 aa, Artificial Sequence
  Internal Standard Peptide Sequence to quantitate RAS-RAF-MAPK Pathway protein
  MOD_RES (9)..(9): heavy isotope labeled amino acid
KPISAIPAR

SEQ ID NO: 1238 - protein, 20 aa, Artificial Sequence
  Internal Standard Peptide Sequence to quantitate RAS-RAF-MAPK Pathway protein
  MOD_RES (20)..(20): heavy isotope labeled amino acid
IPDEGDTTPEKPEDPSALSK

SEQ ID NO: 1239 - protein, 23 aa, Artificial Sequence
  Internal Standard Peptide Sequence to quantitate RAS-RAF-MAPK Pathway protein
  MOD_RES (23)..(23): heavy isotope labeled amino acid
SALSAASAFAIATAGANEGTPNK

SEQ ID NO: 1240 - protein, 25 aa, Artificial Sequence
  Internal Standard Peptide Sequence to quantitate RAS-RAF-MAPK Pathway protein
  MOD_RES (25)..(25): heavy isotope labeled amino acid
SALSAASAFAIATAGANEGTPNKEK

SEQ ID NO: 1241 - protein, 12 aa, Artificial Sequence
  Internal Standard Peptide Sequence to quantitate RAS-RAF-MAPK Pathway protein
  MOD_RES (12)..(12): heavy isotope labeled amino acid
HSQDFETNDELK

SEQ ID NO: 1242 - protein, 14 aa, Artificial Sequence
  Internal Standard Peptide Sequence to quantitate RAS-RAF-MAPK Pathway protein
  MOD_RES (13)..(13): carbamidomethylated cysteine
  MOD_RES (14)..(14): heavy isotope labeled amino acid
HSQDFETNDELKCK

SEQ ID NO: 1243 - protein, 12 aa, Artificial Sequence
  Internal Standard Peptide Sequence to quantitate RAS-RAF-MAPK Pathway protein
  MOD_RES (12)..(12): heavy isotope labeled amino acid
YIHLVQIVETEK

SEQ ID NO: 1244 - protein, 12 aa, Artificial Sequence
  Internal Standard Peptide Sequence to quantitate RAS-RAF-MAPK Pathway protein
  MOD_RES (12)..(12): heavy isotope labeled amino acid
HQLEDQDTEIER

SEQ ID NO: 1245 - protein, 7 aa, Artificial Sequence
  Internal Standard Peptide Sequence to quantitate RAS-RAF-MAPK Pathway protein
  MOD_RES (5)..(5): carbamidomethylated cysteine
  MOD_RES (7)..(7): heavy isotope labeled amino acid
HFLICTR

SEQ ID NO: 1246 - protein, 15 aa, Artificial Sequence
  Internal Standard Peptide Sequence to quantitate RAS-RAF-MAPK Pathway protein
  MOD_RES (10)..(10): carbamidomethylated cysteine
  MOD_RES (15)..(15): heavy isotope labeled amino acid
AAWMSDISQCVDNIR

SEQ ID NO: 1247 - protein, 15 aa, Artificial Sequence
  Internal Standard Peptide Sequence to quantitate RAS-RAF-MAPK Pathway protein
  MOD_RES (15)..(15): heavy isotope labeled amino acid
FLSIDFLNTFLHTYR

SEQ ID NO: 1248 - protein, 11 aa, Artificial Sequence
  Internal Standard Peptide Sequence to quantitate RAS-RAF-MAPK Pathway protein
  MOD_RES (11)..(11): heavy isotope labeled amino acid
IFTTAAVVLGK

SEQ ID NO: 1249 - protein, 11 aa, Artificial Sequence
  Internal Standard Peptide Sequence to quantitate RAS-RAF-MAPK Pathway protein
  MOD_RES (11)..(11): heavy isotope labeled amino acid
FSSPPPLAVSR

SEQ ID NO: 1250 - protein, 20 aa, Artificial Sequence
  Internal Standard Peptide Sequence to quantitate RAS-RAF-MAPK Pathway protein
  MOD_RES (20)..(20): heavy isotope labeled amino acid
GLSSPEQSPGTVEENVDNPR

SEQ ID NO: 1251 - protein, 10 aa, Artificial Sequence
  Internal Standard Peptide Sequence to quantitate RAS-RAF-MAPK Pathway protein
  MOD_RES (10)..(10): heavy isotope labeled amino acid
AVLESAPADR

SEQ ID NO: 1252 - protein, 18 aa, Artificial Sequence
  Internal Standard Peptide Sequence to quantitate RAS-RAF-MAPK Pathway protein
  MOD_RES (17)..(17): carbamidomethylated cysteine
  MOD_RES (18)..(18): heavy isotope labeled amino acid
AGVESSPAADTTELSPCR

SEQ ID NO: 1253 - protein, 12 aa, Artificial Sequence
  Internal Standard Peptide Sequence to quantitate RAS-RAF-MAPK Pathway protein
  MOD_RES (12)..(12): heavy isotope labeled amino acid
HAQDFELNNELK

SEQ ID NO: 1254 - protein, 13 aa, Artificial Sequence
  Internal Standard Peptide Sequence to quantitate RAS-RAF-MAPK Pathway protein
  MOD_RES (13)..(13): heavy isotope labeled amino acid
ALSQDDQDDIHLK

SEQ ID NO: 1255 - protein, 14 aa, Artificial Sequence
  Internal Standard Peptide Sequence to quantitate RAS-RAF-MAPK Pathway protein
  MOD_RES (14)..(14): heavy isotope labeled amino acid
SIPYEEFLGQGWMK

SEQ ID NO: 1256 - protein, 8 aa, Artificial Sequence
  Internal Standard Peptide Sequence to quantitate RAS-RAF-MAPK Pathway protein
  MOD_RES (8)..(8): heavy isotope labeled amino acid
EILQNVAR

SEQ ID NO: 1257 - protein, 11 aa, Artificial Sequence
  Internal Standard Peptide Sequence to quantitate RAS-RAF-MAPK Pathway protein
  MOD_RES (3)..(3): carbamidomethylated cysteine
  MOD_RES (11)..(11): heavy isotope labeled amino acid
LLCDIGHSEEK

SEQ ID NO: 1258 - protein, 8 aa, Artificial Sequence
  Internal Standard Peptide Sequence to quantitate RAS-RAF-MAPK Pathway protein
  MOD_RES (8)..(8): heavy isotope labeled amino acid
LALLNEAK

SEQ ID NO: 1259 - protein, 11 aa, Artificial Sequence
  Internal Standard Peptide Sequence to quantitate RAS-RAF-MAPK Pathway protein
  MOD_RES (11)..(11): heavy isotope labeled amino acid
YLIQDSSILQK

SEQ ID NO: 1260 - protein, 11 aa, Artificial Sequence
  Internal Standard Peptide Sequence to quantitate RAS-RAF-MAPK Pathway protein
  MOD_RES (11)..(11): heavy isotope labeled amino acid
ILAPYTDFHYR

SEQ ID NO: 1261 - protein, 11 aa, Artificial Sequence
  Internal Standard Peptide Sequence to quantitate RAS-RAF-MAPK Pathway protein
  MOD_RES (11)..(11): heavy isotope labeled amino acid
LSDGFVAAEAK

SEQ ID NO: 1262 - protein, 11 aa, Artificial Sequence
  Internal Standard Peptide Sequence to quantitate RAS-RAF-MAPK Pathway protein
  MOD_RES (11)..(11): heavy isotope labeled amino acid
LYANLDLDFAK

SEQ ID NO: 1263 - protein, 10 aa, Artificial Sequence
  Internal Standard Peptide Sequence to quantitate RAS-RAF-MAPK Pathway protein
  MOD_RES (10)..(10): heavy isotope labeled amino acid
LTVSSLDYSR

SEQ ID NO: 1264 - protein, 10 aa, Artificial Sequence
  Internal Standard Peptide Sequence to quantitate RAS-RAF-MAPK Pathway protein
  MOD_RES (9)..(9): carbamidomethylated cysteine
  MOD_RES (10)..(10): heavy isotope labeled amino acid
ILTAATDACR

SEQ ID NO: 1265 - protein, 17 aa, Artificial Sequence
  Internal Standard Peptide Sequence to quantitate RAS-RAF-MAPK Pathway protein
  MOD_RES (3)..(16): carbamidomethylated cysteine
  MOD_RES (17)..(17): heavy isotope labeled amino acid
TGCHLLEVQNIITELCR

SEQ ID NO: 1266 - protein, 17 aa, Artificial Sequence
  Internal Standard Peptide Sequence to quantitate RAS-RAF-MAPK Pathway protein
  MOD_RES (17)..(17): heavy isotope labeled amino acid
HIEDTGSTPSIGENDLK

SEQ ID NO: 1267 - protein, 16 aa, Artificial Sequence
  Internal Standard Peptide Sequence to quantitate RAS-RAF-MAPK Pathway protein
  MOD_RES (16)..(16): heavy isotope labeled amino acid
TSHYLTPQSNHLSLSK

SEQ ID NO: 1268 - protein, 17 aa, Artificial Sequence
  Internal Standard Peptide Sequence to quantitate RAS-RAF-MAPK Pathway protein
  MOD_RES (17)..(17): heavy isotope labeled amino acid
SNSVSLVPPGSSHTLPR

SEQ ID NO: 1269 - protein, 8 aa, Artificial Sequence
  Internal Standard Peptide Sequence to quantitate RAS-RAF-MAPK Pathway protein
  MOD_RES (8)..(8): heavy isotope labeled amino acid
DIPYFQTK

SEQ ID NO: 1270 - protein, 18 aa, Artificial Sequence
  Internal Standard Peptide Sequence to quantitate RAS-RAF-MAPK Pathway protein
  MOD_RES (18)..(18): heavy isotope labeled amino acid
AFAHDAGGLPSGTGGLVK

SEQ ID NO: 1271 - protein, 9 aa, Artificial Sequence
  Internal Standard Peptide Sequence to quantitate RAS-RAF-MAPK Pathway protein
  MOD_RES (9)..(9): heavy isotope labeled amino acid
STELLLGVK

SEQ ID NO: 1272 - protein, 9 aa, Artificial Sequence
  Internal Standard Peptide Sequence to quantitate RAS-RAF-MAPK Pathway protein
  MOD_RES (9)..(9): heavy isotope labeled amino acid
EISITHHVK

SEQ ID NO: 1273 - protein, 13 aa, Artificial Sequence
  Internal Standard Peptide Sequence to quantitate RAS-RAF-MAPK Pathway protein
  MOD_RES (13)..(13): heavy isotope labeled amino acid
DILADVNHPFVVK

SEQ ID NO: 1274 - protein, 11 aa, Artificial Sequence
  Internal Standard Peptide Sequence to quantitate RAS-RAF-MAPK Pathway protein
  MOD_RES (11)..(11): heavy isotope labeled amino acid
HVFYSTIDWNK

SEQ ID NO: 1275 - protein, 17 aa, Artificial Sequence
  Internal Standard Peptide Sequence to quantitate RAS-RAF-MAPK Pathway protein
  MOD_RES (17)..(17): heavy isotope labeled amino acid
GFSFVATGLMEDDGKPR

SEQ ID NO: 1276 - protein, 16 aa, Artificial Sequence
  Internal Standard Peptide Sequence to quantitate RAS-RAF-MAPK Pathway protein
  MOD_RES (16)..(16): heavy isotope labeled amino acid
APQAPLHSVVQQLHGK

SEQ ID NO: 1277 - protein, 11 aa, Artificial Sequence
  Internal Standard Peptide Sequence to quantitate RAS-RAF-MAPK Pathway protein
  MOD_RES (11)..(11): heavy isotope labeled amino acid
NLVFSDGYVVK

SEQ ID NO: 1278 - protein, 9 aa, Artificial Sequence
  Internal Standard Peptide Sequence to quantitate RAS-RAF-MAPK Pathway protein
  MOD_RES (9)..(9): heavy isotope labeled amino acid
ATNMEYAVK

SEQ ID NO: 1279 - protein, 11 aa, Artificial Sequence
  Internal Standard Peptide Sequence to quantitate RAS-RAF-MAPK Pathway protein
  MOD_RES (11)..(11): heavy isotope labeled amino acid
EASFVLHTIGK

SEQ ID NO: 1280 - protein, 13 aa, Artificial Sequence
  Internal Standard Peptide Sequence to quantitate RAS-RAF-MAPK Pathway protein
  MOD_RES (13)..(13): heavy isotope labeled amino acid
TVEYLHSQGVVHR

SEQ ID NO: 1281 - protein, 20 aa, Artificial Sequence
  Internal Standard Peptide Sequence to quantitate RAS-RAF-MAPK Pathway protein
  MOD_RES (18)..(18): carbamidomethylated cysteine
  MOD_RES (20)..(20): heavy isotope labeled amino acid
DLKPSNILYVDESGNPECLR

SEQ ID NO: 1282 - protein, 9 aa, Artificial Sequence
  Internal Standard Peptide Sequence to quantitate RAS-RAF-MAPK Pathway protein
  MOD_RES (9)..(9): heavy isotope labeled amino acid
VLGQGSYGK

SEQ ID NO: 1283 - protein, 13 aa, Artificial Sequence
  Internal Standard Peptide Sequence to quantitate RAS-RAF-MAPK Pathway protein
  MOD_RES (13)..(13): heavy isotope labeled amino acid
DILAEVNHPFIVK

SEQ ID NO: 1284 - protein, 12 aa, Artificial Sequence
  Internal Standard Peptide Sequence to quantitate RAS-RAF-MAPK Pathway protein
  MOD_RES (12)..(12): heavy isotope labeled amino acid
LGAGIDGVEEIK

SEQ ID NO: 1285 - protein, 13 aa, Artificial Sequence
  Internal Standard Peptide Sequence to quantitate RAS-RAF-MAPK Pathway protein
  MOD_RES (13)..(13): heavy isotope labeled amino acid
LGAGIDGVEEIKR

SEQ ID NO: 1286 - protein, 14 aa, Artificial Sequence
  Internal Standard Peptide Sequence to quantitate RAS-RAF-MAPK Pathway protein
  MOD_RES (14)..(14): heavy isotope labeled amino acid
HPFFVTIDWNTLYR

SEQ ID NO: 1287 - protein, 19 aa, Artificial Sequence
  Internal Standard Peptide Sequence to quantitate RAS-RAF-MAPK Pathway protein
  MOD_RES (19)..(19): heavy isotope labeled amino acid
TPTDSPGVPPSANAHHLFR

SEQ ID NO: 1288 - protein, 9 aa, Artificial Sequence
  Internal Standard Peptide Sequence to quantitate RAS-RAF-MAPK Pathway protein
  MOD_RES (9)..(9): heavy isotope labeled amino acid
ATDTEYAVK

SEQ ID NO: 1289 - protein, 11 aa, Artificial Sequence
  Internal Standard Peptide Sequence to quantitate RAS-RAF-MAPK Pathway protein
  MOD_RES (7)..(7): carbamidomethylated cysteine
  MOD_RES (11)..(11): heavy isotope labeled amino acid
EASDVLCTITK

SEQ ID NO: 1290 - protein, 16 aa, Artificial Sequence
  Internal Standard Peptide Sequence to quantitate RAS-RAF-MAPK Pathway protein
  MOD_RES (16)..(16): heavy isotope labeled amino acid
YALSGGNWDSISDAAK

SEQ ID NO: 1291 - protein, 12 aa, Artificial Sequence
  Internal Standard Peptide Sequence to quantitate RAS-RAF-MAPK Pathway protein
  MOD_RES (12)..(12): heavy isotope labeled amino acid
LEPVLSSNLAQR

SEQ ID NO: 1292 - protein, 11 aa, Artificial Sequence
  Internal Standard Peptide Sequence to quantitate RAS-RAF-MAPK Pathway protein
  MOD_RES (11)..(11): heavy isotope labeled amino acid
HSFFSTIDWNK

SEQ ID NO: 1293 - protein, 26 aa, Artificial Sequence
  Internal Standard Peptide Sequence to quantitate RAS-RAF-MAPK Pathway protein
  MOD_RES (26)..(26): heavy isotope labeled amino acid
EIHPPFKPATGRPEDTFYFDPEFTAK

SEQ ID NO: 1294 - protein, 16 aa, Artificial Sequence
  Internal Standard Peptide Sequence to quantitate RAS-RAF-MAPK Pathway protein
  MOD_RES (16)..(16): heavy isotope labeled amino acid
DSPGIPPSANAHQLFR

SEQ ID NO: 1295 - protein, 12 aa, Artificial Sequence
  Internal Standard Peptide Sequence to quantitate RAS-RAF-MAPK Pathway protein
  MOD_RES (12)..(12): heavy isotope labeled amino acid
NSIQFTDGYEVK

SEQ ID NO: 1296 - protein, 9 aa, Artificial Sequence
  Internal Standard Peptide Sequence to quantitate RAS-RAF-MAPK Pathway protein
  MOD_RES (9)..(9): heavy isotope labeled amino acid
ATNMEFAVK

SEQ ID NO: 1297 - protein, 11 aa, Artificial Sequence
  Internal Standard Peptide Sequence to quantitate RAS-RAF-MAPK Pathway protein
  MOD_RES (11)..(11): heavy isotope labeled amino acid
EASAVLFTITK

SEQ ID NO: 1298 - protein, 13 aa, Artificial Sequence
  Internal Standard Peptide Sequence to quantitate RAS-RAF-MAPK Pathway protein
  MOD_RES (13)..(13): heavy isotope labeled amino acid
TVEYLHAQGVVHR

SEQ ID NO: 1299 - protein, 20 aa, Artificial Sequence
  Internal Standard Peptide Sequence to quantitate RAS-RAF-MAPK Pathway protein
  MOD_RES (20)..(20): heavy isotope labeled amino acid
DLKPSNILYVDESGNPESIR

SEQ ID NO: 1300 - protein, 8 aa, Artificial Sequence
  Internal Standard Peptide Sequence to quantitate RAS-RAF-MAPK Pathway protein
  MOD_RES (8)..(8): heavy isotope labeled amino acid
LTAALVLR

SEQ ID NO: 1301 - protein, 17 aa, Artificial Sequence
  Internal Standard Peptide Sequence to quantitate RAS-RAF-MAPK Pathway protein
  MOD_RES (17)..(17): heavy isotope labeled amino acid
HPWIVHWDQLPQYQLNR

SEQ ID NO: 1302 - protein, 12 aa, Artificial Sequence
  Internal Standard Peptide Sequence to quantitate RAS-RAF-MAPK Pathway protein
  MOD_RES (12)..(12): heavy isotope labeled amino acid
GAMAATYSALNR

SEQ ID NO: 1303 - protein, 11 aa, Artificial Sequence
  Internal Standard Peptide Sequence to quantitate RAS-RAF-MAPK Pathway protein
  MOD_RES (11)..(11): heavy isotope labeled amino acid
NQSPVLEPVGR

SEQ ID NO: 1304 - protein, 10 aa, Artificial Sequence
  Internal Standard Peptide Sequence to quantitate RAS-RAF-MAPK Pathway protein
  MOD_RES (10)..(10): heavy isotope labeled amino acid
VGIENFELLK

SEQ ID NO: 1305 - protein, 19 aa, Artificial Sequence
  Internal Standard Peptide Sequence to quantitate RAS-RAF-MAPK Pathway protein
  MOD_RES (19)..(19): heavy isotope labeled amino acid
DLKPENLLFTDENDNLEIK

SEQ ID NO: 1306 - protein, 9 aa, Artificial Sequence
  Internal Standard Peptide Sequence to quantitate RAS-RAF-MAPK Pathway protein
  MOD_RES (9)..(9): heavy isotope labeled amino acid
EFVADETER

SEQ ID NO: 1307 - protein, 13 aa, Artificial Sequence
  Internal Standard Peptide Sequence to quantitate RAS-RAF-MAPK Pathway protein
  MOD_RES (13)..(13): heavy isotope labeled amino acid
DSPFYQHYDLDLK

SEQ ID NO: 1308 - protein, 10 aa, Artificial Sequence
  Internal Standard Peptide Sequence to quantitate RAS-RAF-MAPK Pathway protein
  MOD_RES (2)..(2): carbamidomethylated cysteine
  MOD_RES (10)..(10): heavy isotope labeled amino acid
LCEGHPNIVK

SEQ ID NO: 1309 - protein, 13 aa, Artificial Sequence
  Internal Standard Peptide Sequence to quantitate RAS-RAF-MAPK Pathway protein
  MOD_RES (12)..(12): carbamidomethylated cysteine
  MOD_RES (13)..(13): heavy isotope labeled amino acid
DKPLGEGSFSICR

SEQ ID NO: 1310 - protein, 13 aa, Artificial Sequence
  Internal Standard Peptide Sequence to quantitate RAS-RAF-MAPK Pathway protein
  MOD_RES (13)..(13): heavy isotope labeled amino acid
DLIQGLLTVDPNK

SEQ ID NO: 1311 - protein, 26 aa, Artificial Sequence
  Internal Standard Peptide Sequence to quantitate RAS-RAF-MAPK Pathway protein
  MOD_RES (26)..(26): heavy isotope labeled amino acid
AAGLEPVGHYEEVELTETSVNVGPER

SEQ ID NO: 1312 - protein, 10 aa, Artificial Sequence
  Internal Standard Peptide Sequence to quantitate RAS-RAF-MAPK Pathway protein
  MOD_RES (5)..(5): carbamidomethylated cysteine
  MOD_RES (10)..(10): heavy isotope labeled amino acid
IGPHCFELLR

SEQ ID NO: 1313 - protein, 7 aa, Artificial Sequence
  Internal Standard Peptide Sequence to quantitate RAS-RAF-MAPK Pathway protein
  MOD_RES (7)..(7): heavy isotope labeled amino acid
NILESVK

SEQ ID NO: 1314 - protein, 16 aa, Artificial Sequence
  Internal Standard Peptide Sequence to quantitate RAS-RAF-MAPK Pathway protein
  MOD_RES (16)..(16): heavy isotope labeled amino acid
HPFIVELAYAFQTGGK

SEQ ID NO: 1315 - protein, 13 aa, Artificial Sequence
  Internal Standard Peptide Sequence to quantitate RAS-RAF-MAPK Pathway protein
  MOD_RES (13)..(13): heavy isotope labeled amino acid
IGGGPGDAADVQR

SEQ ID NO: 1316 - protein, 21 aa, Artificial Sequence
  Internal Standard Peptide Sequence to quantitate RAS-RAF-MAPK Pathway protein
  MOD_RES (8)..(8): carbamidomethylated cysteine
  MOD_RES (21)..(21): heavy isotope labeled amino acid
VDPPFRPCLQSEEDVSQFDTR

SEQ ID NO: 1317 - protein, 8 aa, Artificial Sequence
  Internal Standard Peptide Sequence to quantitate RAS-RAF-MAPK Pathway protein
  MOD_RES (8)..(8): heavy isotope labeled amino acid
EGFSFQPK

SEQ ID NO: 1318 - protein, 11 aa, Artificial Sequence
  Internal Standard Peptide Sequence to quantitate RAS-RAF-MAPK Pathway protein
  MOD_RES (11)..(11): heavy isotope labeled amino acid
ALETIGANLQK

SEQ ID NO: 1319 - protein, 9 aa, Artificial Sequence
  Internal Standard Peptide Sequence to quantitate RAS-RAF-MAPK Pathway protein
  MOD_RES (9)..(9): heavy isotope labeled amino acid
IWQNFDSAR

SEQ ID NO: 1320 - protein, 13 aa, Artificial Sequence
  Internal Standard Peptide Sequence to quantitate RAS-RAF-MAPK Pathway protein
  MOD_RES (13)..(13): heavy isotope labeled amino acid
LYSLLSDPIPEVR

SEQ ID NO: 1321 - protein, 9 aa, Artificial Sequence
  Internal Standard Peptide Sequence to quantitate RAS-RAF-MAPK Pathway protein
  MOD_RES (9)..(9): heavy isotope labeled amino acid
SVSSYGNIR

SEQ ID NO: 1322 - protein, 18 aa, Artificial Sequence
  Internal Standard Peptide Sequence to quantitate RAS-RAF-MAPK Pathway protein
  MOD_RES (18)..(18): heavy isotope labeled amino acid
VLDTSSLTQSAPASPTNK

SEQ ID NO: 1323 - protein, 9 aa, Artificial Sequence
  Internal Standard Peptide Sequence to quantitate RAS-RAF-MAPK Pathway protein
  MOD_RES (9)..(9): heavy isotope labeled amino acid
LDDQIFLNR

SEQ ID NO: 1324 - protein, 21 aa, Artificial Sequence
  Internal Standard Peptide Sequence to quantitate RAS-RAF-MAPK Pathway protein
  MOD_RES (11)..(17): carbamidomethylated cysteine
  MOD_RES (21)..(21): heavy isotope labeled amino acid
VQDIPTGADSCVTSLSCDSHR

SEQ ID NO: 1325 - protein, 13 aa, Artificial Sequence
  Internal Standard Peptide Sequence to quantitate RAS-RAF-MAPK Pathway protein
  MOD_RES (13)..(13): heavy isotope labeled amino acid
SLIVAGLGDGSIR

SEQ ID NO: 1326 - protein, 9 aa, Artificial Sequence
  Internal Standard Peptide Sequence to quantitate RAS-RAF-MAPK Pathway protein
  MOD_RES (2)..(2): carbamidomethylated cysteine
  MOD_RES (9)..(9): heavy isotope labeled amino acid
YCQYAFDLK

SEQ ID NO: 1327 - protein, 13 aa, Artificial Sequence
  Internal Standard Peptide Sequence to quantitate RAS-RAF-MAPK Pathway protein
  MOD_RES (1)..(5): carbamidomethylated cysteine
  MOD_RES (13)..(13): heavy isotope labeled amino acid
CDSVCVNPYHYER

SEQ ID NO: 1328 - protein, 11 aa, Artificial Sequence
  Internal Standard Peptide Sequence to quantitate RAS-RAF-MAPK Pathway protein
  MOD_RES (2)..(2): carbamidomethylated cysteine
  MOD_RES (11)..(11): heavy isotope labeled amino acid
FCLGQLSNVHR

SEQ ID NO: 1329 - protein, 8 aa, Artificial Sequence
  Internal Standard Peptide Sequence to quantitate RAS-RAF-MAPK Pathway protein
  MOD_RES (8)..(8): heavy isotope labeled amino acid
GEGDVWVR

SEQ ID NO: 1330 - protein, 16 aa, Artificial Sequence
  Internal Standard Peptide Sequence to quantitate RAS-RAF-MAPK Pathway protein
  MOD_RES (1)..(1): carbamidomethylated cysteine
  MOD_RES (16)..(16): heavy isotope labeled amino acid
CLSDHAVFVQSYYLDR

SEQ ID NO: 1331 - protein, 8 aa, Artificial Sequence
  Internal Standard Peptide Sequence to quantitate RAS-RAF-MAPK Pathway protein
  MOD_RES (8)..(8): heavy isotope labeled amino acid
IYPSAYIK

SEQ ID NO: 1332 - protein, 8 aa, Artificial Sequence
  Internal Standard Peptide Sequence to quantitate RAS-RAF-MAPK Pathway protein
  MOD_RES (8)..(8): heavy isotope labeled amino acid
GWGPDYPR

SEQ ID NO: 1333 - protein, 15 aa, Artificial Sequence
  Internal Standard Peptide Sequence to quantitate RAS-RAF-MAPK Pathway protein
  MOD_RES (15)..(15): heavy isotope labeled amino acid
IAIENGDQPLSAELK

SEQ ID NO: 1334 - protein, 13 aa, Artificial Sequence
  Internal Standard Peptide Sequence to quantitate RAS-RAF-MAPK Pathway protein
  MOD_RES (13)..(13): heavy isotope labeled amino acid
LFSANDVENIFSR

SEQ ID NO: 1335 - protein, 10 aa, Artificial Sequence
  Internal Standard Peptide Sequence to quantitate RAS-RAF-MAPK Pathway protein
  MOD_RES (10)..(10): heavy isotope labeled amino acid
IVDIHELSVK

SEQ ID NO: 1336 - protein, 11 aa, Artificial Sequence
  Internal Standard Peptide Sequence to quantitate RAS-RAF-MAPK Pathway protein
  MOD_RES (11)..(11): heavy isotope labeled amino acid
LDHTFEQIPSR

SEQ ID NO: 1337 - protein, 9 aa, Artificial Sequence
  Internal Standard Peptide Sequence to quantitate RAS-RAF-MAPK Pathway protein
  MOD_RES (9)..(9): heavy isotope labeled amino acid
VTPTFHHWK

SEQ ID NO: 1338 - protein, 13 aa, Artificial Sequence
  Internal Standard Peptide Sequence to quantitate RAS-RAF-MAPK Pathway protein
  MOD_RES (9)..(9): carbamidomethylated cysteine
  MOD_RES (13)..(13): heavy isotope labeled amino acid
AIEDISQGCPESK

SEQ ID NO: 1339 - protein, 22 aa, Artificial Sequence
  Internal Standard Peptide Sequence to quantitate RAS-RAF-MAPK Pathway protein
  MOD_RES (22)..(22): heavy isotope labeled amino acid
NEAEGADDLQANEEDSSSSLVK

SEQ ID NO: 1340 - protein, 16 aa, Artificial Sequence
  Internal Standard Peptide Sequence to quantitate RAS-RAF-MAPK Pathway protein
  MOD_RES (16)..(16): heavy isotope labeled amino acid
DHLFQQETVVTSEPYR

SEQ ID NO: 1341 - protein, 15 aa, Artificial Sequence
  Internal Standard Peptide Sequence to quantitate RAS-RAF-MAPK Pathway protein
  MOD_RES (15)..(15): heavy isotope labeled amino acid
SSNIRPSPFEDLNAR

SEQ ID NO: 1342 - protein, 12 aa, Artificial Sequence
  Internal Standard Peptide Sequence to quantitate RAS-RAF-MAPK Pathway protein
  MOD_RES (12)..(12): heavy isotope labeled amino acid
HVSFQDEDEIVR

SEQ ID NO: 1343 - protein, 7 aa, Artificial Sequence
  Internal Standard Peptide Sequence to quantitate RAS-RAF-MAPK Pathway protein
  MOD_RES (7)..(7): heavy isotope labeled amino acid
TQPSSLK

SEQ ID NO: 1344 - protein, 8 aa, Artificial Sequence
  Internal Standard Peptide Sequence to quantitate RAS-RAF-MAPK Pathway protein
  MOD_RES (8)..(8): heavy isotope labeled amino acid
FNHEENVR

SEQ ID NO: 1345 - protein, 17 aa, Artificial Sequence
  Internal Standard Peptide Sequence to quantitate RAS-RAF-MAPK Pathway protein
  MOD_RES (17)..(17): heavy isotope labeled amino acid
DDSSGGWFPQEGGGISR

SEQ ID NO: 1346 - protein, 9 aa, Artificial Sequence
  Internal Standard Peptide Sequence to quantitate RAS-RAF-MAPK Pathway protein
  MOD_RES (6)..(6): carbamidomethylated cysteine
  MOD_RES (9)..(9): heavy isotope labeled amino acid
LVVLECYVR

SEQ ID NO: 1347 - protein, 11 aa, Artificial Sequence
  Internal Standard Peptide Sequence to quantitate RAS-RAF-MAPK Pathway protein
  MOD_RES (8)..(8): carbamidomethylated cysteine
  MOD_RES (11)..(11): heavy isotope labeled amino acid
TISSPTSCEHR

SEQ ID NO: 1348 - protein, 12 aa, Artificial Sequence
  Internal Standard Peptide Sequence to quantitate RAS-RAF-MAPK Pathway protein
  MOD_RES (12)..(12): heavy isotope labeled amino acid
EKIWMTGYEDYR

SEQ ID NO: 1349 - protein, 10 aa, Artificial Sequence
  Internal Standard Peptide Sequence to quantitate RAS-RAF-MAPK Pathway protein
  MOD_RES (10)..(10): heavy isotope labeled amino acid
IWMTGYEDYR

SEQ ID NO: 1350 - protein, 16 aa, Artificial Sequence
  Internal Standard Peptide Sequence to quantitate RAS-RAF-MAPK Pathway protein
  MOD_RES (16)..(16): heavy isotope labeled amino acid
GKYPDPSEDADSSYVR

SEQ ID NO: 1351 - protein, 14 aa, Artificial Sequence
  Internal Standard Peptide Sequence to quantitate RAS-RAF-MAPK Pathway protein
  MOD_RES (14)..(14): heavy isotope labeled amino acid
YPDPSEDADSSYVR

SEQ ID NO: 1352 - protein, 20 aa, Artificial Sequence
  Internal Standard Peptide Sequence to quantitate RAS-RAF-MAPK Pathway protein
  MOD_RES (20)..(20): heavy isotope labeled amino acid
HDYNYPYVDSSDFGLGEDPK

SEQ ID NO: 1353 - protein, 11 aa, Artificial Sequence
  Internal Standard Peptide Sequence to quantitate RAS-RAF-MAPK Pathway protein
  MOD_RES (3)..(3): carbamidomethylated cysteine
  MOD_RES (11)..(11): heavy isotope labeled amino acid
GHCQDAPDSVR

SEQ ID NO: 1354 - protein, 14 aa, Artificial Sequence
  Internal Standard Peptide Sequence to quantitate RAS-RAF-MAPK Pathway protein
  MOD_RES (14)..(14): heavy isotope labeled amino acid
EIQPTAILSLDQIK

SEQ ID NO: 1355 - protein, 13 aa, Artificial Sequence
  Internal Standard Peptide Sequence to quantitate RAS-RAF-MAPK Pathway protein
  MOD_RES (13)..(13): heavy isotope labeled amino acid
GSNEYTEGPSVVK

SEQ ID NO: 1356 - protein, 15 aa, Artificial Sequence
  Internal Standard Peptide Sequence to quantitate RAS-RAF-MAPK Pathway protein
  MOD_RES (15)..(15): heavy isotope labeled amino acid
HTSHLGHAVLPSNAR

SEQ ID NO: 1357 - protein, 8 aa, Artificial Sequence
  Internal Standard Peptide Sequence to quantitate RAS-RAF-MAPK Pathway protein
  MOD_RES (3)..(6): carbamidomethylated cysteine
  MOD_RES (8)..(8): heavy isotope labeled amino acid
FICEQCGK

SEQ ID NO: 1358 - protein, 11 aa, Artificial Sequence
  Internal Standard Peptide Sequence to quantitate RAS-RAF-MAPK Pathway protein
  MOD_RES (11)..(11): heavy isotope labeled amino acid
SPPTRPVPGHR

SEQ ID NO: 1359 - protein, 16 aa, Artificial Sequence
  Internal Standard Peptide Sequence to quantitate RAS-RAF-MAPK Pathway protein
  MOD_RES (16)..(16): heavy isotope labeled amino acid
DALTQQVHVLSLDQIR

SEQ ID NO: 1360 - protein, 8 aa, Artificial Sequence
  Internal Standard Peptide Sequence to quantitate RAS-RAF-MAPK Pathway protein
  MOD_RES (8)..(8): heavy isotope labeled amino acid
LHGLPEHR

SEQ ID NO: 1361 - protein, 17 aa, Artificial Sequence
  Internal Standard Peptide Sequence to quantitate RAS-RAF-MAPK Pathway protein
  MOD_RES (17)..(17): heavy isotope labeled amino acid
LLGSSFSSGPVADGIIR

SEQ ID NO: 1362 - protein, 9 aa, Artificial Sequence
  Internal Standard Peptide Sequence to quantitate RAS-RAF-MAPK Pathway protein
  MOD_RES (9)..(9): heavy isotope labeled amino acid
EDLGLHAYR

SEQ ID NO: 1363 - protein, 13 aa, Artificial Sequence
  Internal Standard Peptide Sequence to quantitate RAS-RAF-MAPK Pathway protein
  MOD_RES (13)..(13): heavy isotope labeled amino acid
SELKPGELKPLSK

SEQ ID NO: 1364 - protein, 11 aa, Artificial Sequence
  Internal Standard Peptide Sequence to quantitate RAS-RAF-MAPK Pathway protein
  MOD_RES (11)..(11): heavy isotope labeled amino acid
LQHSQVHSSAR

SEQ ID NO: 1365 - protein, 17 aa, Artificial Sequence
  Internal Standard Peptide Sequence to quantitate RAS-RAF-MAPK Pathway protein
  MOD_RES (16)..(16): carbamidomethylated cysteine
  MOD_RES (17)..(17): heavy isotope labeled amino acid
STHASNDYVERPPAPCK

SEQ ID NO: 1366 - protein, 16 aa, Artificial Sequence
  Internal Standard Peptide Sequence to quantitate RAS-RAF-MAPK Pathway protein
  MOD_RES (16)..(16): heavy isotope labeled amino acid
LLASITPSPSGQSIIR

SEQ ID NO: 1367 - protein, 14 aa, Artificial Sequence
  Internal Standard Peptide Sequence to quantitate RAS-RAF-MAPK Pathway protein
  MOD_RES (14)..(14): heavy isotope labeled amino acid
QALSSPSLIVQTHK

SEQ ID NO: 1368 - protein, 19 aa, Artificial Sequence
  Internal Standard Peptide Sequence to quantitate RAS-RAF-MAPK Pathway protein
  MOD_RES (1)..(17): carbamidomethylated cysteine
  MOD_RES (19)..(19): heavy isotope labeled amino acid
CLCSAESLLDYGTCLCCVK

SEQ ID NO: 1369 - protein, 14 aa, Artificial Sequence
  Internal Standard Peptide Sequence to quantitate RAS-RAF-MAPK Pathway protein
  MOD_RES (14)..(14): heavy isotope labeled amino acid
LQHPLTILPIDQVK

SEQ ID NO: 1370 - protein, 21 aa, Artificial Sequence
  Internal Standard Peptide Sequence to quantitate RAS-RAF-MAPK Pathway protein
  MOD_RES (21)..(21): heavy isotope labeled amino acid
TSHVENDYIDNPSLALTTGPK

SEQ ID NO: 1371 - protein, 14 aa, Artificial Sequence
  Internal Standard Peptide Sequence to quantitate RAS-RAF-MAPK Pathway protein
  MOD_RES (14)..(14): heavy isotope labeled amino acid
TRGGAPELAPTPAR

SEQ ID NO: 1372 - protein, 12 aa, Artificial Sequence
  Internal Standard Peptide Sequence to quantitate RAS-RAF-MAPK Pathway protein
  MOD_RES (12)..(12): heavy isotope labeled amino acid
GGAPELAPTPAR

SEQ ID NO: 1373 - protein, 30 aa, Artificial Sequence; identical to SEQ ID NO: 673
  Internal Standard Peptide Sequence to quantitate RAS-RAF-MAPK Pathway protein
  MOD_RES (1)..(1): carbamidomethylated cysteine
  MOD_RES (30)..(30): heavy isotope labeled amino acid

SEQ ID NO: 1374 - protein, 17 aa, Artificial Sequence
  Internal Standard Peptide Sequence to quantitate RAS-RAF-MAPK Pathway protein
  MOD_RES (17)..(17): heavy isotope labeled amino acid
LLDHMAPPPVADQASPR

SEQ ID NO: 1375 - protein, 10 aa, Artificial Sequence
  Internal Standard Peptide Sequence to quantitate RAS-RAF-MAPK Pathway protein
  MOD_RES (4)..(4): carbamidomethylated cysteine
  MOD_RES (10)..(10): heavy isotope labeled amino acid
VVHCQPLDLK

SEQ ID NO: 1376 - protein, 10 aa, Artificial Sequence
  Internal Standard Peptide Sequence to quantitate RAS-RAF-MAPK Pathway protein
  MOD_RES (5)..(8): carbamidomethylated cysteine
  MOD_RES (10)..(10): heavy isotope labeled amino acid
HFLLCEACGK

SEQ ID NO: 1377 - protein, 8 aa, Artificial Sequence
  Internal Standard Peptide Sequence to quantitate RAS-RAF-MAPK Pathway protein
  MOD_RES (7)..(7): carbamidomethylated cysteine
  MOD_RES (8)..(8): heavy isotope labeled amino acid
HTNSVICK

SEQ ID NO: 1378 - protein, 14 aa, Artificial Sequence
  Internal Standard Peptide Sequence to quantitate RAS-RAF-MAPK Pathway protein
  MOD_RES (14)..(14): heavy isotope labeled amino acid
FLQESNVLYQHNLR

SEQ ID NO: 1379 - protein, 7 aa, Artificial Sequence
  Internal Standard Peptide Sequence to quantitate RAS-RAF-MAPK Pathway protein
  MOD_RES (1)..(1): carbamidomethylated cysteine
  MOD_RES (7)..(7): heavy isotope labeled amino acid
CLWEESR

SEQ ID NO: 1380 - protein, 26 aa, Artificial Sequence
  Internal Standard Peptide Sequence to quantitate RAS-RAF-MAPK Pathway protein
  MOD_RES (26)..(26): heavy isotope labeled amino acid
LLQTAATAAQQGGQANHPTAAVVTEK

SEQ ID NO: 1381 - protein, 7 aa, Artificial Sequence
  Internal Standard Peptide Sequence to quantitate RAS-RAF-MAPK Pathway protein
  MOD_RES (7)..(7): heavy isotope labeled amino acid
SAFVVER

SEQ ID NO: 1382 - protein, 9 aa, Artificial Sequence
  Internal Standard Peptide Sequence to quantitate RAS-RAF-MAPK Pathway protein
  MOD_RES (9)..(9): heavy isotope labeled amino acid
FPELNYQLK

SEQ ID NO: 1383 - protein, 9 aa, Artificial Sequence
  Internal Standard Peptide Sequence to quantitate RAS-RAF-MAPK Pathway protein
  MOD_RES (9)..(9): heavy isotope labeled amino acid
FNILGTNTK

SEQ ID NO: 1384 - protein, 13 aa, Artificial Sequence
  Internal Standard Peptide Sequence to quantitate RAS-RAF-MAPK Pathway protein
  MOD_RES (13)..(13): heavy isotope labeled amino acid
GLSIEQLTTLAEK

SEQ ID NO: 1385 - protein, 14 aa, Artificial Sequence
  Internal Standard Peptide Sequence to quantitate RAS-RAF-MAPK Pathway protein
  MOD_RES (14)..(14): heavy isotope labeled amino acid
AILSTKPPGTFLLR

SEQ ID NO: 1386 - protein, 11 aa, Artificial Sequence
  Internal Standard Peptide Sequence to quantitate RAS-RAF-MAPK Pathway protein
  MOD_RES (11)..(11): heavy isotope labeled amino acid
TQIQSVEPYTK

SEQ ID NO: 1387 - protein, 11 aa, Artificial Sequence
  Internal Standard Peptide Sequence to quantitate RAS-RAF-MAPK Pathway protein
  MOD_RES (11)..(11): heavy isotope labeled amino acid
IDSTEVIYQPR

SEQ ID NO: 1388 - protein, 20 aa, Artificial Sequence
  Internal Standard Peptide Sequence to quantitate RAS-RAF-MAPK Pathway protein
  MOD_RES (19)..(19): carbamidomethylated cysteine
  MOD_RES (20)..(20): heavy isotope labeled amino acid
ISDLGVAEALHPFAADDTCR

SEQ ID NO: 1389 - protein, 18 aa, Artificial Sequence
  Internal Standard Peptide Sequence to quantitate RAS-RAF-MAPK Pathway protein
  MOD_RES (9)..(9): carbamidomethylated cysteine
  MOD_RES (18)..(18): heavy isotope labeled amino acid
GSYAIPGDCGPPLSDLLK

SEQ ID NO: 1390 - protein, 14 aa, Artificial Sequence
  Internal Standard Peptide Sequence to quantitate RAS-RAF-MAPK Pathway protein
  MOD_RES (14)..(14): heavy isotope labeled amino acid
NVIQLVDVLYNEEK

SEQ ID NO: 1391 - protein, 9 aa, Artificial Sequence
  Internal Standard Peptide Sequence to quantitate RAS-RAF-MAPK Pathway protein
  MOD_RES (9)..(9): heavy isotope labeled amino acid
TYQGSYGFR

SEQ ID NO: 1392 - protein, 10 aa, Artificial Sequence
  Internal Standard Peptide Sequence to quantitate RAS-RAF-MAPK Pathway protein
  MOD_RES (10)..(10): heavy isotope labeled amino acid
LGFLHSGTAK

SEQ ID NO: 1393 - protein, 12 aa, Artificial Sequence
  Internal Standard Peptide Sequence to quantitate RAS-RAF-MAPK Pathway protein
  MOD_RES (4)..(4): carbamidomethylated cysteine
  MOD_RES (12)..(12): heavy isotope labeled amino acid
SVTCTYSPALNK

SEQ ID NO: 1394 - protein, 17 aa, Artificial Sequence
  Internal Standard Peptide Sequence to quantitate RAS-RAF-MAPK Pathway protein
  MOD_RES (2)..(2): carbamidomethylated cysteine
  MOD_RES (17)..(17): heavy isotope labeled amino acid
TCPVQLWVDSTPPPGTR

SEQ ID NO: 1395 - protein, 15 aa, Artificial Sequence
  Internal Standard Peptide Sequence to quantitate RAS-RAF-MAPK Pathway protein
  MOD_RES (1)..(1): carbamidomethylated cysteine
  MOD_RES (15)..(15): heavy isotope labeled amino acid
CSDSDGLAPPQHLIR

SEQ ID NO: 1396 - protein, 7 aa, Artificial Sequence
  Internal Standard Peptide Sequence to quantitate RAS-RAF-MAPK Pathway protein
  MOD_RES (7)..(7): heavy isotope labeled amino acid
VEYLDDR

SEQ ID NO: 1397 - protein, 19 aa, Artificial Sequence
  Internal Standard Peptide Sequence to quantitate RAS-RAF-MAPK Pathway protein
  MOD_RES (19)..(19): heavy isotope labeled amino acid
RPILTIITLEDSSGNLLGR

SEQ ID NO: 1398 - protein, 13 aa, Artificial Sequence
  Internal Standard Peptide Sequence to quantitate RAS-RAF-MAPK Pathway protein
  MOD_RES (13)..(13): heavy isotope labeled amino acid
ALPNNTSSSPQPK

SEQ ID NO: 1399 - protein, 9 aa, Artificial Sequence
  Internal Standard Peptide Sequence to quantitate RAS-RAF-MAPK Pathway protein
  MOD_RES (9)..(9): heavy isotope labeled amino acid
ELNEALELK

SEQ ID NO: 1400 - protein, 13 aa, Artificial Sequence
  Internal Standard Peptide Sequence to quantitate RAS-RAF-MAPK Pathway protein
  MOD_RES (9)..(9): phosphorylated serine
  MOD_RES (13)..(13): heavy isotope labeled amino acid
ALPNNTSSSPQPK

(19) United States
(12) Patent Application Publication
(10) Pub. No.: US 2020/0355695 A1
(43) Pub. Date: Nov. 12, 2020

(54) DETECTION AND QUANTIFICATION OF RAS-RAF-MAPK PATHWAY PROTEINS

(71) Applicant: PIERCE BIOTECHNOLOGY, INC., Carlsbad, CA (US)

(72) Inventors: Bhavinkumar PATEL, Loves Park, IL (US); John ROGERS, Rockton, IL (US)

(21) Appl. No.: 16/547,695

(22) PCT Filed: Feb. 27, 2018
(86) PCT No.: PCT/US2018/019998

§ 371 Date: Aug. 22, 2019

Related U.S. Application Data
(60) Provisional application No. 62/465,102, filed on Feb. 28, 2017.

(51) Int. Cl. G01N 33/574; G01N 33/68

(52) U.S. Cl. CPC G01N 33/57496; G01N 33/6848

(57) ABSTRACT

This disclosure relates to the field of mass spectrometry analysis. In some embodiments, the disclosure relates to compositions and methods for detecting and quantifying proteins in the RAS-RAF-MAPK pathway by immunoprecipitation enrichment followed by mass spectrometry analysis.

### CROSS-REFERENCE TO RELATED APPLICATIONS

This application is a 371 National Stage filing from PCT/US2018/019998, filed Feb. 27, 2018 which claims the benefit of priority of U.S. Provisional Application No. 62/465,102, filed Feb. 28, 2017, which are incorporated by reference in their entirety for any purpose.

### SEQUENCE LISTING

The instant application contains a Sequence Listing which has been submitted electronically in ASCII format and is hereby incorporated by reference in its entirety. Said ASCII copy, created on Feb. 22, 2018, is named 2018-02-22 01129-0062-00PCT_Seq_List_ST25 and is 485 kilobytes in size.

### FIELD OF THE INVENTION

This disclosure relates to the field of detection and quantification of RAS-RAF-MAPK pathway proteins, including adjacent pathway proteins, by immunoprecipitation and mass spectrometry.

### BACKGROUND

The RAS-RAF-MAPK and AKT-mTOR signaling pathways are key regulators of several aspects of normal cell growth and malignant transformation such as cell survival, differentiation, proliferation, metabolism, and motility in response to extracellular cues. These two pathways extensively engage in cross-talk in order to both positively and negatively regulate each other. Because of its importance in cancer, the RAS-MAPK pathway targets have been a focus for drug targeting for last two decades. Rational therapies that target the key regulators of RAS pathway might inhibit tumor growth, survival and spread. See Mendoza M C; Er E E; Blenis J.; Trends Biochem Sci. 2011 June; 36(6):320-8.

A major limitation in the detection and quantitation of RAS-RAF-MAPK pathway proteins is the lack of rigorously validated methods and reagents. Currently, only semi-quantitative results from Western blotting, ELISA, and Luminex assays are available. Mass spectrometry (MS) is increasingly becoming the detection methodology of choice for assaying protein abundance and post-translational modifications. However, to date, MS has not been successful in quantifying RAS-RAF-MAPK pathway proteins, possibly due to their low abundance and significant post-translational modification profiles.

Immunoenrichment or Immunoprecipitation (IP) is commonly used upstream of MS as an enrichment tool for low-abundant protein targets. See, Gingras et al., Nat. Rev. Mol. Cell. Biol., August 2007, 8 (8), 645-54; and Can, S. A. et al., Mol. Cell. Proteomics March 2014, 13 (3), 907-17. The identification of appropriate antibodies for use in IP upstream of MS is important, as not all antibodies that bind to protein will be effective immunoprecipitation tools, and further, not all antibodies that are effective immunoprecipitation tools will lead to successful identification via MS.

### SUMMARY

The present disclosure provides reagents and methods for detecting and quantifying RAS-RAF-MAPK pathway proteins via immunoprecipitation (IP), mass spectrometry (MS), and immunoprecipitation followed by mass spectrometry (IP-MS).

In some embodiments, methods for immunoprecipitating a RAS-RAF-MAPK pathway protein are provided, comprising contacting a biological sample with any one of the antibodies recited in Table 1. In some embodiments, the antibodies useful in the IP methods comprise the antibodies recited in Table 6. The methods may comprise washing the contacted biological sample to enrich for antibody-protein conjugates. Further methods include detecting the antibody-protein conjugates to determine the RAS-RAF-MAPK pathway protein in the biological sample. In some embodiments, the antibody is labelled. In some embodiments, a detection reagent is provided to the enriched antibody-protein conjugate. In some embodiments the label is biotin and the detection reagent is streptavidin.

In some embodiments the IP is single-plex. In some embodiments the IP is multi-plex. The antibodies useful in multi-plex IP may comprise the antibodies of Table 6.

In some embodiments a method for detecting RAS-RAF-MAPK pathway proteins via MS is provided, comprising isolating proteins from a biological sample, digesting the isolated proteins, assaying the digested proteins via mass spectrometry to determine the presence of a peptide for RAS-RAF-MAPK pathway protein(s), and determining the identity of one or more RAS-RAF-MAPK pathway protein(s) in the sample. In some embodiments, the peptide for RAS-RAF-MAPK pathway protein(s) comprises a sequence of SEQ ID NO: 1-SEQ ID NO: 1400. In some embodiments, the peptide is less than or equal to 40 amino acids in length. In some embodiments, the peptide for RAS-RAF-MAPK pathway protein(s) consists of a sequence of SEQ ID NO: 1-SEQ ID NO: 1400. The peptides of SEQ ID NO: 1-SEQ ID NO: 700 may be labelled. In some embodiments the label on SEQ ID NO: 1-SEQ ID NO: 700 differs from the label shown on the peptides of SEQ ID NO: 701-SEQ ID NO: 1400.

In some embodiments a method for quantifying RAS-RAF-MAPK pathway proteins via MS is provided, comprising isolating proteins from a biological sample, digesting the isolated proteins, assaying the digested proteins via mass spectrometry to determine the presence of a peptide for RAS-RAF-MAPK pathway protein(s), and determining the quantity of one or more RAS-RAF-MAPK pathway protein(s) in the sample. In some embodiments, the peptide for RAS-RAF-MAPK pathway protein(s) comprises a sequence of SEQ ID NO: 1-SEQ ID NO: 1400. In some embodiments, the peptide is less than or equal to 40 amino acids in length. In some embodiments, the peptide for RAS-RAF-MAPK pathway protein(s) consists of a sequence of SEQ ID NO: 1-SEQ ID NO: 1400. The peptides of SEQ ID NO: 1-SEQ ID NO: 700 may be labelled. In some embodiments the label on SEQ ID NO: 1-SEQ ID NO: 700 differs from the label shown on the peptides of SEQ ID NO: 701-SEQ ID NO: 1400. In some embodiments, the peptide comprises or consists of a peptide selected from the peptides shown in Table 5 (SEQ ID Nos: 13, 15, 18, 28, 31, 47, 48, 51, 127, 128, 132, 133, 141, 142, 143, 144, 171, 173, 177, 180, 181, 182, 183, 190, 192, 213, 219, 220, 224, 226, 231, 233, 234, 235, 236, 258, 270, 295, 297, 300, 301, 302, 304, 306, 308, 310, 347, 348, 353, 362, 392, 393, 405, 416, 429, 443, 446, 451, 467, 483, 487, 501, 502, 506, 507, 510, 512, 521, 522, 523, 528, 572, 580, 588, 591, 598, 603, 615, 617, 635, 636, 655, 657, 660, 662, 670, 678, 683, 691, 692, 696, 698, and 699).

In some embodiments a method for detecting RAS-RAF-MAPK pathway proteins via IP-MS is provided, comprising treating a biological sample with at least one antibody capable of immunoprecipitating RAS-RAF-MAPK target pathway protein(s) from a biological sample, digesting the isolated proteins, assaying the digested proteins via mass spectrometry to determine the presence of a peptide for RAS-RAF-MAPK pathway protein(s), and determining the identity of one or more RAS-RAF-MAPK pathway protein(s) in the sample. In some embodiments, the peptide for RAS-RAF-MAPK pathway protein(s) comprises a sequence of SEQ ID NO: 1-SEQ ID NO: 1400. In some embodiments the peptide is less than or equal to 40 amino acids in length. In some embodiments, the peptide for RAS-RAF-MAPK pathway protein(s) consists of a sequence of SEQ ID NO: 1-SEQ ID NO: 1400. The peptides of SEQ ID NO: 1-SEQ ID NO: 700 may be labelled. In some embodiments the label on SEQ ID NO: 1-SEQ ID NO: 700 differs from the label shown on the peptides of SEQ ID NO: 701-SEQ ID NO: 1400. In some embodiments, the peptide comprises or consists of a peptide selected from the peptides shown in Table 5 (SEQ ID Nos: 13, 15, 18, 28, 31, 47, 48, 51, 127, 128, 132, 133, 141, 142, 143, 144, 171, 173, 177, 180, 181, 182, 183, 190, 192, 213, 219, 220, 224, 226, 231, 233, 234, 235, 236, 258, 270, 295, 297, 300, 301, 302, 304, 306, 308, 310, 347, 348, 353, 362, 392, 393, 405, 416, 429, 443, 446, 451, 467, 483, 487, 501, 502, 506, 507, 510, 512, 521, 522, 523, 528, 572, 580, 588, 591, 598, 603, 615, 617, 635, 636, 655, 657, 660, 662, 670, 678, 683, 691, 692, 696, 698, and 699).

In some embodiments a method for quantifying RAS-RAF-MAPK pathway proteins via IP-MS is provided, comprising treating a biological sample with at least one antibody capable of immunoprecipitating RAS-RAF-MAPK target pathway protein(s) from a biological sample, digesting the isolated proteins, assaying the digested proteins via mass spectrometry to determine the presence of a peptide for RAS-RAF-MAPK pathway protein(s), and determining the quantity of one or more RAS-RAF-MAPK pathway protein(s) in the sample. In some embodiments, the peptide for RAS-RAF-MAPK pathway protein(s) comprises a sequence of SEQ ID NO: 1-SEQ ID NO: 1400. In some embodiments the peptide is less than or equal to 40 amino acids in length. In some embodiments, the peptide for RAS-RAF-MAPK pathway protein(s) consists of a sequence of SEQ ID NO: 1-SEQ ID NO: 1400. The peptides of SEQ ID NO: 1-SEQ ID NO: 700 may be labelled. In some embodiments the label on SEQ ID NO: 1-SEQ ID NO: 700 differs from the label shown on the peptides of SEQ ID NO: 701-SEQ ID NO: 1400. In some embodiments, the peptide comprises or consists of a peptide selected from the peptides shown in Table 5 (SEQ ID Nos: 13, 15, 18, 28, 31, 47, 48, 51, 127, 128, 132, 133, 141, 142, 143, 144, 171, 173, 177, 180, 181, 182, 183, 190, 192, 213, 219, 220, 224, 226, 231, 233, 234, 235, 236, 258, 270, 295, 297, 300, 301, 302, 304, 306, 308, 310, 347, 348, 353, 362, 392, 393, 405, 416, 429, 443, 446, 451, 467, 483, 487, 501, 502, 506, 507, 510, 512, 521, 522, 523, 528, 572, 580, 588, 591, 598, 603, 615, 617, 635, 636, 655, 657, 660, 662, 670, 678, 683, 691, 692, 696, 698, and 699).

In some embodiments the RAS-RAF-MAPK pathway target protein is phosphorylated.

Methods for determining the ratio of phosphorylated to non-phosphorylated RAS-RAF-MAPK pathway proteins are provided, comprising any of the above IP, MS, or MS-IP methods, wherein a further step of determining the ratio of phosphorylated to non-phosphorylated protein is provided. In some embodiments, the method is an MS-IP method comprising treating a biological sample with one or more antibodies capable of immunoprecipitating one or more phosphorylated RAS-RAF-MAPK pathway proteins, and separately treating the same biological sample with one or more antibodies capable of immunoprecipitating at least one or more of the same or different non-phosphorylated RAS-RAF-MAPK pathway proteins; digesting the immunoprecipitated RAS-RAF-MAPK pathway proteins; adding a first and a second detectably labelled internal standard peptide of known amount to the digested proteins, wherein the first internal standard peptide has the same amino acid sequence as a phosphorylated RAS-RAF-MAPK pathway peptide used to identify the phosphorylated protein, and the second internal standard peptide has the same amino acid sequence as the non-phosphorylated RAS-RAF-MAPK pathway peptide used to identify the non-phosphorylated protein; assaying the digested protein and internal standards via mass spectrometry to determine the presence and amount of phosphorylated and non-phosphorylated RAS-RAF-MAPK pathway proteins, wherein the RAS-RAF-MAPK pathway peptide comprises a peptide of SEQ ID NO: 1-SEQ ID NO: 1400, and is less than or equal to 40 amino acids in length; determining the quantity of RAS-RAF-MAPK phosphorylated and non-phosphorylated pathway proteins in the sample, and determining the ratio of phosphorylated to non-phosphorylated pathway proteins. In some embodiments, the peptide for RAS-RAF-MAPK pathway protein(s) consists of a sequence of SEQ ID NO: 1-SEQ ID NO: 1400. The peptides of SEQ ID NO: 1-SEQ ID NO: 700 may be labelled. In some embodiments the label on SEQ ID NO: 1-SEQ ID NO: 700 differs from the label shown on the peptides of SEQ ID NO: 701-SEQ ID NO: 1400. In some embodiments, the peptide comprises or consists of a peptide selected from the peptides shown in Table 5 (SEQ ID Nos: 13, 15, 18, 28, 31, 47, 48, 51, 127, 128, 132, 133, 141, 142, 143, 144, 171, 173, 177, 180, 181, 182, 183, 190, 192, 213, 219, 220, 224, 226, 231, 233, 234, 235, 236, 258, 270, 295, 297, 300, 301, 302, 304, 306, 308, 310, 347, 348, 353, 362, 392, 393, 405, 416, 429, 443, 446, 451, 467, 483, 487, 501, 502, 506, 507, 510, 512, 521, 522, 523, 528, 572, 580, 588, 591, 598, 603, 615, 617, 635, 636, 655, 657, 660, 662, 670, 678, 683, 691, 692, 696, 698, and 699).

In some embodiments, the biological sample is human. In some embodiments, the biological sample is non-human. In some embodiments, the biological sample is mammalian. In some embodiments, the biological sample is from rat, mouse, guinea pig, hamster, cow, pig, horse, goat, sheep, dog, cat, or non-human primate.

In embodiments utilizing a RAS-RAF-MAPK pathway peptide, the peptide may be modified with a detectable label. The detectable label may comprise an isotope, such as a heavy isotope, such as those known to those of skill in the art, including ¹³C, ¹⁵N, ²H and ¹⁸O. In some embodiments, the modified/labelled peptide comprises a peptide of SEQ ID NO: 701-1400. In some embodiments the peptide is less than or equal to 40 amino acids in length. In some embodiments the modified/labelled peptide consists of a peptide of SEQ ID NO: 701-1400. In some embodiments the modified/labelled peptide consists of a peptide of SEQ ID NO: 701-1400, wherein the peptide is further modified.

In some embodiments, the antibody for IP is chosen from the antibodies recited in Table 1. In some embodiments, the antibody for IP is an antibody having the six CDRs of any of the antibodies of Table 1. The antibody may be capable of immunoprecipitating more than one RAS-RAF-MAPK pathway protein. In some embodiments the antibody is labelled or capable of being labelled. The label may be any label known to those of skill in the art including enzymatic and fluorescent labels, such as biotin. In some embodiments more than one antibody is used in a multi-plex IP. In some embodiments, the multi-plex IP comprises the antibodies of Table 6. In some embodiments, the antibodies for multi-plex IP is an antibody having the six CDRs of any of the antibodies in Table 6.

In some embodiments, two or more antibodies are utilized to analyze one biological sample. For example, a first antibody is capable of immunoprecipitating a phosphorylated RAS-RAF-MAPK pathway protein, and a second antibody is capable of immunoprecipitating a nonphosphorylated version of the RAS-RAF-MAPK pathway protein precipitated by the first antibody. In such an instance, a single biological sample may be divided into two aliquots and the first antibody used to immunoprecipitate the first aliquot and the second antibody used to immunoprecipitate the second aliquot. This is appropriate when the second antibody can precipitate both the phospohorylated and non-phosphorylated versions of the RAS-RAF-MAPK protein; it is also appropriate if the second antibody can only precipitate the non-phosphorylated versions. In another instance, the phosphorylated version of the RAS-RAF-MAPK pathway protein may be immunoprecipitated from the sample first (using the first antibody) and evaluated, and then in a later step the non-phosphorylated RAS-RAF-MAPK pathway protein may be immunoprecipitated (using the second antibody) and evaluated. This approach is appropriate if the second antibody can precipitate both the phosphorylated and non-phosphorylated versions of the RAS-RAF-MAPK protein. In some embodiments, a single antibody is capable of immunoprecipitating a phosphorylated and non-phosphorylated RAS-RAF-MAPK pathway protein.

In some embodiments, the immunoprecipitation comprises treating a sample with a labelled antibody capable of binding to a RAS-RAF-MAPK pathway protein to provide a labelled antibody-protein conjugate. The method may further comprise contacting the labelled antibody-protein conjugate with a capture agent capable of binding to the labelled antibody to isolate the pathway protein from the sample. The label may be biotin and the capture agent may be streptavidin.

The quantity of a RAS-RAF-MAPK pathway protein may be determined by adding an internal standard peptide of known amount to the digested protein prior to mass spectrometry. In some embodiments, the internal standard peptide has the same amino acid sequence as the RAS-RAF-MAPK pathway peptide. In some embodiments, the internal standard is detectably labeled. The method may further comprises determining the quantity of a RAS-RAF-MAPK pathway peptide by comparison to the internal standard.

In some embodiments, the internal standard peptide comprises a sequence of SEQ ID NO: 1-SEQ ID NO: 1400. In some embodiments the peptide is less than or equal to 40 amino acids in length. In some embodiments, the peptide consists of a sequence of SEQ ID NO: 1-SEQ ID NO: 1400. The peptides of SEQ ID NO: 1-SEQ ID NO: 700 may be labelled. In some embodiments the label on SEQ ID NO: 1-SEQ ID NO: 700 differs from the label shown on the peptides of SEQ ID NO: 701-SEQ ID NO: 1400. In some embodiments, the peptide comprises or consists of a peptide selected from the peptides shown in Table 5 (SEQ ID Nos: 13, 15, 18, 28, 31, 47, 48, 51, 127, 128, 132, 133, 141, 142, 143, 144, 171, 173, 177, 180, 181, 182, 183, 190, 192, 213, 219, 220, 224, 226, 231, 233, 234, 235, 236, 258, 270, 295, 297, 300, 301, 302, 304, 306, 308, 310, 347, 348, 353, 362, 392, 393, 405, 416, 429, 443, 446, 451, 467, 483, 487, 501, 502, 506, 507, 510, 512, 521, 522, 523, 528, 572, 580, 588, 591, 598, 603, 615, 617, 635, 636, 655, 657, 660, 662, 670, 678, 683, 691, 692, 696, 698, and 699).

In some embodiments, quantifying the RAS-RAF-MAPK pathway protein comprises comparing an amount of a RAS-RAF-MAPK pathway peptide in the sample to an amount of the same RAS-RAF-MAPK pathway peptide in a control sample.

Quantifying a RAS-RAF-MAPK pathway protein may comprise comparing an amount of a RAS-RAF-MAPK pathway peptide to an internal standard peptide of known amount, wherein both the peptide in the biological sample and the internal standard peptide comprise SEQ ID NO: 1-SEQ ID NO: 1400, wherein the standard peptide is detectably labeled, and wherein the peptide is less than or equal to 40 amino acids long. In some embodiments, the standard peptide consists of a sequence of SEQ ID NO: 1-SEQ ID NO: 1400. The peptides of SEQ ID NO: 1-SEQ ID NO: 700 may be labelled. In some embodiments the label on SEQ ID NO: 1-SEQ ID NO: 700 differs from the label shown on the peptides of SEQ ID NO: 701-SEQ ID NO: 1400. In some embodiments, the peptide comprises or consists of a peptide selected from the peptides shown in Table 5 (SEQ ID Nos: 13, 15, 18, 28, 31, 47, 48, 51, 127, 128, 132, 133, 141, 142, 143, 144, 171, 173, 177, 180, 181, 182, 183, 190, 192, 213, 219, 220, 224, 226, 231, 233, 234, 235, 236, 258, 270, 295, 297, 300, 301, 302, 304, 306, 308, 310, 347, 348, 353, 362, 392, 393, 405, 416, 429, 443, 446, 451, 467, 483, 487, 501, 502, 506, 507, 510, 512, 521, 522, 523, 528, 572, 580, 588, 591, 598, 603, 615, 617, 635, 636, 655, 657, 660, 662, 670, 678, 683, 691, 692, 696, 698, and 699).

In some embodiments the mass spectrometry is selected from tandem mass spectrometry and discovery mass spectrometry. The targeted mass spectrometry may be selected from multiple reaction monitoring (MRM), selected reaction monitoring (SRM), and parallel reaction monitoring (PRM), or combinations thereof.

In some embodiments the biological sample is selected from isolated human cells, plasma, serum, whole blood, CSF, urine, sputum, tissue, and tumorous tissue.

In some embodiments, the method further comprises quantifying the relative amount of RAS-RAF-MAPK pathway protein. In some embodiments, the method further comprises quantifying the absolute amount of RAS-RAF-MAPK pathway protein.

In some embodiments, the digesting comprises a protease or chemical digest. In some embodiments the digestion may be single or sequential. The protease digestion may comprise trypsin, chymotrypsin, AspN, GluC, LysC, LysN, ArgC, proteinase K, pepsin, clostripain, elastase, GluC biocarb, LysC/P, LysN promisc, protein endopeptidase, staph protease or thermolysin.

The chemical cleavage may comprise CNBr, iodosobenzoate or formic acid.

In some embodiments the digestion is a protease digest with trypsin.

In some embodiments the methods further comprise desalting after digestion and prior to mass spectrometry.

The RAS-RAF-MAPK pathway protein may be chosen from proteins listed in Table 4.

In some embodiments, the RAS-RAF-MAPK pathway protein is phosphorylated.

In some embodiments, the concentration of RAS-RAF-MAPK protein that may be detected ranges from about 0.04 fmol to about 2000 fmol.

In some embodiments, the lower limit of detection is no more than about 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, or 1.23 fmol. The lower limit of detection may be from of about 0.04-1.23 fmol.

In some embodiments the lower limit of quantification is no more than about 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, 0.25, 0.30, 0.35, 0.40, 0.45, 0.50, 0.55, 0.60, 0.65, 0.70, or 11.11 fmol. The lower limit of quantification may be from about 0.04-11.11 fmol.

Kits comprising one or more antibodies capable of immunoprecipitating a RAS-RAF-MAPK pathway protein are encompassed.

Kits comprising one or more antibodies capable of immunoprecipitating a RAS-RAF-MAPK pathway protein, and reagents useful for performing mass spectrometry to detect a RAS-RAF-MAPK pathway protein are also provided.

Also encompassed are kits comprising one or more antibodies capable of immunoprecipitating a RAS-RAF-MAPK pathway target protein, and reagents useful for performing mass spectrometry to quantify a RAS-RAF-MAPK pathway protein.

The antibody to be included in the kit may be selected from any one or more of the antibodies recited in Table 1. In some embodiments the antibody is labelled or capable of being labelled. The label may be any label known to those of skill in the art including enzymatic and fluorescent labels, such as biotin. In some embodiments, the kit comprises more than one antibody. In some embodiments, the kit comprises two or more of the antibodies selected from the antibodies recited in Table 6.

The kits may further comprise a RAS-RAF-MAPK pathway peptide. In some embodiments, the peptide comprises a sequence of SEQ ID NO: 1-SEQ ID NO: 1400. In some embodiments the peptide is less than or equal to 40 amino acids in length. In some embodiments, the peptide consists of a sequence of SEQ ID NO: 1-SEQ ID NO: 1400. The peptides of SEQ ID NO: 1-SEQ ID NO: 700 may be labelled. In some embodiments the label on SEQ ID NO: 1-SEQ ID NO: 700 differs from the label shown on the peptides of SEQ ID NO: 701-SEQ ID NO: 1400. In some embodiments, the peptide comprises or consists of a peptide selected from the peptides shown in Table 5 (SEQ ID Nos: 13, 15, 18, 28, 31, 47, 48, 51, 127, 128, 132, 133, 141, 142, 143, 144, 171, 173, 177, 180, 181, 182, 183, 190, 192, 213, 219, 220, 224, 226, 231, 233, 234, 235, 236, 258, 270, 295, 297, 300, 301, 302, 304, 306, 308, 310, 347, 348, 353, 362, 392, 393, 405, 416, 429, 443, 446, 451, 467, 483, 487, 501, 502, 506, 507, 510, 512, 521, 522, 523, 528, 572, 580, 588, 591, 598, 603, 615, 617, 635, 636, 655, 657, 660, 662, 670, 678, 683, 691, 692, 696, 698, and 699).

In some embodiments, the kit may comprise at least one peptide selected from peptides of SEQ ID NO: 701-SEQ ID NO: 1400, wherein the peptide is less than or equal to 40 amino acids. In one embodiment, the kit comprises at least one peptide consisting of SEQ ID NO: 701-SEQ ID NO: 1400.

The peptides provided in the kit may be detectably labeled or capable of being modified to be detectably labeled. In some embodiments, the kit may comprise at least one peptide selected from peptides of SEQ ID NO: 1-SEQ ID NO: 700, wherein the peptide is detectably labeled or capable of being modified to be detectably labeled.

In some embodiments, the kit further comprises a protease or chemical agent capable of digesting an immunoprecipitated protein sample. The protease agent may be trypsin, chymotrypsin, AspN, GluC, LysC, LysN, ArgC, proteinase K, pepsin, clostripain, elastase, GluC biocarb, LysC/P, LysN promisc, protein endopeptidase, Staph protease or thermolysin. The chemical agent may be CNBr, iodosobenzoate or formic acid.

In some embodiments, the kit may comprise some or all of the following items: single or multiple biotinylated antibody mix, streptavidin magnetic beads (such as Pierce Strepavidin Magnetic Beads), IP-MS cell lysis buffer, IP-MS wash buffer(s), IP-MS elution buffer, positive control lysates, low binding tubes, reduction/alkylation reagents for MS sample prep, digestion buffer for MS sample prep, trypsin, buffer to stop trypsin digestion (such as 10% trifluoroacetic acid), AQUA (Absolute QUAntification) heavy peptide mix, diluent for peptides (such as 0.1% trifluoroacetic acid/5% acetonitrile), matrix (light crude peptides spiked into 6 proteins digest mix), and memory device (such as a USB stick) including the instrument method, skyline software template, and instructions.

The kits may be utilized to detect RAS-RAF-MAPK pathway proteins listed in Table 4.

The RAS-RAF-MAPK protein to be detected and quantified by the kits may be phosphorylated.

### BRIEF DESCRIPTION OF THE FIGURES

FIG. 1 shows one representative workflow for an immunoprecipitation-enriched mass spectrometry assay to identify RAS-RAF-MAPK pathway proteins.

FIG. 2 shows results from an experiment to enrich for low abundant RAS-RAF-MAPK pathway proteins from HCT116 or BT549 cells.

FIG. 3 shows quantitation limits of peptides for RAS-RAF-MAPK pathway proteins.

FIG. 4 shows the results of a multiplex immunoprecipitation plus nanoLC-MS/MS assay for 12 RAS-RAF-MAPK pathway proteins panel 1.

FIG. 5 shows the results of a multiplex immunoprecipitation plus nanoLC-MS/MS assay for 12 RAS-RAF-MAPK pathway proteins panel 2.

FIG. 6 shows the results of a multiplex immunoprecipitation plus nanoLC-MS/MS assay for 13 RAS-RAF-MAPK pathway proteins Panel 3.

FIG. 7 shows a summary of RAS-RAF-MAPK pathway proteins identified and quantified in HCT116 cell lines, with and without immunoprecipitation enrichment, using the mass spec methods.

### DETAILED DESCRIPTION

This description and exemplary embodiments should not be taken as limiting. For the purposes of this specification and appended claims, unless otherwise indicated, all numbers expressing quantities, percentages, or proportions, and other numerical values used in the specification and claims, are to be understood as being modified in all instances by the term “about,” to the extent they are not already so modified. Accordingly, unless indicated to the contrary, the numerical parameters set forth in the following specification and attached claims are approximations that may vary depending upon the desired properties sought to be obtained. At the very least, and not as an attempt to limit the application of the doctrine of equivalents to the scope of the claims, each numerical parameter should at least be construed in light of the number of reported significant digits and by applying ordinary rounding techniques.

It is noted that, as used in this specification and the appended claims, the singular forms “a,” “an,” and “the,” and any singular use of any word, include plural referents unless expressly and unequivocally limited to one referent. As used herein, the term “include” and its grammatical variants are intended to be non-limiting, such that recitation of items in a list is not to the exclusion of other like items that can be substituted or added to the listed items.

As used herein, an “RAS-RAF-MAPK pathway protein” includes, but is not limited to, BRAF (UniProtKB—P15056), HRAS (UniProtKB—P01112), KRAS (UniProtKB P01116), NRAS (UniProtKB—P01111), MAP2K1 (UniProtKB—Q02750), MAP2K2 (UniProtKB—P36507), MAPK1 (UniProtKB—P28482), MAPK3 (UniProtKB—P27361), MAPK8 (UniProtKB—P45983), MAPK9 (UniProtKB—P45984), PIK3CA (UniProtKB—P42336), RPS6KA1 (also known as p90S6K) (UniProtKB—Q15418), and TP53 (UniProtKB—P04637).

As used herein “protein”, “peptide”, and “polypeptide” are used interchangeably throughout to mean a chain of amino acids wherein each amino acid is connected to the next by a peptide bond. In some embodiments, when a chain of amino acids consists of about two to forty amino acids, the term “peptide” is used. However, the term “peptide” should not be considered limiting unless expressly indicated.

The term “antibody” is used in the broadest sense and encompasses various antibody structures, including but not limited to monoclonal antibodies, polyclonal antibodies, multispecific antibodies (such as bispecific antibodies), and antibody fragments so long as they exhibit the desired immunoprecipitating activity. As such, the term antibody includes, but is not limited to, fragments that are capable of binding to an antigen, such as Fv, single-chain Fv (scFv), Fab, Fab′, di-scFv, sdAb (single domain antibody) and (Fab′)₂ (including a chemically linked F(ab′)₂). Papain digestion of antibodies produces two identical antigen-binding fragments, called “Fab” fragments, each with a single antigen-binding site, and a residual “Fc” fragment. Pepsin treatment yields a F(ab′)₂ fragment that has two antigen-binding sites. The term antibody also includes, but is not limited to, chimeric antibodies, humanized antibodies, and antibodies of various species such as mouse, goat, horse, sheep, chicken, etc. Furthermore, for all antibody constructs provided herein, variants having the sequences from other organisms are also contemplated, such as CDR-grafted antibodies or chimeric antibodies. Antibody fragments also include either orientation of single chain scFvs, tandem di-scFv, diabodies, tandem tri-sdcFv, minibodies, etc. Antibody fragments also include nanobodies (sdAb, an antibody having a single, monomeric domain, such as a pair of variable domains of heavy chains, without a light chain). An antibody fragment can be referred to as being a specific species in some embodiments (for example, human scFv or a mouse scFv). This denotes the sequences of at least part of the non-CDR regions, rather than the source of the construct. The antibodies are referred to by reference to name and catalog reference. The skilled artisan, holding this name and catalog information, is capable of determining the sequence of the antibody, and therefore this application encompasses any antibody having at least partial sequence of a reference antibody so long as the antibody maintains its ability to immunoprecipitate its antigen protein. In some embodiments, the antibodies comprise antibodies having the same CDRs as the antibodies provided in Table 1 or Table 6.

“Immunoprecipitate” or “immunoprecipitation” refers to any antibody-driven enrichment step, whether or not an actual precipitate is formed. Thus, immunoprecipitation includes any immunoenrichment step.

Mass spectrometry (MS) is a primary technique for analysis of proteins on the basis of their mass-to-charge ratio (m/z). MS techniques generally include ionization of compounds and optional fragmentation of the resulting ions, as well as detection and analysis of the m/z of the ions and/or fragment ions followed by calculation of corresponding ionic masses. A “mass spectrometer” generally includes an ionizer and an ion detector. “Mass spectrometry,” “mass spec,” “mass spectroscopy,” and “MS” are used interchangeably throughout.

“Targeted mass spectrometry,” also referred to herein as “targeted mass spec,” “targeted MS,” and “tMS” increases the speed, sensitivity, and quantitative precision of mass spec analysis. Non-targeted mass spectrometry, sometimes referred to as “data-dependent scanning,” “discovery MS,” and “dMS” and targeted mass spec are alike in that in each, analytes (proteins, small molecules, or peptides) are infused or eluted from a reversed phase column attached to a liquid chromatography instrument and converted to gas phase ions by electrospray ionization. Analytes are fragmented in the mass spec (a process known as tandem MS or MS/MS), and fragment and parent masses are used to establish the identity of the analyte. Discovery MS analyzes the entire content of the MS/MS fragmentation spectrum. In contrast, in targeted mass spectrometry, a reference spectrum is used to guide analysis to only a few selected fragment ions rather than the entire content.

“Multiple reaction monitoring,” “MRM,” “selected reaction monitoring,” and “SRM” are used interchangeably throughout to refer to a type of targeted mass spectrometry that relies on a unique scanning mode accessible on triple-quadrupole (QQQ) instruments. See, e.g., Chambers et al., Expert Rev. Proteomics, 1-12 (2014).

“Parallel Reaction Monitoring,” and “PRM” are used interchangeably herein to describe another type of targeted mass spec wherein the second mass analyzer used in SRM (quadrupole) is substituted by a high resolution orbitrap mass analyzer in PRM. Unlike SRM, which allows the measuring of one single transition at a given point in time, PRM allows parallel monitoring in one MS/MS spectrum. PRM also allows for the separation of ions with close m/z values (i.e., within a 10 ppm range), and may therefore allow for lower limits of detection and quantification (LOD or LLOD and LOQ or LLOQ).

The methods disclosed herein may be applied to any type of MS analysis. The disclosure is not limited by the specific equipment or analysis used. The use of any equipment with the intent of analyzing the m/z of a sample would be included in the definition of mass spectrometry. Non-limiting examples of MS analysis and/or equipment that may be used include electrospray ionization, ion mobility, time-of-flight, tandem, ion trap, MRM, SRM, MRM/SRM, PRM, and Orbitrap. The disclosure is neither limited by the type of ionizer or detector used in the MS analysis nor by the specific configuration of the MS. The disclosure is not limited to use with specific equipment or software. The disclosure is not limited to the equipment and software described in the Examples.

In some embodiments, methods of immunoprecipitating a RAS-RAF-MAPK pathway protein are provided, comprising contacting a biological sample with at least one antibody recited in Table 1. The immunoprecipitating method may be single-plex or multi-plex. A “single-plex” IP utilizes one antibody per assay, whereas a “multi-plex” IP utilizes more than one antibody per assay.

In some embodiments, an IP-MS method for detecting and quantifying phosphorylated and non-phosphorylated RAS-RAF-MAPK pathway proteins is provided. The methods may comprise contacting a biological sample with at least one antibody recited in Table 1, digesting the immunoprecipitated protein(s), and assaying the digested proteins via mass spectrometry. The IP and MS may be single-plex or multi-plex. A “single-plex” MS refers to monitoring a single peptide in a single MS run, whereas a “multi-plex” MS refers to monitoring more than one target peptides in a single MS run.

Table 1 provides a listing of antibodies useful in the IP and IP-MS methods. Table 2 provides a listing of antibodies that are known to bind to their antigen RAS-RAF-MAPK protein, but were found to be less useful in the IP and IP-MS methods.

TABLE 1
List of IP to MS validated antibodies
for RAS-RAF-MAPK Pathway Proteins

| Antibody Name | Company/Catalog Number |
|---|---|
| AKT1/AKT2/AKT3 Antibody | Thermo Fisher Scientific/44-609G |
| AKT1/AKT2/AKT3 Antibody | Millipore/07-416 |
| ALK Antibody | Cell Signaling Technology/3633S |
| ALK Antibody | Cell Signaling Technology/3333S |
| ALK Antibody | Thermo Fisher Scientific/MA1-90059 |
| ALK Antibody | Thermo Fisher Scientific/35-4300 |
| ALK Antibody | Thermo Fisher Scientific/51-3900 |
| ARAF Antibody | Thermo Fisher Scientific/PA1-21440 |
| ARAF Antibody | Thermo Fisher Scientific/PA5-17657 |
| BRAF Antibody | Cell Signaling Technology/14814S |
| BRAF Antibody | Thermo Fisher Scientific/PA5-14926 |
| BRAF Antibody | Thermo Fisher Scientific/PA5-14929 |
| BRAF Antibody | Thermo Fisher Scientific/MA5-15000 |
| CALM1 Antibody | Millipore/05-173 |
| CAMK2A Antibody | Thermo Fisher Scientific/PA5-17095 |
| CCND1 Antibody | Thermo Fisher Scientific/MA1-34820 |
| CCND1 Antibody | Thermo Fisher Scientific/PA1-37530 |
| CCND1 Antibody | Thermo Fisher Scientific/MA5-14512 |
| Cyclin D1 Antibody | Cell Signaling Technology/2922S |
| Cyclin D1 Antibody | Millipore/ABE52 |
| Cyclin D1 Antibody | Thermo Fisher Scientific/MA1-12296 |
| Cyclin D1 Antibody | Thermo Fisher Scientific/PA1-29132 |
| Cyclin D1 Antibody | Thermo Fisher Scientific/MA1-26755 |
| Cyclin D1 Antibody | Thermo Fisher Scientific/AHF0082 |
| Cyclin D1 Antibody | Thermo Fisher Scientific/33-3500 |
| Cyclin D1 Antibody | Thermo Fisher Scientific/PA5-32373 |
| Cyclin D1/Bcl-1 Antibody | Thermo Fisher Scientific/PA5-16232 |
| CDH1 Antibody | Cell Signaling Technology/14472S |
| CDH1 Antibody | Thermo Fisher Scientific/13-5700 |
| CDH1 Antibody | Thermo Fisher Scientific/13-1700 |
| CDH1 Antibody | Thermo Fisher Scientific/33-4000 |
| CDH1 Antibody | Thermo Fisher Scientific/PA5-32282 |
| CDH1 Antibody | Thermo Fisher Scientific/PA5-32178 |
| CDH1 Antibody | Thermo Fisher Scientific/MA1-06300 |
| CDH1 Antibody | Thermo Fisher Scientific/MA5-14458 |
| CDH1 Antibody | Thermo Fisher Scientific/MA5-12547 |
| CDH2 Antibody | Thermo Fisher Scientific/PA1-20359 |
| CDH2 Antibody | Thermo Fisher Scientific/MA1-159 |
| CDH2 Antibody | Thermo Fisher Scientific/MA5-16324 |
| CDK2 Antibody | Thermo Fisher Scientific/MA5-13725 |
| CDK2 Antibody | Thermo Fisher Scientific/AHZ0142 |
| CDKN1A/p21 Antibody | Cell Signaling Technology/8831S |
| CDKN1A/p21 Antibody | Millipore/05-655 |
| CDKN1A/p21 Antibody | Millipore/05-345 |
| CDKN1A/p21 Antibody | Thermo Fisher Scientific/PA5-25960 |
| CDKN1A/p21 Antibody | Thermo Fisher Scientific/PA5-12644 |
| CDKN1A/p21 Antibody | Thermo Fisher Scientific/MA5-14949 |
| CDKN1A/p21 Antibody | Thermo Fisher Scientific/MA1-4800 |
| CDKN1A/p21 Antibody | Thermo Fisher Scientific/MA1-12303 |
| CDKN1A/p21 Antibody | Thermo Fisher Scientific/MA1-19271 |
| CDKN1A/p21 Antibody | Thermo Fisher Scientific/PA1-30399 |
| CDKN1A/p21 Antibody | Thermo Fisher Scientific/MA1-91243 |
| CDKN1A/p21 Antibody | Thermo Fisher Scientific/MA1-33926 |
| CDKN1A/p21 Antibody | Thermo Fisher Scientific/AHZ0422 |
| CDKN1A/p21 Antibody | Thermo Fisher Scientific/710182 |
| CDKN1A/p21 Antibody | Thermo Fisher Scientific/701151 |
| CDKN1B Antibody | Cell Signaling Technology/3688S |
| CDKN1B Antibody | Thermo Fisher Scientific/PA5-18480 |
| CDKN1B Antibody | Thermo Fisher Scientific/PA5-17946 |
| CDKN1B Antibody | Thermo Fisher Scientific/PA5-27188 |
| CDKN1B Antibody | Thermo Fisher Scientific/PA5-16717 |
| CDKN1B Antibody | Thermo Fisher Scientific/PA5-32530 |
| CDKN1B Antibody | Thermo Fisher Scientific/PA1-38351 |
| CDKN2A/p14ARF Antibody | Abcam/ab11048 |
| CDKN2A/p14ARF Antibody | Thermo Fisher Scientific/PA1-31409 |
| CDKN2A/p14ARF Antibody | Thermo Fisher Scientific/MA1-20219 |
| CDKN2A/p14ARF Antibody | Thermo Fisher Scientific/MA5-14260 |
| CDKN2A/p14ARF Antibody | Thermo Fisher Scientific/PA1-46021 |
| CDKN2A/p14ARF Antibody | Thermo Fisher Scientific/PA1-16662 |
| CDKN2A/p14ARF Antibody | Thermo Fisher Scientific/PA1-16639 |
| CDKN2A/p14ARF Antibody | Thermo Fisher Scientific/PA5-32529 |
| CDKN2A/p14ARF Antibody | Thermo Fisher Scientific/PA1-38334 |
| CDKN2A/p16INK4a Antibody | Abcam/ab16123 |
| CDKN2A/p16INK4a Antibody | Abcam/ab125282 |
| CDKN2A/p16INK4a Antibody | Thermo Fisher Scientific/PA1-9025 |
| CDKN2A/p16INK4a Antibody | Thermo Fisher Scientific/PA5-20379 |
| CTNNA1 Antibody | Thermo Fisher Scientific/MA1-2000 |
| CTNNA1 Antibody | Thermo Fisher Scientific/13-9700 |
| CTNNB1 Antibody | Cell Signaling Technology/9582S |
| CTNNB1 Antibody | Cell Signaling Technology/8814S |
| CTNNB1 Antibody | Thermo Fisher Scientific/PA5-19469 |
| CTNNB1 Antibody | Thermo Fisher Scientific/MA1-10056 |
| CTNNB1 Antibody | Thermo Fisher Scientific/MA1-34999 |
| CTNNB1 Antibody | Thermo Fisher Scientific/PA5-16762 |
| CTNNB1 Antibody | Thermo Fisher Scientific/PA5-16429 |
| CTNNB1 Antibody | Thermo Fisher Scientific/MA1-301 |
| CTNNB1 Antibody | Thermo Fisher Scientific/MA1-300 |
| CTNNB1 Antibody | Thermo Fisher Scientific/MA1-2001 |
| CTNNB1 Antibody | Thermo Fisher Scientific/13-8400 |
| CTNNB1 Antibody | Thermo Fisher Scientific/71-2700 |
| CTNNB1 Antibody | Thermo Fisher Scientific/44206M |
| CTNNB1 Antibody | Thermo Fisher Scientific/PA1-37255 |
| CTNNB1 Antibody | Thermo Fisher Scientific/MA5-14461 |
| DDR2 Antibody | Cell Signaling Technology/12133S |
| DUSP22 Antibody | Thermo Fisher Scientific/PA5-33488 |
| DUSP4 Antibody | Cell Signaling Technology/5149 |
| DUSP4 Antibody | Thermo Fisher Scientific/PA5-15549 |
| DUSP6 Antibody | Thermo Fisher Scientific/PA5-21570 |
| DUSP6 Antibody | Thermo Fisher Scientific/PA5-17107 |
| EGFR Antibody | Thermo Fisher Scientific/MA5-13697 |
| EGFR Antibody | Thermo Fisher Scientific/MA5-13319 |
| EGFR Antibody | Thermo Fisher Scientific/MA5-13269 |
| EGFR Antibody | Thermo Fisher Scientific/MA5-13070 |
| EGFR Antibody | Thermo Fisher Scientific/MA5-13048 |
| EGFR Antibody | Thermo Fisher Scientific/MA5-12880 |
| EGFR Antibody | Thermo Fisher Scientific/MA5-12875 |
| EGFR Antibody | Thermo Fisher Scientific/PA1-1110 |
| EGFR Antibody | Thermo Fisher Scientific/700308 |
| EGFR Antibody | Thermo Fisher Scientific/700994 |
| EGFR Antibody | Thermo Fisher Scientific/MA5-13877 |
| EGFR Antibody | Thermo Fisher Scientific/MA5-13343 |
| EGFR Antibody | Thermo Fisher Scientific/MA5-14485 |
| EIF2A Antibody | Proteintech/11233-1-AP |
| EIF2A Antibody | Abcam/ab102786 |
| EIF2A Antibody | Thermo Fisher Scientific/PA5-34795 |
| EIF4E Antibody | Cell Signaling Technology/9742S |
| EIF4E Antibody | Thermo Fisher Scientific/PA5-11797 |
| EIF4E Antibody | Thermo Fisher Scientific/MA1-089 |
| EIF4EBP1 Antibody | Cell Signaling Technology/9644S |
| EIF4EBP1 Antibody | Cell Signaling Technology/4923S |
| EIF4EBP1 Antibody | Thermo Fisher Scientific/PA5-11830 |
| EIF4EBP1 Antibody | Thermo Fisher Scientific/PA5-28947 |
| EIF4EBP1 Antibody | Thermo Fisher Scientific/PA5-20135 |
| ERBB3 Antibody | Cell Signaling Technology/12708S |
| ERBB3 Antibody | Thermo Fisher Scientific/PA1-30497 |
| ERBB3 Antibody | Thermo Fisher Scientific/MA5-13053 |
| ERBB3 Antibody | Thermo Fisher Scientific/MA5-13040 |
| ERBB3 Antibody | Thermo Fisher Scientific/MA5-12867 |
| ERBB3 Antibody | Thermo Fisher Scientific/MA5-12675 |
| ERBB3 Antibody | Thermo Fisher Scientific/MA5-16349 |
| ERBB3 Antibody | Thermo Fisher Scientific/PA1-86644 |
| ERBB3 Antibody | Thermo Fisher Scientific/PA1-9040 |
| ERBB3 Antibody | Thermo Fisher Scientific/PA5-32346 |
| ERBB4 Antibody | Cell Signaling Technology/4795S |
| ERBB4 Antibody | Thermo Fisher Scientific/PA1-20922 |
| ERBB4 Antibody | Thermo Fisher Scientific/MA5-12888 |
| ERBB4 Antibody | Thermo Fisher Scientific/PA5-16642 |
| ERBB4 Antibody | Thermo Fisher Scientific/PA5-32347 |
| ESR1 Antibody | Cell Signaling Technology/8644S |
| ESR1 Antibody | Millipore/06-935 |
| ESR1 Antibody | Thermo Fisher Scientific/MA1-12692 |
| ESR1 Antibody | Thermo Fisher Scientific/MA1-23214 |
| ESR1 Antibody | Thermo Fisher Scientific/MA1-25623 |
| ESR1 Antibody | Thermo Fisher Scientific/PA1-309 |
| ESR1 Antibody | Thermo Fisher Scientific/MA5-13065 |
| ESR1 Antibody | Thermo Fisher Scientific/PA5-16440 |
| ESR1 Antibody | Thermo Fisher Scientific/MA5-13191 |
| ESR1 Antibody | Thermo Fisher Scientific/MA5-13304 |
| ESR1 Antibody | Thermo Fisher Scientific/PA5-32397 |
| FBXO7 Antibody | Novus Bio/NBP1-55051 |
| FBXO7 Antibody | Millipore/ABN1038 |
| FBXO7 Antibody | Millipore/MABN447 |
| FBXO7 Antibody | Thermo Fisher Scientific/PA5-42300 |
| FBXO7 Antibody | Thermo Fisher Scientific/PA5-27589 |
| FGFR1 Antibody | Cell Signaling Technology/9740S |
| FGFR1 Antibody | Thermo Fisher Scientific/PA5-14650 |
| FGFR1 Antibody | Thermo Fisher Scientific/PA5-14648 |
| FGFR3 Antibody | Thermo Fisher Scientific/MA5-14843 |
| c-Fos Antibody | Thermo Fisher Scientific/PA5-17489 |
| c-fos Antibody | Thermo Fisher Scientific/MA5-15055 |
| c-Fos Antibody | Thermo Fisher Scientific/MA1-21190 |
| c-Fos Antibody | Thermo Fisher Scientific/38-4950 |
| c-Fos Antibody | Thermo Fisher Scientific/PA1-37437 |
| FOXO1 Antibody | Thermo Fisher Scientific/82358 |
| FOXO1 Antibody | Thermo Fisher Scientific/MA5-14846 |
| FOXO1 Antibody | Thermo Fisher Scientific/PA5-18696 |
| FOXO1 Antibody | Thermo Fisher Scientific/PA5-32412 |
| FOXO3 Antibody | Cell Signaling Technology/12829S |
| FOXO3 Antibody | Thermo Fisher Scientific/PA5-19519 |
| FOXO3 Antibody | Thermo Fisher Scientific/PA1-9042 |
| FOXO3 Antibody | Thermo Fisher Scientific/PA1-31322 |
| FOXO3 Antibody | Thermo Fisher Scientific/PA1-805 |
| FOXO3 Antibody | Thermo Fisher Scientific/PA5-23191 |
| FOXO3 Antibody | Thermo Fisher Scientific/PA5-32413 |
| HER-2/ErbB2 Antibody | Thermo Fisher Scientific/PA1-12361 |
| HER-2/ErbB2 Antibody | Thermo Fisher Scientific/PA1-37426 |
| HER-2/ErbB2 Antibody Antibody | Thermo Fisher Scientific/PA5-16305 |
| HER-2/ErbB2 Antibody (3B5) | Thermo Fisher Scientific/MA5-13675 |
| Antibody | |
| HER-2/ErbB2 Antibody (9G6.10) | Thermo Fisher Scientific/MA5-12759 |
| Antibody | |
| HER-2/ErbB2 Antibody | Thermo Fisher Scientific/MA5-14057 |
| (e2-4001 + 3B5) Antibody | |
| HER-2/ErbB2 Antibody | Thermo Fisher Scientific/MA5-15050 |
| (K.929.9) Antibody | |
| HER-2/ErbB2 Antibody (L26) | Thermo Fisher Scientific/MA5-13679 |
| Antibody | |
| HER-2/ERBB-2 Antibody | Thermo Fisher Scientific/MA1-12691 |
| HER-2/ERBB-2 Antibody | Thermo Fisher Scientific/AHO1011 |
| IQGAP1 Antibody | Abcam/ab33542 |
| IQGAP1 Antibody | Thermo Fisher Scientific/33-8900 |
| JAK1 Antibody | Cell Signaling Technology/3332S |
| JAK1 Antibody | Cell Signaling Technology/3344S |
| JAK2 Antibody | Cell Signaling Technology/3230S |
| JAK2 Antibody | Thermo Fisher Scientific/AHO1352 |
| JAK2 Antibody | Thermo Fisher Scientific/44-406G |
| MAP2K1 Antibody | Thermo Fisher Scientific/MA5-15093 |
| MAP2K1 Antibody | Thermo Fisher Scientific/PA1-29035 |
| MAP2K2 Antibody | Abcam/ab28834 |
| MAP2K2 Antibody | Thermo Fisher Scientific/PA5-17678 |
| MAP2K2 Antibody | Thermo Fisher Scientific/PA5-27635 |
| MAPK1 Antibody | Thermo Fisher Scientific/PA1-37640 |
| MAPK1 Antibody | Thermo Fisher Scientific/PA5-32396 |
| MAPK1 Antibody | Abcam/ab7948 |
| MAPK1 Antibody | Thermo Fisher Scientific/PA1-30388 |
| MAPK1 Antibody | Thermo Fisher Scientific/PA1-21016 |
| MAPK1/3 Antibody | Proteintech/66192-1-Ig |
| MAPK11 Antibody | Abcam/ab80116 |
| MAPK8 Antibody | Thermo Fisher Scientific/LF-PA0047 |
| MAPK8 Antibody | Thermo Fisher Scientific/35-9800 |
| MAPK9 Antibody | Thermo Fisher Scientific/PA5-28664 |
| MAPK9 Antibody | Thermo Fisher Scientific/PA5-17634 |
| MAPK9 Antibody | Thermo Fisher Scientific/MA1-21411 |
| MET Antibody | Cell Signaling Technology/8198S |
| MET Antibody | Thermo Fisher Scientific/PA5-17148 |
| MET Antibody | Thermo Fisher Scientific/MA5-14847 |
| MET Antibody | Thermo Fisher Scientific/PA5-27235 |
| MET Antibody | Thermo Fisher Scientific/PA1-29042 |
| MET Antibody | Thermo Fisher Scientific/PA1-28026 |
| MET Antibody | Thermo Fisher Scientific/71-8000 |
| MET Antibody | Thermo Fisher Scientific/37-0100 |
| MET Antibody | Thermo Fisher Scientific/18-7366 |
| MET Antibody | Thermo Fisher Scientific/700261 |
| MET Antibody | Thermo Fisher Scientific/PA5-32357 |
| MTOR Antibody | Thermo Fisher Scientific/PA1-188 |
| MTOR Antibody | Thermo Fisher Scientific/PA1-518 |
| NF1 Antibody | Abcam/ab17963 |
| NF1 Antibody | Millipore/05-1430 |
| NF1 Antibody | Millipore/07-730 |
| NF1 Antibody | Thermo Fisher Scientific/PA1-46246 |
| NOTCH 1 Antibody | Cell Signaling Technology/3608S |
| NOTCH 1 Antibody | Thermo Fisher Scientific/41-3500 |
| NOTCH 1 Antibody | Thermo Fisher Scientific/PA5-32522 |
| PAK1 Antibody | Thermo Fisher Scientific/PA5-17798 |
| PAK1 Antibody | Thermo Fisher Scientific/71-9300 |
| PAK1, 2, 3 Antibody | Thermo Fisher Scientific/PA5-17789 |
| pan MAPK8/9 Antibody | Proteintech/51153-1-AP |
| pan MAPK8/9 Antibody | Thermo Fisher Scientific/AHO1362 |
| Pan-Cadherin Antibody | Thermo Fisher Scientific/PA5-17526 |
| Pan-Cadherin Antibody | Thermo Fisher Scientific/MA5-15036 |
| Pan-Cadherin Antibody | Thermo Fisher Scientific/PA5-16766 |
| Pan-Cadherin Antibody | Thermo Fisher Scientific/PA5-16481 |
| Pan-Cadherin Antibody | Thermo Fisher Scientific/71-7100 |
| Pan-Cadherin Antibody | Thermo Fisher Scientific/PA1-37199 |
| PARP1 Antibody | Thermo Fisher Scientific/MA5-15031 |
| PARP1 Antibody | Thermo Fisher Scientific/PA5-16561 |
| PGR Antibody | Cell Signaling Technology/8757S |
| PGR Antibody | Thermo Fisher Scientific/MA1-12626 |
| PGR Antibody | Thermo Fisher Scientific/MA5-12658 |
| PGR Antibody | Thermo Fisher Scientific/MA5-12642 |
| PGR Antibody | Thermo Fisher Scientific/PA5-16709 |
| PGR Antibody | Thermo Fisher Scientific/MA1-411 |
| PGR Antibody | Thermo Fisher Scientific/MA5-14842 |
| PGR Antibody | Thermo Fisher Scientific/PA1-38491 |
| PGR Antibody | Thermo Fisher Scientific/PA5-17180 |
| PGR Antibody | Thermo Fisher Scientific/MA5-14841 |
| PI3KCA Antibody | Millipore/09-481 |
| PI3KCA Antibody | Millipore/07-658 |
| PI3KCA Antibody | Thermo Fisher Scientific/MA5-14870 |
| PIK3R2 Antibody | Thermo Fisher Scientific/MA1-90048 |
| PSMG1 Antibody | Cell Signaling Technology/13378S |
| PSMG1 Antibody | Abcam/ab83527 |
| PSMG1 Antibody | Thermo Fisher Scientific/PA5-19076 |
| RAF1 Antibody | Millipore/07-396 |
| RAF1 Antibody | Thermo Fisher Scientific/PA5-28652 |
| RAF1 Antibody | Thermo Fisher Scientific/MA5-17162 |
| Ras- pan Antibody | Thermo Fisher Scientific/MA1-012 |
| RASA1 Antibody | Abcam/ab2922 |
| RASA1 Antibody | Millipore/06-157 |
| RASA1 Antibody | Thermo Fisher Scientific/MA4-001 |
| RASGRF1 Antibody | Proteintech/12958-1-AP |
| RASGRF2 Antibody | Origene/TA590561 |
| RASGRF2 Antibody | Abcam/ab121577 |
| Ras-N Antibody | Cell Signaling Technology/3965S |
| Ras-N Antibody | Thermo Fisher Scientific/PA5-18888 |
| Ras-N Antibody | Thermo Fisher Scientific/PA5-34560 |
| Ras-v-H Antibody | Thermo Fisher Scientific/33-7200 |
| RICTOR Antibody | Cell Signaling Technology/9476S |
| RICTOR Antibody | Abcam/ab70373 |
| RICTOR Antibody | Thermo Fisher Scientific/PA5-14107 |
| RICTOR Antibody | Thermo Fisher Scientific/PA5-17451 |
| RICTOR Antibody | Thermo Fisher Scientific/MA5-15006 |
| RICTOR Antibody | Thermo Fisher Scientific/PA5-18370 |
| RICTOR Antibody | Thermo Fisher Scientific/PA5-21582 |
| RICTOR Antibody | Thermo Fisher Scientific/PA1-41209 |
| RPS6KB2 Antibody | Cell Signaling Technology/14130S |
| RPS6KB2 Antibody | Abcam/ab131526 |
| RPS6KB2 Antibody | Abcam/ab76960 |
| RPS6KB2 Antibody | Abcam/ab37699 |
| RPS6KB2 Antibody | Thermo Fisher Scientific/PA5-15234 |
| Raptor Antibody | Millipore/09-217 |
| Raptor Antibody | Cell Signaling Technology/2280S |
| Raptor Antibody | Millipore/05-1470 |
| Raptor Antibody | Thermo Fisher Scientific/42-4000 |
| Raptor Antibody | Thermo Fisher Scientific/PA5-20125 |
| RSK1 (RPS6KA1) Antibody | Cell Signaling Technology/8408S |
| RSK1 (RPS6KA1) Antibody | Thermo Fisher Scientific/PA5-23665 |
| RSK1 (RPS6KA1) Antibody | Thermo Fisher Scientific/PA5-17168 |
| RSK1 (RPS6KA1) Antibody | Thermo Fisher Scientific/PA5-29215 |
| RSK1 (RPS6KA1) Antibody | Thermo Fisher Scientific/PA5-21699 |
| RSK2 (RPS6KA3) Antibody | Millipore/06-918 |
| RSK2 (RPS6KA3) Antibody | Thermo Fisher Scientific/PA5-17169 |
| RSK2 (RPS6KA3) Antibody | Thermo Fisher Scientific/PA5-29214 |
| RSK2 (RPS6KA3) Antibody | Thermo Fisher Scientific/38-6800 |
| RSK2 (RPS6KA3) Antibody | Thermo Fisher Scientific/PA1-4694 |
| RSK2 + RSK3 Antibody | Thermo Fisher Scientific/MA5-17212 |
| SMAD3 Antibody | Thermo Fisher Scientific/51-1500 |
| SMAD4 Antibody | R&D Systems/AF2097-SP |
| SMAD4 Antibody | Thermo Fisher Scientific/PA5-14845 |
| SMAD4 Antibody | Thermo Fisher Scientific/PA5-16191 |
| SMAD4 Antibody | Thermo Fisher Scientific/PA5-34806 |
| SMAD4 Antibody | Thermo Fisher Scientific/PA1-31458 |
| SMAD4 Antibody | Thermo Fisher Scientific/MA5-14300 |
| SMAD4 Antibody | Thermo Fisher Scientific/701682 |
| SOS1 Antibody | Cell Signaling Technology/12409 |
| SOS1 Antibody | Cell Signaling Technology/5890S |
| SOS1 Antibody | Millipore/07-337 |
| SPRED1 Antibody | Millipore/ABS186 |
| SPRED1 Antibody | Genetex/GTX85376 |
| SPRED1 Antibody | Thermo Fisher Scientific/PA5-20618 |
| SPRED1 Antibody | Thermo Fisher Scientific/PA5-20617 |
| SPRED2 Antibody | Millipore/ABS198 |
| SPRY1 Antibody | Cell Signaling Technology/13013S |
| SPRY1 Antibody | Thermo Fisher Scientific/PA5-18289 |
| SPRY1 Antibody | Thermo Fisher Scientific/40-1800 |
| SPRY2 Antibody | Millipore/07-524 |
| SPRY2 Antibody | Cell Signaling Technology/14954S |
| SPRY3 Antibody | LifeSpan BioSciences/LS-C335646 |
| SPRY4 Antibody | Abcam/ab82298 |
| SPRY4 Antibody | Thermo Fisher Scientific/PA5-26959 |
| SPRY4 Antibody | Thermo Fisher Scientific/PA5-14349 |
| STAT1 Antibody | Thermo Fisher Scientific/AHO0832 |
| STAT3 Antibody | Cell Signaling Technology/9139S |
| STAT3 Antibody | Thermo Fisher Scientific/PA5-18562 |
| STAT3 Antibody | Thermo Fisher Scientific/PA1-9105 |
| STAT3 Antibody | Thermo Fisher Scientific/MA1-13042 |
| STAT3 Antibody | Thermo Fisher Scientific/13-7000 |
| STK11 Antibody | Thermo Fisher Scientific/MA5-14942 |
| TP53 Antibody | Thermo Fisher Scientific/MA1-12648 |
| TP53 Antibody | Thermo Fisher Scientific/MA1-12549 |
| TP53 Antibody | Thermo Fisher Scientific/MA1-19055 |
| TP53 Antibody | Thermo Fisher Scientific/MA5-15244 |
| TP53 Antibody | Thermo Fisher Scientific/MA5-14067 |
| TP53 Antibody | Thermo Fisher Scientific/MA5-12453 |
| TP53 Antibody | Thermo Fisher Scientific/MA5-11296 |
| TP53 Antibody | Thermo Fisher Scientific/AHO0152 |
| TP53 Antibody | Thermo Fisher Scientific/MA5-12557 |
| TP53 Antibody | Thermo Fisher Scientific/13-4100 |
| TP53 Antibody | Thermo Fisher Scientific/13-4000 |
| TP53 Antibody | Thermo Fisher Scientific/49-1031 |
| TP53 Antibody | Thermo Fisher Scientific/MA5-14467 |
| TP53 Antibody | Thermo Fisher Scientific/MA5-14516 |
| Phospho-TP53 (pSer15) | Thermo Fisher Scientific/700439 |
| Antibody | |
| Phospho-TP53 (pSer15) | Thermo Fisher Scientific/MA5-15229 |
| Antibody | |
| Phospho-MAPK1/3 | Thermo Fisher Scientific/MA5-15173 |
| (pThr202/pTyr204) Antibody | |
| Phospho-MAPK1/3 | Thermo Fisher Scientific/PA1-26678 |
| (pThr202/pTyr204) Antibody | |
| Phospho-MAPK1/3 | Cell Signaling Technology/9101 |
| (pThr202/pTyr204) Antibody | |
| Phospho-RAF1 (pSer259) | Thermo Fisher Scientific/44502 |
| Antibody | |
| Phospho-RAF1 (pSer259) | Cell Signaling Technology/9421 |
| Antibody | |
| Phospho-RPS6KA3 (pSer380) | Thermo Fisher Scientific/44928G |
| Antibody | |
| Phospho-RPS6KA3 (pSer380) | Cell Signaling Technology/11989 |
| Antibody | |
| Phospho-MAPK8/9 | Cell Signaling Technology/9251 |
| pThr183/pTyr185 Antibody | |
| Phospho-MAPK8/9 | Thermo Fisher Scientific/MA5-14943 |
| pThr183/pTyr185 Antibody | |
| Phospho-c-Raf (Ser338) Antibody | Thermo Fisher Scientific/MA5-15176 |
| Phospho-MEK1 (Ser298) Antibody | Cell Signaling Technology/98195 |
| Phospho-MEK1/MEK2 (Ser217, | Thermo Fisher Scientific/MA5-15016 |
| Ser221) Antibody | |
| Phospho-ATF2 (Thr71) Antibody | Thermo Fisher Scientific/MA5-15088 |
| Phospho-ATF-2 (Thr71) Antibody | Cell Signaling Technology/9221 |
| Phospho-SAPK/JNK | Cell Signaling Technology/4668 |
| (Thr183/Tyr185) Antibody | |

TABLE 2
List of IP to MS less successful antibodies
for RAS-RAF-MAPK Pathway Proteins

| Antibody Name | Company/Catalog Number |
|---|---|
| ALK Antibody | Thermo Fisher Scientific/PA5-14564 |
| ARAF Antibody | Thermo Fisher Scientific/PA5-14924 |
| ARAF Antibody | Thermo Fisher Scientific/PA5-21354 |
| BRAF Antibody | Thermo Fisher Scientific/PA5-19690 |
| BRAF Antibody | Thermo Fisher Scientific/PA5-14933 |
| BRAF Antibody | Thermo Fisher Scientific/PA5-14928 |
| BRAF Antibody | Thermo Fisher Scientific/MA5-15495 |
| BRAF Antibody | Thermo Fisher Scientific/MA5-15317 |
| BRAF Antibody | Millipore/07-453 |
| CALM1 Antibody | Thermo Fisher Scientific/PA5-11662 |
| CALM1 Antibody | Thermo Fisher Scientific/MA3-917 |
| CALM1 Antibody | Abcam/ab2860 |
| CAMK2A Antibody | Thermo Fisher Scientific/PA5-14315 |
| CAMK2A Antibody | Thermo Fisher Scientific/PA5-14033 |
| CAMK2A Antibody | Thermo Fisher Scientific/A16650 |
| CAMK2A Antibody | Thermo Fisher Scientific/A14012 |
| CAMK2A Antibody | Thermo Fisher Scientific/PA5-38239 |
| CAMK2A Antibody | Thermo Fisher Scientific/PA5-19128 |
| CAMK2A Antibody | Thermo Fisher Scientific/13-7300 |
| CAMK2A Antibody | Abcam/ab22609 |
| CAMK2A Antibody | Abcam/ab50202 |
| CAMK2A Antibody | Proteintech/20666-1-AP |
| CCND1 Antibody | Thermo Fisher Scientific/PA5-24173 |
| CCND1 Antibody | Thermo Fisher Scientific/PA5-12255 |
| Cyclin D1 Antibody | Thermo Fisher Scientific/MA1-10324 |
| Cyclin D1/Bcl-1 Antibody | Thermo Fisher Scientific/PA5-16777 |
| Cyclin D1/Bcl-1 Antibody | Thermo Fisher Scientific/PA5-16607 |
| Cyclin D1/Bcl-1 Antibody | Thermo Fisher Scientific/MA5-12707 |
| Cyclin D1/Bcl-1 Antibody | Thermo Fisher Scientific/710428 |
| Cyclin D1/Bcl-1 Antibody | Thermo Fisher Scientific/701421 |
| CDH1 Antibody | Thermo Fisher Scientific/MA1-10192 |
| CDH1 Antibody | Thermo Fisher Scientific/MA5-14408 |
| CDH1 Antibody | Thermo Fisher Scientific/MA5-11496 |
| CDH1 Antibody | Thermo Fisher Scientific/710161 |
| CDH1 Antibody | Thermo Fisher Scientific/701134 |
| CDH1 Antibody | Thermo Fisher Scientific/PA5-11548 |
| CDH1 Antibody | Thermo Fisher Scientific/PA5-11549 |
| CDH1 Antibody | Thermo Fisher Scientific/MA5-17013 |
| CDH1 Antibody | Thermo Fisher Scientific/MA1-06302 |
| CDH1 Antibody | Thermo Fisher Scientific/MA1-06304 |
| CDH1 Antibody | Thermo Fisher Scientific/MA1-37202 |
| CDH1 Antibody | Thermo Fisher Scientific/PA5-32339 |
| CDH1 Antibody | Thermo Fisher Scientific/MA1-34228 |
| CDH2 Antibody | Thermo Fisher Scientific/PA5-19486 |
| CDH2 Antibody | Thermo Fisher Scientific/PA5-29570 |
| CDH2 Antibody | Thermo Fisher Scientific/MA1-2002 |
| CDH2 Antibody | Thermo Fisher Scientific/33-3900 |
| CDH2 Antibody | Thermo Fisher Scientific/PA5-32283 |
| CDKN1A/p21 Antibody | Thermo Fisher Scientific/PA5-34805 |
| CDKN1A/p21 Antibody | Thermo Fisher Scientific/PA5-14456 |
| CDKN1A/p21 Antibody | Thermo Fisher Scientific/PA5-12647 |
| CDKN1A/p21 Antibody | Thermo Fisher Scientific/PA5-12646 |
| CDKN1A/p21 Antibody | Thermo Fisher Scientific/PA1-4185 |
| CDKN1A/p21 Antibody | Thermo Fisher Scientific/MA1-91045 |
| CDKN1A/p21 Antibody | Thermo Fisher Scientific/33-7000 |
| CDKN1B Antibody | Thermo Fisher Scientific/MA1-12302 |
| CDKN1B Antibody | Thermo Fisher Scientific/AHZ0452 |
| CDKN2A/p14ARF Antibody | Thermo Fisher Scientific/PA1-16661 |
| CDKN2A/p14ARF Antibody | Thermo Fisher Scientific/PA1-31212 |
| CDKN2A/p14ARF Antibody | Thermo Fisher Scientific/PA1-30670 |
| CDKN2A/p14ARF Antibody | Thermo Fisher Scientific/PA1-29914 |
| CDKN2A/p14ARF Antibody | Thermo Fisher Scientific/PA1-28218 |
| CDKN2A/p16INK4a Antibody | Thermo Fisher Scientific/MA5-17093 |
| CTNNA1 Antibody | Thermo Fisher Scientific/71-1200 |
| CTNNB1 Antibody | Thermo Fisher Scientific/44207M |
| CTNNB1 Antibody | Thermo Fisher Scientific/MA5-15569 |
| DDR2 Antibody | Thermo Fisher Scientific/PA5-14751 |
| DDR2 Antibody | Thermo Fisher Scientific/PA5-27752 |
| DDR2 Antibody | Thermo Fisher Scientific/PA5-27730 |
| DDR2 Antibody | Thermo Fisher Scientific/PA1-31993 |
| DUSP1 Antibody | Thermo Fisher Scientific/PA5-15593 |
| DUSP1 Antibody | Thermo Fisher Scientific/PA5-17973 |
| DUSP22 Antibody | Thermo Fisher Scientific/PA5-29427 |
| DUSP22 Antibody | Thermo Fisher Scientific/PA5-33489 |
| DUSP22 Antibody | Thermo Fisher Scientific/PA5-33487 |
| DUSP22 Antibody | Proteintech/51005-2-AP |
| DUSP22 Antibody | Abnova/H00056940-D01 |
| DUSP4 Antibody | Thermo Fisher Scientific/MA5-17225 |
| DUSP4 Antibody | Abcam/ab72593 |
| DUSP4 Antibody | Abcam/ab60229 |
| DUSP6 Antibody | Thermo Fisher Scientific/PA5-22162 |
| DUSP6 Antibody | Thermo Fisher Scientific/PA5-15552 |
| DUSP6 Antibody | Thermo Fisher Scientific/PA1-28321 |
| DUSP6 Antibody | Proteintech/10433-1-AP |
| EIF2A Antibody | Thermo Fisher Scientific/PA5-41916 |
| EIF2A Antibody | Thermo Fisher Scientific/PA5-29593 |
| EIF2A Antibody | Thermo Fisher Scientific/PA5-27366 |
| EIF2A Antibody | Thermo Fisher Scientific/MA1-079 |
| EIF2A Antibody | Thermo Fisher Scientific/AHO1182 |
| EIF2A Antibody | Thermo Fisher Scientific/AHO0802 |
| EIF4E Antibody | Thermo Fisher Scientific/PA5-24582 |
| EIF4E Antibody | Thermo Fisher Scientific/PA5-41751 |
| EIF4EBP1 Antibody | Thermo Fisher Scientific/PA5-30679 |
| EIF4EBP1 Antibody | Thermo Fisher Scientific/AHO1382 |
| EIF4EBP1 Antibody | Thermo Fisher Scientific/MA5-15314 |
| ERBB3 Antibody | Thermo Fisher Scientific/MA5-13008 |
| ERBB3 Antibody | Thermo Fisher Scientific/MA1-860 |
| ERBB3 Antibody | Thermo Fisher Scientific/PA5-14636 |
| ERBB3 Antibody | Thermo Fisher Scientific/PA1-31989 |
| ERBB3 Antibody | Thermo Fisher Scientific/MA1-22819 |
| ERBB4 Antibody | Thermo Fisher Scientific/PA5-29272 |
| ERBB4 Antibody | Thermo Fisher Scientific/PA5-14638 |
| ERBB4 Antibody | Thermo Fisher Scientific/PA5-14637 |
| ERBB4 Antibody | Thermo Fisher Scientific/PA1-20923 |
| ERBB4 Antibody | Thermo Fisher Scientific/PA5-16789 |
| ERBB4 Antibody | Thermo Fisher Scientific/MA5-13789 |
| ERBB4 Antibody | Thermo Fisher Scientific/MA5-13016 |
| ERBB4 Antibody | Thermo Fisher Scientific/MA1-861 |
| ERBB4 Antibody | Thermo Fisher Scientific/PA5-32758 |
| ERBB4 Antibody | Millipore/05-478 |
| ESR1 Antibody | Thermo Fisher Scientific/PA1-308 |
| ESR1 Antibody | Thermo Fisher Scientific/MA1-12600 |
| ESR1 Antibody | Thermo Fisher Scientific/MA5-12328 |
| ESR1 Antibody | Thermo Fisher Scientific/417000 |
| ESR1 Antibody | Thermo Fisher Scientific/MA5-15268 |
| ESR1 Antibody | Thermo Fisher Scientific/PA5-34577 |
| ESR1 Antibody | Thermo Fisher Scientific/PA1-37651 |
| ESR1 Antibody | Thermo Fisher Scientific/49-1002 |
| ESR1 Antibody | Thermo Fisher Scientific/MA1-310 |
| FGFR1 Antibody | Thermo Fisher Scientific/PA5-28564 |
| FGFR1 Antibody | Thermo Fisher Scientific/PA5-25979 |
| FGFR1 Antibody | Thermo Fisher Scientific/PA5-24590 |
| FGFR1 Antibody | Thermo Fisher Scientific/PA5-14649 |
| FGFR1 Antibody | Thermo Fisher Scientific/PA5-14646 |
| FGFR1 Antibody | Thermo Fisher Scientific/PA5-18344 |
| FGFR1 Antibody | Thermo Fisher Scientific/PA5-27139 |
| FGFR1 Antibody | Thermo Fisher Scientific/PA1-24762 |
| FGFR1 Antibody | Thermo Fisher Scientific/MA1-90880 |
| FGFR1 Antibody | Thermo Fisher Scientific/MA1-26256 |
| FGFR1 Antibody | Thermo Fisher Scientific/13-3100 |
| FGFR1 Antibody | Thermo Fisher Scientific/PA5-32403 |
| FGFR1 Antibody | Thermo Fisher Scientific/PA1-37682 |
| FGFR1 Antibody | Millipore/05-1134 |
| FGFR1 Antibody | Millipore/05-149 |
| FGFR2 Antibody | Thermo Fisher Scientific/PA5-14654 |
| FGFR2 Antibody | Thermo Fisher Scientific/PA5-14653 |
| FGFR2 Antibody | Thermo Fisher Scientific/PA5-14652 |
| FGFR2 Antibody | Thermo Fisher Scientific/PA5-14651 |
| FGFR2 Antibody | Thermo Fisher Scientific/PA5-18914 |
| FGFR2 Antibody | Thermo Fisher Scientific/PA5-29426 |
| FGFR2 Antibody | Thermo Fisher Scientific/PA1-31990 |
| FGFR2 Antibody | Thermo Fisher Scientific/PA1-24764 |
| FGFR2 Antibody | Thermo Fisher Scientific/PA1-24763 |
| FGFR2 Antibody | Thermo Fisher Scientific/PA5-32404 |
| FGFR2 Antibody | Thermo Fisher Scientific/PA1-37685 |
| FGFR3 Antibody | Thermo Fisher Scientific/PA5-19782 |
| FGFR3 Antibody | Thermo Fisher Scientific/PA5-14655 |
| FGFR3 Antibody | Thermo Fisher Scientific/PA5-34574 |
| FGFR3 Antibody | Thermo Fisher Scientific/PA5-32405 |
| FGFR3 Antibody | Thermo Fisher Scientific/PA1-37687 |
| c-fos Antibody | Thermo Fisher Scientific/PA5-13531 |
| c-Fos Antibody | Thermo Fisher Scientific/PA1-24330 |
| c-Fos Antibody | Thermo Fisher Scientific/PA1-830 |
| c-Fos Antibody | Thermo Fisher Scientific/OSC00011G |
| c-Fos Antibody | Thermo Fisher Scientific/MA5-17076 |
| c-Fos Antibody | Genetex/GTX27963 |
| FOXO1 Antibody | Thermo Fisher Scientific/PA5-23132 |
| FOXO1 Antibody | Thermo Fisher Scientific/MA1-23236 |
| FOXO1 Antibody | Thermo Fisher Scientific/MA1-23234 |
| FOXO1 Antibody | Thermo Fisher Scientific/MA1-23232 |
| FOXO1 Antibody | Thermo Fisher Scientific/MA1-23230 |
| FOXO1 Antibody | Thermo Fisher Scientific/PA1-804 |
| FOXO1 Antibody | Thermo Fisher Scientific/MA5-17078 |
| FOXO1 Antibody | Thermo Fisher Scientific/PA5-20972 |
| FOXO1/3 Antibody | Thermo Fisher Scientific/PA5-38276 |
| FOXO1/3/4 Antibody | Thermo Fisher Scientific/PA5-36084 |
| FOXO1/3/4 Antibody | Thermo Fisher Scientific/PA5-38274 |
| FOXO3 Antibody | Thermo Fisher Scientific/PA5-27145 |
| FOXO3 Antibody | Thermo Fisher Scientific/PA5-20973 |
| HER-2/ErbB2 Antibody | Thermo Fisher Scientific/PA5-14635 |
| HER-2/ErbB2 Antibody | Thermo Fisher Scientific/PA5-14634 |
| HER-2/ErbB2 Antibody | Thermo Fisher Scientific/PA5-14632 |
| HER-2/ErbB2 Antibody | Thermo Fisher Scientific/MA5-16724 |
| HER-2/ErbB2 Antibody | Thermo Fisher Scientific/PA5-16774 |
| Antibody | |
| HER-2/ErbB2 Antibody | Thermo Fisher Scientific/PA5-20740 |
| Antibody | |
| HER-2/ErbB2 Antibody | Thermo Fisher Scientific/700635 |
| (40H87L57), ABfinity ™ | |
| Rabbit Monoclonal Antibody | |
| HER-2/ErbB2 Antibody | Thermo Fisher Scientific/701399 |
| (7H3L20), ABfinity ™ | |
| Rabbit Monoclonal Antibody | |
| HER-2/ErbB2 Antibody | Thermo Fisher Scientific/710452 |
| (7HCLC), ABfinity ™ Rabbit | |
| Oligoclonal Antibody | |
| HER-2/ErbB2 Antibody (ICR55) | Thermo Fisher Scientific/MA1-82367 |
| Antibody | |
| HER-2/ErbB2 Antibody | Thermo Fisher Scientific/MA5-11976 |
| (L87 + 2ERB19) Antibody | |
| HER-2/ErbB2 Antibody (N12) | Thermo Fisher Scientific/MA5-12998 |
| Antibody | |
| HER-2/ErbB2 Antibody (N28) | Thermo Fisher Scientific/MA5-13032 |
| Antibody | |
| IQGAP1 Antibody | Thermo Fisher Scientific/PA5-19189 |
| IQGAP1 Antibody | Thermo Fisher Scientific/PA5-34368 |
| IQGAP1 Antibody | Cell Signaling Technology/2293S |
| JAK1 Antibody | Thermo Fisher Scientific/PA5-34811 |
| JAK1 Antibody | Thermo Fisher Scientific/PA5-27583 |
| JAK2 Antibody | Thermo Fisher Scientific/PA5-11267 |
| JAK2 Antibody | Thermo Fisher Scientific/PA5-34649 |
| KSR1 Antibody | Thermo Fisher Scientific/PA5-14937 |
| KSR1 Antibody | Thermo Fisher Scientific/PA5-14027 |
| KSR1 Antibody | Thermo Fisher Scientific/MA1-25285 |
| KSR1 Antibody | Abcam/ab14516 |
| MAP2K1 Antibody | Thermo Fisher Scientific/PA5-27693 |
| MAP2K1 Antibody | Thermo Fisher Scientific/MA1-095 |
| MAP2K1 Antibody | Thermo Fisher Scientific/PA5-15161 |
| MAP2K1 Antibody | Thermo Fisher Scientific/PA5-17677 |
| MAP2K1 Antibody | Thermo Fisher Scientific/PA1-21730 |
| MAP2K1 Antibody | Thermo Fisher Scientific/PA5-16556 |
| MAP2K1 Antibody | Thermo Fisher Scientific/13-3500 |
| MAP2K1 Antibody | Thermo Fisher Scientific/710446 |
| MAP2K2 Antibody | Thermo Fisher Scientific/PA5-29291 |
| MAP2K2 Antibody | Thermo Fisher Scientific/PA5-27640 |
| MAP2K2 Antibody | Thermo Fisher Scientific/700829 |
| MAP2K2 Antibody | Thermo Fisher Scientific/PA5-15167 |
| MAP2K2 Antibody | Thermo Fisher Scientific/PA1-29852 |
| MAP2K2 Antibody | Thermo Fisher Scientific/PA1-41411 |
| MAP2K2 Antibody | Abcam/ab71828 |
| MAP2K2 Antibody | Cell Signaling Technology/9147S |
| MAPK1 Antibody | Thermo Fisher Scientific/PA5-14088 |
| MAPK1 Antibody | Thermo Fisher Scientific/PA5-14414 |
| MAPK1 Antibody | Thermo Fisher Scientific/PA1-29167 |
| MAPK1 Antibody | Thermo Fisher Scientific/PA5-18329 |
| MAPK1 Antibody | Thermo Fisher Scientific/PA5-17710 |
| MAPK1 Antibody | Thermo Fisher Scientific/PA1-28196 |
| MAPK1 Antibody | Thermo Fisher Scientific/PA5-29636 |
| MAPK1 Antibody | Thermo Fisher Scientific/MA1-099 |
| MAPK1 Antibody | Thermo Fisher Scientific/LF-MA0193 |
| MAPK1 Antibody | Thermo Fisher Scientific/MA5-15304 |
| MAPK1/3 Antibody | Thermo Fisher Scientific/MA5-15134 |
| MAPK1/3 Antibody | Thermo Fisher Scientific/82380 |
| MAPK1/3 Antibody | Thermo Fisher Scientific/61-7400 |
| MAPK1/3 Antibody | Thermo Fisher Scientific/44-654G |
| MAPK1/3 Antibody | Thermo Fisher Scientific/13-6200 |
| MAPK1/3 Antibody | Thermo Fisher Scientific/710318 |
| MAPK1/3 Antibody | Thermo Fisher Scientific/710214 |
| MAPK1/3 pan Antibody | Thermo Fisher Scientific/PA1-25987 |
| MAPK1/3 pan Antibody | Thermo Fisher Scientific/MA5-15227 |
| MAPK11 Antibody | Thermo Fisher Scientific/PA5-29302 |
| MAPK11 Antibody | Thermo Fisher Scientific/PA5-14425 |
| MAPK11 Antibody | Thermo Fisher Scientific/PA5-14424 |
| MAPK11 Antibody | Thermo Fisher Scientific/PA5-14050 |
| MAPK11 Antibody | Thermo Fisher Scientific/PA1-29850 |
| MAPK11 Antibody | Thermo Fisher Scientific/33-8700 |
| MAPK11 Antibody | Thermo Fisher Scientific/PA1-41154 |
| MAPK3 Antibody | Thermo Fisher Scientific/PA1-30387 |
| MAPK3 Antibody | Thermo Fisher Scientific/MA1-13041 |
| MAPK3 Antibody | Thermo Fisher Scientific/13-8600 |
| MAPK3 Antibody | Thermo Fisher Scientific/701183 |
| MAPK3 Antibody | Thermo Fisher Scientific/PA5-14413 |
| MAPK3 Antibody | Thermo Fisher Scientific/PA1-21015 |
| MAPK3 Antibody | Thermo Fisher Scientific/PA5-32911 |
| MAPK3 Antibody | Thermo Fisher Scientific/PA5-32912 |
| MAPK3 Antibody | Thermo Fisher Scientific/PA5-32913 |
| MAPK3 Antibody | Thermo Fisher Scientific/PA1-41413 |
| MAPK3 Antibody | Thermo Fisher Scientific/PA1-9041 |
| MAPK3 Antibody | Thermo Fisher Scientific/PA1-37638 |
| MAPK3 Antibody | Thermo Fisher Scientific/PA5-32395 |
| MAPK8 Antibody | Thermo Fisher Scientific/PA5-21343 |
| MAPK8 Antibody | Thermo Fisher Scientific/PA5-25151 |
| MAPK8 Antibody | Thermo Fisher Scientific/PA5-14418 |
| MAPK8 Antibody | Thermo Fisher Scientific/PA5-17889 |
| MAPK8 Antibody | Thermo Fisher Scientific/44-690G |
| MAPK8 Antibody | Thermo Fisher Scientific/710075 |
| MAPK8 Antibody | Thermo Fisher Scientific/MA5-17117 |
| MAPK9 Antibody | Thermo Fisher Scientific/PA5-28262 |
| MAPK9 Antibody | Thermo Fisher Scientific/MA5-15183 |
| MAPK9 Antibody | Thermo Fisher Scientific/AHO1162 |
| MAPK9 Antibody | Thermo Fisher Scientific/PA1-41234 |
| MET Antibody | Thermo Fisher Scientific/MA5-15198 |
| MET Antibody | Thermo Fisher Scientific/MA5-23760 |
| NF1 Antibody | Thermo Fisher Scientific/MA1-085 |
| NF1 Antibody | Thermo Fisher Scientific/MA1-084 |
| NF1 Antibody | LifeSpan BioSciences/LS-C2721 |
| NOTCH 1 Antibody | Thermo Fisher Scientific/MA1-91405 |
| NOTCH 1 Antibody | Thermo Fisher Scientific/MA5-11961 |
| NOTCH 1 Antibody | Thermo Fisher Scientific/PA5-23181 |
| NOTCH 1 Antibody | Thermo Fisher Scientific/MA1-81888 |
| PAK1 Antibody | Thermo Fisher Scientific/PA5-29256 |
| PAK1 Antibody | Thermo Fisher Scientific/PA5-29224 |
| PAK1 Antibody | Thermo Fisher Scientific/PA5-34696 |
| PAK1 Antibody | Thermo Fisher Scientific/PA5-15115 |
| PAK1 Antibody | Thermo Fisher Scientific/PA5-18557 |
| PAK1 Antibody | Thermo Fisher Scientific/PA1-28385 |
| PAK1, 2 Antibody | Thermo Fisher Scientific/PA5-36918 |
| PAK1, 2 Antibody | Thermo Fisher Scientific/PA5-38693 |
| PAK1, 2, 3 Antibody | Thermo Fisher Scientific/PA5-36102 |
| PAK1, 2, 3 Antibody | Thermo Fisher Scientific/PA5-38311 |
| PAK1, 2, 3 Antibody | Thermo Fisher Scientific/PA5-38310 |
| pan MAP2K Antibody | Thermo Fisher Scientific/PA1-10034 |
| pan MAP2K Antibody | Thermo Fisher Scientific/PA5-31917 |
| pan MAP2K Antibody | Thermo Fisher Scientific/MA5-15162 |
| pan MAP2K Antibody | Thermo Fisher Scientific/MA5-15135 |
| pan MAP2K Antibody | Thermo Fisher Scientific/PA5-31918 |
| pan MAPK8/9 Antibody | BD Biosciences/554285 |
| Pan-Cadherin Antibody | Thermo Fisher Scientific/PA5-19479 |
| PARP1 Antibody | Thermo Fisher Scientific/PA5-34803 |
| PARP1 Antibody | Thermo Fisher Scientific/PA5-34802 |
| PARP1 Antibody | Thermo Fisher Scientific/PA5-13386 |
| PARP1 Antibody | Thermo Fisher Scientific/PA5-27219 |
| PARP1 Antibody | Thermo Fisher Scientific/PA5-16452 |
| PARP1 Antibody | Thermo Fisher Scientific/A21969 |
| PARP1 Antibody | Thermo Fisher Scientific/436400 |
| PARP1 Antibody | Thermo Fisher Scientific/MA1-41020 |
| PARP1 Antibody | Thermo Fisher Scientific/PA5-32561 |
| PARP1 Antibody | Thermo Fisher Scientific/PA1-38414 |
| PGR Antibody | Thermo Fisher Scientific/MA5-12653 |
| PGR Antibody | Thermo Fisher Scientific/MA1-412 |
| PGR Antibody | Thermo Fisher Scientific/PA5-16435 |
| PGR Antibody | Thermo Fisher Scientific/PA5-31314 |
| PGR Antibody | Thermo Fisher Scientific/MA1-23267 |
| PGR Antibody | Thermo Fisher Scientific/MA1-23268 |
| PGR Antibody | Thermo Fisher Scientific/PA5-34849 |
| PGR Antibody | Thermo Fisher Scientific/PA5-17226 |
| PI3KCA Antibody | Thermo Fisher Scientific/PA5-15246 |
| PI3KCA Antibody | Thermo Fisher Scientific/PA5-18610 |
| PI3KCA Antibody | Thermo Fisher Scientific/PA5-27192 |
| PI3KCA Antibody | Thermo Fisher Scientific/MA5-17149 |
| PIK3R1 Antibody | Thermo Fisher Scientific/710400 |
| PI3KR2 Antibody | Thermo Fisher Scientific/PA5-15262 |
| PSMG1 Antibody | Thermo Fisher Scientific/PA5-31298 |
| PSMG1 Antibody | Abgent/AI12626 |
| PSMG1 Antibody | Acris/TA338919 |
| PSMG1 Antibody | Acris/TA338920 |
| RAF1 Antibody | Thermo Fisher Scientific/PA5-29333 |
| RAF1 Antibody | Thermo Fisher Scientific/PA5-14941 |
| RAF1 Antibody | Thermo Fisher Scientific/MA1-90087 |
| RAF1 Antibody | Thermo Fisher Scientific/MA1-21482 |
| RAF1 Antibody | Thermo Fisher Scientific/PA5-20970 |
| RAF1 Antibody | Cell Signaling Technology/53745S |
| Ras- H Antibody | Thermo Fisher Scientific/PA5-14863 |
| Ras- H Antibody | Thermo Fisher Scientific/MA1-19726 |
| Ras- H Antibody | Thermo Fisher Scientific/PA5-22392 |
| Ras- K Antibody | Thermo Fisher Scientific/MA1-91046 |
| Ras- K Antibody | Thermo Fisher Scientific/415700 |
| RASGRF1 Antibody | Thermo Fisher Scientific/PA5-14531 |
| RASGRF1 Antibody | Abcam/ab190081 |
| RASGRF2 Antibody | Thermo Fisher Scientific/PA5-28867 |
| RASGRF2 Antibody | Thermo Fisher Scientific/PA5-28857 |
| RASGRF2 Antibody | Abcam/ab176008 |
| Ras-N Antibody | Thermo Fisher Scientific/PA5-14833 |
| RICTOR Antibody | Thermo Fisher Scientific/PA5-14104 |
| RPS6KB2 Antibody | Thermo Fisher Scientific/PA5-26010 |
| RPS6KB2 Antibody | Thermo Fisher Scientific/PA5-15235 |
| Raptor Antibody | Thermo Fisher Scientific/PA5-20126 |
| RSK1 (RPS6KA1) Antibody | Thermo Fisher Scientific/LF-MA0183 |
| RSK1 (RPS6KA1) Antibody | Thermo Fisher Scientific/PA5-15135 |
| RSK1 (RPS6KA1) Antibody | Thermo Fisher Scientific/AHO1472 |
| RSK1 (RPS6KA1) Antibody | Thermo Fisher Scientific/MA5-15341 |
| RSK1 (RPS6KA1) Antibody | Thermo Fisher Scientific/PA5-20134 |
| RSK1 + RSK2 + RSK3 | Thermo Fisher Scientific/PA5-17680 |
| Antibody | |
| RSK1 + RSK2 + RSK3 | Thermo Fisher Scientific/MA5-15040 |
| Antibody | |
| RSK2 (RPS6KA3) Antibody | Thermo Fisher Scientific/PA5-29280 |
| RSK2 (RPS6KA3) Antibody | Thermo Fisher Scientific/PA5-15140 |
| RSK2 (RPS6KA3) Antibody | Thermo Fisher Scientific/MA5-15920 |
| RSK3 (RPS6KA2) Antibody | Thermo Fisher Scientific/PA5-29222 |
| SMAD4 Antibody | Thermo Fisher Scientific/PA5-35330 |
| SMAD4 Antibody | Thermo Fisher Scientific/710714 |
| SOS1 Antibody | Thermo Fisher Scientific/MA5-17234 |
| SPRED1 Antibody | Millipore/MABN478 |
| SPRED2 Antibody | Thermo Fisher Scientific/PA5-31038 |
| SPRED2 Antibody | Thermo Fisher Scientific/PA5-20625 |
| SPRED2 Antibody | Abcam/ab50535 |
| SPRED2 Antibody | Abcam/ab58127 |
| SPRY1 Antibody | Thermo Fisher Scientific/PA5-43582 |
| SPRY1 Antibody | Abcam/ab75492 |
| SPRY2 Antibody | Thermo Fisher Scientific/PA5-43584 |
| SPRY2 Antibody | Thermo Fisher Scientific/PA5-43583 |
| SPRY3 Antibody | Abcam/ab180037 |
| SPRY3 Antibody | Novus Bio/AF5735 |
| SPRY3 Antibody | ProSci/30-623 |
| SPRY4 Antibody | Thermo Fisher Scientific/PA5-31401 |
| SPRY4 Antibody | Thermo Fisher Scientific/40-9300 |
| STAT1 Antibody | Thermo Fisher Scientific/MA1-037 |
| STAT3 Antibody | Thermo Fisher Scientific/710077 |
| STAT3 Antibody | Thermo Fisher Scientific/PA1-86605 |
| STK11 Antibody | Thermo Fisher Scientific/PA5-14074 |
| STK11 Antibody | Thermo Fisher Scientific/PA5-14072 |
| STK11 Antibody | Thermo Fisher Scientific/MA5-14831 |
| STK11 Antibody | Thermo Fisher Scientific/PA1-12767 |
| STK11 Antibody | Thermo Fisher Scientific/PA5-20325 |
| TP53 Antibody | Thermo Fisher Scientific/PA5-27822 |
| TP53 Antibody | Thermo Fisher Scientific/MA1-7629 |
| TP53 Antibody | Thermo Fisher Scientific/701270 |
| TP53 Antibody | Thermo Fisher Scientific/710294 |
| Phospho-p38 MAPK | Cell Signaling Technology/4511 |
| (Thr180/Tyr182) Antibody | |
| Phospho-p38 MAPK | Cell Signaling Technology/9211 |
| (Thr180/Tyr182) Antibody | |
| Phospho-p38 MAPK | Cell Signaling Technology/4631 |
| (Thr180/Tyr182) Antibody | |
| Phospho-p38 MAPK | Cell Signaling Technology/9215 |
| (Thr180/Tyr182) Antibody | |
| Phospho-p38 MAPK | Cell Signaling Technology/9216 |
| (Thr180/Tyr182) Antibody | |
| Phospho-MEK3/MEK6 (Ser189, | Thermo Fisher Scientific/PA5-17887 |
| Ser207) Antibody | |
| Phospho-MKK3 (Ser189)/ | Cell Signaling Technology/12280 |
| MKK6 (Ser207) Antibody | |
| Phospho-MKK3 (Ser189)/ | Cell Signaling Technology/9236 |
| MKK6 (Ser207) Antibody | |
| Phospho-MSK1 (Thr581) | Cell Signaling Technology/9595 |
| Antibody | |
| Phospho-MAPKAPK2 (Thr334) | Cell Signaling Technology/3007 |
| Antibody | |
| Phospho-MAPKAPK2 (Thr334) | Thermo Fisher Scientific/PA5-17791 |
| Antibody | |
| Phospho-SEK1/MKK4 (Ser257) | Cell Signaling Technology/4514 |
| Antibody | |
| Phospho-SEK1/MKK4 | Cell Signaling Technology/9156 |
| (Ser257/Thr261) Antibody | |

The immunoprecipitated RAS-RAF-MAPK pathway proteins may be reduced and alkylated prior to fragmentation (e.g., digestion). Samples that have been reduced and alkylated may comprises modifications, such as to cysteine residues (e.g., CAM). Where a RAS-RAF-MAPK peptide of SEQ ID NO: 1-1400 shows modification resulting from, for example, reduction/alkylation, the non-modified peptide is also encompassed. For example, in each instance where a RAS-RAF-MAPK pathway peptide of SEQ ID NO: 1-1400 is referred to, also encompassed are unmodified peptides of SEQ ID NO: 1-1400.

The samples may optionally be desalted prior to analysis by mass spectrometry. Both enzymatic and chemical digestion is encompassed. Enzymatic digestion includes, but is not limited to, digestion with a protease such as, for example, trypsin, chymotrypsin, AspN, GluC, LysC, LysN, ArgC, proteinase K, pepsin, Clostripain, Elastase, GluC biocarb, LysC/P, LysN Promisc, Protein Endopeptidase, Staph Protease or thermolysin. Chemical digestion includes use of, for example, CNBr, iodosobenzoate and formic acid.

In some embodiments, after fragmentation (e.g., digestion), peptide samples are analyzed by mass spectrometry (MS), and the resulting spectra are compared with theoretical spectra from known proteins to determine the peptides and proteins in a sample. For RAS-RAF-MAPK pathway proteins, discovery MS is cumbersome and time consuming and is not a viable clinical method. Therefore, the inventors have identified novel peptides that associate with RAS-RAF-MAPK pathway proteins for use in the IP-MS methods. Use of these peptides in targeted MS, and IP-targeted MS methods allows quantitation of even low abundant RAS-RAF-MAPK proteins. Moreover, use of these peptides in targeted MS, and in IP-targeted MS methods, allows quantitation of phosphorylated RAS-RAF-MAPK proteins.

Theoretically, peptides useful in MS to detect and quantify RAS-RAF-MAPK pathway proteins can be designed by use of computer software and the like. Surprisingly, however, many of these potential peptide sequences are unsuitable or ineffective for use in MS-based assays, including SRM/MRM and PRM. Because it was not possible to predict the most suitable peptides for MS analysis, it was necessary to experimentally identify modified and unmodified peptides to develop as clinical reagents. To complicate the analysis, it was discovered that certain peptides useful when assaying typical samples were not predictive when assaying samples that had undergone immunoprecipitation.

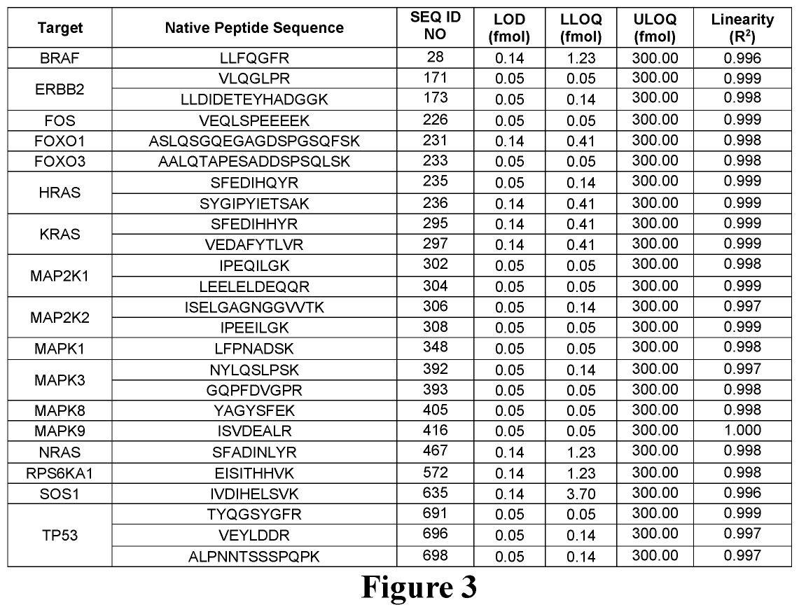

Typically, targeted MS is performed by quantifying specific unique peptides of the protein. In some embodiments, known amounts of isotope-labeled (e.g., heavy isotope-labeled) versions of these targeted peptides can be used as internal standards for absolute quantitation. In some instances, proteins of interest are not detectable even after identifying unique peptide standards. The combination of specific antibodies with specific target peptides has allowed the inventors to improve the sensitivity of detection of RAS-RAF-MAPK pathway proteins by MS, and has allowed for lower levels of detection and lower levels of quantification than ever previously seen. See, e.g., FIG. 3.

In some embodiments, the RAS-RAF-MAPK pathway peptides provided in the kits, and useful in the described methods, are listed in Table 3. SEQ ID Nos: 1-700 are native peptide sequences useful in identifying the RAS-RAF-MAPK pathway proteins recited in the “Target ID” column. Certain peptide sequences are phosphorylated at certain residues as shown in parentheses “(PO3H2)” following the modified residue.

Certain peptides are modified at cysteine residues as shown by “(CAM)” following the modified residue. The “CAM” post-translational modification is well known to those of skill in the art to mean carbamidomethylation, resulting from alkylation of the protein/peptide. The peptides may be as shown in Table 3, or may be non-modified version of these peptides lacking carbamidomethylation.

TABLE 3
List of peptides to quantitate RAS-RAF-MAPK Pathway proteins

| | | SEQ | | SEQ |
|---|---|---|---|---|
| | | ID | Internal Standard Peptide | ID |
| Target ID | Native Peptide Sequence | NO: | Sequence | NO: |
| ALK_1 | TSSISDLK | 1 | TSSISDL[K(13C6; 15N2)] | 701 |
| ALK_2 | TSSISDLKEVPR | 2 | TSSISDLKEVP[R(13C6; 15N4)] | 702 |
| ALK_3 | FNHQNIVR | 3 | FNHQNIV[R(13C6; 15N4)] | 703 |
| ALK_4 | DIA[C(CAM)]G[C(CAM)] | 4 | DIA[C(CAM)]G[C(CAM)] | 704 |
| | QYLEENHFIHR | | QYLEENHFIH[R(13C6; 15N4)] | |
| ALK_5 | N[C(CAM)]LLT[C(CAM)] | 5 | N[C(CAM)]LLT[C(CAM)]PGPG | 705 |
| | PGPGR | | [R(13C6; 15N4)] | |
| ALK_6 | SNQEVLEFVTSGGR | 6 | SNQEVLEFVTSGG[R(13C6; | 706 |
| | | | 15N4)] | |
| ALK_7 | N[C(CAM)]PGPVYR | 7 | N[C(CAM)]PGPVY[R(13C6; | 707 |
| | | | 15N4)] | |
| ALK_8 | DPEGVPPLLVSQQAK | 8 | DPEGVPPLLVSQQA[K(13C6; | 708 |
| | | | 15N2)] | |
| ALK_9 | EEERSPAAPPPLPTTSSGK | 9 | EEERSPAAPPPLPTTSSG[K(13C6; | 709 |
| | | | 15N2)] | |
| ALK_10 | SPAAPPPLPTTSSGK | 10 | SPAAPPPLPTTSSG[K(13C6; | 710 |
| | | | 15N2)] | |
| ALK_11 | GNLGLEGS[C(CAM)]TVPPN | 11 | GNLGLEGS[C(CAM)]TVPPNVATG | 711 |
| | VATGR | | [R(13C6; 15N4)] | |
| ARAF_1 | TQADELPA[C(CAM)]LLSAA | 12 | TQADELPA[C(CAM)]LLSAA | 712 |
| | R | | [R(13C6; 15N4)] | |
| ARAF_2 | IGTGSFGTVFR | 13 | IGTGSFGTVF[R(13C6; 15N4)] | 713 |
| ARAF_3 | IGDFGLATVK | 14 | IGDFGLATV[K(13C6; 15N2)] | 714 |
| ARAF_4 | GLNQD[C(CAM)][C(CAM)] | 15 | GLNQD[C(CAM)][C(CAM)] | 715 |
| | VVYR | | VVY[R(13C6; 15N4)] | |
| ARAF_5 | SNNIFLHEGLTVK | 16 | SNNIFLHEGLTV[K(13C6; | 716 |
| | | | 15N2)] | |
| ARAF_6 | VPTV[C(CAM)]VDMSTNR | 17 | VPTV[C(CAM)]VDMSTN[R(13C6; | 717 |
| | | | 15N4)] | |
| ARAF_7 | VSQPTAEQAQAFK | 18 | VSQPTAEQAQAF[K(13C6; | 718 |
| | | | 15N2)] | |
| ARAF_8 | TQH[C(CAM)]DPEHFPFPAPA | 19 | TQH[C(CAM)]DPEHFPFPAPANAPLQ | 719 |
| | NAPLQR | | [R(13C6; 15N4)] | |
| ATF2_1 | NDSVIVADQTPTPTR | 20 | NDSVIVADQTPTPT[R(13C6; | 720 |
| | | | 15N4)] | |
| ATF2_2 | IEEPSVVETTHQDSPLPHPES | 21 | IEEPSVVETTHQDSPLPHPESTTSDE | 721 |
| | TTSDEK | | [K(13C6; 15N2)] | |
| ATF2_3 | TQSEESRPQSLQQPATSTTET | 22 | TQSEESRPQSLQQPATSTTETP | 722 |
| | PASPAHTTPQTQSTSGR | | ASPAHTTPQTQSTSG[R(13C6; | |
| | | | 15N4)] | |
| ATF2_4 | N[C(CAM)]EEVGLFNELASPF | 23 | N[C(CAM)]EEVGLFNELASPFE | 723 |
| | ENEFK | | NEF[K(13C6; 15N2)] | |
| ATF2_5 | VWVQSLEK | 24 | VWVQSLE[K(13C6; 15N2)] | 724 |
| ATF2_6 | EVPLAQTAQPTSAIVR | 25 | EVPLAQTAQPTSAIV[R(13C6; | 725 |
| | | | 15N4)] | |
| BRAF_1 | SSSAPNVHINTIEPVNIDDLIR | 26 | SSSAPNVHINTIEPVNIDDLI | 726 |
| | | | [R(13C6; 15N4)] | |
| BRAF_2 | AGFQTEDFSLYA[C(CAM)] | 27 | AGFQTEDFSLYA[C(CAM)] | 727 |
| | ASPK | | ASP[K(13C6; 15N2)] | |
| BRAF_3 | LLFQGFR | 28 | LLFQGF[R(13C6; 15N4)] | 728 |
| BRAF_4 | TPIQAGGYGAFPVH | 29 | TPIQAGGYGAFP[V(13C5; | 729 |
| | | | 15N1)]H | |
| BRAF_5 | LTQEHIEALLDK | 30 | LTQEHIEALLD[K(13C6; 15N2)] | 730 |
| BRAFV600 | IGDFGLATEK | 31 | IGDFGLATE[K(13C6; 15N2)] | 731 |
| E_1 | | | | |
| CALM1_1 | EAFSLFDKDGDGTITTK | 32 | EAFSLFDKDGDGTITT[K(13C6; | 732 |
| | | | 15N2)] | |
| CALM1_2 | ELGTVMR | 33 | ELGTVM[R(13C6; 15N4)] | 733 |
| CALM1_3 | DTDSEEEIREAFR | 34 | DTDSEEEIREAF[R(13C6; | 734 |
| | | | 15N4)] | |
| CALM1_4 | VFDKDGNGYISAAELR | 35 | VFDKDGNGYISAAEL[R(13C6; | 735 |
| | | | 15N4)] | |
| CALM1_5 | DGNGYISAAELR | 36 | DGNGYISAAEL[R(13C6; 15N4)] | 736 |
| CALM1_6 | HVMTNLGEK | 37 | HVMTNLGE[K(13C6; 15N2)] | 737 |
| CALM1_7 | EADIDGDGQVNYEEFVQMM | 38 | EADIDGDGQVNYEEFVQMMT | 738 |
| | TAK | | A[K(13C6; 15N2)] | |
| CAMK2A_ | FTEEYQLFEELGK | 39 | FTEEYQLFEELG[K(13C6; | 739 |
| 1 | | | 15N2)] | |
| CAMK2A_ | VLAGQEYAAK | 40 | VLAGQEYAA[K(13C6; 15N2)] | 740 |
| 2 | | | | |
| CAMK2A_ | ITAAEALK | 41 | ITAAEAL[K(13C6; 15N2)] | 741 |
| 3 | | | | |
| CAMK2A_ | VTEQLIEAISNGDFESYTK | 42 | VTEQLIEAISNGDFESYT[K(13C6; | 742 |
| 4 | | | 15N2)] | |
| CAMK2A_ | FYFENLWSR | 43 | FYFENLWS[R(13C6; 15N4)] | 743 |
| 5 | | | | |
| CAMK2A_ | ITQYLDAGGIPR | 44 | ITQYLDAGGIP[R(13C6; 15N4)] | 744 |
| 6 | | | | |
| CAMK2A_ | WQIVHFHR | 45 | WQIVHFH[R(13C6; 15N4)] | 745 |
| 7 | | | | |
| CAMK2A_ | SGAPSVLPH | 46 | SGAPSVL[P(13C5; 15N1)]H | 746 |
| 8 | | | | |
| CCND1_1 | AYPDANLLNDR | 47 | AYPDANLLND[R(13C6; 15N4)] | 747 |
| CCND1_2 | AEET[C(CAM)]APSVSYFK | 48 | AEET[C(CAM)]APSVSYF[K(13C6; | 748 |
| | | | 15N2)] | |
| CCND1_3 | FLSLEPVK | 49 | FLSLEPV[K(13C6; 15N2)] | 749 |
| CCND1_4 | FLSLEPVKK | 50 | FLSLEPVK[K(13C6; 15N2)] | 750 |
| CCND1_5 | ETIPLTAEK | 51 | ETIPLTAE[K(13C6; 15N2)] | 751 |
| CCND1_6 | KHAQTFVAL[C(CAM)]ATDV | 52 | KHAQTFVAL[C(CAM)]ATDV | 752 |
| | K | | [K(13C6; 15N2)] | |
| CCND1_7 | HAQTFVAL[C(CAM)]ATDVK | 53 | HAQTFVAL[C(CAM)]ATDV | 753 |
| | | | [K(13C6; 15N2)] | |
| CCND1_8 | SPNNFLSYYR | 54 | SPNNFLSYY[R(13C6; 15N4)] | 754 |
| CCND1_9 | A[C(CAM)]QEQIEALLESSLR | 55 | A[C(CAM)]QEQIEALLESSL | 755 |
| | | | [R(13C6; 15N4)] | |
| CCND1_10 | AAEEEEEEEEEVDLA[C(CAM)] | 56 | AAEEEEEEEEEVDLA[C(CAM)] | 756 |
| | TPTDVR | | TPTDV[R(13C6; 15N4)] | |
| CDH1_1 | VNFED[C(CAM)]TGR | 57 | VNFED[C(CAM)]M[R(13C6; | 757 |
| | | | 15N4) | |
| CDH1_2 | DWVIPPIS[C(CAM)]PENEK | 58 | DWVIPPIS[C(CAM)]PENE | 758 |
| | | | [K(13C6; 15N2)] | |
| CDH1_3 | VFYSITGQGADTPPVGVFIIE | 59 | VFYSITGQGADTPPVGVFIIE | 759 |
| | R | | [R(13C6; 15N4)] | |
| CDH1_4 | NTGVISVVTTGLDR | 60 | NTGVISVVTTGLD[R(13C6; | 760 |
| | | | 15N4)] | |
| CDH1_5 | GQVPENEANVVITTLK | 61 | GQVPENEANVVITTL[K(13C6; | 761 |
| | | | 15N2)] | |
| CDH1_6 | DTANWLEINPDTGAISTR | 62 | DTANWLEINPDTGAIST[R(13C6; | 762 |
| | | | 15N4)] | |
| CDH1_7 | TIFF[C(CAM)]ER | 63 | TIFF[C(CAM)]E[R(13C6; 15N4)] | 763 |
| CDH1_8 | GLDARPEVTR | 64 | GLDARPEVT[R(13C6; 15N4)] | 764 |
| CDH1_9 | YLPRPANPDEIGNFIDENLK | 65 | YLPRPANPDEIGNFIDENL | 765 |
| | | | [K(13C6; 15N2)] | |
| CDH2_1 | DWVIPPINLPENSR | 66 | DWVIPPINLPENS[R(13C6; | 766 |
| | | | 15N4)] | |
| CDH2_2 | GPFPQELVR | 67 | GPFPQELV[R(13C6; 15N4)] | 767 |
| CDH2_3 | FAIQTDPNSNDGLVTVVKPI | 68 | FAIQTDPNSNDGLVTVVKPIDF | 768 |
| | DFETNR | | ETN[R(13C6; 15N4)] | |
| CDH2_4 | LSDPANWLK | 69 | LSDPANWL[K(13C6; 15N2)] | 769 |
| CDH2_5 | IDPVNGQITTIAVLDR | 70 | IDPVNGQITTIAVLD[R(13C6; | 770 |
| | | | 15N4)] | |
| CDH2_6 | SAAPHPGDIGDFINEGLK | 71 | SAAPHPGDIGDFINEGL[K(13C6; | 771 |
| | | | 15N2)] | |
| CDKN1A_1 | RLFGPVDSEQLSR | 72 | RLFGPVDSEQLS[R(13C6; | 772 |
| | | | 15N4)] | |
| CDKN1A_2 | LFGPVDSEQLSR | 73 | LFGPVDSEQLS[R(13C6; 15N4)] | 773 |
| CDKN1A_3 | ERWNFDFVTETPLEGDFAW | 74 | ERWNFDFVTETPLEGDFAWE | 774 |
| | ER | | [R(13C6; 15N4)] | |
| CDKN1A_4 | WNFDFVTETPLEGDFAWER | 75 | WNFDFVTETPLEGDFAWE | 775 |
| | | | [R(13C6; 15N4)] | |
| CDKN1A_5 | LYLPTGPR | 76 | LYLPTGP[R(13C6; 15N4)] | 776 |
| CDKN1A_6 | RPGTSPALLQGTAEEDHVDL | 77 | RPGTSPALLQGTAEEDHVDLS | 777 |
| | SLS[C(CAM)]TLVPR | | LS[C(CAM)]TLVP[R(13C6; | |
| | | | 15N4)] | |
| CDKN1A_7 | SGEQAEGSPGGPGDSQGR | 78 | SGEQAEGSPGGPGDSQG[R(13C6; | 778 |
| | | | 15N4)] | |
| CDKN1B_1 | VSNGSPSLER | 79 | VSNGSPSLE[R(13C6; 15N4)] | 779 |
| CDKN1B_2 | NLFGPVDHEELTR | 80 | NLFGPVDHEELT[R(13C6; | 780 |
| | | | 15N4)] | |
| CDKN1B_3 | WNFDFQNHKPLEGK | 81 | WNFDFQNHKPLEG[K(13C6; | 781 |
| | | | 15N2)] | |
| CDKN1B_4 | YEWQEVEK | 82 | YEWQEVE[K(13C6; 15N2)] | 782 |
| CDKN1B_5 | GSLPEFYYRPPRPPK | 83 | GSLPEFYYRPPRPP[K(13C6; | 783 |
| | | | 15N2)] | |
| CDKN1B_6 | TDPSDSQTGLAEQ[C(CAM)] | 84 | TDPSDSQTGLAEQ[C(CAM)] | 784 |
| | AGIR | | AGI[R(13C6; 15N4)] | |
| CDKN1B_7 | TEENVSDGSPNAGSVEQTPK | 85 | TEENVSDGSPNAGSVEQTP | 785 |
| | | | [K(13C6; 15N2)] | |
| CDKN2A_1 | ALLEAGALPNAPNSYGR | 86 | ALLEAGALPNAPNSYG[R(13C6; | 786 |
| | | | 15N4)] | |
| CDKN2A_1 | RPIQVMMMGSAR | 87 | RPIQVMMMGSA[R(13C6; | 787 |
| | | | 15N4)] | |
| CDKN2A_2 | LPVDLAEELGHR | 88 | LPVDLAEELGH[R(13C6; 15N4)] | 788 |
| CDKN2A_3 | IDAAEGPSDIPD | 89 | IDAAEGPSDI[P(13C5; 15N1)]D | 789 |
| CREB1_1 | ILNDLSSDAPGVPR | 90 | ILNDLSSDAPGVP[R(13C6; | 790 |
| | | | 15N4)] | |
| CREB1_2 | TAPTSTIAPGVVMASSPALPT | 91 | TAPTSTIAPGVVMASSPALPTQ | 791 |
| | QPAEEAAR | | PAEEAA[R(13C6; 15N4)] | |
| CTNNB1_1 | HAVVNLINYQDDAELATR | 92 | HAVVNLINYQDDAELAT | 792 |
| | | | [R(13C6; 15N4)] | |
| CTNNB1_2 | AIPELTKLLNDEDQVVVNK | 93 | AIPELTKLLNDEDQVVVN | 793 |
| | | | [K(13C6; 15N2)] | |
| CTNNB1_3 | LLNDEDQVVVNK | 94 | LLNDEDQVVVN[K(13C6; | 794 |
| | | | 15N2)] | |
| CTNNB1_4 | [C(CAM)]TAGTLHNLSHHR | 95 | [C(CAM)]TAGTLHNLSHH | 795 |
| | | | [R(13C6; 15N4)] | |
| CTNNB1_5 | SGGIPALVK | 96 | SGGIPALV[K(13C6; 15N2)] | 796 |
| CTNNB1_6 | NLSDAATK | 97 | NLSDAAT[K(13C6; 15N2)] | 797 |
| CTNNB1_7 | AGDREDITEPAI[C(CAM)] | 98 | AGDREDITEPAI[C(CAM)]AL | 798 |
| | ALR | | [R(13C6; 15N4)] | |
| CTNNB1_8 | EDITEPAI[C(CAM)]ALR | 99 | EDITEPAI[C(CAM)IAL[R(13C6; | 799 |
| | | | 15N4)] | |
| CTNNB1_9 | LHYGLPVVVK | 100 | LHYGLPVVV[K(13C6; 15N2)] | 800 |
| CTNNB1_10 | LLHPPSHWPLIK | 101 | LLHPPSHWPLI[K(13C6; 15N2)] | 801 |
| CTNNB1_11 | NLAL[C(CAM)]PANHAPLR | 102 | NLAL[C(CAM)]PANHAPL | 802 |
| | | | [R(13C6; 15N4)] | |
| CTNNB1_12 | NLAL[C(CAM)]PANHAPLRE | 103 | NLAL[C(CAM)]PANHAPLREQ | 803 |
| | QGAIPR | | GAIP[R(13C6; 15N4)] | |
| CTNNB1_13 | LVQLLVR | 104 | LVQLLV[R(13C6; 15N4)] | 804 |
| CTNNB1_14 | EAAEAIEAEGATAPLTELLH | 105 | EAAEAIEAEGATAPLTELLHS | 805 |
| | SR | | [R(13C6; 15N4)] | |
| CTNNB1_15 | NEGVATYAAAVLFR | 106 | NEGVATYAAAVLF[R(13C6; | 806 |
| | | | 15N4)] | |
| DDR2_1 | DLEPPIVAR | 107 | DLEPPIVA[R(13C6; 15N4)] | 807 |
| DDR2_2 | IFPLRPDYQEPSR | 108 | IFPLRPDYQEPS[R(13C6; 15N4)] | 808 |
| DDR2_3 | TVSYTNLK | 109 | TVSYTNL[K(13C6; 15N2)] | 809 |
| DDR2_4 | NLYSGDYYR | 110 | NLYSGDYY[R(13C6; 15N4)] | 810 |
| DDR2_5 | YLSSLNFVHR | 111 | YLSSLNFVH[R(13C6; 15N4)] | 811 |
| DDR2_6 | DVAVEEFPR | 112 | DVAVEEFP[R(13C6; 15N4)] | 812 |
| DDR2_7 | SSSPSEQGSNSTYDR | 113 | SSSPSEQGSNSTYD[R(13C6; | 813 |
| | | | 15N4)] | |
| DDR2_8 | FVTVPLHHR | 114 | FVTVPLHH[R(13C6; 15N4)] | 814 |
| DDR2_9 | HEPPNSSSSDVR | 115 | HEPPNSSSSDV[R(13C6; 15N4)] | 815 |
| DDR2_10 | QTYLPQPAI[C(CAM)] | 116 | QTYLPQPAI[C(CAM)]PDSVY | 816 |
| | PDSVYK | | [K(13C6; 15N2)] | |
| DUSP22_1 | ILPGLYIGNFK | 117 | ILPGLYIGNF[K(13C6; 15N2)] | 817 |
| DUSP22_2 | VTHILSVHDSARPMLEGVK | 118 | VTHILSVHDSARPMLEGV | 818 |
| | | | [K(13C6; 15N2)] | |
| DUSP22_3 | YL[C(CAM)]IPAADSPSQNLTR | 119 | YL[C(CAM)]IPAADSPSQNLT | 819 |
| | | | [R(13C6; 15N4)] | |
| DUSP22_4 | FIHE[C(CAM)]R | 120 | FIHE[C(CAM)][R(13C6; 15N4)] | 820 |
| DUSP22_5 | GES[C(CAM)]LVH[C(CAM)] | 121 | GES[C(CAM)]LVH[C(CAM)] | 821 |
| | LAGVSR | | LAGVS[R(13C6; 15N4)] | |
| DUSP22_6 | S[C(CAM)]ANPNVGFQR | 122 | S[C(CAM)]ANPNVGFQ[R(13C6; | 822 |
| | | | 15N4)] | |
| DUSP22_7 | EEYGESPLQDAEEAK | 123 | EEYGESPLQDAEEA[K(13C6; | 823 |
| | | | 15N2)] | |
| DUSP4_1 | DENGGGAGGSGSHGTLGLP | 124 | DENGGGAGGSGSHGTLGLPSG | 824 |
| | SGGK | | G[K(13C6; 15N2)] | |
| DUSP4_2 | [C(CAM)]LLLD[C(CAM)] | 125 | [C(CAM)]LLLD[C(CAM)] | 825 |
| | RPFLAHSAGYILGSVNVR | | RPFLAHSAGYILGSVNV[R(13C6; | |
| | | | 15N4)] | |
| DUSP4_3 | LRSGLYSAVIVYDER | 126 | LRSGLYSAVIVYDE[R(13C6; | 826 |
| | | | 15N4)] | |
| DUSP4_4 | SGLYSAVIVYDER | 127 | SGLYSAVIVYDE[R(13C6; | 827 |
| | | | 15N4)] | |
| DUSP4_5 | FSSEYPEF[C(CAM)]SK | 128 | FSSEYPEF[C(CAM)]S[K(13C6; | 828 |
| | | | 15N2)] | |
| DUSP4_6 | VRLEEAFEFVK | 129 | VRLEEAFEFV[K(13C6; 15N2)] | 829 |
| DUSP4_7 | LEEAFEFVK | 130 | LEEAFEFV[K(13C6; 15N2)] | 830 |
| DUSP6_1 | TVAWLNEQLELGNER | 131 | TVAWLNEQLELGNE[R(13C6; | 831 |
| | | | 15N4)] | |
| DUSP6_2 | AFYLEGGFSK | 132 | AFYLEGGFS[K(13C6; 15N2)] | 832 |
| DUSP6_3 | ISSDSSSDIESDLDR | 133 | ISSDSSSDIESDLD[R(13C6; | 833 |
| | | | 15N4)] | |
| DUSP6_4 | DSTNLDVLEEFGIK | 134 | DSTNLDVLEEFGI[K(13C6; | 834 |
| | | | 15N2)] | |
| DUSP6_5 | YILNVTPNLPNLFENAGEFK | 135 | YILNVTPNLPNLFENAGEF | 835 |
| | | | [K(13C6; 15N2)] | |
| DUSP6_6 | N[C(CAM)]GVLVH[C(CAM)] | 136 | N[C(CAM)]GVLVH[C(CAM)] | 836 |
| | LAGISR | | LAGIS[R(13C6; 15N4)] | |
| DUSP6_7 | TLGLSSP[C(CAM)]DNR | 137 | TLGLSSP[C(CAM)]DN[R(13C6; | 837 |
| | | | 15N4)] | |
| EGFR_1 | NLQEILHGAVR | 138 | NLQEILHGAV[R(13C6; 15N4)] | 838 |
| EGFR_2 | IPLENLQIIR | 139 | IPLENLQII[R(13C6; 15N4)] | 839 |
| EGFR_3 | LTQLGTFEDHFLSLQR | 140 | LTQLGTFEDHFLSLQ[R(13C6; | 840 |
| | | | 15N4)] | |
| EGFR_4 | YSFGAT[C(CAM)]VK | 141 | YSFGAT[C(CAM)]V[K(13C6; | 841 |
| | | | 15N2)] | |
| EGFR_5 | YLVIQGDER | 142 | YLVIQGDE[R(13C6; 15N4)] | 842 |
| EGFR_6 | ELVEPLTPSGEAPNQALLR | 143 | ELVEPLTPSGEAPNQALL | 843 |
| | | | [R(13C6; 15N4)] | |
| EGFR_7 | GSHQISLDNPDYQQDFFPK | 144 | GSHQISLDNPDYQQDFFP | 844 |
| | | | [K(13C6; 15N2)] | |
| EGFR_8 | ELVEPL[pT]PSGEAPNQALL | 145 | ELVEPL[pT]PSGEAPNQALL | 845 |
| | R | | [R(13C6; 15N4)] | |
| EGFR_9 | GSHQI[pS]LDNPDYQQDFFP | 146 | GSHQI[pS]LDNPDYQQDFFP | 846 |
| | K | | [K(13C6; 15N2)] | |
| EIF2A_1 | DGTLFAWGNGEK | 147 | DGTLFAWGNGE[K(13C6; | 847 |
| | | | 15N2)] | |
| EIF2A_2 | VNIISVTNK | 148 | VNIISVTN[K(13C6; 15N2)] | 848 |
| EIF2A_3 | AV[C(CAM)]LEFSPK | 149 | AV[C(CAM)]LEFSP[K(13C6; | 849 |
| | | | 15N2)] | |
| EIF2A_4 | NTVLATWQPYTTSK | 150 | NTVLATWQPYTTS[K(13C6; | 850 |
| | | | 15N2)] | |
| EIF2A_5 | DGTAGIPNLQLYDVK | 151 | DGTAGIPNLQLYDV[K(13C6; | 851 |
| | | | 15N2)] | |
| EIF2A_6 | NVNNEVHFFENNNFNTIANK | 152 | NVNNEVHFFENNNFNTIAN | 852 |
| | | | [K(13C6; 15N2)] | |
| EIF2A_7 | INDFVLSPGPQPYK | 153 | INDFVLSPGPQPY[K(13C6; | 853 |
| | | | 15N2)] | |
| EIF2A_8 | LYQYPNFAGPHAALANK | 154 | LYQYPNFAGPHAALAN[K(13C6; | 854 |
| | | | 15N2) | |
| EIF2A_9 | ATIFNLK | 155 | ATIFNL[K(13C6; 15N2)] | 855 |
| EIF2A_10 | [C(CAM)]DPVFDFGTGPR | 156 | [C(CAM)]DPVFDFGTGP[R(13C6; | 856 |
| | | | 15N4)] | |
| EIF2A_11 | NAAYYSPHGHILVLAGFGNL | 157 | NAAYYSPHGHILVLAGFGNL | 857 |
| | R | | R(13C6; 15N4) | |
| EIF2A_12 | TITYQAVPSEVPNEEPK | 158 | TITYQAVPSEVPNEEP[K(13C6; | 858 |
| | | | 15N2)] | |
| EIF2A_13 | VATAYRPPALR | 159 | VATAYRPPAL[R(13C6; 15N4)] | 859 |
| EIF4E_1 | WLITLNK | 160 | WLITLN[K(13C6; 15N2)] | 860 |
| EIF4E_2 | IAIWTTE[C(CAM)]ENR | 161 | IAIWTTE[C(CAM)]EN[R(13C6; | 861 |
| | | | 15N4)] | |
| EIF4E_3 | EAVTHIGR | 162 | EAVTHIG[R(13C6; 15N4)] | 862 |
| EIF4E_4 | IVIGYQSHADTATK | 163 | IVIGYQSHADTAT[K(13C6; | 863 |
| | | | 15N2)] | |
| EIF4E_5 | TESNQEVANPEHYIK | 164 | TESNQEVANPEHYI[K(13C6; | 864 |
| | | | 15N2)] | |
| EIF4E_6 | TWQANLR | 165 | TWQANL[R(13C6; 15N4)] | 865 |
| EIF4E_7 | DGIEPMWEDEK | 166 | DGIEPMWEDE[K(13C6; 15N2)] | 866 |
| ERBB2_1 | NPQL[C(CAM)]YQDTILWK | 167 | NPQL[C(CAM)]YQDTILW[K(13C6; | 867 |
| | | | 15N2)] | |
| ERBB2_2 | [C(CAM)]WGESSED[C(CAM)] | 168 | [C(CAM)]WGESSED[C(CAM)] | 868 |
| | QSLTR | | QSLT[R(13C6; 15N4)] | |
| ERBB2_3 | AVTSANIQEFAG[C(CAM)]K | 169 | AVTSANIQEFAG[C(CAM)] | 869 |
| | | | [K(13C6; 15N2)] | |
| ERBB2_4 | GQE[C(CAM)]VEE[C(CAM)]R | 170 | GQE[C(CAM)]VEE[C(CAM)] | 870 |
| | | | [R(13C6; 15N4)] | |
| ERBB2_5 | VLQGLPR | 171 | VLQGLP[R(13C6; 15N4)] | 871 |
| ERBB2_6 | GIWIPDGENVK | 172 | GIWIPDGENV[K(13C6; 15N2)] | 872 |
| ERBB2_7 | LLDIDETEYHADGGK | 173 | LLDIDETEYHADGG[K(13C6; | 873 |
| | | | 15N2)] | |
| ERBB2_8 | ELVSEFSR | 174 | ELVSEFS[R(13C6; 15N4)] | 874 |
| ERBB2_9 | FVVIQNEDLGPASPLDSTFY | 175 | FVVIQNEDLGPASPLDSTFY | 875 |
| | R | | [R(13C6; 15N4)] | |
| ERBB2_10 | SGGGDLTLGLEPSEEEAPR | 176 | SGGGDLTLGLEPSEEEAP | 876 |
| | | | [R(13C6; 15N4)] | |
| ERBB2_11 | GLQSLPTHDPSPLQR | 177 | GLQSLPTHDPSPLQ[R(13C6; | 877 |
| | | | 15N4)] | |
| ERBB2_12 | FPDEEGA[C(CAM)]QP[C(CAM) | 178 | FPDEEGA[C(CAM)]QP[C(CAM)] | 878 |
| | WIN[C(CAM)]THS[C(CAM)] | | PIN[C(CAM)]THS[C(CAM)] | |
| | VDLDDK | | VDLDD[K(13C6; 15N2)] | |
| ERBB2_13 | NNQLALTLIDTNR | 179 | NNQLALTLIDTN[R(13C6; | 879 |
| | | | 15N4)] | |
| ERBB2_14 | GPLPTD[C(CAM)][C(CAM)] | 180 | GPLPTD[C(CAM)][C(CAM)] | 880 |
| | HEQ[C(CAM)]AAG[C(CAM)] | | HEQ[C(CAM)]AAG[C(CAM)]TGP | |
| | TGPK | | [K(13C6; 15N2)] | |
| ERBB3_1 | G[C(CAM)]LASESSEGHVTG | 181 | G[C(CAM)]LASESSEGHVTGSE | 881 |
| | SEAELQEK | | AELQE[K(13C6; 15N2)] | |
| ERBB3_2 | GGV[C(CAM)]VTH[C(CAM)] | 182 | GGV[C(CAM)]VTH[C(CAM)] | 882 |
| | NFLNGEPR | | NFLNGEP[R(13C6; 15N4)] | |
| ERBB4_1 | LSSLSDLEQQYR | 183 | LSSLSDLEQQY[R(13C6; 15N4)] | 883 |
| ERBB4_2 | DGNFGLQELGLK | 184 | DGNFGLQELGL[K(13C6; | 884 |
| | | | 15N2)] | |
| ERBB4_3 | FL[C(CAM)]YADTIHWQDIV | 185 | FL[C(CAM)]YADTIHWQDIV | 885 |
| | R | | [R(13C6; 15N4)] | |
| ERBB4_4 | [C(CAM)]PHNFVVDSSS | 186 | [C(CAM)]PHNFVVDSSS[C(CAM)] | 886 |
| | [C(CAM)]VR | | V[R(13C6; 15N4)] | |
| ERBB4_5 | FLETELVEPLTPSGTAPNQA | 187 | FLETELVEPLTPSGTAPNQAQL | 887 |
| | QLR | | [R(13C6; 15N4)] | |
| ERBB4_6 | ILNETTGPK | 188 | ILNETTGP[K(13C6; 15N2)] | 888 |
| ERBB4_7 | ELAAEFSR | 189 | ELAAEFS[R(13C6; 15N4)] | 889 |
| ERBB4_8 | DGGFAAEQGVSVPYR | 190 | DGGFAAEQGVSVPY[R(13C6; | 890 |
| | | | 15N4)] | |
| ERBB4_9 | KPVAPHVQEDSSTQR | 191 | KPVAPHVQEDSSTQ[R(13C6; | 891 |
| | | | 15N4)] | |
| ERBB4_10 | YSADPTVFAPER | 192 | YSADPTVFAPE[R(13C6; 15N4)] | 892 |
| ERBB4_11 | STLQHPDYLQEYSTK | 193 | STLQHPDYLQEYST[K(13C6; | 893 |
| | | | 15N2)] | |
| ERBB4_12 | NLTEILNGGVYVDQNK | 194 | NLTEILNGGVYVDQN[K(13C6; | 894 |
| | | | 15N2)] | |
| ESR1_1 | Y[C(CAM)]AV[C(CAM)] | 195 | Y[C(CAM)]AV[C(CAM)]NDYAS | 895 |
| | NDYASGYHYGVWS[C(CAM)] | | GYHYGVWS[C(CAM)]EG | |
| | EG[C(CAM)]K | | [C(CAM)][K(13C6; 15N2)] | |
| ESR1_2 | LLFAPNLLLDR | 196 | LLFAPNLLLD[R(13C6; 15N4)] | 896 |
| ESR1_3 | AGLTLQQQHQR | 197 | AGLTLQQQHQ[R(13C6; 15N4)] | 897 |
| ESR1_4 | LAQLLLILSHIR | 198 | LAQLLLILSHI[R(13C6; 15N4)] | 898 |
| ESR1_5 | GGASVEETDQSHLATAGSTS | 199 | GGASVEETDQSHLATAGSTSS | 899 |
| | SHSLQK | | HSLQ[K(13C6; 15N2)] | |
| ESR1_6 | AANLWPSPLMIK | 200 | AANLWPSPLMI[K(13C6; 15N2)] | 900 |
| FBXO7_1 | RTWPLEVPETEPTLGHLR | 201 | RTWPLEVPETEPTLGHL[R(13C6; | 901 |
| | | | 15N4)] | |
| FBXO7_2 | TWPLEVPETEPTLGHLR | 202 | TWPLEVPETEPTLGHL[R(13C6; | 902 |
| | | | 15N4)] | |
| FBXO7_3 | LQLLPESFI[C(CAM)]K | 203 | LQLLPESFI[C(CAM)][K(13C6; | 903 |
| | | | 15N2)] | |
| FBXO7_4 | LGENVANIYK | 204 | LGENVANIY[K(13C6; 15N2)] | 904 |
| FBXO7_5 | LGENVANIYKDLQK | 205 | LGENVANIYKDLQ[K(13C6; | 905 |
| | | | 15N2)] | |
| FBXO7_6 | SVLSLSAV[C(CAM)]R | 206 | SVLSLSAV[C(CAM)][R(13C6; | 906 |
| | | | 15N4)] | |
| FBXO7_7 | VQDTDWK | 207 | VQDTDW[K(13C6; 15N2)] | 907 |
| FBXO7_8 | VQDTDWKELYR | 208 | VQDTDWKELY[R(13C6; 15N4)] | 908 |
| FBXO7_9 | FDPVGPLPGPNPILPGR | 209 | FDPVGPLPGPNPILPG[R(13C6; | 909 |
| | | | 15N4)] | |
| FGFR1_1 | DGVQLAESNR | 210 | DGVQLAESN[R(13C6; 15N4)] | 910 |
| FGFR1_2 | EFKPDHR | 211 | EFKPDH[R(13C6; 15N4)] | 911 |
| FGFR1_3 | SPHRPILQAGLPANK | 212 | SPHRPILQAGLPAN[K(13C6; | 912 |
| | | | 15N2)] | |
| FGFR1_4 | VYSDPQPHIQWLK | 213 | VYSDPQPHIQWL[K(13C6; | 913 |
| | | | 15N2)] | |
| FGFR1_5 | IGPDNLPYVQILK | 214 | IGPDNLPYVQIL[K(13C6; | 914 |
| | | | 15N2)] | |
| FGFR1_6 | [C(CAM)]PSSGTPNPTLR | 215 | [C(CAM)]PSSGTPNPTL[R(13C6; | 915 |
| | | | 15N4)] | |
| FGFR1_7 | HPAQLANGGLK | 216 | HPAQLANGGL[K(13C6; 15N2)] | 916 |
| FGFR1_8 | RPPGLEY[C(CAM)]YNPSHN | 217 | RPPGLEY[C(CAM)]YNPSHNPE | 917 |
| | PEEQLSSK | | EQLSS[K(13C6; 15N2)] | |
| FGFR2_1 | TVLIGEYLQIK | 218 | TVLIGEYLQI[K(13C6; 15N2)] | 918 |
| FGFR2_2 | YGPDGLPYLK | 219 | YGPDGLPYL[K(13C6; 15N2)] | 919 |
| FGFR3_1 | LQVLNASHEDSGAYS[C(CAM)] | 220 | LQVLNASHEDSGAYS[C(CAM)] | 920 |
| | R | | [R(13C6; 15N4)] | |
| FGFR3_2 | LLAVPAANTVR | 221 | LLAVPAANTV[R(13C6; 15N4)] | 921 |
| FGFR4_1 | LEIASFLPEDAGR | 222 | LEIASFLPEDAG[R(13C6; | 922 |
| | | | 15N4)] | |
| FGFR4_2 | HIVINGSSFGADGFPYVQVL | 223 | HIVINGSSFGADGFPYVQVL | 923 |
| | K | | [K(13C6; 15N2)] | |
| FOS_1 | APHPFGVPAPSAGAYSR | 224 | APHPFGVPAPSAGAYS[R(13C6; | 924 |
| | | | 15N4)] | |
| FOS_2 | GKVEQLSPEEEEK | 225 | GKVEQLSPEEEE[K(13C6; | 925 |
| | | | 15N2)] | |
| FOS_3 | VEQLSPEEEEK | 226 | VEQLSPEEEE[K(13C6; 15N2)] | 926 |
| FOS_4 | TEPFDDFLFPASSRPSGSETA | 227 | TEPFDDFLFPASSRPSGSETA | 927 |
| | R | | ([R13C6; 15N4)] | |
| FOS_5 | SALQTEIANLLK | 228 | SALQTEIANLL[K(13C6; 15N2)] | 928 |
| FOS_6 | ELTDTLQAETDQLEDEK | 229 | ELTDTLQAETDQLEDE[K(13C6; | 929 |
| | | | 15N2)] | |
| FOS_7 | VEQLSPEEEEKR | 230 | VEQLSPEEEEK[R(13C6; 15N4)] | 930 |
| FOXO1_1 | ASLQSGQEGAGDSPGSQFSK | 231 | ASLQSGQEGAGDSPGSQFS | 931 |
| | | | [K(13C6; 15N2)] | |
| FOXO1_2 | LD[C(CAM)]DMESIIR | 232 | LD[C(CAM)]DMESII[R(13C6; | 932 |
| | | | 15N4)] | |
| FOXO3_1 | AALQTAPESADDSPSQLSK | 233 | AALQTAPESADDSPSQLS[K(13C6; | 933 |
| | | | 15N2)] | |
| FOXO3_2 | SSDELDAWTDFR | 234 | SSDELDAWTDF[R(13C6; | 934 |
| | | | 15N4)] | |
| HRAS_1 | SFEDIHQYR | 235 | SFEDIHQY[R(13C6; 15N4)] | 935 |
| HRAS_2 | SYGIPYIETSAK | 236 | SYGIPYIETSA[K(13C6; 15N2)] | 936 |
| HRAS_3 | LNPPDESGPG[C(CAM)]MS | 237 | LNPPDESGPG[C(CAM)]MS | 937 |
| | [C(CAM)]K | | [C(CAM)][K(13C6; 15N2)] | |
| HSPB1_1 | LFDQAFGLPR | 238 | LFDQAFGLP[R(13C6; 15N4)] | 938 |
| HSPB1_2 | LATQSNEITIPVTFESR | 239 | LATQSNEITIPVTFES[R(13C6; | 939 |
| | | | 15N4)] | |
| HSPB1_3 | VSLDVNHFAPDELTVK | 240 | VSLDVNHFAPDELTV[K(13C6; | 940 |
| | | | 15N2)] | |
| HSPB1_4 | GPSWDPFR | 241 | GPSWDPF[R(13C6; 15N4)] | 941 |
| HSPB1_5 | DGVVEITGK | 242 | DGVVEITG[K(13C6; 15N2)] | 942 |
| HSPB1_6 | VPFSLLR | 243 | VPFSLL[R(13C6; 15N4)] | 943 |
| HSPB1_7 | AQLGGPEAAK | 244 | AQLGGPEAA[K(13C6; 15N2)] | 944 |
| IQGAP1_1 | LGNFFSPK | 245 | LGNFFSP[K(13C6; 15N2)] | 945 |
| IQGAP1_2 | ATGLHFR | 246 | ATGLHF[R(13C6; 15N4)] | 946 |
| IQGAP1_3 | LGLAPQIQDLYGK | 247 | LGLAPQIQDLYG[K(13C6; | 947 |
| | | | 15N2)] | |
| IQGAP1_4 | ALQSPALGLR | 248 | ALQSPALGL[R(13C6; 15N4)] | 948 |
| IQGAP1_5 | ELATLQR | 249 | ELATLQ[R(13C6; 15N4)] | 949 |
| IQGAP1_6 | ALESGDVNTVWK | 250 | ALESGDVNTVW[K(13C6; | 950 |
| | | | 15N2)] | |
| IQGAP1_7 | TLQALQIPAAK | 251 | TLQALQIPAA[K(13C6; 15N2)] | 951 |
| IQGAP1_8 | EEYLLLR | 252 | EEYLLL[R(13C6; 15N4)] | 952 |
| IQGAP1_9 | LPYDVTPEQALAHEEVK | 253 | LPYDVTPEQALAHEEV[K(13C6; | 953 |
| | | | 15N2)] | |
| IQGAP1_10 | IIGNLLYYR | 254 | IIGNLLYY[R(13C6; 15N4)] | 954 |
| IQGAP1_11 | LQQTYAALNSK | 255 | LQQTYAALNS[K(13C6; 15N2)] | 955 |
| IQGAP1_12 | ATFYGEQVDYYK | 256 | ATFYGEQVDYY[K(13C6; | 956 |
| | | | 15N2)] | |
| IQGAP1_13 | T[C(CAM)]LDNLASK | 257 | T[C(CAM)]LDNLAS[K(13C6; | 957 |
| | | | 15N2)] | |
| JAK1_1 | LGSGEYTAEEL[C(CAM)]IR | 258 | LGSGEYTAEEL[C(CAM)]I | 958 |
| | | | [R(13C6; 15N4)] | |
| JAK1_2 | ISPL[C(CAM)]HNLFALYDEN | 259 | ISPL[C(CAM)]HNLFALYDENT | 959 |
| | TK | | [K(13C6; 15N2)] | |
| JAK1_3 | YIPETLNK | 260 | YIPETLN[K(13C6; 15N2)] | 960 |
| JAK1_4 | YLATLETLTK | 261 | YLATLETLT[K(13C6; 15N2)] | 961 |
| JAK1_5 | NFQIEVQK | 262 | NFQIEVQ[K(13C6; 15N2)] | 962 |
| JAK1_6 | EISNLLVATK | 263 | EISNLLVAT[[K(13C6; 15N2)] | 963 |
| JAK1_7 | EISNLLVATKK | 264 | EISNLLVATK[K(13C6; 15N2)] | 964 |
| JAK1_8 | KDLVQGEHLGR | 265 | KDLVQGEHLG[R(13C6; 15N4)] | 965 |
| JAK1_9 | DLVQGEHLGR | 266 | DLVQGEHLG[R(13C6; 15N4)] | 966 |
| JAK1_10 | EGIDSE[C(CAM)]GPFIK | 267 | EGIDSE[C(CAM)]GPFI[K(13C6; | 967 |
| | | | 15N2)] | |
| JAK1_11 | LSDPGIPITVLSR | 268 | LSDPGIPITVLS[R(13C6; 15N4)] | 968 |
| JAK1_12 | IPWIAPE[C(CAM)]VEDSK | 269 | IPWIAPE[C(CAM)]VEDS[K(13C6; | 969 |
| | | | 15N2)] | |
| JAK1_13 | YDPEGDNTGEQVAVK | 270 | YDPEGDNTGEQVAV[K(13C6; | 970 |
| | | | 15N2)] | |
| JAK1_14 | NLYHENIVK | 271 | NLYHENIV[K(13C6; 15N2)] | 971 |
| JAK1_15 | NVLVESEHQVK | 272 | NVLVESEHQV+[K(13C6; 15N2)] | 972 |
| JAK2_1 | HDFVHGWIK | 273 | HDFVHGWI[K(13C6; 15N2)] | 973 |
| JAK2_2 | IQDYHILTR | 274 | IQDYHILT[R(13C6; 15N4)] | 974 |
| JAK2_3 | NLEIELSSLR | 275 | NLEIELSSL[R(13C6; 15N4)] | 975 |
| JAK2_4 | AGNQTGLYVLR | 276 | AGNQTGLYVL[R(13C6; 15N4)] | 976 |
| JAK2_5 | YFLTFAVER | 277 | YFLTFAVE[R(13C6; 15N4)] | 977 |
| JAK2_6 | NENEEYNLSGTK | 278 | NENEEYNLSGT[K(13C6; 15N2)] | 978 |
| JAK2_7 | SDNIIFQFTK | 279 | SDNIIFQFT[K(13C6; 15N2)] | 979 |
| JAK2_8 | EVGDYGQLHETEVLLK | 280 | EVGDYGQLHETEVLL[K(13C6; | 980 |
| | | | 15N2)] | |
| JAK2_9 | LSDPGISITVLPK | 281 | LSDPGISITVLP[K(13C6; 15N2)] | 981 |
| JAK2_10 | IPWVPPE[C(CAM)]IENPK | 282 | IPWVPPE[C(CAM)]IENP[K(13C6; | 982 |
| | | | 15N2)] | |
| JAK2_11 | FLQQLGK | 283 | FLQQLG[K(13C6; 15N2)] | 983 |
| JAK2_12 | YDPLQDNTGEVVAVK | 284 | YDPLQDNTGEVVAV[K(13C6; | 984 |
| | | | 15N2)] | |
| JAK2_13 | SLQHDNIVK | 285 | SLQHDNIV[K(13C6; 15N2)] | 985 |
| JAK2_14 | LLQYTSQI[C(CAM)]K | 286 | LLQYTSQI[C(CAM)][K(13C6; | 986 |
| | | | 15N2)] | |
| JAK2_15 | NILVENENR | 287 | NILVENEN[R(13C6; 15N4)] | 987 |
| JAK2_16 | ENDQTPLAIYNSISYK | 288 | ENDQTPLAIYNSISY[K(13C6; | 988 |
| | | | 15N2)] | |
| JAK2_17 | EPGESPIFWYAPESLTESK | 289 | EPGESPIFWYAPESLTES[K(13C6; | 989 |
| | | | 15N2)] | |
| JUN_l | NVTDEQEGFAEGFVR | 290 | NVTDEQEGFAEGFV[R(13C6; | 990 |
| | | | 15N4)] | |
| JUN_2 | NSDLLTSPDVGLLK | 291 | NSDLLTSPDVGLL[K(13C6; | 991 |
| | | | 15N2)] | |
| JUN_3 | AQNSELASTANMLR | 292 | AQNSELASTANML[R(13C6; | 992 |
| | | | 15N4)] | |
| JUN_4 | LIIQSSNGHITTTPTPTQFL | 293 | LIIQSSNGHITTTPTPTQFL | 993 |
| | [C(CAM)]PK | | [C(CAM)]P[K(13C6; 15N2)] | |
| JUN_5 | EEPQTVPEMPGETPPLSPIDM | 294 | EEPQTVPEMPGETPPLSPIDME | 994 |
| | ESQER | | SQE[R(13C6; 15N4)] | |
| KRAS_1 | SFEDIHHYR | 295 | SFEDIHHY[R(13C6; 15N4)] | 995 |
| KRAS_2 | DSEDVPMVLVGNK | 296 | DSEDVPMVLVGN[K(13C6; | 996 |
| | | | 15N2)] | |
| KRAS_3 | VEDAFYTLVR | 297 | VEDAFYTLV[R(13C6; 15N4)] | 997 |
| KRAS.2b_1 | QGVDDAFYTLVR | 298 | QGVDDAFYTLV[R(13C6; | 998 |
| | | | 15N4)] | |
| KSR1_1 | LSHDWL[C(CAM)]YLAPEIV | 299 | LSHDWL[C(CAM)]YLAPEIV | 999 |
| | R | | [R(13C6; 15N4)] | |
| KSR1_2 | ADVLEAHEAEAEEPEAGK | 300 | ADVLEAHEAEAEEPEAG[K(13C6; | 1000 |
| | | | 15N2)] | |
| MAP2K1_1 | ISELGAGNGGVVFK | 301 | ISELGAGNGGVVF[K(13C6; | 1001 |
| | | | 15N2)] | |
| MAP2K1_2 | IPEQILGK | 302 | IPEQILG[K(13C6; 15N2)] | 1002 |
| MAP2K1_3 | VSHKPSGLVMAR | 303 | VSHKPSGLVMA[R(13C6; | 1003 |
| | | | 15N4)] | |
| MAP2K1_4 | LEELELDEQQR | 304 | LEELELDEQQ[R(13C6; 15N4)] | 1004 |
| MAP2K1_5 | VSIAVIK | 305 | VSIAVI[K(13C6; 15N2)] | 1005 |
| MAP2K2_1 | ISELGAGNGGVVTK | 306 | ISELGAGNGGVVT[K(13C6; | 1006 |
| | | | 15N2)] | |
| MAP2K2_2 | VQHRPSGLIMAR | 307 | VQHRPSGLIMA[R(13C6; 15N4)] | 1007 |
| MAP2K2_3 | IPEEILGK | 308 | IPEEILG[K(13C6; 15N2)] | 1008 |
| MAP2K2_4 | SEVEEVDFAGWL[C(CAM)]K | 309 | SEVEEVDFAGWL[C(CAM)] | 1009 |
| | | | [K(13C6; 15N2)] | |
| MAP2K2_5 | LNQPGTPTR | 310 | LNQPGTPT[R(13C6; 15N4)] | 1010 |
| MAP2K3_1 | FSPEFVDFTAQ[C(CAM)]LR | 311 | FSPEFVDFTAQ[C(CAM)]L | 1011 |
| | | | [R(13C6; 15N4)] | |
| MAP2K3_2 | FPYESWGTPFQQLK | 312 | FPYESWGTPFQQL[K(13C6; | 1012 |
| | | | 15N2)] | |
| MAP2K3_3 | DVKPSNVLINK | 313 | DVKPSNVLIN[K(13C6; 15N2)] | 1013 |
| MAP2K3_4 | TFITIGDR | 314 | TFITIGD[R(13C6; 15N4)] | 1014 |
| MAP2K3_5 | TDIAAFVK | 315 | TDIAAFV[K(13C6; 15N2)] | 1015 |
| MAP2K3_6 | NFEVEADDLVTISELGR | 316 | NFEVEADDLVTISELG[R(13C6; | 1016 |
| | | | 15N4)] | |
| MAP2K4_1 | L[C(CAM)]DFGISGQLVDSIA | 317 | L[C(CAM)]DFGISGQLVDSIA | 1017 |
| | K | | [K(13C6; 15N2)] | |
| MAP2K4_2 | FTLNPNPTGVQNPHIER | 318 | FTLNPNPTGVQNPHIE[R(13C6; | 1018 |
| | | | 15N4)] | |
| MAP2K4_3 | WNSVFDQLTQVVK | 319 | WNSVFDQLTQVV[K(13C6; | 1019 |
| | | | 15N2)] | |
| MAP2K4_4 | YVYSVLDDVIPEEILGK | 320 | YVYSVLDDVIPEEILG[K(13C6; | 1020 |
| | | | 15N2)] | |
| MAP2K4_5 | DIKPSNILLDR | 321 | DIKPSNILLD[R(13C6; 15N4)] | 1021 |
| MAP2K4_6 | GDPPQLSNSEER | 322 | GDPPQLSNSEE[R(13C6; 15N4)] | 1022 |
| MAP2K4_7 | AVEVA[C(CAM)]YV[C(CAM)] | 323 | AVEVA[C(CAM)]YV[C(CAM)] | 1023 |
| | K | | [K(13C6; 15N2)] | |
| MAP2K6_1 | DVKPSNVLINALGQVK | 324 | DVKPSNVLINALGQV[K(13C6; | 1024 |
| | | | 15N2)] | |
| MAP2K6_2 | EAFEQPQTSSTPPR | 325 | EAFEQPQTSSTPP[R(13C6; | 1025 |
| | | | 15N4)] | |
| MAP2K6_3 | A[C(CAM)]ISIGNQNFEVK | 326 | A[C(CAM)]ISIGNQNFEV[K(13C6; | 1026 |
| | | | 15N2)] | |
| MAP2K6_4 | GTDVASFVK | 327 | GTDVASFV[K(13C6; 15N2)] | 1027 |
| MAP2K6_5 | IAVSIVK | 328 | IAVSIV[K(13C6; 15N2)] | 1028 |
| MAP2K6_6 | FPYDSWGTPFQQLK | 329 | FPYDSWGTPFQQL[K(13C6; | 1029 |
| | | | 15N2)] | |
| MAP2K6_7 | GQTIPEDILGK | 330 | GQTIPEDILG[K(13C6; 15N2)] | 1030 |
| MAP2K6_8 | FSAEFVDFTSQ[C(CAM)]LK | 331 | FSAEFVDFTSQ[C(CAM)]L | 1031 |
| | | | C[K(136; 15N2)] | |
| MAP2K7_1 | SPSSESSPQHPTPPARPR | 332 | SPSSESSPQHPTPPARP[R(13C6; | 1032 |
| | | | 15N4)] | |
| MAP2K7_2 | TDFEVLTK | 333 | TDFEVLT[K(13C6; 15N2)] | 1033 |
| MAP2K7_3 | DVKPSNILLDER | 334 | DVKPSNILLDE[R(13C6; 15N4)] | 1034 |
| MAP2K7_4 | LLEHSFIK | 335 | LLEHSFI[K(13C6; 15N2)] | 1035 |
| MAP2K7_5 | TSGVLSQPHLPFFR | 336 | TSGVLSQPHLPFF[R(13C6; | 1036 |
| | | | 15N4)] | |
| MAP2K7_6 | IDPPDPTKPDYDIR | 337 | IDPPDPTKPDYDI[R(13C6; | 1037 |
| | | | 15N4)] | |
| MAP2K7_7 | AASSLEQK | 338 | AASSLEQ[K(13C6; 15N2)] | 1038 |
| MAP2K7_8 | L[C(CAM)]DFGISGR | 339 | L[C(CAM)]DFGISG[R(13C6; | 1039 |
| | | | 15N4)] | |
| MAPK1_1 | GQVFDVGPR | 340 | GQVFDVGP[R(13C6; 15N4)] | 1040 |
| MAPK1_2 | FRHENIIGINDIIR | 341 | FRHENIIGINDII[R(13C6; 15N4)] | 1041 |
| MAPK1_3 | HENIIGINDIIR | 342 | HENIIGINDII[R(13C6; 15N4)] | 1042 |
| MAPK1_4 | TQHLSNDHI[C(CAM)] | 343 | TQHLSNDHI[C(CAM)]YFLYQIL | 1043 |
| | YFLYQILR | | [R(13C6; 15N4)] | |
| MAPK1_5 | DLKPSNLLLNTT[C(CAM)] | 344 | DLKPSNLLLNTT[C(CAM)]DL | 1044 |
| | DLK | | [K(13C6; 15N2)] | |
| MAPK1_6 | VADPDHDHTGFLTEYVATR | 345 | VADPDHDHTGFLTEYVAT[R(13C6; | 1045 |
| | | | 15N4)] | |
| MAPK1_7 | NYLLSLPHK | 346 | NYLLSLPH[K(13C6; 15N2)] | 1046 |
| MAPK1_8 | NKVPWNR | 347 | NKVPWN[R(13C6; 15N4)] | 1047 |
| MAPK1_9 | LFPNADSK | 348 | LFPNADS[K(13C6; 15N2)] | 1048 |
| MAPK1_10 | ALDLLDK | 349 | ALDLLD[K(13C6; 15N2)] | 1049 |
| MAPK1_11 | MLTFNPHK | 350 | MLTFNPH[K(13C6; 15N2)] | 1050 |
| MAPK1_12 | MLTFNPHKR | 351 | MLTFNPHK[R(13C6; 15N4)] | 1051 |
| MAPK1_13 | LKELIFEETAR | 352 | LKELIFEETA[R(13C6; 15N4)] | 1052 |
| MAPK11_1 | TVWEVPQR | 353 | TVWEVPQ[R(13C6; 15N4)] | 1053 |
| MAPK11_2 | LQGLRPVGSGAYGSV[C(CAM)] | 354 | LQGLRPVGSGAYGSV[C(CAM)] | 1054 |
| | SAYDAR | | SAYDA[R(13C6; 15N4)] | |
| MAPK11_3 | [C(CAM)]QALSDEHVQFLVY | 355 | [C(CAM)]QALSDEHVQFLVYQLL | 1055 |
| | QLLR | | [R(13C6; 15N4)] | |
| MAPK11_4 | YIHSAGIIHR | 356 | YIHSAGIIH[R(13C6; 15N4)] | 1056 |
| MAPK11_5 | DLKPSNVAVNED[C(CAM)] | 357 | DLKPSNVAVNED[C(CAM)]EL | 1057 |
| | ELR | | [R(13C6; 15N4)] | |
| MAPK11_6 | ALFPGSDYIDQLK | 358 | ALFPGSDYIDQL[K(13C6; | 1058 |
| | | | 15N2)] | |
| MAPK11_7 | ALFPGSDYIDQLKR | 359 | ALFPGSDYIDQLK[R(13C6; | 1059 |
| | | | 15N4)] | |
| MAPK11_8 | IMEVVGTPSPEVLAK | 360 | IMEVVGTPSPEVLA[K(13C6; | 1060 |
| | | | 15N2)] | |
| MAPK11_9 | TYIQSLPPMPQK | 361 | TYIQSLPPMPQ[K(13C6; 15N2)] | 1061 |
| MAPK11_10 | DLSSIFR | 362 | DLSSIF[R(13C6; 15N4)] | 1062 |
| MAPK11_11 | GANPLAIDLLGR | 363 | GANPLAIDLLG[R(13C6; 15N4)] | 1063 |
| MAPK11_12 | MLVLDSDQR | 364 | MLVLDSDQ[R(13C6; 15N4)] | 1064 |
| MAPK11_13 | LSRPFQSLIHAR | 365 | LSRPFQSLIHA[R(13C6; 15N4)] | 1065 |
| MAPK12_1 | YDDSFDDVDR | 366 | YDDSFDDVD[R(13C6; 15N4)] | 1066 |
| MAPK12_2 | DFASILTNASPLAVNLLEK | 367 | DFASILTNASPLAVNLLE[K(13C6; | 1067 |
| | | | 15N2)] | |
| MAPK12_3 | VTAGEALAHPYFESLHDTED | 368 | VTAGEALAHPYFESLHDTEDE | 1068 |
| | EPQVQK | | PQVQ[K(13C6; 15N2)] | |
| MAPK12_4 | YIHAAGIIHR | 369 | YIHAAGIIH[R(13C6; 15N4)] | 1069 |
| MAPK12_5 | VTGTPPAEFVQR | 370 | VTGTPPAEFVQ[R(13C6; 15N4)] | 1070 |
| MAPK12_6 | LYRPFQSELFAK | 371 | LYRPFQSELFA[K(13C6; 15N2)] | 1071 |
| MAPK12_7 | DLQPVGSGAYGAV[C(CAM)] | 372 | DLQPVGSGAYGAV[C(CAM)] | 1072 |
| | SAVDGR | | SAVDG[R(13C6; 15N4)] | |
| MAPK12_8 | EVLSFKPPR | 373 | EVLSFKPP[R(13C6; 15N4)] | 1073 |
| MAPK13_1 | ASPQAADLLEK | 374 | ASPQAADLLE[K(13C6; 15N2)] | 1074 |
| MAPK13_2 | SYIQSLPQTPR | 375 | SYIQSLPQTP[R(13C6; 15N4)] | 1075 |
| MAPK13_3 | VTGVPGTEFVQK | 376 | VTGVPGTEFVQ[K(13C6; | 1076 |
| | | | 15N2)] | |
| MAPK13_4 | YIHSAGVVHR | 377 | YIHSAGVVH[R(13C6; 15N4)] | 1077 |
| MAPK13_5 | TYVSPTHVGSGAYGSV | 378 | TYVSPTHVGSGAYGSV[C(CAM)] | 1078 |
| | [C(CAM)]SAIDK | | SAID[K(13C6; 15N2)] | |
| MAPK13_6 | DYLDQLTQILK | 379 | DYLDQLTQIL[K(13C6; 15N2)] | 1079 |
| MAPK13_7 | DFTQLFPR | 380 | DFTQLFP[R(13C6; 15N4)] | 1080 |
| MAPK13_8 | LTVDEWK | 381 | LTVDEW[K(13C6; 15N2)] | 1081 |
| MAPK14_1 | DLKPSNLAVNED[C(CAM)] | 382 | DLKPSNLAVNED[C(CAM)]EL | 1082 |
| | ELK | | [K(13C6; 15N2)] | |
| MAPK14_2 | DLLIDEWK | 383 | DLLIDEW[K(13C6; 15N2)] | 1083 |
| MAPK14_3 | HENVIGLLDVFTPAR | 384 | HENVIGLLDVFTPA[R(13C6; | 1084 |
| | | | 15N4)] | |
| MAPK14_4 | TLFPGTDHIDQLK | 385 | TLFPGTDHIDQL[K(13C6; | 1085 |
| | | | 15N2)] | |
| MAPK14_5 | YIHSADIIHR | 386 | YIHSADIIH[R(13C6; 15N4)] | 1086 |
| MAPK14_6 | LVGTPGAELLK | 387 | LVGTPGAELL[K(13C6; 15N2)] | 1087 |
| MAPK14_7 | LTDDHVQFLIYQILR | 388 | LTDDHVQFLIYQIL[R(13C6; | 1088 |
| | | | 15N4)] | |
| MAPK14_8 | YQNLSPVGSGAYGSV[C(CAM)] | 389 | YQNLSPVGSGAYGSV[C(CAM)] | 1089 |
| | AAFDTK | | AAFDT[K(13C6; 15N2)] | |
| MAPK14_9 | TIWEVPER | 390 | TIWEVPE[R(13C6; 15N4)] | 1090 |
| MAPK3_1 | ALDLLDR | 391 | ALDLLD[R(13C6; 15N4)] | 1091 |
| MAPK3_2 | NYLQSLPSK | 392 | NYLQSLPS[K(13C6; 15N2)] | 1092 |
| MAPK3_3 | GQPFDVGPR | 393 | GQPFDVGP[R(13C6; 15N4)] | 1093 |
| MAPK3_4 | YTQLQYIGEGAYGMVSSAY | 394 | YTQLQYIGEGAYGMVSSAYDHV | 1094 |
| | DHVR | | [R(13C6; 15N4)] | |
| MAPK3_5 | EIQILLR | 395 | EIQILL[R(13C6; 15N4)] | 1095 |
| MAPK3_6 | DLKPSNLLINTT[C(CAM)] | 396 | DLKPSNLLINTT[C(CAM)]DL | 1096 |
| | DLK | | [K(13C6; 15N2)] | |
| MAPK3_7 | IADPEHDHTGFLTEYVATR | 397 | IADPEHDHTGFLTEYVAT[R(13C6; | 1097 |
| | | | 15N4)] | |
| MAPK3_8 | LKELIFQETAR | 398 | LKELIFQETA[R(13C6; 15N4)] | 1098 |
| MAPK3_9 | ELIFQETAR | 399 | ELIFQETA[R(13C6; 15N4)] | 1099 |
| MAPK3_10 | FQPGVLEAP | 400 | FQPGVLEA[P(13C5; 15N1)] | 1100 |
| MAPK3_11 | LGQYGFPNPEWSEVSEDAK | 401 | LGQYGFPNPEWSEVSEDA | 1101 |
| | | | [K(13C6; 15N2)] | |
| MAPK3_12 | DIGTAIQFLHSHNIAHR | 402 | DIGTAIQFLHSHNIAH[R(13C6; | 1102 |
| | | | 15N4)] | |
| MAPK8_1 | YQNLKPIGSGAQGIV[C(CAM)] | 403 | YQNLKPIGSGAQGIV[C(CAM)] | 1103 |
| | AAAYDAILER | | AAYDAILE[R(13C6; 15N4)] | |
| MAPK8_2 | NIIGLLNVFTPQK | 404 | NIIGLLNVFTPQ[K(13C6; 15N2)] | 1104 |
| MAPK8_3 | YAGYSFEK | 405 | YAGYSFE[K(13C6; 15N2)] | 1105 |
| MAPK8_4 | LFPDVLFPADSEHNK | 406 | LFPDVLFPADSEHN[K(13C6; | 1106 |
| | | | 15N2)] | |
| MAPK8_5 | EVMDLEER | 407 | EVMDLEE[R(13C6; 15N4)] | 1107 |
| MAPK8_6 | DNNFYSVEIGDSTFTVLK | 408 | DNNFYSVEIGDSTFTVL[K(13C6; | 1108 |
| | | | 15N2)] | |
| MAPK8_7 | TYVENRPK | 409 | TYVENRP[K(13C6; 15N2)] | 1109 |
| MAPK8_8 | ISVDEALQHPYINVWYDPSE | 410 | ISVDEALQHPYINVWYDPSEA | 1110 |
| | AEAPPPK | | EAPPP[K(13C6; 15N2)] | |
| MAPK9_1 | [C(CAM)]DSQFYSVQVADST | 411 | [C(CAM)]DSQFYSVQVADSTFTVL | 1111 |
| | FTVLK | | [K(13C6; 15N2)] | |
| MAPK9_2 | [C(CAM)]DSQFYSVQVADST | 412 | [C(CAM)]DSQFYSVQVADSTFT | 1112 |
| | FTVLKR | | VLK[R(13C6; 15N4)] | |
| MAPK9_3 | TA[C(CAM)]TNFMMTPYVVT | 413 | TA[C(CAM)]TNFMMTPYVVT | 1113 |
| | R | | [R(13C6; 15N4)] | |
| MAPK9_4 | G[C(CAM)]VIFQGTDHIDQW | 414 | G[C(CAM)]VIFQGTDHIDQWN | 1114 |
| | NK | | [K(13C6; 15N2)] | |
| MAPK9_5 | FEELFPDWIFPSESER | 415 | FEELFPDWIFPSESE[R(13C6; | 1115 |
| | | | 15N4)] | |
| MAPK9_6 | ISVDEALR | 416 | ISVDEAL[R(13C6; 15N4)] | 1116 |
| MAPK9_7 | HPYITVWYDPAEAEAPPPQI | 417 | HPYITVWYDPAEAEAPPPQIYD | 1117 |
| | YDAQLEER | | AQLEE[R(13C6; 15N4)] | |
| MAPK9_8 | VIEQLGTPSAEFMK | 418 | VIEQLGTPSAEFM[K(13C6; | 1118 |
| | | | 15N2)] | |
| MAPK9_9 | EVMDWEER | 419 | EVMDWEE[R(13C6; 15N4)] | 1119 |
| MAPK9_10 | EHAIEEWK | 420 | EHAIEEW[K(13C6; 15N2)] | 1120 |
| MAPKAPK2_ | SIGEAIQYLHSINIAHR | 421 | SIGEAIQYLHSINIAH[R(13C6; | 1121 |
| 1 | | | 15N4)] | |
| MAPKAPK2_ | IVDVYENLYAGR | 422 | IVDVYENLYAG[R(13C6; | 1122 |
| 2 | | | 15N4)] | |
| MAPKAPK2_ | VPQTPLHTSR | 423 | VPQTPLHTS[R(13C6; 15N4)] | 1123 |
| 3 | | | | |
| MAPKAPK2_ | ETTSHNSLTTP[C(CAM)] | 424 | ETTSHNSLTTP[C(CAM)] | 1124 |
| 4 | YTPYYVAPEVLGPEK | | YTPYYVAPEVLGPE[K(13C6; 15N2)] | |
| MAPKAPK2_ | VLQIFNK | 425 | VLQIFN[K(13C6; 15N2)] | 1125 |
| 5 | | | | |
| MAPKAPK2_ | NAIIDDYK | 426 | NAIIDDY[K(13C6; 15N2)] | 1126 |
| 6 | | | | |
| MAPKAPK2_ | ASQ[C(CAM)]PHIVR | 427 | ASQ[C(CAM)]PHIV[R(13C6; | 1127 |
| 7 | | | 15N4)] | |
| MAPKAPK2_ | VTSQVLGLGINGK | 428 | VTSQVLGLGING[K(13C6; | 1128 |
| 8 | | | 15N2)] | |
| MET_1 | TGPVLEHPD[C(CAM)]FP | 429 | TGPVLEHPD[C(CAM)]FP[C(CAM)] | 1129 |
| | [C(CAM)]QD[C(CAM)]SSK | | QD[C(CAM)]SS[K(13C6; | |
| | | | 15N2)] | |
| MET_2 | ETLDAQTFHTR | 430 | ETLDAQTFHT[R(13C6; 15N4)] | 1130 |
| MET_3 | YVNDFFNK | 431 | YVNDFFN[K(13C6; 15N2)] | 1131 |
| MET_4 | TEFTTALQR | 432 | TEFTTALQ[R(13C6; 15N4)] | 1132 |
| MET_5 | GDLTIANLGTSEGR | 433 | GDLTIANLGTSEG[R(13C6; | 1133 |
| | | | 15N4)] | |
| MET_6 | VFPNSAPLEGGTR | 434 | VFPNSAPLEGGT[R(13C6; | 1134 |
| | | | 15N4)] | |
| MET_7 | SFISGGSTITGVGK | 435 | SFISGGSTITGVG[K(13C6; | 1135 |
| | | | 15N2)] | |
| MET_8 | NLNSVSVPR | 436 | NLNSVSVP[R(13C6; 15N4)] | 1136 |
| MET_9 | DLGSELVR | 437 | DLGSELV[R(13C6; 15N4)] | 1137 |
| MET_10 | DLIGFGLQVAK | 438 | DLIGFGLQVA[K(13C6; 15N2)] | 1138 |
| MET_11 | VADFGLAR | 439 | VADFGLA[R(13C6; 15N4)] | 1139 |
| MET_12 | EVFNILQAAYVSKPGAQLAR | 440 | EVFNILQAAYVSKPGAQLA | 1140 |
| | | | [R(13C6; 15N4)] | |
| NF1_1 | VAQLAVINSLEK | 441 | VAQLAVINSLE[K(13C6; 15N2)] | 1141 |
| NF1_2 | I[C(CAM)]LAQNSPSTFHYVL | 442 | I[C(CAM)]LAQNSPSTFHYVLV | 1142 |
| | VNSLHR | | NSLH[R(13C6; 15N4)] | |
| NF1_3 | IDAVY[C(CAM)]HSVELR | 443 | IDAVY[C(CAM)]HSVEL[R(13C6; | 1143 |
| | | | 15N4)] | |
| NF1_4 | LLL[C(CAM)]NPR | 444 | LLL[C(CAM)]NP[R(13C6; | 1144 |
| | | | 15N4)] | |
| NF1_5 | HL[C(CAM)]EEADIR | 445 | HL[C(CAM)]EEADI[R(13C6; | 1145 |
| | | | 15N4)] | |
| NF1_6 | LILNYPK | 446 | LILNYP[K(13C6; 15N2)] | 1146 |
| NF1_7 | LLDPLLR | 447 | LLDPLL[R(13C6; 15N4)] | 1147 |
| NF1_8 | SV[C(CAM)]H[C(CAM)] | 448 | SV[C(CAM)]H[C(CAM)]LYQAT | 1148 |
| | LYQAT[C(CAM)]HSLLNK | | [C(CAM)]HSLLN[K(13C6; | |
| | | | 15N2)] | |
| NF1_9 | TLSIFYQAGTSK | 449 | TLSIFYQAGTS[K(13C6; 15N2)] | 1149 |
| NF1_10 | AGNPIFYYVAR | 450 | AGNPIFYYVA[R(13C6; 15N4)] | 1150 |
| NF1_11 | LVFID[C(CAM)]PGK | 451 | LVFID[C(CAM)]PG[K(13C6; | 1151 |
| | | | 15N2)] | |
| NF1_12 | VGSTAVQVTSAER | 452 | VGSTAVQVTSAE[R(13C6; | 1152 |
| | | | 15N4)] | |
| NF1_13 | SAAVIAFR | 453 | SAAVIAF[R(13C6; 15N4)] | 1153 |
| NF1_14 | FAFQYNPSLQPR | 454 | FAFQYNPSLQP[R(13C6; 15N4)] | 1154 |
| NF1_15 | GSEGYLAATYPTVGQTSPR | 455 | GSEGYLAATYPTVGQTSP | 1155 |
| | | | [R(13C6; 15N4)] | |
| NF1_16 | ISSSQQHPHLR | 456 | ISSSQQHPHL[R(13C6; 15N4)] | 1156 |
| NF1_17 | VFPVVHNLLDSK | 457 | VFPVVHNLLDS[K(13C6; 15N2)] | 1157 |
| NOTCH1_1 | HGGT[C(CAM)]HNEVGSYR | 458 | HGGT[C(CAM)]HNEVGSY[R(13C6; | 1158 |
| | | | 15N4)] | |
| NOTCH1_2 | VASFY[C(CAM)]E[C(CAM)] | 459 | VASFY[C(CAM)]E[C(CAM)] | 1159 |
| | PHGR | | PHG[R(13C6; 15N4)] | |
| NOTCH1_3 | GTQGVH[C(CAM)]EINVDD | 460 | GTQGVH[C(CAM)]EINVDD | 1160 |
| | [C(CAM)]NPPVDPVSR | | [C(CAM)]NPPVDPVS[R(13C6; | |
| | | | 15N4)] | |
| NOTCH1_4 | AEGQ[C(CAM)]NPLYDQY | 461 | AEGQ[C(CAM)]NPLYDQY | 1161 |
| | [C(CAM)]K | | [C(CAM)][K(13C6; 15N2)] | |
| NOTCH1_5 | AAEGWAAPDALLGQVK | 462 | AAEGWAAPDALLGQV[K(13C6; | 1162 |
| | | | 15N2)] | |
| NOTCH1_6 | FEEPVVLPDLDDQTDHR | 463 | FEEPVVLPDLDDQTDH[R(13C6; | 1163 |
| | | | 15N4)] | |
| NOTCH1_7 | TGETALHLAAR | 464 | TGETALHLAA[R(13C6; 15N4)] | 1164 |
| NOTCH1_8 | LLDEYNLVR | 465 | LLDEYNLV[R(13C6; 15N4)] | 1165 |
| NRAS_1 | TGEGFL[C(CAM)]VFAINNSK | 466 | TGEGFL[C(CAM)]VFAINNS | 1166 |
| | | | [K(13C6; 15N2)] | |
| NRAS_2 | SFADINLYR | 467 | SFADINLY[R(13C6; 15N4)] | 1167 |
| NRAS_3 | QAHELAK | 468 | QAHELA[K(13C6; 15N2)] | 1168 |
| NRAS_4 | QGVEDAFYTLVR | 469 | QGVEDAFYTLV[R(13C6; | 1169 |
| | | | 15N4)] | |
| NRAS_5 | LNSSDDGTQG[C(CAM)] | 470 | LNSSDDGTQG[C(CAM)]MGLP | 1170 |
| | MGLP[C(CAM)]VVM | | [C(CAM)]V[V(13C5; 15N1)]M | |
| NRAS_6 | SYGIPFIETSAK | 471 | SYGIPFIETSA[K(13C6; 15N2)] | 1171 |
| PAK1_1 | NTSTMIGAGSK | 472 | NTSTMIGAGS[K(13C6; 15N2)] | 1172 |
| PAK1_2 | DAGTLNHGSKPLPPNPEEK | 473 | DAGTLNHGSKPLPPNPEE[K(13C6; | 1173 |
| | | | 15N2)] | |
| PAK1_3 | SILPGDK | 474 | SILPGD[K(13C6; 15N2)] | 1174 |
| PAK1_4 | SAEDYNSSNALNVK | 475 | SAEDYNSSNALNV[K(13C6; | 1175 |
| | | | 15N2)] | |
| PAK1_5 | SVIEPLPVTPTR | 476 | SVIEPLPVTPT[R(13C6; 15N4)] | 1176 |
| PAK1_6 | DVATSPISPTENNTTPPDALT | 477 | DVATSPISPTENNTTPPDALT | 1177 |
| | R | | [R(13C6; 15N4)] | |
| PAK1_7 | IGQGASGTVYTAMDVATGQ | 478 | IGQGASGTVYTAMDVATGQE | 1178 |
| | EVAIK | | VAI[K(13C6; 15N2)] | |
| PAK1_8 | E[C(CAM)]LQALEFLHSNQVI | 479 | E[C(CAM)]LQALEFLHSNQVIH | 1179 |
| | HR | | [R(13C6; 15N4)] | |
| PAK1_9 | ELLQHQFLK | 480 | ELLQHQFL[K(13C6; 15N2)] | 1180 |
| PAK1_10 | IAKPLSSLTPLIAAAK | 481 | IAKPLSSLTPLIAAA[K(13C6; | 1181 |
| | | | 15N2)] | |
| PARP1_1 | HPDVEVDGFSELR | 482 | HPDVEVDGFSEL[R(13C6; | 1182 |
| | | | 15N4)] | |
| PARP1_2 | TLGDFAAEYAK | 483 | TLGDFAAEYA[K(13C6; 15N2)] | 1183 |
| PARP1_3 | EELGFRPEYSASQLK | 484 | EELGFRPEYSASQL[K(13C6; | 1184 |
| | | | 15N2)] | |
| PARP1_4 | ELLIFNK | 485 | ELLIFN[K(13C6; 15N2)] | 1185 |
| PARP1_5 | SDAYY[C(CAM)]TGDVTAW | 486 | SDAYY[C(CAM)]TGDVTAWT | 1186 |
| | TK | | [K(13C6; 15N2)] | |
| PARP1_6 | VVSEDFLQDVSASTK | 487 | VVSEDFLQDVSAST[K(13C6; | 1187 |
| | | | 15N2)] | |
| PARP1_7 | AEPVEVVAPR | 488 | AEPVEVVAP[R(13C6; 15N4)] | 1188 |
| PARP1_8 | VFSATLGLVDIVK | 489 | VFSATLGLVDIV[K(13C6; | 1189 |
| | | | 15N2)] | |
| PARP1_9 | KPPLLNNADSVQAK | 490 | KPPLLNNADSVQA[K(13C6; | 1190 |
| | | | 15N2)] | |
| PARP1_10 | TTNFAGILSQGLR | 491 | TTNFAGILSQGL[R(13C6; | 1191 |
| | | | 15N4)] | |
| PGR_1 | TQDQQSLSDVEGAYSR | 492 | TQDQQSLSDVEGAYS[R(13C6; | 1192 |
| | | | 15N4)] | |
| PGR_2 | AEGAPPQQGPFAPPP[C(CAM)] | 493 | AEGAPPQQGPFAPPP[C(CAM)] | 1193 |
| | K | | [K(13C6; 15N2)] | |
| PGR_3 | APGASG[C(CAM)]LLPR | 494 | APGASG[C(CAM)]LLP[R(13C6; | 1194 |
| | | | 15N4)] | |
| PGR_4 | ND[C(CAM)]IVDK | 495 | ND[C(CAM)]IVD[K(13C6; | 1195 |
| | | | 15N2)] | |
| PGR_5 | LLDNLHDLVK | 496 | LLDNLHDLV[K(13C6; 15N2)] | 1196 |
| PIK3CA_1 | INHD[C(CAM)]VPEQVIAEAI | 497 | INHD[C(CAM)]VPEQVIAEAI | 1197 |
| | R | | [R(13C6; 15N4)] | |
| PIK3CA_2 | L[C(CAM)]LSI[C(CAM)]SVK | 498 | L[C(CAM)]LSI[C(CAM)]SV | 1198 |
| | | | [K(13C6; 15N2)] | |
| PIK3CA_3 | LINLTDILK | 499 | LINLTDIL[K(13C6; 15N2)] | 1199 |
| PIK3CA_4 | TGIYHGGEPL[C(CAM)]DNV | 500 | TGIYHGGEPL[C(CAM)]DNVNTQ | 1200 |
| | NTQR | | [R(13C6; 15N4)] | |
| PIK3CA_5 | L[C(CAM)]VLEYQGK | 501 | L[C(CAM)]VLEYQG[K(13C6; | 1201 |
| | | | 15N2)] | |
| PIK3CA_6 | SLWVINSALR | 502 | SLWVINSAL[R(13C6; 15N4)] | 1202 |
| PIK3R1_1 | FSAASSDNTENLIK | 503 | FSAASSDNTENLI[K(13C6; | 1203 |
| | | | 15N2)] | |
| PIK3R1_2 | IFEEQ[C(CAM)]QTQER | 504 | IFEEQ[C(CAM)]QTQE[R(13C6; | 1204 |
| | | | 15N4)] | |
| PIK3R1_3 | YGFSDPLTFSSVVELINHYR | 505 | YGFSDPLTFSSVVELINHY | 1205 |
| | | | [R(13C6; 15N4)] | |
| PIK3R1_3 | TQSSSNLAELR | 506 | TQSSSNLAEL[R(13C6; 15N4)] | 1206 |
| PIK3R1_4 | TWNVGSSNR | 507 | TWNVGSSN[R(13C6; 15N4)] | 1207 |
| PIK3R2_1 | ALGATFGPLLLR | 508 | ALGATFGPLLL[R(13C6; 15N4)] | 1208 |
| PIK3R2_2 | SFLLALPAPLVTPEASAEAR | 509 | SFLLALPAPLVTPEASAEA | 1209 |
| | | | [R(13C6; 15N4)] | |
| PIK3R2_3 | AALQALGVAEGGER | 510 | AALQALGVAEGGE[R(13C6; | 1210 |
| | | | 15N4)] | |
| PIK3R2_4 | TDWSLSDVDQWDTAALAD | 511 | TDWSLSDVDQWDTAALADGI | 1211 |
| | GIK | | [K(13C6; 15N2)] | |
| PIK3R2_5 | EYDQLYEEYTR | 512 | EYDQLYEEYT[R(13C6; 15N4)] | 1212 |
| PSMG1_1 | TSLEVSLLEK | 513 | TSLEVSLLE[K(13C6; 15N2)] | 1213 |
| PSMG1_2 | LWNEW[C(CAM)]R | 514 | LWNEW[C(CAM)][R(13C6; | 1214 |
| | | | 15N4)] | |
| PSMG1_3 | TTDTTHLSSTEAF[C(CAM)] | 515 | TTDTTHLSSTEAF[C(CAM)] | 1215 |
| | VFYHLK | | VFYHL[K(13C6; 15N2)] | |
| PSMG1_4 | VFGS[C(CAM)]PR | 516 | VFGS[C(CAM)]P[R(13C6; | 1216 |
| | | | 15N4)] | |
| PSMG1_5 | HVTDYK | 517 | HVTDY[K(13C6; 15N2)] | 1217 |
| PSMG1_6 | TSESTGSLPSPFLR | 518 | TSESTGSLPSPFL[R(13C6; | 1218 |
| | | | 15N4)] | |
| PSMG1_7 | LDLITVEAFKPILSTR | 519 | LDLITVEAFKPILST[R(13C6; | 1219 |
| | | | 15N4)] | |
| PSMG1_8 | NIPQSTEILK | 520 | NIPQSTEIL[K(13C6; 15N2)] | 1220 |
| RAF1_1 | YSTPHAFTFNTSSPSSEGSLS | 521 | YSTPHAFTFNTSSPSSEGSLSQ | 1221 |
| | QR | | [R(13C6; 15N4)] | |
| RAF1_2 | AAHTEDINA[C(CAM)]TLTTS | 522 | AAHTEDINA[C(CAM)]TLTTSP | 1222 |
| | PR | | [R(13C6; 15N4)] | |
| RASA1_1 | LLYPVAPPEPVEDR | 523 | LLYPVAPPEPVED[R(13C6; | 1223 |
| | | | 15N4)] | |
| RASA1_2 | VPDTDEISFLK | 524 | VPDTDEISFL[K(13C6; 15N2)] | 1224 |
| RASA1_3 | YYNSIGDIIDHYR | 525 | YYNSIGDIIDHY[R(13C6; | 1225 |
| | | | 15N4)] | |
| RASA1_4 | GHATDEWFLLSSHIPLK | 526 | GHATDEWFLLSSHIPL[K(13C6; | 1226 |
| | | | 15N2)] | |
| RASA1_5 | VVSGFVFLR | 527 | VVSGFVFL[R(13C6; 15N4)] | 1227 |
| RASA1_6 | LI[C(CAM)]PAILNPR | 528 | LI[C(CAM)]PAILNP[R(13C6; | 1228 |
| | | | 15N4)] | |
| RASA1_7 | DLAALHEI[C(CAM)]VAHSD | 529 | DLAALHEI[C(CAM)]VAHSDEL | 1229 |
| | ELR | | [R(13C6; 15N4)] | |
| RASA1_8 | LLAITELLQQK | 530 | LLAITELLQQ[K(13C6; 15N2)] | 1230 |
| RASGRF1_ | YLHLLQIVETEK | 531 | YLHLLQIVETE[K(13C6; 15N2)] | 1231 |
| 1 | | | | |
| RASGRF1_ | AEITSLLK | 532 | AEITSLL[K(13C6; 15N2)] | 1232 |
| 2 | | | | |
| RASGRF1_ | TIIQDYIR | 533 | TIIQDYI[R(13C6; 15N4)] | 1233 |
| 3 | | | | |
| RASGRF1_ | HLII[C(CAM)]TR | 534 | HLII[C(CAM)15N4)] | 1234 |
| 4 | | | | |
| RASGRF1_ | AAWTSDISQ[C(CAM)]VDNI | 535 | AAWTSDISQ[C(CAM)]VDNI | 1235 |
| 5 | R | | [R(13C6; 15N4)] | |
| RASGRF1_ | SDASLY[C(CAM)]DDVDIR | 536 | SDASLY[C(CAM)]DDVDI[R(13C6; | 1236 |
| 6 | | | 15N4)] | |
| RASGRF1_ | KPISAIPAR | 537 | KPISAIPA[R(13C6; 15N4)] | 1237 |
| 7 | | | | |
| RASGRF1_ | IPDEGDTTPEKPEDPSALSK | 538 | IPDEGDTTPEKPEDPSALS[K(13C6; | 1238 |
| 8 | | | 15N2)] | |
| RASGRF1_ | SALSAASAFAIATAGANEGT | 539 | SALSAASAFAIATAGANEGTPN | 1239 |
| 9 | PNK | | [K(13C6; 15N2)] | |
| RASGRF1_ | SALSAASAFAIATAGANEGT | 540 | SALSAASAFAIATAGANEGTP | 1240 |
| 10 | PNKEK | | NKE[K(13C6; 15N2)] | |
| RASGRF1_ | HSQDFETNDELK | 541 | HSQDFETNDEL[K(13C6; 15N2)] | 1241 |
| 11 | | | | |
| RASGRF1_ | HSQDFETNDELK[C(CAM)]K | 542 | HSQDFETNDELK[C(CAM)] | 1242 |
| 12 | | | [K(13C6; 15N2) | |
| RASGRF2_ | YIHLVQIVETEK | 543 | YIHLVQIVETE[K(13C6; 15N2)] | 1243 |
| 1 | | | | |
| RASGRF2_ | HQLEDQDTEIER | 544 | HQLEDQDTEIE[R(13C6; 15N4)] | 1244 |
| 2 | | | | |
| RASGRF2_ | HFLI[C(CAM)]TR | 545 | HFLI[C(CAM)15N4)] | 1245 |
| 3 | | | | |
| RASGRF2_ | AAWMSDISQ[C(CAM)] | 546 | AAWMSDISQ[C(CAM)]VDNI | 1246 |
| 4 | VDNIR | | [R(13C6; 15N4)] | |
| RASGRF2_ | FLSIDFLNTFLHTYR | 547 | FLSIDFLNTFLHTY[R(13C6; | 1247 |
| 5 | | | 15N4)] | |
| RASGRF2_ | IFTTAAVVLGK | 548 | IFTTAAVVLG[K(13C6; 15N2)] | 1248 |
| 6 | | | | |
| RASGRF2_ | FSSPPPLAVSR | 549 | FSSPPPLAVS[R(13C6; 15N4)] | 1249 |
| 7 | | | | |
| RASGRF2_ | GLSSPEQSPGTVEENVDNPR | 550 | GLSSPEQSPGTVEENVDNP | 1250 |
| 8 | | | [R(13C6; 15N4)] | |
| RASGRF2_ | AVLESAPADR | 551 | AVLESAPAD[R(13C6; 15N4)] | 1251 |
| 9 | | | | |
| RASGRF2_ | AGVESSPAADTTELSP | 552 | AGVESSPAADTTELSP[C(CAM)] | 1252 |
| 10 | [C(CAM)]R | | [R(13C6; 15N4)] | |
| RASGRF2_ | HAQDFELNNELK | 553 | HAQDFELNNEL[K(13C6; | 1253 |
| 11 | | | 15N2)] | |
| RASGRF2_ | ALSQDDQDDIHLK | 554 | ALSQDDQDDIHL[K(13C6; | 1254 |
| 12 | | | 15N2)] | |
| RASGRF2_ | SIPYEEFLGQGWMK | 555 | SIPYEEFLGQGWM[K(13C6; | 1255 |
| 13 | | | 15N2)] | |
| RICTOR_1 | EILQNVAR | 556 | EILQNVA[R(13C6; 15N4)] | 1256 |
| RICTOR_2 | LL[C(CAM)]DIGHSEEK | 557 | LL[C(CAM)]DIGHSEE[K(13C6; | 1257 |
| | | | 15N2)] | |
| RICTOR_3 | LALLNEAK | 558 | LALLNEA[K(13C6; 15N2)] | 1258 |
| RICTOR_4 | YLIQDSSILQK | 559 | YLIQDSSILQ[K(13C6; 15N2)] | 1259 |
| RICTOR_5 | ILAPYTDFHYR | 560 | ILAPYTDFHY[R(13C6; 15N4)] | 1260 |
| RICTOR_6 | LSDGFVAAEAK | 561 | LSDGFVAAEA[K(13C6; 15N2)] | 1261 |
| RICTOR_7 | LYANLDLDFAK | 562 | LYANLDLDFA[K(13C6; 15N2)] | 1262 |
| RICTOR_8 | LTVSSLDYSR | 563 | LTVSSLDYS[R(13C6; 15N4)] | 1263 |
| RICTOR_9 | ILTAATDA[C(CAM)]R | 564 | ILTAATDA[C(CAM)][R(13C6; | 1264 |
| | | | 15N4)] | |
| RICTOR_10 | TG[C(CAM)]HLLEVQNIITEL | 565 | TG[C(CAM)]HLLEVQNIITEL | 1265 |
| | [C(CAM)]R | | [C(CAM)][R(13C6; 15N4)] | |
| RICTOR_11 | HIEDTGSTPSIGENDLK | 566 | HIEDTGSTPSIGENDL[K(13C6; | 1266 |
| | | | 15N2)] | |
| RICTOR_12 | TSHYLTPQSNHLSLSK | 567 | TSHYLTPQSNHLSLS[K(13C6; | 1267 |
| | | | 15N2)] | |
| RICTOR_13 | SNSVSLVPPGSSHTLPR | 568 | SNSVSLVPPGSSHTLP[R(13C6; | 1268 |
| | | | 15N4)] | |
| RICTOR_14 | DIPYFQTK | 569 | DIPYFQT[K(13C6; 15N2)] | 1269 |
| RICTOR_15 | AFAHDAGGLPSGTGGLVK | 570 | AFAHDAGGLPSGTGGLV | 1270 |
| | | | [K(13C6; 15N2)] | |
| RICTOR_1 | STELLLGVK | 571 | STELLLGV[K(13C6; 15N2)] | 1271 |
| 6 | | | | |
| RPS6KA1_ | EISITHHVK | 572 | EISITHHV[K(13C6; 15N2)] | 1272 |
| 1 | | | | |
| RPS6KA1_ | DILADVNHPFVVK | 573 | DILADVNHPFVV[K(13C6; | 1273 |
| 2 | | | 15N2)] | |
| RPS6KA1_ | HVFYSTIDWNK | 574 | HVFYSTIDWN[K(13C6; 15N2)] | 1274 |
| 3 | | | | |
| RPS6KA1_ | GFSFVATGLMEDDGKPR | 575 | GFSFVATGLMEDDGKP[R(13C6; | 1275 |
| 4 | | | 15N4)] | |
| RPS6KA1_ | APQAPLHSVVQQLHGK | 576 | APQAPLHSVVQQLHG[K(13C6; | 1276 |
| 5 | | | 15N2)] | |
| RPS6KA1_ | NLVFSDGYVVK | 577 | NLVFSDGYVV[K(13C6; 15N2)] | 1277 |
| 6 | | | | |
| RPS6KA1_ | ATNMEYAVK | 578 | ATNMEYAV[K(13C6; 15N2)] | 1278 |
| 7 | | | | |
| RPS6KA1_ | EASFVLHTIGK | 579 | EASFVLHTIG[K(13C6; 15N2)] | 1279 |
| 8 | | | | |
| RPS6KA1_ | TVEYLHSQGVVHR | 580 | TVEYLHSQGVVH[R(13C6; | 1280 |
| 9 | | | 15N4)] | |
| RPS6KA1_ | DLKPSNILYVDESGNPE | 581 | DLKPSNILYVDESGNPE | 1281 |
| 10 | [C(CAM)]LR | | [C(CAM)]L[R(13C6; 15N4)] | |
| RPS6KA2_ | VLGQGSYGK | 582 | VLGQGSYG[K(13C6; 15N2)] | 1282 |
| 1 | | | | |
| RPS6KA2_ | DILAEVNHPFIVK | 583 | DILAEVNHPFIV[K(13C6; | 1283 |
| 2 | | | 15N2)] | |
| RPS6KA2_ | LGAGIDGVEEIK | 584 | LGAGIDGVEEI[K(13C6; 15N2)] | 1284 |
| 3 | | | | |
| RPS6KA2_ | LGAGIDGVEEIKR | 585 | LGAGIDGVEEIK[R(13C6; | 1285 |
| 4 | | | 15N4)] | |
| RPS6KA2_ | HPFFVTIDWNTLYR | 586 | HPFFVTIDWNTLY[R(13C6; | 1286 |
| 5 | | | 15N4)] | |
| RPS6KA2_ | TPTDSPGVPPSANAHHLFR | 587 | TPTDSPGVPPSANAHHLF | 1287 |
| 6 | | | [R(13C6; 15N4)] | |
| RPS6KA2_ | ATDTEYAVK | 588 | ATDTEYAV[K(13C6; 15N2)] | 1288 |
| 7 | | | | |
| RPS6KA2_ | EASDVL[C(CAM)]TITK | 589 | EASDVL[C(CAM)]TIT[K(13C6; | 1289 |
| 8 | | | 15N2)] | |
| RPS6KA2_ | YALSGGNWDSISDAAK | 590 | YALSGGNWDSISDAA[K(13C6; | 1290 |
| 9 | | | 15N2)] | |
| RPS6KA2_ | LEPVLSSNLAQR | 591 | LEPVLSSNLAQ[R(13C6; 15N4)] | 1291 |
| 10 | | | | |
| RPS6KA3_ | HSFFSTIDWNK | 592 | HSFFSTIDWN[K(13C6; 15N2)] | 1292 |
| 1 | | | | |
| RPS6KA3_ | EIHPPFKPATGRPEDTFYFDP | 593 | EIHPPFKPATGRPEDTFYFDPEF | 1293 |
| 2 | EFTAK | | TA[K(13C6; 15N2)] | |
| RPS6KA3_ | DSPGIPPSANAHQLFR | 594 | DSPGIPPSANAHQLF[R(13C6; | 1294 |
| 3 | | | 15N4)] | |
| RPS6KA3_ | NSIQFTDGYEVK | 595 | NSIQFTDGYEV[K(13C6; 15N2)] | 1295 |
| 4 | | | | |
| RPS6KA3_ | ATNMEFAVK | 596 | ATNMEFAV[K(13C6; 15N2)] | 1296 |
| 5 | | | | |
| RPS6KA3_ | EASAVLFTITK | 597 | EASAVLFTIT[K(13C6; 15N2)] | 1297 |
| 6 | | | | |
| RPS6KA3_ | TVEYLHAQGVVHR | 598 | TVEYLHAQGVVH[R(13C6; | 1298 |
| 7 | | | 15N4)] | |
| RPS6KA3_ | DLKPSNILYVDESGNPESIR | 599 | DLKPSNILYVDESGNPESI | 1299 |
| 8 | | | [R(13C6; 15N4)] | |
| RPS6KA3_ | LTAALVLR | 600 | LTAALVL[R(13C6; 15N4)] | 1300 |
| 9 | | | | |
| RPS6KA3_ | HPWIVHWDQLPQYQLNR | 601 | HPWIVHWDQLPQYQLN[R(13C6; | 1301 |
| 10 | | | 15N4)] | |
| RPS6KA3_ | GAMAATYSALNR | 602 | GAMAATYSALN[R(13C6; | 1302 |
| 11 | | | 15N4)] | |
| RPS6KA3_ | NQSPVLEPVGR | 603 | NQSPVLEPVG[R(13C6; 15N4)] | 1303 |
| 12 | | | | |
| RPS6KA5_ | VGIENFELLK | 604 | VGIENFELL[K(13C6; 15N2)] | 1304 |
| 1 | | | | |
| RPS6KA5_ | DLKPENLLFTDENDNLEIK | 605 | DLKPENLLFTDENDNLEI | 1305 |
| 2 | | | [K(13C6; 15N2)] | |
| RPS6KA5_ | EFVADETER | 606 | EFVADETE[R(13C6; 15N4)] | 1306 |
| 3 | | | | |
| RPS6KA5_ | DSPFYQHYDLDLK | 607 | DSPFYQHYDLDL[K(13C6; | 1307 |
| 4 | | | 15N2)] | |
| RPS6KA5_ | L[C(CAM)]EGHPNIVK | 608 | L[C(CAM)]EGHPNIV[K(13C6; | 1308 |
| 5 | | | 15N2)] | |
| RPS6KA5_ | DKPLGEGSFSI[C(CAM)]R | 609 | DKPLGEGSFSI[C(CAM)][R(13C6; | 1309 |
| 6 | | | 15N4)] | |
| RPS6KA5_ | DLIQGLLTVDPNK | 610 | DLIQGLLTVDPN[K(13C6; | 1310 |
| 7 | | | 15N2)] | |
| RPS6KB2_ | AAGLEPVGHYEEVELTETSV | 611 | AAGLEPVGHYEEVELTETSVN | 1311 |
| 1 | NVGPER | | VGPE[R(13C6; 15N4)] | |
| RPS6KB2_ | IGPH[C(CAM)]FELLR | 612 | IGPH[C(CAM)]FELL[R(13C6; | 1312 |
| 2 | | | 15N4)] | |
| RPS6KB2_ | NILESVK | 613 | NILESV[K(13C6; 15N2)] | 1313 |
| 3 | | | | |
| RPS6KB2_ | HPFIVELAYAFQTGGK | 614 | HPFIVELAYAFQTGG[K(13C6; | 1314 |
| 4 | | | 15N2)] | |
| RPS6KB2_ | IGGGPGDAADVQR | 615 | IGGGPGDAADVQ[R(13C6; | 1315 |
| 5 | | | 15N4)] | |
| RPS6KB2_ | VDPPFRP[C(CAM)]LQSEEDV | 616 | VDPPFRP[C(CAM)]LQSEEDVS | 1316 |
| 6 | SQFDTR | | QFDT[R(13C6; 15N4)] | |
| RPS6KB2_ | EGFSFQPK | 617 | EGFSFQP[K(13C6; 15N2)] | 1317 |
| 7 | | | | |
| RPTOR_1 | ALETIGANLQK | 618 | ALETIGANLQ[K(13C6; 15N2)] | 1318 |
| RPTOR_2 | IWQNFDSAR | 619 | IWQNFDSA[R(13C6; 15N4)] | 1319 |
| RPTOR_3 | LYSLLSDPIPEVR | 620 | LYSLLSDPIPEV[R(13C6; 15N4)] | 1320 |
| RPTOR_4 | SVSSYGNIR | 621 | SVSSYGNI[R(13C6; 15N4)] | 1321 |
| RPTOR_5 | VLDTSSLTQSAPASPTNK | 622 | VLDTSSLTQSAPASPTN[K(13C6; | 1322 |
| | | | 15N2)] | |
| RPTOR_6 | LDDQIFLNR | 623 | LDDQIFLN[R(13C6; 15N4)] | 1323 |
| RPTOR_7 | VQDIPTGADS[C(CAM)]VTSLS | 624 | VQDIPTGADS[C(CAM)]VTSLS | 1324 |
| | [C(CAM)]DSHR | | [C(CAM)]DSH[R(13C6; 15N4)] | |
| RPTOR_8 | SLIVAGLGDGSIR | 625 | SLIVAGLGDGSI[R(13C6; | 1325 |
| | | | 15N4)] | |
| SMAD4_1 | Y[C(CAM)]QYAFDLK | 626 | Y[C(CAM)]QYAFDL[K(13C6; | 1326 |
| | | | 15N2)] | |
| SMAD4_2 | [C(CAM)]DSV[C(CAM)] | 627 | [C(CAM)]DSV[C(CAM)]VNPYHYE | 1327 |
| | VNPYHYER | | [R(13C6; 15N4)] | |
| SMAD4_3 | F[C(CAM)]LGQLSNVHR | 628 | F[C(CAM)]LGQLSNVH[R(13C6; | 1328 |
| | | | 15N4)] | |
| SMAD4_4 | GEGDVWVR | 629 | GEGDVWV[R(13C6; 15N4)] | 1329 |
| SMAD4_5 | [C(CAM)]LSDHAVFVQSYYL | 630 | [C(CAM)]LSDHAVFVQSYYLD | 1330 |
| | DR | | [R(13C6; 15N4)] | |
| SMAD4_6 | IYPSAYIK | 631 | IYPSAYI[K(13C6; 15N2)] | 1331 |
| SMAD4_7 | GWGPDYPR | 632 | GWGPDYP[R(13C6; 15N4)] | 1332 |
| SOS1_1 | IAIENGDQPLSAELK | 633 | IAIENGDQPLSAEL[K(13C6; | 1333 |
| | | | 15N2)] | |
| SOS1_2 | LFSANDVENIFSR | 634 | LFSANDVENIFS[R(13C6; | 1334 |
| | | | 15N4)] | |
| SOS1_3 | IVDIHELSVK | 635 | IVDIHELSV[K(13C6; 15N2)] | 1335 |
| SOS1_4 | LDHTFEQIPSR | 636 | LDHTFEQIPS[R(13C6; 15N4)] | 1336 |
| SPRED1_1 | VTPTFHHWK | 637 | VTPTFHHW[K(13C6; 15N2)] | 1337 |
| SPRED1_2 | AIEDISQG[C(CAM)]PESK | 638 | AIEDISQG[C(CAM)]PES[K(13C6; | 1338 |
| | | | 15N2)] | |
| SPRED1_3 | NEAEGADDLQANEEDSSSSL | 639 | NEAEGADDLQANEEDSSSSLV | 1339 |
| | VK | | [K(13C6; 15N2)] | |
| SPRED1_4 | DHLFQQETVVTSEPYR | 640 | DHLFQQETVVTSEPY[R(13C6; | 1340 |
| | | | 15N4)] | |
| SPRED1_5 | SSNIRPSPFEDLNAR | 641 | SSNIRPSPFEDLNA[R(13C6; | 1341 |
| | | | 15N4)] | |
| SPRED1_6 | HVSFQDEDEIVR | 642 | HVSFQDEDEIV[R(13C6; 15N4)] | 1342 |
| SPRED1_7 | TQPSSLK | 643 | TQPSSL[K(13C6; 15N2)] | 1343 |
| SPRED1_8 | FNHEENVR | 644 | FNHEENV[R(13C6; 15N4)] | 1344 |
| SPRED2_1 | DDSSGGWFPQEGGGISR | 645 | DDSSGGWFPQEGGGIS[R(13C6; | 1345 |
| | | | 15N4)] | |
| SPRED2_2 | LVVLE[C(CAM)]YVR | 646 | LVVLE[C(CAM)]YV[R(13C6; | 1346 |
| | | | 15N4)] | |
| SPRED2_3 | TISSPTS[C(CAM)]EHR | 647 | TISSPTS[C(CAM)]EH[R(13C6; | 1347 |
| | | | 15N4)] | |
| SPRED2_4 | EKIWMTGYEDYR | 648 | EKIWMTGYEDY[R(13C6; | 1348 |
| | | | 15N4)] | |
| SPRED2_5 | IWMTGYEDYR | 649 | IWMTGYEDY[R(13C6; 15N4)] | 1349 |
| SPRED2_6 | GKYPDPSEDADSSYVR | 650 | GKYPDPSEDADSSYV[R(13C6; | 1350 |
| | | | 15N4)] | |
| SPRED2_7 | YPDPSEDADSSYVR | 651 | YPDPSEDADSSYV[R(13C6; | 1351 |
| | | | 15N4)] | |
| SPRED2_8 | HDYNYPYVDSSDFGLGEDP | 652 | HDYNYPYVDSSDFGLGEDP | 1352 |
| | K | | [K(13C6; 15N2)] | |
| SPRED2_9 | GH[C(CAM)]QDAPDSVR | 653 | GH[C(CAM)]QDAPDSV[R(13C6; | 1353 |
| | | | 15N4)] | |
| SPRY1_1 | EIQPTAILSLDQIK | 654 | EIQPTAILSLDQI[K(13C6; | 1354 |
| | | | 15N2)] | |
| SPRY1_2 | GSNEYTEGPSVVK | 655 | GSNEYTEGPSVV[K(13C6; | 1355 |
| | | | 15N2)] | |
| SPRY1_3 | HTSHLGHAVLPSNAR | 656 | HTSHLGHAVLPSNA[R(13C6; | 1356 |
| | | | 15N4)] | |
| SPRY1_4 | FI[C(CAM)]EQ[C(CAM)]GK | 657 | FI[C(CAM)]EQ[C(CAM)]G | 1357 |
| | | | [K(13C6; 15N2)] | |
| SPRY1_5 | SPPTRPVPGHR | 658 | SPPTRPVPGH[R(13C6; 15N4)] | 1358 |
| SPRY2_1 | DALTQQVHVLSLDQIR | 659 | DALTQQVHVLSLDQI[R(13C6; | 1359 |
| | | | 15N4)] | |
| SPRY2_2 | LHGLPEHR | 660 | LHGLPEH[R(13C6; 15N4)] | 1360 |
| SPRY2_3 | LLGSSFSSGPVADGIIR | 661 | LLGSSFSSGPVADGII[R(13C6; | 1361 |
| | | | 15N4)] | |
| SPRY2_4 | EDLGLHAYR | 662 | EDLGLHAY[R(13C6; 15N4)] | 1362 |
| SPRY2_5 | SELKPGELKPLSK | 663 | SELKPGELKPLS[K(13C6; | 1363 |
| | | | 15N2)] | |
| SPRY2_6 | LQHSQVHSSAR | 664 | LQHSQVHSSA[R(13C6; 15N4)] | 1364 |
| SPRY3_1 | STHASNDYVERPPAP | 665 | STHASNDYVERPPAP[C(CAM)] | 1365 |
| | [C(CAM)]K | | [K(13C6; 15N2)] | |
| SPRY3_2 | LLASITPSPSGQSIIR | 666 | LLASITPSPSGQSII[R(13C6; | 1366 |
| | | | 15N4)] | |
| SPRY3_3 | QALSSPSLIVQTHK | 667 | QALSSPSLIVQTH[K(13C6; | 1367 |
| | | | 15N2)] | |
| SPRY3_4 | [C(CAM)]L[C(CAM)]SAESLL | 668 | [C(CAM)]L[C(CAM)]SAESLLD | 1368 |
| | DYGT[C(CAM)]L[C(CAM)] | | YGT[C(CAM)]L[C(CAM)] | |
| | [C(CAM)]VK | | [C(CAM)]V[K(13C6; 15N2)] | |
| SPRY4_1 | LQHPLTILPIDQVK | 669 | LQHPLTILPIDQV[K(13C6; | 1369 |
| | | | 15N2)] | |
| SPRY4_2 | TSHVENDYIDNPSLALTTGP | 670 | TSHVENDYIDNPSLALTTGP | 1370 |
| | K | | [K(13C6; 15N2)] | |
| SPRY4_3 | TRGGAPELAPTPAR | 671 | TRGGAPELAPTPA[R(13C6; | 1371 |
| | | | 15N4)] | |
| SPRY4_4 | GGAPELAPTPAR | 672 | GGAPELAPTPA[R(13C6; 15N4)] | 1372 |
| SPRY4_5 | [C(CAM)]DQDVTHHWISFSG | 673 | [C(CAM)]DQDVTHHWISFSGRP | 1373 |
| | RPSSVSSSSSTSSDQR | | SSVSSSSSTSSDQ[R(13C6; | |
| | | | 15N4)] | |
| SPRY4_6 | LLDHMAPPPVADQASPR | 674 | LLDHMAPPPVADQASP[R(13C6; | 1374 |
| | | | 15N4)] | |
| SPRY4_7 | VVH[C(CAM)]QPLDLK | 675 | VVH[C(CAM)]QPLDL[K(13C6; | 1375 |
| | | | 15N2)] | |
| SPRY4_8 | HFLL[C(CAM)]EA[C(CAM)] | 676 | HFLL[C(CAM)]EA[C(CAM)]G | 1376 |
| | GK | | [K(13C6; 15N2)] | |
| SPRY4_9 | HTNSVI[C(CAM)]K | 677 | HTNSVI[C(CAM)][K(13C6; | 1377 |
| | | | 15N2)] | |
| STAT3_1 | FLQESNVLYQHNLR | 678 | FLQESNVLYQHNL[R(13C6; | 1378 |
| | | | 15N4)] | |
| STAT3_2 | [C(CAM)]LWEESR | 679 | [C(CAM)]LWEES[R(13C6; | 1379 |
| | | | 15N4)] | |
| STAT3_3 | LLQTAATAAQQGGQANHPT | 680 | LLQTAATAAQQGGQANHPTA | 1380 |
| | AAVVTEK | | AVVTE[K(13C6; 15N2)] | |
| STAT3_4 | SAFVVER | 681 | SAFVVE[R(13C6; 15N4)] | 1381 |
| STAT3_5 | FPELNYQLK | 682 | FPELNYQL[K(13C6; 15N2)] | 1382 |
| STAT3_6 | FNILGTNTK | 683 | FNILGTNT[K(13C6; 15N2)] | 1383 |
| STAT3_7 | GLSIEQLTTLAEK | 684 | GLSIEQLTTLAE[K(13C6; | 1384 |
| | | | 15N2)] | |
| STAT3_8 | AILSTKPPGTFLLR | 685 | AILSTKPPGTFLL[R(13C6; | 1385 |
| | | | 15N4)] | |
| STAT3_9 | TQIQSVEPYTK | 686 | TQIQSVEPYT[K(13C6; 15N2)] | 1386 |
| STK11_1 | IDSTEVIYQPR | 687 | IDSTEVIYQP[R(13C6; 15N4)] | 1387 |
| STK11_2 | ISDLGVAEALHPFAADDT | 688 | ISDLGVAEALHPFAADDT | 1388 |
| | [C(CAM)]R | | [C(CAM)][R(13C6; 15N4)] | |
| STK11_3 | GSYAIPGD[C(CAM)]GPPLSD | 689 | GSYAIPGD[C(CAM)]GPPLSDLL | 1389 |
| | LLK | | [K(13C6; 15N2)] | |
| STK11_4 | NVIQLVDVLYNEEK | 690 | NVIQLVDVLYNEE[K(13C6; | 1390 |
| | | | 15N2)] | |
| TP53_1 | TYQGSYGFR | 691 | TYQGSYGF[R(13C6; 15N4)] | 1391 |
| TP53_2 | LGFLHSGTAK | 692 | LGFLHSGTA[K(13C6; 15N2)] | 1392 |
| TP53_3 | SVT[C(CAM)]TYSPALNK | 693 | SVT[C(CAM)]TYSPALN[K(13C6; | 1393 |
| | | | 15N2)] | |
| TP53_4 | T[C(CAM)]PVQLWVDSTPPP | 694 | T[C(CAM)]PVQLWVDSTPPPGT | 1394 |
| | GTR | | [R(13C6; 15N4)] | |
| TP53_5 | [C(CAM)]SDSDGLAPPQHLIR | 695 | [C(CAM)]SDSDGLAPPQHLI | 1395 |
| | | | [R(13C6; 15N4)] | |
| TP53_6 | VEYLDDR | 696 | VEYLDD[R(13C6; 15N4)] | 1396 |
| TP53_7 | RPILTIITLEDSSGNLLGR | 697 | RPILTIITLEDSSGNLLG[R(13C6; | 1397 |
| | | | 15N4)] | |
| TP53_8 | ALPNNTSSSPQPK | 698 | ALPNNTSSSPQP[K(13C6; | 1398 |
| | | | 15N2)] | |
| TP53_9 | ELNEALELK | 699 | ELNEALEL[K(13C6; 15N2)] | 1399 |
| TP53_10 | ALPNNTSS[S(PO3H2)]PQPK | 700 | ALPNNTSS[S(PO3H2)]PQP | 1400 |
| | | | [K(13C6; 15N2)] | |

In some embodiments, the peptides reagents, and the peptides useful in the methods, are recited in Table 5 (SEQ ID Nos: 13, 15, 18, 28, 31, 47, 48, 51, 127, 128, 132, 133, 141, 142, 143, 144, 171, 173, 177, 180, 181, 182, 183, 190, 192, 213, 219, 220, 224, 226, 231, 233, 234, 235, 236, 258, 270, 295, 297, 300, 301, 302, 304, 306, 308, 310, 347, 348, 353, 362, 392, 393, 405, 416, 429, 443, 446, 451, 467, 483, 487, 501, 502, 506, 507, 510, 512, 521, 522, 523, 528, 572, 580, 588, 591, 598, 603, 615, 617, 635, 636, 655, 657, 660, 662, 670, 678, 683, 691, 692, 696, 698, and 699). In some embodiments, the peptides of Table 5 are useful in multi-plex MS methods.

In some embodiments, protein samples are denatured or solubilized before fragmentation.

In some embodiments, the fragmentation protocol uses chemical cleavage. In some embodiments, the chemical cleavage uses CNBr. In some embodiments, the fragmentation protocol is done using an enzyme. In some embodiments, the fragmentation protocol uses MS-grade commercially available proteases. Examples of proteases that may be used to digest samples include trypsin, endoproteinase GluC, endoproteinase ArgC, pepsin, chymotrypsin, LysN protease, LysC protease, GluC protease, AspN protease, proteinase K, and thermolysin. In some embodiments, a mixture of different proteases are used and the individual results are combined together after the digestion and analysis. In some embodiments, the digestion is incomplete in order to see larger, overlapping peptides. In some embodiments, the antibody digestion is performed with IdeS, IdeZ, pepsin, or papain to generate large antibody domains for “middle-down” protein characterization. In some embodiments, the fragmentation protocol uses trypsin that is modified. In some embodiments, a protein:protease ratio (w/w) of 10:1, 20:1, 25:1, 50:1, 66:1, or 100:1 may be used. In some embodiments, the trypsin used is at a concentration of about 100 ng/ml-1 mg/ml, or about 100 ng/ml-500 μg/ml, or about 100 ng/ml-100 μg/ml, or about 1 μg/ml-1 mg/ml, or about 1 μg/ml-500 μg/ml, or about 1 μg/ml-100 μg/ml, or about 10 μg/mg-1 mg/ml, or about 10 μg/mg-500 μg/ml, or about 10 μg/mg-100 μg/ml. In some embodiments, the digestion step is for about 10 minutes to 48 hours, or about 30 minutes to 48 hours, or about 30 minutes to 24 hours, or about 30 minutes to 16 hours, or about 1 hour to 48 hours, or about 1 hour to 24 hours, or about 1 hour to 16 hours, or about 1 to 8 hours, or about 1 to 6 hours, or about 1 to 4 hours. In some embodiments, the digestion step is incubated at a temperature from about 20° C. to 45° C., or from about 20° C. to 40° C., or from about 22° C. to 40° C., or from about 25° C. to 37° C. In some embodiments, the digestion step is incubated at about 37° C. or 30° C. In some embodiments, a step is included to end the digestion step. The step to end the digestion protocol may be addition of a stop solution or a step of spinning or pelleting of a sample. In some embodiments, the digestion is followed by guanidination.

In some embodiments, the fragmentation protocol includes use of protein gels. In some embodiments, the fragmentation protocol comprises in-gel digestion. An exemplary commercially available kit for performing in-gel digestion is the In-Gel Tryptic Digestion Kit (Thermo Fisher Cat #89871).

In some embodiments, the fragmentation protocol is carried out in solution. An exemplary commercially available kit for performing in-solution digestion is the In-Solution Tryptic Digestion and Guanidination Kit (Thermo Fisher Cat #89895).

In some embodiments, the fragmentation protocol uses beads. In some embodiments, the fragmentation protocol comprises on-bead digestion. In some embodiments, agarose beads or Protein G beads are used. In some embodiments, magnetic beads are used.

In some embodiments, protein samples are separated using liquid chromatography before MS analysis. In some embodiments, fragmented samples are separated using liquid chromatography before MS analysis.

The IP and IP-MS methods described herein are capable of detecting phosphorylated RAS-RAF-MAPK pathway proteins, including those described in Table 4.

TABLE 4
List of Total RAS-RAF-MAPK Pathway Target Proteins

| Target | | Uniprot | Target | | Uniprot |
|---|---|---|---|---|---|
| ID No. | Target Name | Entry | ID No. | Target Name | Entry |
| 1 | ALK | Q9UM73 | 40 | MAPK1 | P28482 |
| 2 | ARAF | P10398 | 41 | MAPK11 | Q15759 |
| 3 | BRAF | P15056 | 42 | MAPK3 | P27361 |
| 4 | CALM1 | P62158 | 43 | MAPK8 | P45983 |
| 5 | CAMK2A | Q9UQM7 | 44 | MAPK9 | P45984 |
| 6 | CCND1 | P24385 | 45 | MET | P08581 |
| 7 | CDH1 | P12830 | 46 | NF1 | P21359 |
| 8 | CDH2 | P19022 | 47 | NOTCH1 | P46531 |
| 9 | CDK2 | P24941 | 48 | NRAS | P01111 |
| 10 | CDKN1A | P38936 | 49 | PAK1 | Q13153 |
| 11 | CDKN1B | P46527 | 50 | PARP1 | P09874 |
| 12 | CDKN2A | Q8N726 | 51 | PGR | P06401 |
| 13 | CTNNA1 | P35221 | 52 | PIK3CA | P42336 |
| 14 | CTNNB1 | P35222 | 53 | PIK3R1 | P27986 |
| 15 | DDR2 | Q16832 | 54 | PIK3R2 | O00459 |
| 16 | DUSP22 | Q9NRW4 | 55 | PSMG1 | O95456 |
| 17 | DUSP4 | Q13115 | 56 | RAF1 | P04049 |
| 18 | DUSP6 | Q16828 | 57 | RASA1 | P20936 |
| 19 | EGFR | P00533 | 58 | RASGRF1 | Q13972 |
| 20 | EIF2A | Q9BY44 | 59 | RASGRF2 | O14827 |
| 21 | EIF4E | P06730 | 60 | RICTOR | Q6R327 |
| 22 | EIF4EBP1 | Q13541 | 61 | RPS6KA1 | Q15418 |
| 23 | ERBB2 | P04626 | 62 | RPS6KA2 | Q15349 |
| 24 | ERBB3 | P21860 | 63 | RPS6KA3 | P51812 |
| 25 | ERBB4 | Q15303 | 64 | RPS6KB2 | Q9UBS0 |
| 26 | ESR1 | P03372 | 65 | RPTOR | Q8N122 |
| 27 | FBXO7 | Q9Y3I1 | 66 | SMAD3 | P84022 |
| 28 | FGFR1 | P11362 | 67 | SMAD4 | Q13485 |
| 29 | FGFR3 | P22607 | 68 | SOS1 | Q07889 |
| 30 | FOS | P01100 | 69 | SPRED1 | Q7Z699 |
| 31 | FOXO1 | Q12778 | 70 | SPRED2 | Q7Z698 |
| 32 | FOXO3 | O43524 | 71 | SPRY1 | O43609 |
| 33 | HRAS | P01112 | 72 | SPRY2 | O43597 |
| 34 | IQGAP1 | P46940 | 73 | SPRY3 | O43610 |
| 35 | JAK1 | P23458 | 74 | SPRY4 | Q9C004 |
| 36 | JAK2 | O60674 | 75 | STAT1 | P42224 |
| 37 | KRAS | P01116 | 76 | STAT3 | P40763 |
| 38 | MAP2K1 | Q02750 | 77 | STK11 | Q15831 |
| 39 | MAP2K2 | P36507 | 78 | TP53 | P04637 |
| 79 | ATF2 | P15336 | 80 | MAPK12 | P53778 |
| 81 | MAPK13 | O15264 | 82 | MAPK14 | Q16539 |
| 83 | MAP2K3 | P46734 | 84 | MAP2K6 | P52564 |
| 85 | RPS6KA5 | O75582 | 86 | MAPKAPK2 | P49137 |

In some embodiments, the RAS-RAF-MAPK pathway peptides used in the MS methods described herein have limits of detection considered useful in clinical and research methods. See, e.g, Table 5. In some embodiments, the RAS-RAF-MAPK pathway peptides used in the MS and IP-MS methods comprise or consist of the peptides described in Table 5. In some embodiments, the peptides of Table 5 are detectably labelled.

TABLE 5
Lower Limit of Quantitation of Peptides for RAS-RAF-MAPK Pathway Proteins

| | | | Lower |
|---|---|---|---|
| | | | limit of |
| | | SEQ ID | Quantitation |
| Target ID | Native Peptide Sequence | NO: | (fmol) |
| ARAF_2 | IGTGSFGTVFR | 13 | 0.14 |
| ARAF_4 | GLNQD[C(CAM)][C(CAM)]VVYR | 15 | 0.05 |
| ARAF_7 | VSQPTAEQAQAFK | 18 | 0.14 |
| BRAF_3 | LLFQGFR | 28 | 1.23 |
| BRAFV600E_ | IGDFGLATEK | 31 | 0.14 |
| 1 | | | |
| CCND1_1 | AYPDANLLNDR | 47 | 0.14 |
| CCND1_2 | AEET[C(CAM)]APSVSYFK | 48 | 0.05 |
| CCND1_5 | ETIPLTAEK | 51 | 0.05 |
| DUSP4_4 | SGLYSAVIVYDER | 127 | 0.41 |
| DUSP4_5 | FSSEYPEF[C(CAM)]SK | 128 | 0.05 |
| DUSP6_2 | AFYLEGGFSK | 132 | 3.70 |
| DUSP6_3 | ISSDSSSDIESDLDR | 133 | 0.05 |
| EGFR_4 | YSFGAT[C(CAM)]VK | 141 | 0.05 |
| EGFR_5 | YLVIQGDER | 142 | 0.05 |
| EGFR_6 | ELVEPLTPSGEAPNQALLR | 143 | 0.41 |
| EGFR_7 | GSHQISLDNPDYQQDFFPK | 144 | 1.23 |
| ERBB2_5 | VLQGLPR | 171 | 0.05 |
| ERBB2_7 | LLDIDETEYHADGGK | 173 | 0.14 |
| ERBB2_11 | GLQSLPTHDPSPLQR | 177 | 0.41 |
| ERBB2_14 | GPLPTD[C(CAM)][C(CAM)]HEQ[C(CAM)]AAG[C | 180 | 1.23 |
| | (CAM)]TGPK | | |
| ERBB3_1 | G[C(CAM)]LASESSEGHVTGSEAELQEK | 181 | 0.41 |
| ERBB3_2 | GGV[C(CAM)]VTH[C(CAM)]NFLNGEPR | 182 | 33.33 |
| ERBB4_1 | LSSLSDLEQQYR | 183 | 0.41 |
| ERBB4_8 | DGGFAAEQGVSVPYR | 190 | 0.05 |
| ERBB4_10 | YSADPTVFAPER | 192 | 0.05 |
| FGFR1_4 | VYSDPQPHIQWLK | 213 | 11.11 |
| FGFR2_2 | YGPDGLPYLK | 219 | 0.14 |
| FGFR3_1 | LQVLNASHEDSGAYS[C(CAM)]R | 220 | 0.41 |
| FOS_1 | APHPFGVPAPSAGAYSR | 224 | 3.70 |
| FOS_3 | VEQLSPEEEEK | 226 | 0.05 |
| FOXO1_1 | ASLQSGQEGAGDSPGSQFSK | 231 | 0.41 |
| FOXO3_1 | AALQTAPESADDSPSQLSK | 233 | 0.05 |
| FOXO3_2 | SSDELDAWTDFR | 234 | 11.11 |
| HRAS_1 | SFEDIHQYR | 235 | 0.14 |
| HRAS_2 | SYGIPYIETSAK | 236 | 0.41 |
| JAK1_1 | LGSGEYTAEEL[C(CAM)]IR | 258 | 1.23 |
| JAK1_13 | YDPEGDNTGEQVAVK | 270 | 0.14 |
| KRAS_1 | SFEDIHHYR | 295 | 0.41 |
| KRAS_3 | VEDAFYTLVR | 297 | 0.41 |
| KSR1_2 | ADVLEAHEAEAEEPEAGK | 300 | 0.14 |
| MAP2K1_1 | ISELGAGNGGVVFK | 301 | 0.41 |
| MAP2K1_2 | IPEQILGK | 302 | 0.05 |
| MAP2K1_4 | LEELELDEQQR | 304 | 0.05 |
| MAP2K2_1 | ISELGAGNGGVVTK | 306 | 0.14 |
| MAP2K2_3 | IPEEILGK | 308 | 0.05 |
| MAP2K2_5 | LNQPGTPTR | 310 | 0.41 |
| MAPK1_8 | NKVPWNR | 347 | 11.11 |
| MAPK1_9 | LFPNADSK | 348 | 0.05 |
| MAPK11_1 | TVWEVPQR | 353 | 11.11 |
| MAPK11_10 | DLSSIFR | 362 | 0.05 |
| MAPK3_2 | NYLQSLPSK | 392 | 0.14 |
| MAPK3_3 | GQPFDVGPR | 393 | 0.05 |
| MAPK8_3 | YAGYSFEK | 405 | 0.05 |
| MAPK9_6 | ISVDEALR | 416 | 0.05 |
| MET_1 | TGPVLEHPD[C(CAM)]FP[C(CAM)]QD[C(CAM)]SSK | 429 | 0.05 |
| NF1_3 | IDAVY[C(CAM)]HSVELR | 443 | 0.14 |
| NF1_6 | LILNYPK | 446 | 0.41 |
| NF1_11 | LVFID[C(CAM)]PGK | 451 | 0.05 |
| NRAS_2 | SFADINLYR | 467 | 1.23 |
| PARP1_2 | TLGDFAAEYAK | 483 | 0.05 |
| PARP1_6 | VVSEDFLQDVSASTK | 487 | 1.23 |
| PIK3CA_5 | L[C(CAM)]VLEYQGK | 501 | 0.14 |
| PIK3CA_6 | SLWVINSALR | 502 | 1.23 |
| PIK3R1_3 | TQSSSNLAELR | 506 | 0.14 |
| PIK3R1_4 | TWNVGSSNR | 507 | 0.14 |
| PIK3R2_3 | AALQALGVAEGGER | 510 | 0.14 |
| PIK3R2_5 | EYDQLYEEYTR | 512 | 0.41 |
| RAF1_1 | YSTPHAFTFNTSSPSSEGSLSQR | 521 | 3.70 |
| RAF1_2 | AAHTEDINA[C(CAM)]TLTTSPR | 522 | 0.41 |
| RASA1_1 | LLYPVAPPEPVEDR | 523 | 0.41 |
| RASA1_6 | LI[C(CAM)]PAILNPR | 528 | 0.41 |
| RPS6KA1_1 | EISITHHVK | 572 | 1.23 |
| RPS6KA1_9 | TVEYLHSQGVVHR | 580 | 1.23 |
| RPS6KA2_7 | ATDTEYAVK | 588 | 0.14 |
| RPS6KA2_10 | LEPVLSSNLAQR | 591 | 0.05 |
| RPS6KA3_7 | TVEYLHAQGVVHR | 598 | 1.23 |
| RPS6KA3_12 | NQSPVLEPVGR | 603 | 0.05 |
| RPS6KB2_5 | IGGGPGDAADVQR | 615 | 0.05 |
| RPS6KB2_7 | EGFSFQPK | 617 | 0.05 |
| SOS1_3 | IVDIHELSVK | 635 | 3.70 |
| SOS1_4 | LDHTFEQIPSR | 636 | 0.05 |
| SPRY1_2 | GSNEYTEGPSVVK | 655 | 0.14 |
| SPRY1_4 | FI[C(CAM)]EQ[C(CAM)]GK | 657 | 0.14 |
| SPRY2_2 | LHGLPEHR | 660 | 3.70 |
| SPRY2_4 | EDLGLHAYR | 662 | 0.05 |
| SPRY4_2 | TSHVENDYIDNPSLALTTGPK | 670 | 3.70 |
| STAT3_1 | FLQESNVLYQHNLR | 678 | 3.70 |
| STAT3_6 | FNILGTNTK | 683 | 0.05 |
| TP53_1 | TYQGSYGFR | 691 | 0.05 |
| TP53_2 | LGFLHSGTAK | 692 | 1.23 |
| TP53_6 | VEYLDDR | 696 | 0.14 |
| TP53_8 | ALPNNTSSSPQPK | 698 | 0.14 |
| TP53_9 | ELNEALELK | 699 | 0.14 |

TABLE 6
List of antibodies for multi-plex IP, single-plex IP (+/−MS).

| Target | Vendor | IP Antibody # |
|---|---|---|
| ALK | Thermo Fisher Scientific | 35-4300 |
| ALK | Thermo Fisher Scientific | 51-3900 |
| AKT1/AKT2/AKT3 | Thermo Fisher Scientific | 44-609G |
| AKT1/AKT2/AKT3 | Millipore | 07-416 |
| ARAF | Thermo Fisher Scientific | PA1-21440 |
| ARAF | Thermo Fisher Scientific | PA5-17657 |
| BRAF | Cell Signaling Technology | 14814 |
| BRAF | Thermo Fisher Scientific | MA5-15000 |
| CALM1 | Millipore | 05-173 |
| CCND1 | Cell Signaling Technology | 2922 |
| CCND1 | Millipore | ABE52 |
| CDKN1A/p21 | Thermo Fisher Scientific | PA1-30399 |
| CDKN1B | Cell Signaling Technology | 3688 |
| CDKN1B | Thermo Fisher Scientific | PA5-27188 |
| CDKN2A/p16INK4a | Abcam | ab16123 |
| CTNNB1 | Thermo Fisher Scientific | 71-2700 |
| DUSP22 | Thermo Fisher Scientific | PA5-33488 |
| DUSP4 | Cell Signaling Technology | 5149 |
| DUSP6 | Thermo Fisher Scientific | PA5-21570 |
| EGFR | Thermo Fisher Scientific | PA1-1110 |
| EIF4E | Cell Signaling Technology | 9742 |
| EIF4EBP1 | Cell Signaling Technology | 9644 |
| ERBB2 | Thermo Fisher Scientific | MA5-12759 |
| ERBB2 | Thermo Fisher Scientific | MA5-14057 |
| ERBB3 | Thermo Fisher Scientific | MA5-13053 |
| ERBB4 | Thermo Fisher Scientific | PA5-16642 |
| ESR1 | Thermo Fisher Scientific | MA1-25623 |
| ESR1 | Thermo Fisher Scientific | PA5-16440 |
| FBXO7 | Millipore | ABN1038 |
| FBXO7 | Novus Bio | NBP1-55051 |
| FOS | Thermo Fisher Scientific | MA5-15055 |
| FOS | Thermo Fisher Scientific | PA5-17489 |
| FOXO1 | Thermo Fisher Scientific | MA5-14846 |
| FOXO3 | Cell Signaling Technology | 12829 |
| HRAS | Thermo Fisher Scientific | 33-7200 |
| KRAS/HRAS/NRAS | Thermo Fisher Scientific | MA1-012 |
| IQGAP1 | Thermo Fisher Scientific | 33-8900 |
| JAK1 | Cell Signaling Technology | 3332 |
| JAK1 | Cell Signaling Technology | 3344 |
| JAK2 | Cell Signaling Technology | 3230 |
| MAP2K1 | Thermo Fisher Scientific | MA5-15093 |
| MAP2K2 | Abcam | ab28834 |
| MAPK1 | Thermo Fisher Scientific | PA1-21016 |
| MAPK1/MAPK3 | Proteintech | 66192-1-Ig |
| MAPK11 | Abcam | ab80116 |
| pan MAPK8/9 | Proteintech | 51153-1-AP |
| MAPK9 | Thermo Fisher Scientific | PA5-28664 |
| MET | Thermo Fisher Scientific | PA1-28026 |
| MET | Thermo Fisher Scientific | PA5-17148 |
| MTOR | Thermo Fisher Scientific | PA1-188 |
| MTOR | Thermo Fisher Scientific | PA1-518 |
| NF1 | Millipore | 07-730 |
| NF1 | Abcam | ab17963 |
| NOTCH 1 | Cell Signaling Technology | 3608 |
| PAK1 | Thermo Fisher Scientific | 71-9300 |
| Pan-Cadherin | Thermo Fisher Scientific | 71-7100 |
| Pan-Cadherin | Thermo Fisher Scientific | PA1-37199 |
| PARP1 | Thermo Fisher Scientific | MA5-15031 |
| PIK3CA | Millipore | 09-481 |
| PIK3R2 | Thermo Fisher Scientific | MA1-90048 |
| PSMG1 | Cell Signaling Technology | 13378 |
| RAF1 | Millipore | 07-396 |
| RAF1 | Thermo Fisher Scientific | PA5-28652 |
| RASA1 | Abcam | ab2922 |
| RASA1 | Thermo Fisher Scientific | MA4-001 |
| RICTOR | Cell Signaling Technology | 9476 |
| RICTOR | Abcam | ab70373 |
| RSK1 (RPS6KA1) | Cell Signaling Technology | 8408 |
| RSK1 (RPS6KA1) | Thermo Fisher Scientific | PA5-21699 |
| RSK2 (RPS6KA3) | Thermo Fisher Scientific | 38-6800 |
| RPS6KB1 | Abgent | AP3289g |
| RPS6KB2 | Abcam | ab131526 |
| RPS6KB2 | Abcam | ab37699 |
| RPTOR | Millipore | 09-217 |
| RPTOR | Thermo Fisher Scientific | 42-4000 |
| SMAD4 | R&D Systems | AF2097-P |
| SMAD4 | Thermo Fisher Scientific | PA5-14845 |
| SOS1 | Cell Signaling Technology | 5890 |
| SOS1 | Cell Signaling Technology | 12409 |
| SPRED1 | Millipore | ABS186 |
| SPRED1 | Thermo Fisher Scientific | PA5-20617 |
| SPRED2 | Millipore | ABS198 |
| SPRY1 | Cell Signaling Technology | 13013 |
| SPRY1 | Thermo Fisher Scientific | 40-1800 |
| SPRY2 | Cell Signaling Technology | 14954 |
| SPRY2 | Millipore | 07-524 |
| SPRY4 | Thermo Fisher Scientific | PA5-14349 |
| SPRY4 | Thermo Fisher Scientific | PA5-26959 |
| STAT3 | Cell Signaling Technology | 9139 |
| STAT3 | Thermo Fisher Scientific | 13-7000 |
| STK11 | Thermo Fisher Scientific | MA5-14942 |
| TP53 | Thermo Fisher Scientific | MA1-12648 |
| Phospho-TP53 pSer15 | Thermo Fisher Scientific | 700439 |
| Phospho-TP53 pSer15 | Thermo Fisher Scientific | MA5-15229 |
| Phospho-MAPK1/3 | Thermo Fisher Scientific | MA5-15173 |
| pThr202/pTyr204 | | |
| Phospho-MAPK1/3 | Thermo Fisher Scientific | PA1-26678 |
| pThr202/pTyr204 | | |
| Phospho-MAPK1/3 | Cell Signaling Technology | 9101 |
| pThr202/pTyr204 | | |
| Phospho-RAF1 pSer259 | Thermo Fisher Scientific | 44502 |
| Phospho-RAF1 pSer259 | Cell Signaling Technology | 9421 |
| Phospho-RAF1 pSer259 | Thermo Fisher Scientific | MA5-15176 |
| Phospho-RPS6KA3 | Thermo Fisher Scientific | 44928G |
| pSer380 | | |
| Phospho-RPS6KA3 | Cell Signaling Technology | 11989 |
| pSer380 | | |
| Phospho-MAPK8/9 | Cell Signaling Technology | 9251 |
| pThr183/pTyr185 | | |
| Phospho-MAPK8/9 | Thermo Fisher Scientific | MA5-14943 |
| pThr183/pTyr185 | | |
| Phospho-MEK1/2 | Thermo Fisher Scientific | MA5-15016 |
| Ser217/221 | | |
| Phospho-MEK1 Ser298 | Cell Signaling Technology | 98195 |
| Phospho-ATF-2 Thr71 | Cell Signaling Technology | 5112 |

In one instance, total TP53 and phosphorylated TP53 are evaluated together. In some embodiments, KRAS, HRAS, and NRAS are evaluated together. In other embodiments, EGFR, SOS1, ARAF, BRAF, RAF1, KRAS, HRAS, and NRAS, RPS6KA1, PIK3R1, PIK3R2 and PIK3CA are evaluated together. In some embodiments, ERBB2, SOS1, ARAF, BRAF, RAF1, KRAS, HRAS, and NRAS, RPS6KA1, PIK3R1, PIK3R2 and PIK3CA are evaluated together. In some embodiments, SPRY1, SPRY2, DUSP4, RASA1, NF1, SPRED1, SPRED2, KRAS, HRAS, and NRAS are evaluated together. In some embodiments, MAPK1/3 and TP53 are evaluated together (including either total protein or phosphorylated forms, such as, for example MAPK1/3 (including phosphorylated form pThr202/pTyr204) and TP53 (including phosphorylated form pSer15)). In some embodiments, PIK3R1, PIK3R2, PIK3CA, ARAF, BRAF, RAF1, KRAS, HRAS, NRAS, RPS6KA1, RPS6KB1, AKT1, AKT2, AKT3, MTOR, phosphor-TP53 (pSer15), phosphor-MAPK1/3 (pThr202/pTyr204) and phospho-MAPK8/9 (pThr183/pTyr185) are evaluated together. Any of the antibodies referenced in Table 6 corresponding to these proteins may be employed in a multi-plex IP format, for example, as in the groups set forth in Table 7.

TABLE 7
Selected combinations of antibodies for multi-plex
IP (+/−MS) (when more than one antibody is
listed for a given protein, any one may be chosen).

| Combi- | | | IP |
|---|---|---|---|
| nation | Target | Vendor | Antibody # |
| A | TP53 | Thermo Fisher Scientific | MA1-12648 |
| | Phospho-TP53 pSer15 | Thermo Fisher Scientific | 700439 |
| | Phospho-TP53 pSer15 | Thermo Fisher Scientific | MA5-15229 |
| B | HRAS | Thermo Fisher Scientific | 33-7200 |
| | KRAS/HRAS/NRAS | Thermo Fisher Scientific | MA1-012 |
| C | EGFR | Thermo Fisher Scientific | PA1-1110 |
| | SOS1 | Cell Signaling | 5890 |
| | | Technology | |
| | SOS1 | Cell Signaling | 12409 |
| | | Technology | |
| | ARAF | Thermo Fisher Scientific | PA1-21440 |
| | ARAF | Thermo Fisher Scientific | PA5-17657 |
| | BRAF | Cell Signaling | 14814 |
| | | Technology | |
| | BRAF | Thermo Fisher Scientific | MA5-15000 |
| | RAF1 | Millipore | 07-396 |
| | RAF1 | Thermo Fisher Scientific | PA5-28652 |
| | HRAS | Thermo Fisher Scientific | 33-7200 |
| | KRAS/HRAS/NRAS | Thermo Fisher Scientific | MA1-012 |
| | RSK1 (RPS6KA1) | Cell Signaling | 8408 |
| | | Technology | |
| | RSK1 (RPS6KA1) | Thermo Fisher Scientific | PA5-21699 |
| | PIK3R1 | Thermo Fisher Scientific | MA1-90048 |
| | PIK3R2 | Thermo Fisher Scientific | MA1-90048 |
| | PIK3CA | Millipore | 09-481 |
| D | ERBB2 | Thermo Fisher Scientific | MA5-12759 |
| | ERBB2 | Thermo Fisher Scientific | MA5-14057 |
| | SOS1 | Cell Signaling | 5890 |
| | | Technology | |
| | SOS1 | Cell Signaling | 12409 |
| | | Technology | |
| | ARAF | Thermo Fisher Scientific | PA1-21440 |
| | ARAF | Thermo Fisher Scientific | PA5-17657 |
| | BRAF | Cell Signaling | 14814 |
| | | Technology | |
| | BRAF | Thermo Fisher Scientific | MA5-15000 |
| | RAF1 | Millipore | 07-396 |
| | RAF1 | Thermo Fisher Scientific | PA5-28652 |
| | HRAS | Thermo Fisher Scientific | 33-7200 |
| | KRAS/HRAS/NRAS | Thermo Fisher Scientific | MA1-012 |
| | RSK1 (RPS6KA1) | Cell Signaling | 8408 |
| | | Technology | |
| | RSK1 (RPS6KA1) | Thermo Fisher Scientific | PA5-21699 |
| | PIK3R1 | Thermo Fisher Scientific | MA1-90048 |
| | PIK3R2 | Thermo Fisher Scientific | MA1-90048 |
| | PIK3CA | Millipore | 09-481 |
| E | SPRY1 | Cell Signaling | 13013 |
| | | Technology | |
| | SPRY1 | Thermo Fisher Scientific | 40-1800 |
| | SPRY2 | Cell Signaling | 14954 |
| | | Technology | |
| | SPRY2 | Millipore | 07-524 |
| | DUSP4 | Cell Signaling | 5149 |
| | | Technology | |
| | RASA1 | Abcam | ab2922 |
| | RASA1 | Thermo Fisher Scientific | MA4-001 |
| | NF1 | Millipore | 07-730 |
| | NF1 | Abcam | ab17963 |
| | SPRED1 | Millipore | ABS186 |
| | SPRED1 | Thermo Fisher Scientific | PA5-20617 |
| | SPRED2 | Millipore | ABS198 |
| | HRAS | Thermo Fisher Scientific | 33-7200 |
| | KRAS/HRAS/NRAS | Thermo Fisher Scientific | MA1-012 |
| F | Phospho-MAPK1/3 | Thermo Fisher Scientific | MA5-15173 |
| | pThr202/pTyr204 | | |
| | Phospho-MAPK1/3 | Thermo Fisher Scientific | PA1-26678 |
| | pThr202/pTyr204 | | |
| | Phospho-MAPK1/3 | Cell Signaling | 9101 |
| | pThr202/pTyr204 | Technology | |
| | TP53 | Thermo Fisher Scientific | MA1-12648 |
| | Phospho-TP53 pSer15 | Thermo Fisher Scientific | 700439 |
| | Phospho-TP53 pSer15 | Thermo Fisher Scientific | MA5-15229 |
| G | PIK3R1 | Thermo Fisher Scientific | MA1-90048 |
| | PIK3R2 | Thermo Fisher Scientific | MA1-90048 |
| | PIK3CA | Millipore | 09-481 |
| | ARAF | Thermo Fisher Scientific | PA1-21440 |
| | ARAF | Thermo Fisher Scientific | PA5-17657 |
| | BRAF | Cell Signaling | 14814 |
| | | Technology | |
| | BRAF | Thermo Fisher Scientific | MA5-15000 |
| | RAF1 | Millipore | 07-396 |
| | RAF1 | Thermo Fisher Scientific | PA5-28652 |
| | HRAS | Thermo Fisher Scientific | 33-7200 |
| | KRAS/HRAS/NRAS | Thermo Fisher Scientific | MA1-012 |
| | RSK1 (RPS6KA1) | Cell Signaling | 8408 |
| | | Technology | |
| | RSK1 (RPS6KA1) | Thermo Fisher Scientific | PA5-21699 |
| | RPS6KB2 | Abcam | ab131526 |
| | RPS6KB2 | Abcam | ab37699 |
| | AKT1 | Millipore | 07-416 |
| | AKT2 | Millipore | 07-416 |
| | AKT3 | Millipore | 07-416 |
| | MTOR | Thermo Fisher Scientific | PA1-188 |
| | Phospho-TP53 pSer15 | Thermo Fisher Scientific | 700439 |
| | Phospho-TP53 pSer15 | Thermo Fisher Scientific | MA5-15229 |
| | Phospho-MAPK1/3 | Thermo Fisher Scientific | MA5-15173 |
| | pThr202/pTyr204 | | |
| | Phospho-MAPK1/3 | Thermo Fisher Scientific | PA1-26678 |
| | pThr202/pTyr204 | | |
| | Phospho-MAPK1/3 | Cell Signaling | 9101 |
| | pThr202/pTyr204 | Technology | |
| | Phospho-MAPK8/9 | Cell Signaling | 9251 |
| | pThr183/pTyr185 | Technology | |
| | Phospho-MAPK8/9 | Thermo Fisher Scientific | MA5-14943 |
| | pThr183/pTyr185 | | |
| H | Phospho-RAF1 | Thermo Fisher Scientific | MA5-15176 |
| | pSer259 | | |
| | Phospho-MAPK8/9 | Thermo Fisher Scientific | MA5-14943 |
| | pThr183/pTyr185 | | |
| | Phospho-RPS6KA3 | Cell Signaling | 11989 |
| | pSer380 | Technology | |
| | Phospho-MEK1/2 | Thermo Fisher Scientific | MA5-15016 |
| | Ser217/221 | | |
| | Phospho-ATF-2 | Cell Signaling | 5112 |
| | Thr71 | Technology | |
| | Phospho-MAPK8/9 | Thermo Fisher Scientific | MA5-14943 |
| | pThr183/pTyr185 | | |

### EXAMPLES

The following examples are provided to illustrate certain disclosed embodiments and are not to be construed as limiting the scope of this disclosure in any way.

### Example 1—Immunoprecipitation of RAS-RAF-MAPK Pathway Proteins and Discovery-MS

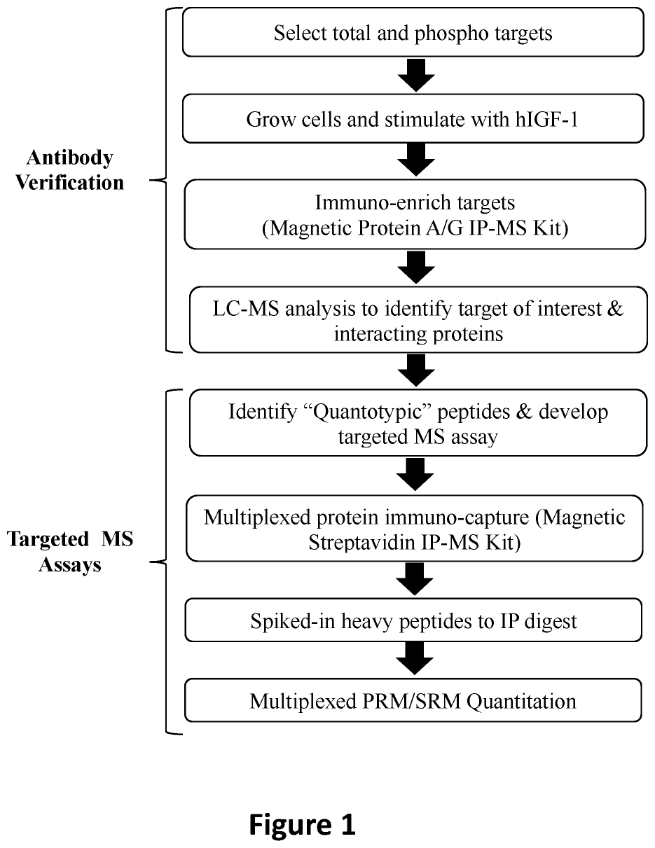

RAS-RAF-MAPK pathway proteins play central roles in diseases including cancer. The identification of RAS-RAF-MAPK pathway proteins, while desired as a means for monitoring disease progression, and as a tool for scientific research, has been limited in part because of the low abundance of RAS-RAF-MAPK pathway proteins, and in part due to a lack of validated methods and reagents. Phosphorylated RAS-RAF-MAPK pathway proteins are particularly important to identify and quantify as a measure of protein activation status, and also as markers for disease progression. As shown in FIG. 1, methods and reagents for detecting RAS-RAF-MAPK pathway proteins, including phosphorylated proteins, and their protein interactions, were designed and tested. Multiplex immunoprecipitation (IP) to MS (mIP-MS) was assessed for the ability to measure total and phosphorylated RAS-RAF-MAPK pathway targets. mIP-MS methods were also compared to existing singleplex immunoassay (Western Blot (WB) and ELISA) and multiplex Luminex assays.

Immunoprecipitation and MS Sample Preparation

The Thermo Scientific™ Pierce MS-Compatible Magnetic IP Kit (Protein A/G) was used to screen and validate antibodies for 78 RAS-RAF-MAPK pathway proteins from 500 μg of cell lysate. Validated antibodies were biotinylated with the Thermo Scientific™ Pierce Antibody Biotinylation Kit for IP. The Thermo Scientific™ Pierce MS-Compatible Magnetic IP Kit (Streptavidin) was used to perform the single or multiplex IPs for target enrichment. IP samples were processed by an in-solution digestion method where IP eluates were reconstituted in 6M Urea, 50 mM triethylammonium bicarbonate (TEAB), pH 8.5 followed by reduction, alkylation and trypsin digestion overnight at 37° C. The digested samples were acidified with trifluoroacetic acid (TFA) before MS analysis.

Immunoprecipitation with Enzyme Elution and MS Sample Preparation

An additional method employing a shorter trypsin digestion was developed (data not shown). Antibodies and appropriate lysate were incubated overnight at 4° C. with rotation. The next day, immune complexes were washed and allowed to incubate with 1.0 μg trypsin in 50 mM TEAB for 1 hour and 37° C. with 800 rpm shaking in a thermomixer. Enzyme derived IP elutions were separated from the beads and the beads were allowed to incubate with an MS compatible elution buffer (Thermo Fisher Scientific, PN: 90409) for 10 minutes at room temperature. This second elution was collected separately. The second elution was dried down in a speedvac and processed with a urea based in-solution digestion method in which IP eluates were reconstituted in 6M Urea, 50 mM TEAB, pH 8.5 followed by reduction, alkylation and trypsin digestion overnight at 37° C. The digested samples were acidified with TFA before MS analysis.

The enzyme-derived IP elution was combined with 25 ng Green Fluorescent Protein (GFP) and exposed to a solution of Tris[2-Carboxyethyl]phosphine (TCEP)/Chloroacetamide at a 1:4 ratio for 5-10 minutes at 95° C. and then allowed to cool briefly before addition of 600 ng trypsin and incubation for 2 hours at 37° C. with 500 rpm in a thermomixer. The trypsin-digested samples were neutralized with 10% TFA and dried down in a speedvac. Samples were reconstituted in 5% acetonitrile (ACN) and 0.1% TFA and taken to LC-MS analysis.

Liquid Chromatography and Mass Spectrometry

Prior to MS analysis, tryptic digest samples were desalted on-line using the Thermo Scientific™ Acclaim™ PepMap 100 C18 Trap Column. For discovery MS, the samples were analyzed by nanoLC-MS/MS using a Thermo Scientific™ Dionex™ UltiMate™ 3000 RSLCnano System and Thermo Scientific™ Q Exactive™ HF Hybrid Quadrupole-Orbitrap Mass Spectrometer. For targeted MS, the samples were analyzed using the UltiMate 3000 RSLCnano System and the Thermo Scientific™ TSQ™ Vantage™ Mass Spectrometer (SRM mode) or the Thermo Scientific™ Q Exactive™ HF Hybrid Quadrupole-Orbitrap Mass Spectrometer (PRM mode).

MS Data Analysis

Discovery MS data were analyzed with Thermo Scientific™ Proteome Discoverer™ 1.4 to assess percent sequence coverage, unique peptides, MS1 intensities, spectral counts and PTMs. The Proteome Discoverer software searches were executed using the Uniprot human protein database. Tryptic peptides with highest MS1 intensity and relevant phosphorylation sites were selected from the discovery data for targeted assay development. For targeted MS data analysis, Thermo Scientific™ Pinpoint software and Skyline software (University of Washington) were used to measure limit of quantitation (LOQ) from the calibration curve and target analyte concentration from unknown samples.

Results

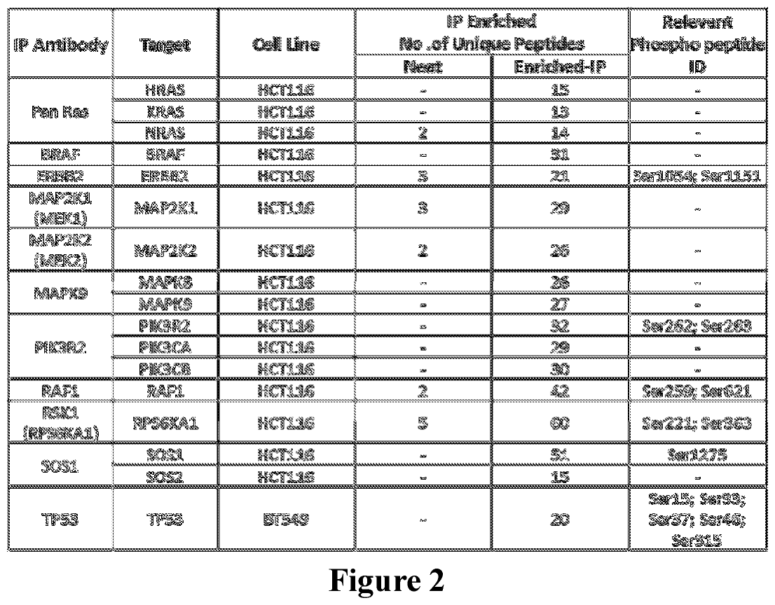

As shown in FIG. 2, RAS-RAF-MAPK pathway proteins were immunoprecipitated from HCT116 or BT549 cell lysates with Thermo Scientific™ Pierce MS-Compatible Magnetic IP Kits (Protein A/G or Streptavidin) for MS analysis. A variety of antibodies were screened to determine effectiveness in both ability to IP RAS-RAF-MAPK pathway proteins, and also for their usefulness when combined with MS. Table 1 (above) provides a list of antibodies validated for use in the IP-MS methods. Table 2 (above) provides a list of antibodies tested, but found to be less successful.

Higher numbers of unique peptides were identified in IP enriched samples as compared to neat (non-IP-enriched) lysate. See FIG. 2. Protein isoforms and interacting protein partners were identified for RAS (K, H, N), JNKs (MAPK8, MAPK9), PI3K (PIK3CA, PIK3CB, PIK3R1, PIK3R2), and SOS1 targets. Relevant phosphor peptides were identified for ERbb2, PIK3R2, RAF1, RPS6KA1, SOS1 and TP53 targets. Candidate quantitative peptides were selected for targeted MS assay development.

Limits of detection (LOD) and lower limits of quantification (LLOQ) were analyzed for RAS-RAF-MAPK pathway proteins, including BRAF, ERBB2, FOS, FOXO1, FOXO3, HRAS, KRAS, MAP2K1, MAP2K2, MAPK1, MAPK3, MAPK8, MAPK9, NRAS, RPS6KA1, SOS1, and TP53. Results are presented in FIG. 3. The assay dynamic range, representing the concentration range between the lower to upper limits of quantification (LLOQ to ULOQ), is the range where protein concentration is measurable with acceptable levels of accuracy and precision. To ensure linearity of the measurement, for each internal standard peptide the linear signal-to-abundance range (LLOQ and ULOQ) was determined from dilution series experiments spanning concentrations of 300-0.05 fmol on column, spiked into a constant light peptide at 36 fmol and 200 ng of equimolar concentration of 6 proteins digest. In some instances, lower limits of detection and quantification may be established, even though these lower limits were not presently tested. Thus, the lower limits are no more than the numbers determined experimentally.

### Example 2—Multiplex IP of RAS-RAF-MAPK Pathway Proteins and Multiplex MS

Multiple RAS-RAF-MAPK pathway protein targets were enriched simultaneously from unstimulated and human insulin-like growth factor (hIGF-1)-stimulated HCT116 lysate with biotinylated antibodies and Thermo Scientific™ Pierce MS-Compatible Magnetic IP Kit (Streptavidin). HCT116 cells were starved in 0.1% charcoal stripped fetal bovine serum (FBS) for 24 hours before stimulation with 100 ng/ml of IGF for 15 minutes. Validated IP-MS antibodies are biotinylated for 29 RAS-RAF-MAPK pathway targets using the Thermo Scientific™ Pierce Antibody Biotinylation Kit for IP (PN: 90407) as recommended in instruction manual. 29 total targets were grouped into three different panels for multi-plex IP. 1 μg of each biotinylated antibody for each panel comprising 10-15 total targets were added simultaneously to 1000 μg of control and IGF stimulated HCT116 cell lysate in duplicate. IP was performed as recommended in the Thermo Scientific™ Pierce MS-Compatible Magnetic IP Kit (Streptavidin) (PN: 90408) with the following modification. 5 micrograms of streptavidin magnetic beads per microgram of biotinylated antibody concentration was used for multi-plex IP.

IP samples were processed by an in-solution digestion method where IP eluates were reconstituted in 6M Urea, 50 mM TEAB, pH 8.5 followed by reduction (5 mM TCEP for 30 minutes at 35° C.), alkylation (20 mM Iodoacetamide in dark at room temperature for 30 minutes) and trypsin digestion overnight at 37° C. The digested samples were acidified with 3.54 of 10% TFA before discovery MS analysis. For discovery MS, the samples were analyzed by nanoLC-MS/MS using a Thermo Scientific™ Dionex™ UltiMate™ 3000 RSLCnano System and Thermo Scientific™ Q Exactive™ HF Hybrid Quadrupole-Orbitrap Mass Spectrometer. Briefly, the digested samples were cleaned on-line using the C18 trap column (Thermo Fisher Scientific, PN: 164564) followed by reversed-phase separation using the analytical C18 column (75 μm i.d.×15 cm, nanoViper, 3 μm particle size, Thermo Fisher Scientific, PN: ES800) with a 2-30% gradient of Buffer B using Buffer A (0.1% formic acid) and Buffer B (0.1% formic acid/99.9% acetonitrile) at 0.300 μL/min.

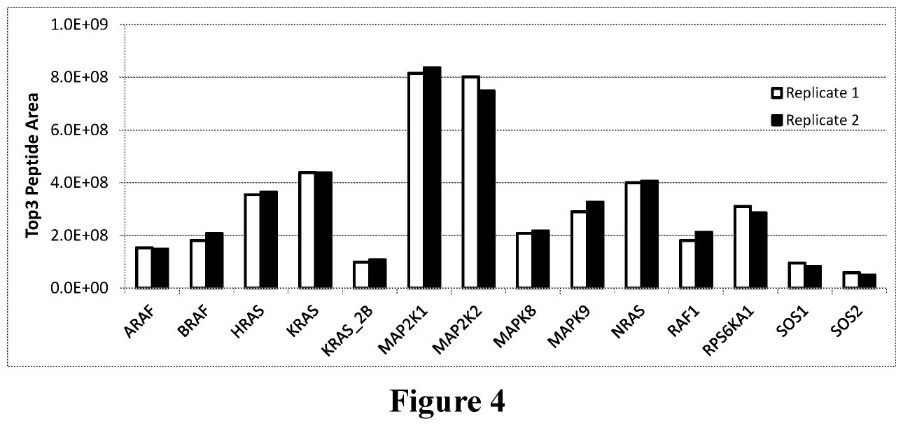

FIG. 4 shows that the IP-nanoLC-MS/MS analysis was able to identify 14 proteins in the multiplex assay panel 1. MS analysis of multiplex assay identified interacting proteins (KRAS-2b, MAPK8, and SOS1) of RAS-RAF-MAPK Pathway Targets. Table 6 provides listings of the antibodies used in this multiplex IP.

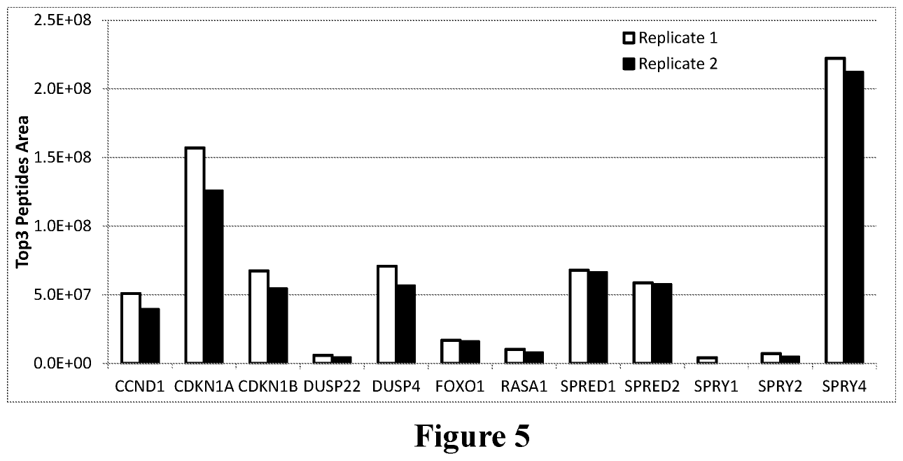

FIG. 5 shows that the IP-nanoLC-MS/MS analysis was able to identify 12 proteins in the multiplex assay panel 2. Table 6 provides listings of the antibodies used in this multiplex IP.

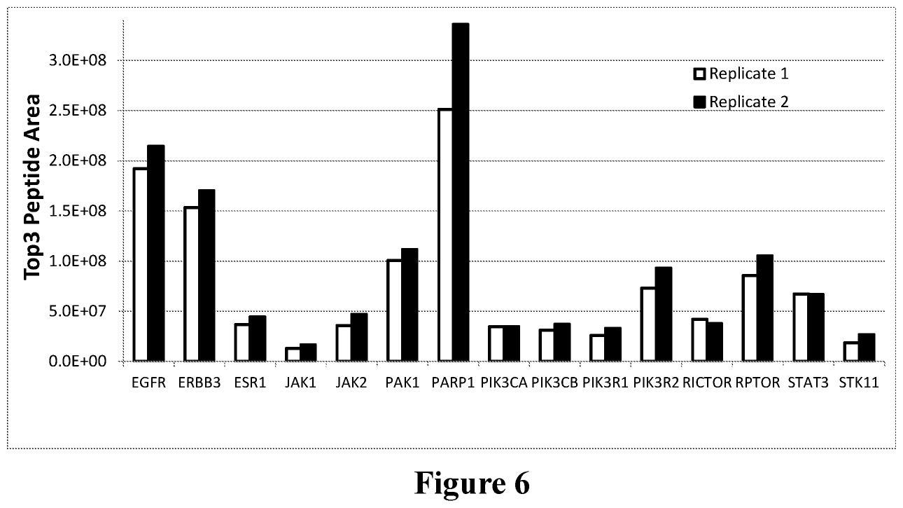

FIG. 6 shows that the IP-nanoLC-MS/MS analysis was able to identify 15 proteins in the multiplex assay panel 3. MS analysis of multiplex assay identified interacting proteins (PIK3CA, PIK3CB, and PIK3R1) of RAS-RAF-MAPK Pathway Targets. Table 6 provides listings of the antibodies used in this multiplex IP.

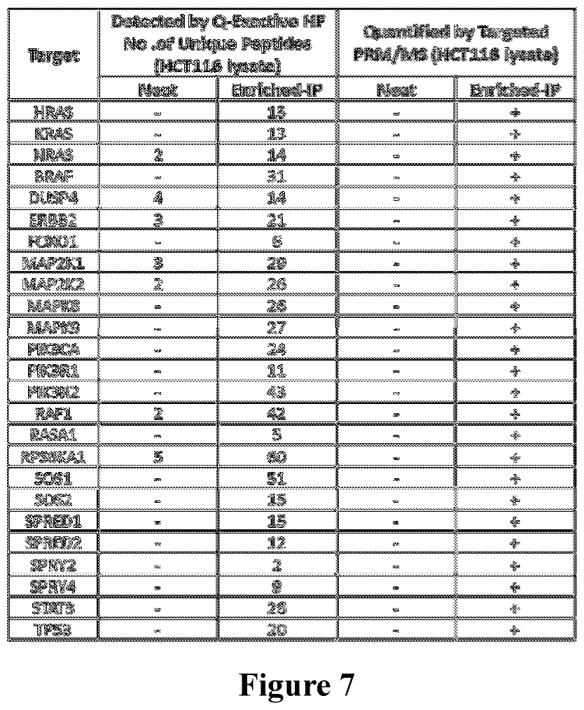

A summary of RAS-RAF-MAPK pathway proteins that were identified and quantified using the IP-MS methods described herein is provided in FIG. 7. Most of the RAS-RAF-MAPK pathway targets were not identified in discovery MS and quantitated by targeted MS (PRM or SRM) without enrichment by immunoprecipitation.

Immunoprecipitation using particular selected antibodies resulted in a higher yield of RAS-RAF-MAPK pathway target proteins and less non-specific binding proteins than MS alone. IP-MS assay was also more successful than other commercially available non-MS assays. Furthermore, IP to MS analysis of the RAS-RAF-MAPK pathway proteins enabled identification of multiple isoforms, relevant protein interactions and phosphorylation sites. Three panels of mIP-tMS assays allowed simultaneous quantitation of 14, 12 and 15 RAS-RAF-MAPK pathway proteins in the low to sub-fmol range from unstimulated and IGF stimulated HCT116 cell lysates. Major advantages of the MS-based assay are high confidence in target identity coupled with simultaneous quantitation of multiple targets, interacting proteins and their phosphophorylated forms.

1. The method of claim 3, further comprising
a. treating a biological sample with at least one antibody capable of immunoprecipitating RAS-RAF-MAPK pathway protein(s) from a biological sample; and
b. digesting the immunoprecipitated target protein(s
c.
d.

2. (canceled)

3. A method for detecting RAS-RAF-MAPK pathway proteins, comprising
a. isolating proteins from a biological sample;
b. digesting the isolated proteins;
c. assaying the digested protein(s) via mass spectrometry to determine the presence or absence of a peptide for RAS-RAF-MAPK pathway protein(s), wherein the peptide for RAS-RAF-MAPK pathway protein(s) comprises a sequence of SEQ ID NO: 1-SEQ ID NO: 1400, wherein the peptide is less than or equal to 40 amino acids in length; and
d. detecting one or more RAS-RAF-MAPK pathway protein(s) in the sample.

4. The method of claim 3, further comprising determining the quantity of RAS-RAF-MAPK pathway protein.

5. The method of claim 3, wherein the RAS-RAF-MAPK pathway protein is phosphorylated or comprises at least one peptide of Table 5.

6. (canceled)

7. The method of claim 3, further comprising
c. adding a first and a second detectably labelled internal standard peptide of known amount to the digested proteins, wherein the first internal standard peptide has the same amino acid sequence as a phosphorylated RAS-RAF-MAPK pathway peptide used to identify the phosphorylated protein, and the second internal standard peptide has the same amino acid sequence as the non-phosphorylated RAS-RAF-MAPK pathway peptide used to identify the non-phosphorylated protein;
d. assaying the digested protein and internal standards via mass spectrometry to determine the presence and amount of phosphorylated and non-phosphorylated RAS-RAF-MAPK pathway peptides, wherein the RAS-RAF-MAPK pathway peptide is chosen from of SEQ ID NO: 1-SEQ ID NO: 1400; and
e. determining the quantity of RAS-RAF-MAPK phosphorylated and non-phosphorylated pathway proteins in the sample, and determining the ratio of phosphorylated to non-phosphorylated target proteins.

8. (canceled)

9. (canceled)

10. (canceled)

11. The method of claim 3, wherein the peptide is modified with a detectable label or an isotope selected from ¹³C, ¹⁵N, ²H and ¹⁸O.

12. (canceled)

13. The method of claim 11, wherein the modified peptide comprises a peptide of SEQ ID NO: 701-1400.

14. The method of claim 1, wherein the antibody is chosen from the antibodies recited in Table 1 or Table 6.

15. (canceled)

16. The method of claim 3, wherein the more than one RAS-RAF-MAPK pathway proteins are chosen from:
a. KRAS, HRAS, and NRAS;
b. EGFR, SOS1, ARAF, BRAF, RAF1, KRAS, HRAS, and NRAS, RPS6KA1, PIK3R1, PIK3R2 and PIK3CA;
c. ERBB2, SOS1, ARAF, BRAF, RAF1, KRAS, HRAS, and NRAS, RPS6KA1, PIK3R1, PIK3R2 and PIK3CA;
d. SPRY1, SPRY2, DUSP4, RASA1, NF1, SPRED1, SPRED2, KRAS, HRAS, and NRAS;
e. MAPK1/3 and TP53 are evaluated together (such as, for example MAPK1/3 phosphorylated form pThr202/pTyr204 and TP53 phosphorylated form pSer15); or
f. PIK3R1, PIK3R2, PIK3CA, ARAF, BRAF, RAF1, KRAS, HRAS, NRAS, RPS6KA1, RPS6KB1, AKT1, AKT2, AKT3, MTOR, phosphor-TP53 (pSer15) and phosphor-MAPK1/3 (pThr202/pTyr204).

17. The method of claim 1, wherein the antibodies used are from combinations B, C, D, E, F, or G in Table 7.

18. The method of claim 1, wherein the method evaluates both non-phosphorylated and phosphorylated versions of at least one RAS-RAF-MAPK pathway protein comprising:
g. employing a first antibody capable of immunoprecipitating phosphorylated version of the RAS-RAF-MAPK pathway protein, and a second antibody capable of immunoprecipitating a phosphorylated and non-phosphorylated RAS-RAF-MAPK pathway protein precipitated by the first antibody or
h. dividing the sample into two aliquots and immunoprecipitating either total or non-phosophorylated RAS-RAF-MAPK pathway protein from a first aliquot and immunoprecipitating a phosphorylated version of the RAS-RAF-MAPK pathway protein from second aliquot.

19. The method of claim 18, wherein total TP53 and phosphorylated TP53 are evaluated together.

20.-24.

25. The method of claim 3, wherein the quantity of a RAS-RAF-MAPK pathway protein is determined by a method comprising comparing an amount of the RAS-RAF-MAPK pathway peptide to an internal standard peptide of known amount, wherein both the peptide in the biological sample and the internal standard peptide are chosen from SEQ ID NO: 1-SEQ ID NO: 701, wherein the standard peptide is detectably labeled.

26. The method of claim 25, wherein the internal standard peptide is chosen from SEQ ID NO: 1-SEQ ID NO: 700 or SEQ ID NO: 701-SEQ ID NO: 1400.

27.-38. (canceled)

39. The method of claim 3, wherein the RAS-RAF-MAPK pathway protein is chosen from ALK (UniProtKB—Q9UM73), ARAF (UniProtKB—P10398), BRAF (UniProtKB—P15056), CALM1 (UniProtKB—P62158), CAMK2A (UniProtKB—Q9UQM7), CCND1 (UniProtKB—P24385), CDH1 (UniProtKB—P12830), CDH2 (UniProtKB—P19022), CDK2 (UniProtKB—P24941), CDKN1A (UniProtKB—P38936), CDKN1B (UniProtKB—P46527), CDKN2A (UniProtKB—Q8N726), CTNNA1 (UniProtKB—P35221), CTNNB1 (UniProtKB—P35222), DDR2 (UniProtKB—Q16832), DUSP22 (UniProtKB—Q9NRW4), DUSP4 (UniProtKB—Q13115), DUSP6 (UniProtKB—Q16828), EGFR (UniProtKB—P00533), EIF2A (UniProtKB—Q9BY44), EIF4E (UniProtKB—P06730), EIF4EBP1 (UniProtKB—Q13541), ERBB2 (UniProtKB—P04626), ERBB3 (UniProtKB—P21860), ERBB4 (UniProtKB—Q15303), ESR1 (UniProtKB—P03372), FBXO7 (UniProtKB—Q9Y3I1), FGFR1 (UniProtKB—P11362), FGFR3 (UniProtKB—P22607), FOS (UniProtKB—P01100), FOXO1 (UniProtKB—Q12778), FOXO3 (UniProtKB—O43524), HRAS (UniProtKB—P01112), IQGAP1 (UniProtKB—P46940), JAK1 (UniProtKB—P23458), JAK2 (UniProtKB—O60674), KRAS (UniProtKB—P01116), MAP2K1 (UniProtKB—Q02750), MAP2K2 (UniProtKB—P36507), MAPK1 (UniProtKB—P28482), MAPK11 (UniProtKB—Q15759), MAPK3 (UniProtKB—P27361), MAPK8 (UniProtKB—P45983), MAPK9 (UniProtKB—P45984), MET (UniProtKB—P08581), NF1 (UniProtKB—P21359), NOTCH1 (UniProtKB—P46531), NRAS (UniProtKB—P01111), PAK1 (UniProtKB—Q13153), PARP1 (UniProtKB—P09874), PGR (UniProtKB—P06401), PIK3CA (UniProtKB—P42336), PIK3R1 (UniProtKB—P27986), PIK3R2 (UniProtKB—O00459), PSMG1 (UniProtKB—O95456), RAF1 (UniProtKB—P04049), RASA1 (UniProtKB—P20936), RASGRF1 (UniProtKB—Q13972), RASGRF2 (UniProtKB—O14827), RICTOR (UniProtKB—Q6R327), RPS6KA1 (UniProtKB—Q15418), RPS6KA2 (UniProtKB—Q15349), RPS6KA3 (UniProtKB—P51812), RPS6KB2 (UniProtKB—Q9UBS0), RPTOR (UniProtKB—Q8N122), SMAD3 (UniProtKB—P84022), SMAD4 (UniProtKB—Q13485), SOS1 (UniProtKB—Q07889), SPRED1 (UniProtKB—Q7Z699), SPRED2 (UniProtKB—Q7Z698), SPRY1 (UniProtKB—O43609), SPRY2 (UniProtKB—O43597), SPRY3 (UniProtKB—O43610), SPRY4 (UniProtKB—Q9C004), STAT1 (UniProtKB—P42224), STAT3 (UniProtKB—P40763), STK11 (UniProtKB—Q15831), TP53 (UniProtKB—P04637), ATF2 (UniProtKB—P15336), MAPK12 (UniProtKB—P53778), MAPK13 (UniProtKB—O15264), MAPK14 (UniProtKB—Q16539), MAP2K3 (UniProtKB—P46734), MAP2K6 (UniProtKB—P52564), RPS6KA5 (UniProtKB—O75582), and MAPKAPK2 (UniProtKB—P49137).

40. (canceled)

41. (canceled)

42. The method of claim 3, wherein the concentration of RAS-RAF-MAPK peptide detected ranges from 0.04 fmol to 2000 fmol.

43. (canceled)

44. The method of claim 3, wherein the lower limit of detection is from 0.04-1.23 fmol.

45. The method of claim 3, wherein the lower limit of quantification is no more than 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, 0.25, 0.30, 0.35, 0.40, 0.45, 0.50, 0.55, 0.60, 0.65, 0.70, or 11.11 fmol.

46. (canceled)

47. A kit comprising one or more antibodies capable of immunoprecipitating a RAS-RAF-MAPK pathway protein.

48.-62. (canceled)

63. A method for immunoprecipitating a RAS-RAF-MAPK pathway protein comprising contacting a biological sample with any one of the antibodies recited in Table 1.
